SEQUENCE LISTING (389 sequences)

SEQ ID NO: 1 - protein, 5 aa, synthetic construct
  REGION 1..5: note = TLA0015-HCDR1
  VARIANT 1: note = Xaa = Ser, Thr or Tyr
XYGVD

SEQ ID NO: 2 - protein, 5 aa, synthetic construct
  REGION 1..5: note = TLA0015-HCDR1-1
SYGVD

SEQ ID NO: 3 - protein, 5 aa, synthetic construct
  REGION 1..5: note = TLA0015-HCDR1-2
YYGVD

SEQ ID NO: 4 - protein, 5 aa, synthetic construct
  REGION 1..5: note = TLA0015-HCDR1-3
TYGVD

SEQ ID NO: 5 - protein, 16 aa, synthetic construct
  REGION 1..16: note = TLA0015-HCDR2
  VARIANT 1: note = Xaa = Arg or Val
  VARIANT 7: note = Xaa = Gly or Lys
  VARIANT 9: note = Xaa = Asp, His or Asn
  VARIANT 11: note = Xaa = Asn or Gln
  VARIANT 12: note = Xaa = Ala, Pro or Ser
XIWGFGXTXYXXALKS

SEQ ID NO: 6 - protein, 16 aa, synthetic construct
  REGION 1..16: note = TLA0015-HCDR2-1
VIWGFGGTNYNSALKS

SEQ ID NO: 7 - protein, 16 aa, synthetic construct
  REGION 1..16: note = TLA0015-HCDR2-2
VIWGFGGTNYQSALKS

SEQ ID NO: 8 - protein, 16 aa, synthetic construct
  REGION 1..16: note = TLA0015-HCDR2-3
VIWGFGGTNYNAALKS

SEQ ID NO: 9 - protein, 16 aa, synthetic construct
  REGION 1..16: note = TLA0015-HCDR2-4
VIWGFGGTNYNPALKS

SEQ ID NO: 10 - protein, 16 aa, synthetic construct
  REGION 1..16: note = TLA0015-HCDR2-5
VIWGFGGTHYNSALKS

SEQ ID NO: 11 - protein, 16 aa, synthetic construct
  REGION 1..16: note = TLA0015-HCDR2-6
RIWGFGGTNYNSALKS

SEQ ID NO: 12 - protein, 16 aa, synthetic construct
  REGION 1..16: note = TLA0015-HCDR2-7
VIWGFGKTNYNSALKS

SEQ ID NO: 13 - protein, 16 aa, synthetic construct
  REGION 1..16: note = TLA0015-HCDR2-8
VIWGFGGTDYNSALKS

SEQ ID NO: 14 - protein, 8 aa, synthetic construct
  REGION 1..8: note = TLA0015-HCDR3
  VARIANT 2: note = Xaa = Asp, Leu or Asn
  VARIANT 3: note = Xaa = Phe or Tyr
GXXDAMDY

SEQ ID NO: 15 - protein, 8 aa, synthetic construct
  REGION 1..8: note = TLA0015-HCDR3-1
GNFDAMDY

SEQ ID NO: 16 - protein, 8 aa, synthetic construct
  REGION 1..8: note = TLA0015-HCDR3-2
GNYDAMDY

SEQ ID NO: 17 - protein, 8 aa, synthetic construct
  REGION 1..8: note = TLA0015-HCDR3-3
GDFDAMDY

SEQ ID NO: 18 - protein, 8 aa, synthetic construct
  REGION 1..8: note = TLA0015-HCDR3-4
GLFDAMDY

SEQ ID NO: 19 - protein, 30 aa, synthetic construct
  REGION 1..30: note = TLA0015-HFR1
  VARIANT 2: note = Xaa = Ile or Val
  VARIANT 5: note = Xaa = Leu or Val
  VARIANT 12: note = Xaa = Ile or Val
  VARIANT 20: note = Xaa = Ile or Leu
  VARIANT 28: note = Xaa = Asp or Ser
  VARIANT 30: note = Xaa = Ile, Lys or Ser
EXQLXESGGGLXQPGGSLRXSCAVSGFXLX

SEQ ID NO: 20 - protein, 30 aa, synthetic construct
  REGION 1..30: note = TLA0015-HFR1-1
EIQLLESGGGLVQPGGSLRLSCAVSGFSLS

SEQ ID NO: 21 - protein, 30 aa, synthetic construct
  REGION 1..30: note = TLA0015-HFR1-2
EIQLLESGGGLVQPGGSLRISCAVSGFSLS

SEQ ID NO: 22 - protein, 30 aa, synthetic construct
  REGION 1..30: note = TLA0015-HFR1-3
EIQLVESGGGLVQPGGSLRISCAVSGFSLS

SEQ ID NO: 23 - protein, 30 aa, synthetic construct
  REGION 1..30: note = TLA0015-HFR1-4
EVQLLESGGGLVQPGGSLRLSCAVSGFSLS

SEQ ID NO: 24 - protein, 30 aa, synthetic construct
  REGION 1..30: note = TLA0015-HFR1-5
EIQLVESGGGLIQPGGSLRLSCAVSGFSLS

SEQ ID NO: 25 - protein, 30 aa, synthetic construct
  REGION 1..30: note = TLA0015-HFR1-6
EIQLVESGGGLIQPGGSLRISCAVSGFSLS

SEQ ID NO: 26 - protein, 30 aa, synthetic construct
  REGION 1..30: note = TLA0015-HFR1-7
EIQLVESGGGLIQPGGSLRISCAVSGFSLI

SEQ ID NO: 27 - protein, 30 aa, synthetic construct
  REGION 1..30: note = TLA0015-HFR1-8
EIQLVESGGGLIQPGGSLRISCAVSGFSLK

SEQ ID NO: 28 - protein, 30 aa, synthetic construct
  REGION 1..30: note = TLA0015-HFR1-9
EIQLVESGGGLIQPGGSLRISCAVSGFDLS

SEQ ID NO: 29 - protein, 14 aa, synthetic construct
  REGION 1..14: note = TLA0015-HFR2
  VARIANT 5: note = Xaa = Ala or Ser
  VARIANT 13: note = Xaa = Leu or Val
WVRQXPGKGLEWXG

SEQ ID NO: 30 - protein, 14 aa, synthetic construct
  REGION 1..14: note = TLA0015-HFR2-1/TLA-0238-HFR2-3
WVRQAPGKGLEWVG

SEQ ID NO: 31 - protein, 14 aa, synthetic construct
  REGION 1..14: note = TLA0015-HFR2-2
WVRQAPGKGLEWLG

SEQ ID NO: 32 - protein, 14 aa, synthetic construct
  REGION 1..14: note = TLA0015-HFR2-3
WVRQSPGKGLEWLG

SEQ ID NO: 33 - protein, 32 aa, synthetic construct
  REGION 1..32: note = TLA0015-HFR3
  VARIANT 2: note = Xaa = Phe or Leu
  VARIANT 6: note = Xaa = Ala, Lys, Arg or Val
  VARIANT 13: note = Xaa = Leu or Val
  VARIANT 32: note = Xaa = Arg or Ser
RXTISXDNSKNTXYLQMNSLRAEDTAVYYCAX

SEQ ID NO: 34 - protein, 32 aa, synthetic construct
  REGION 1..32: note = TLA0015-HFR3-1
RLTISRDNSKNTLYLQMNSLRAEDTAVYYCAS

SEQ ID NO: 35 - protein, 32 aa, synthetic construct
  REGION 1..32: note = TLA0015-HFR3-2
RLTISKDNSKNTVYLQMNSLRAEDTAVYYCAS

SEQ ID NO: 36 - protein, 32 aa, synthetic construct
  REGION 1..32: note = TLA0015-HFR3-3
RFTISRDNSKNTVYLQMNSLRAEDTAVYYCAS

SEQ ID NO: 37 - protein, 32 aa, synthetic construct
  REGION 1..32: note = TLA0015-HFR3-4
RLTISRDNSKNTVYLQMNSLRAEDTAVYYCAS

SEQ ID NO: 38 - protein, 32 aa, synthetic construct
  REGION 1..32: note = TLA0015-HFR3-5
RLTISRDNSKNTVYLQMNSLRAEDTAVYYCAR

SEQ ID NO: 39 - protein, 32 aa, synthetic construct
  REGION 1..32: note = TLA0015-HFR3-6
RLTISVDNSKNTVYLQMNSLRAEDTAVYYCAS

SEQ ID NO: 40 - protein, 32 aa, synthetic construct
  REGION 1..32: note = TLA0015-HFR3-7
RLTISADNSKNTVYLQMNSLRAEDTAVYYCAS

SEQ ID NO: 41 - protein, 11 aa, synthetic construct
  REGION 1..11: note = TLA0015-HFR4-1/TLA-0238-HFR4-2
WGQGTLVTVSS

SEQ ID NO: 42 - protein, 11 aa, synthetic construct
  REGION 1..11: note = TLA0015-LCDR1-1
RASQDISNYLN

SEQ ID NO: 43 - protein, 7 aa, synthetic construct
  REGION 1..7: note = TL0015-LCDR2-1
YTSRLHS

SEQ ID NO: 44 - protein, 9 aa, synthetic construct
  REGION 1..9: note = TL0015-LCDR3-1
QQGNTLPFT

SEQ ID NO: 45 - protein, 23 aa, synthetic construct
  REGION 1..23: note = TL0015-LFR1
  VARIANT 7: note = Xaa = Ser or Thr
  VARIANT 8: note = Xaa = Pro or Thr
  VARIANT 22: note = Xaa = Ser or Thr
DIQMTQXXSSLSASVGDRVTIXC

SEQ ID NO: 46 - protein, 23 aa, synthetic construct
  REGION 1..23: note = TL0015-LFR1-1
DIQMTQTTSSLSASLGDRVTISC

SEQ ID NO: 47 - protein, 23 aa, synthetic construct
  REGION 1..23: note = TL0015-LFR1-2/TLA0241-LFR1-1
DIQMTQSPSSLSASVGDRVTITC

SEQ ID NO: 48 - protein, 15 aa, synthetic construct
  REGION 1..15: note = TL0015-LFR2
  VARIANT 9: note = Xaa = Ala or Val
  VARIANT 10: note = Xaa = Pro or Val
WYQQKPGKXXKLLIY

SEQ ID NO: 49 - protein, 15 aa, synthetic construct
  REGION 1..15: note = TL0015-LFR2-1
WYQQKPDGTVKLLIY

SEQ ID NO: 50 - protein, 15 aa, synthetic construct
  REGION 1..15: note = TL0015-LFR2-2
WYQQKPGKVVKLLIY

SEQ ID NO: 51 - protein, 15 aa, synthetic construct
  REGION 1..15: note = TL0015-LFR2-3
WYQQKPGKAVKLLIY

SEQ ID NO: 52 - protein, 15 aa, synthetic construct
  REGION 1..15: note = TL0015-LFR2-4/TLA0241-LFR2-1
WYQQKPGKAPKLLIY

SEQ ID NO: 53 - protein, 32 aa, synthetic construct
  REGION 1..32: note = TL0015-LFR3
  VARIANT 15: note = Xaa = Phe or Tyr
  VARIANT 27: note = Xaa = Phe or Val
  VARIANT 31: note = Xaa = Phe or Tyr
GVPSRFSGSGSGTDXTLTISSLQPEDXATYXC

SEQ ID NO: 54 - protein, 32 aa, synthetic construct
  REGION 1..32: note = TL0015-LFR3-1
GVPSRFSGSGSGTDYSLTISNLEQEDIATYFC

SEQ ID NO: 55 - protein, 32 aa, synthetic construct
  REGION 1..32: note = TL0015-LFR3-2
GVPSRFSGSGSGTDYTLTISSLQPEDVATYFC

SEQ ID NO: 56 - protein, 32 aa, synthetic construct
  REGION 1..32: note = TL0015-LFR3-3
GVPSRFSGSGSGTDYTLTISSLQPEDFATYFC

SEQ ID NO: 57 - protein, 32 aa, synthetic construct
  REGION 1..32: note = TL0015-LFR3-4/TLA0238-LFR3-4/TLA0241-LFR3-2
GVPSRFSGSGSGTDFTLTISSLQPEDFATYYC

SEQ ID NO: 58 - protein, 10 aa, synthetic construct
  REGION 1..10: note = TL0015-LFR4
  VARIANT 3: note = Xaa = Pro or Gln
FGXGTKLEIK

SEQ ID NO: 59 - protein, 10 aa, synthetic construct
  REGION 1..10: note = TL0015-LFR4-1
FGSGTKLEIK

SEQ ID NO: 60 - protein, 10 aa, synthetic construct
  REGION 1..10: note = TL0015-LFR4-2
FGPGTKLEIK

SEQ ID NO: 61 - protein, 10 aa, synthetic construct
  REGION 1..10: note = TL0015-LFR4-3/TLA0238-LFR4-3
FGQGTKLEIK

SEQ ID NO: 62 - protein, 116 aa, synthetic construct
  REGION 1..116: note = TLA0015-VH-1
QIQLKESGPGLVAPSQSLSITCTVSGFSLSSYGVDWVRQSPGKGLEWLGVIWGFGGTNYNSALKSRLSISKDNSKSQVFLKMNSLQSDDSAIYYCASGNFDAMDYWGQGTSVTVSS

SEQ ID NO: 63 - protein, 116 aa, synthetic construct
  REGION 1..116: note = TLA0015-VH-2
EIQLLESGGGLVQPGGSLRLSCAVSGFSLSSYGVDWVRQAPGKGLEWVGVIWGFGGTNYNSALKSRLTISRDNSKNTLYLQMNSLRAEDTAVYYCASGNFDAMDYWGQGTLVTVSS

SEQ ID NO: 64 - protein, 116 aa, synthetic construct
  REGION 1..116: note = TLA0015-VH-3
EIQLLESGGGLVQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 65 - protein, 116 aa, synthetic construct
  REGION 1..116: note = TLA0015-VH-4
EIQLVESGGGLVQPGGSLRISCAVSGFSLSSYGVDWVRQSPGKGLEWLGVIWGFGGTNYNSALKSRLTISKDNSKNTVYLQMNSLRAEDTAVYYCASGNFDAMDYWGQGTLVTVSS

SEQ ID NO: 66 - protein, 116 aa, synthetic construct
  REGION 1..116: note = TLA0015-VH-5
EVQLLESGGGLVQPGGSLRLSCAVSGFSLSSYGVDWVRQAPGKGLEWLGVIWGFGGTNYNSALKSRFTISRDNSKNTVYLQMNSLRAEDTAVYYCASGNFDAMDYWGQGTLVTVSS

SEQ ID NO: 67 - protein, 116 aa, synthetic construct
  REGION 1..116: note = TLA0015-VH-6
EVQLLESGGGLVQPGGSLRLSCAVSGFSLSSYGVDWVRQAPGKGLEWLGVIWGFGGTNYNSALKSRLTISRDNSKNTVYLQMNSLRAEDTAVYYCASGNFDAMDYWGQGTLVTVSS

SEQ ID NO: 68 - protein, 116 aa, synthetic construct
  REGION 1..116: note = TLA0015-VH-7
EVQLLESGGGLVQPGGSLRLSCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 69 - protein, 116 aa, synthetic construct
  REGION 1..116: note = TLA0015-VH-8
EIQLVESGGGLIQPGGSLRLSCAVSGFSLSSYGVDWVRQAPGKGLEWLGVIWGFGGTNYNSALKSRLTISRDNSKNTVYLQMNSLRAEDTAVYYCARGNFDAMDYWGQGTLVTVSS

SEQ ID NO: 70 - protein, 116 aa, synthetic construct
  REGION 1..116: note = TLA0015-VH-9
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 71 - protein, 116 aa, synthetic construct
  REGION 1..116: note = TLA0015-VH-10
EIQLVESGGGLIQPGGSLRISCAVSGFSLSSYGVDWVRQAPGKGLEWLGVIWGFGGTNYNSALKSRLTISRDNSKNTVYLQMNSLRAEDTAVYYCASGNFDAMDYWGQGTLVTVSS

SEQ ID NO: 72 - protein, 116 aa, synthetic construct
  REGION 1..116: note = TLA0015-VH-11
EIQLVESGGGLIQPGGSLRISCAVSGFSLSSYGVDWVRQAPGKGLEWLGVIWGFGGTNYNSALKSRLTISVDNSKNTVYLQMNSLRAEDTAVYYCASGNFDAMDYWGQGTLVTVSS

SEQ ID NO: 73 - protein, 116 aa, synthetic construct
  REGION 1..116: note = TLA0015-VH-12
EIQLVESGGGLIQPGGSLRISCAVSGFSLSSYGVDWVRQAPGKGLEWLGVIWGFGGTNYQSALKSRLTISKDNSKNTVYLQMNSLRAEDTAVYYCASGNFDAMDYWGQGTLVTVSS

SEQ ID NO: 74 - protein, 116 aa, synthetic construct
  REGION 1..116: note = TLA0015-VH-13
EIQLVESGGGLIQPGGSLRISCAVSGFSLSSYGVDWVRQAPGKGLEWLGVIWGFGGTNYNAALKSRLTISKDNSKNTVYLQMNSLRAEDTAVYYCASGNFDAMDYWGQGTLVTVSS

SEQ ID NO: 75 - protein, 116 aa, synthetic construct
  REGION 1..116: note = TLA0015-VH-14
EIQLVESGGGLIQPGGSLRISCAVSGFSLSSYGVDWVRQAPGKGLEWLGVIWGFGGTNYNSALKSRLTISADNSKNTVYLQMNSLRAEDTAVYYCASGNFDAMDYWGQGTLVTVSS

SEQ ID NO: 76 - protein, 116 aa, synthetic construct
  REGION 1..116: note = TLA0015-VH-15
EIQLVESGGGLIQPGGSLRISCAVSGFSLSYYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 77 - protein, 116 aa, synthetic construct
  REGION 1..116: note = TLA0015-VH-16
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNYDAMDYWGQGTLVTVSS

SEQ ID NO: 78 - protein, 116 aa, synthetic construct
  REGION 1..116: note = TLA0015-VH-17
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGDFDAMDYWGQGTLVTVSS

SEQ ID NO: 79 - protein, 116 aa, synthetic construct
  REGION 1..116: note = TLA0015-VH-18
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGLFDAMDYWGQGTLVTVSS

SEQ ID NO: 80 - protein, 116 aa, synthetic construct
  REGION 1..116: note = TLA0015-VH-19
EIQLVESGGGLIQPGGSLRISCAVSGFSLSTYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 81 - protein, 116 aa, synthetic construct
  REGION 1..116: note = TLA0015-VH-20
EIQLVESGGGLIQPGGSLRISCAVSGFSLISYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 82 - protein, 116 aa, synthetic construct
  REGION 1..116: note = TLA0015-VH-21
EIQLVESGGGLIQPGGSLRISCAVSGFSLSSYGVDWVRQAPGKGLEWLGVIWGFGGTNYNPALKSRLTISKDNSKNTVYLQMNSLRAEDTAVYYCASGNFDAMDYWGQGTLVTVSS

SEQ ID NO: 83 - protein, 116 aa, synthetic construct
  REGION 1..116: note = TLA0015-VH-22
EIQLVESGGGLIQPGGSLRISCAVSGFSLKSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 84 - protein, 116 aa, synthetic construct
  REGION 1..116: note = TLA0015-VH-23
EIQLVESGGGLIQPGGSLRISCAVSGFSLSSYGVDWVRQAPGKGLEWLGVIWGFGGTHYNSALKSRLTISKDNSKNTVYLQMNSLRAEDTAVYYCASGNFDAMDYWGQGTLVTVSS

SEQ ID NO: 85 - protein, 116 aa, synthetic construct
  REGION 1..116: note = TLA0015-VH-24
EIQLVESGGGLIQPGGSLRISCAVSGFSLSSYGVDWVRQAPGKGLEWLGRIWGFGGTNYNSALKSRLTISKDNSKNTVYLQMNSLRAEDTAVYYCASGNFDAMDYWGQGTLVTVSS

SEQ ID NO: 86 - protein, 116 aa, synthetic construct
  REGION 1..116: note = TLA0015-VH-25
EIQLVESGGGLIQPGGSLRISCAVSGFDLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 87 - protein, 116 aa, synthetic construct
  REGION 1..116: note = TLA0015-VH-26
EIQLVESGGGLIQPGGSLRISCAVSGFSLSSYGVDWVRQAPGKGLEWLGVIWGFGKTNYNSALKSRLTISKDNSKNTVYLQMNSLRAEDTAVYYCASGNFDAMDYWGQGTLVTVSS

SEQ ID NO: 88 - protein, 116 aa, synthetic construct
  REGION 1..116: note = TLA0015-VH-27
EIQLVESGGGLIQPGGSLRISCAVSGFSLSSYGVDWVRQAPGKGLEWLGVIWGFGGTDYNSALKSRLTISKDNSKNTVYLQMNSLRAEDTAVYYCASGNFDAMDYWGQGTLVTVSS

SEQ ID NO: 89 - protein, 116 aa, synthetic construct
  REGION 1..116: note = TLA0015-VH-28
EIQLVESGGGLIQPGGSLRISCAVSGFSLSYYGVDWVRQAPGKGLEWLGVIWGFGGTNYNSALKSRLTISKDNSKNTVYLQMNSLRAEDTAVYYCASGNYDAMDYWGQGTLVTVSS

SEQ ID NO: 90 - protein, 116 aa, synthetic construct
  REGION 1..116: note = TLA0015-VH-29
EIQLVESGGGLIQPGGSLRISCAVSGFSLSYYGVDWVRQAPGKGLEWLGVIWGFGGTNYNSALKSRLTISKDNSKNTVYLQMNSLRAEDTAVYYCASGDFDAMDYWGQGTLVTVSS

SEQ ID NO: 91 - protein, 116 aa, synthetic construct
  REGION 1..116: note = TLA0015-VH-30
EIQLVESGGGLIQPGGSLRISCAVSGFSLSYYGVDWVRQAPGKGLEWLGVIWGFGGTNYNPALKSRLTISKDNSKNTVYLQMNSLRAEDTAVYYCASGNFDAMDYWGQGTLVTVSS

SEQ ID NO: 92 - protein, 116 aa, synthetic construct
  REGION 1..116: note = TLA0015-VH-31
EIQLVESGGGLIQPGGSLRISCAVSGFSLSYYGVDWVRQAPGKGLEWLGVIWGFGGTHYNSALKSRLTISKDNSKNTVYLQMNSLRAEDTAVYYCASGNFDAMDYWGQGTLVTVSS

SEQ ID NO: 93 - protein, 116 aa, synthetic construct
  REGION 1..116: note = TLA0015-VH-32
EIQLVESGGGLIQPGGSLRISCAVSGFSLSYYGVDWVRQAPGKGLEWLGRIWGFGGTNYNSALKSRLTISKDNSKNTVYLQMNSLRAEDTAVYYCASGNFDAMDYWGQGTLVTVSS

SEQ ID NO: 94 - protein, 116 aa, synthetic construct
  REGION 1..116: note = TLA0015-VH-33
EIQLVESGGGLIQPGGSLRISCAVSGFDLSYYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 95 - protein, 116 aa, synthetic construct
  REGION 1..116: note = TLA0015-VH-34
EIQLVESGGGLIQPGGSLRISCAVSGFSLISYGVDWVRQAPGKGLEWLGVIWGFGGTNYNSALKSRLTISKDNSKNTVYLQMNSLRAEDTAVYYCASGNYDAMDYWGQGTLVTVSS

SEQ ID NO: 96 - protein, 116 aa, synthetic construct
  REGION 1..116: note = TLA0015-VH-35
EIQLVESGGGLIQPGGSLRISCAVSGFSLSSYGVDWVRQAPGKGLEWLGVIWGFGGTNYNPALKSRLTISKDNSKNTVYLQMNSLRAEDTAVYYCASGNYDAMDYWGQGTLVTVSS

SEQ ID NO: 97 - protein, 116 aa, synthetic construct
  REGION 1..116: note = TLA0015-VH-36
EIQLVESGGGLIQPGGSLRISCAVSGFSLSSYGVDWVRQAPGKGLEWLGVIWGFGGTHYNSALKSRLTISKDNSKNTVYLQMNSLRAEDTAVYYCASGNYDAMDYWGQGTLVTVSS

SEQ ID NO: 98 - protein, 116 aa, synthetic construct
  REGION 1..116: note = TLA0015-VH-37
EIQLVESGGGLIQPGGSLRISCAVSGFSLSSYGVDWVRQAPGKGLEWLGRIWGFGGTNYNSALKSRLTISKDNSKNTVYLQMNSLRAEDTAVYYCASGNYDAMDYWGQGTLVTVSS

SEQ ID NO: 99 - protein, 116 aa, synthetic construct
  REGION 1..116: note = TLA0015-VH-38
EIQLVESGGGLIQPGGSLRISCAVSGFSLSSYGVDWVRQAPGKGLEWLGVIWGFGKTNYNSALKSRLTISKDNSKNTVYLQMNSLRAEDTAVYYCASGNYDAMDYWGQGTLVTVSS

SEQ ID NO: 100 - protein, 116 aa, synthetic construct
  REGION 1..116: note = TLA0015-VH-39
EIQLVESGGGLIQPGGSLRISCAVSGFSLISYGVDWVRQAPGKGLEWLGVIWGFGGTNYNSALKSRLTISKDNSKNTVYLQMNSLRAEDTAVYYCASGDFDAMDYWGQGTLVTVSS

SEQ ID NO: 101 - protein, 116 aa, synthetic construct
  REGION 1..116: note = TLA0015-VH-40
EIQLVESGGGLIQPGGSLRISCAVSGFSLSSYGVDWVRQAPGKGLEWLGVIWGFGGTHYNSALKSRLTISKDNSKNTVYLQMNSLRAEDTAVYYCASGDFDAMDYWGQGTLVTVSS

SEQ ID NO: 102 - protein, 107 aa, synthetic construct
  REGION 1..107: note = TLA0015-VL-1
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGSGTKLEIK

SEQ ID NO: 103 - protein, 107 aa, synthetic construct
  REGION 1..107: note = TLA0015-VL-2
DIQMTQSPSSLSASVGDRVTITCRASQDISNYLNWYQQKPGKVVKLLIYYTSRLHSGVPSRFSGSGSGTDYTLTISSLQPEDVATYFCQQGNTLPFTFGPGTKLEIK

SEQ ID NO: 104 - protein, 107 aa, synthetic construct
  REGION 1..107: note = TLA0015-VL-3
DIQMTQSPSSLSASVGDRVTITCRASQDISNYLNWYQQKPGKAPKLLIYYTSRLHSGVPSRFSGSGSGTDFTLTISSLQPEDFATYYCQQGNTLPFTFGQGTKLEIK

SEQ ID NO: 105 - protein, 107 aa, synthetic construct
  REGION 1..107: note = TLA0015-VL-4
DIQMTQSPSSLSASVGDRVTITCRASQDISNYLNWYQQKPGKAPKLLIYYTSRLHSGVPSRFSGSGSGTDYTLTISSLQPEDFATYFCQQGNTLPFTFGQGTKLEIK

SEQ ID NO: 106 - protein, 107 aa, synthetic construct
  REGION 1..107: note = TLA0015-VL-5
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGQGTKLEIK

SEQ ID NO: 107 - skipped

SEQ ID NO: 108 - protein, 5 aa, synthetic construct
  REGION 1..5: note = TLA-0238-HCDR1-1
NYWMN

SEQ ID NO: 109 - protein, 5 aa, synthetic construct
  REGION 1..5: note = TLA-0238-HCDR1-2
NYWMH

SEQ ID NO: 110 - protein, 5 aa, synthetic construct
  REGION 1..5: note = TLA-0238-HCDR1-3
NYWMS

SEQ ID NO: 111 - protein, 5 aa, synthetic construct
  REGION 1..5: note = TLA-0238-HCDR1-4
NYIMN

SEQ ID NO: 112 - protein, 5 aa, synthetic construct
  REGION 1..5: note = TLA-0238-HCDR1-5
NYLMN

SEQ ID NO: 113 - protein, 5 aa, synthetic construct
  REGION 1..5: note = TLA-0238-HCDR1-6
NYMMN

SEQ ID NO: 114 - protein, 5 aa, synthetic construct
  REGION 1..5: note = TLA-0238-HCDR1-7
NYYMN

SEQ ID NO: 115 - protein, 19 aa, synthetic construct
  REGION 1..19: note = TLA-0238-HCDR2
  VARIANT 15: note = Xaa = Ala, Asp or Glu
  VARIANT 16: note = Xaa = Pro or Ser
QIRLKSDNYATHYAXXVKG

SEQ ID NO: 116 - protein, 19 aa, synthetic construct
  REGION 1..19: note = TLA-0238-HCDR2-1
QIRLKSDNYATHYAESVKG

SEQ ID NO: 117 - protein, 19 aa, synthetic construct
  REGION 1..19: note = TLA-0238-HCDR2-2
QIRLKSDNYATHYADSVKG

SEQ ID NO: 118 - protein, 19 aa, synthetic construct
  REGION 1..19: note = TLA-0238-HCDR2-3
QIRLKSDNYATHYAASVKG

SEQ ID NO: 119 - protein, 19 aa, synthetic construct
  REGION 1..19: note = TLA-0238-HCDR2-4
QIRLKSDNYATHYAAPVKG

SEQ ID NO: 120 - protein, 8 aa, synthetic construct
  REGION 1..8: note = TLA-0238-HCDR3-1
LLLRYRDY

SEQ ID NO: 121 - protein, 30 aa, synthetic construct
  REGION 1..30: note = TLA-0238-HFR1
  VARIANT 1: note = Xaa = Glu or Gln
  VARIANT 3: note = Xaa = Lys or Gln
  VARIANT 4: note = Xaa = Leu or Val
  VARIANT 5: note = Xaa = Glu or Val
  VARIANT 11: note = Xaa = Leu or Val
  VARIANT 13: note = Xaa = Lys or Gln
  VARIANT 16: note = Xaa = Gly or Arg
  VARIANT 17: note = Xaa = Ala or Ser
  VARIANT 19: note = Xaa = Lys or Arg
  VARIANT 23: note = Xaa = Ala or Ile
XVXXXESGGGXVXPGXXLXLSCXASGFTFS

SEQ ID NO: 122 - protein, 30 aa, synthetic construct
  REGION 1..30: note = TLA-0238-HFR1-1
EVKVEESGGGLVQPGGSMKLSCIASGFTFS

SEQ ID NO: 123 - protein, 30 aa, synthetic construct
  REGION 1..30: note = TLA-0238-HFR1-2
EVQVVESGGGLVKPGGSLRLSCAASGFTFS

SEQ ID NO: 124 - protein, 30 aa, synthetic construct
  REGION 1..30: note = TLA-0238-HFR1-3
EVQVVESGGGLVQPGGSLRLSCIASGFTFS

SEQ ID NO: 125 - protein, 30 aa, synthetic construct
  REGION 1..30: note = TLA-0238-HFR1-4
EVQLVESGGGLVQPGGSLRLSCAASGFTFS

SEQ ID NO: 126 - protein, 30 aa, synthetic construct
  REGION 1..30: note = TLA-0238-HFR1-5
QVQLVESGGGVVQPGRSLRLSCAASGFTFS

SEQ ID NO: 127 - protein, 30 aa, synthetic construct
  REGION 1..30: note = TLA-0238-HFR1-6
EVKVEESGGGLVQPGGSLRLSCIASGFTFS

SEQ ID NO: 128 - protein, 30 aa, synthetic construct
  REGION 1..30: note = TLA-0238-HFR1-7
EVKVEESGGGLVQPGGSLKLSCIASGFTFS

SEQ ID NO: 129 - protein, 30 aa, synthetic construct
  REGION 1..30: note = TLA-0238-HFR1-8
EVKVEESGGGLVQPGGALRLSCIASGFTFS

SEQ ID NO: 130 - protein, 30 aa, synthetic construct
  REGION 1..30: note = TLA-0238-HFR1-9
EVKLEESGGGLVQPGGSLKLSCIASGFTFS

SEQ ID NO: 131 - protein, 30 aa, synthetic construct
  REGION 1..30: note = TLA-0238-HFR1-10
EVQLVESGGGLVKPGGSLRLSCAASGFTFS

SEQ ID NO: 132 - protein, 30 aa, synthetic construct
  REGION 1..30: note = TLA-0238-HFR1-11
EVKLEESGGGLVQPGGALRLSCIASGFTFS

SEQ ID NO: 133 - protein, 14 aa, synthetic construct
  REGION 1..14: note = TLA-0238-HFR2
  VARIANT 5: note = Xaa = Ala or Ser
  VARIANT 7: note = Xaa = Glu or Gly
  VARIANT 14: note = Xaa = Ala or Gly
WVRQXPXKGLEWVX

SEQ ID NO: 134 - protein, 14 aa, synthetic construct
  REGION 1..14: note = TLA-0238-HFR2-1
WVRQSPEKGLEWVA

SEQ ID NO: 135 - protein, 14 aa, synthetic construct
  REGION 1..14: note = TLA-0238-HFR2-2
WVRQAPGKGLEWVA

SEQ ID NO: 136 - protein, 32 aa, synthetic construct
  REGION 1..32: note = TLA-0238-HFR3
  VARIANT 1: note = Xaa = Asn or Arg
  VARIANT 3: note = Xaa = Ala or Thr
  VARIANT 8: note = Xaa = Asp or Asn
  VARIANT 9: note = Xaa = Ala or Ser
  VARIANT 11: note = Xaa = Asn or Ser
  VARIANT 12: note = Xaa = Ser or Thr
  VARIANT 13: note = Xaa = Ala, Leu or Val
  VARIANT 21: note = Xaa = Lys or Arg
  VARIANT 22: note = Xaa = Ala, Asp or Thr
  VARIANT 25: note = Xaa = Met or Thr
XFXISRDXXKXXXYLQMNSLXXEDXAVYYCTP

SEQ ID NO: 137 - protein, 32 aa, synthetic construct
  REGION 1..32: note = TLA-0238-HFR3-1
NFTISRDDSKSSVYLQMNNLRAEDTGIYYCTP

SEQ ID NO: 138 - protein, 32 aa, synthetic construct
  REGION 1..32: note = TLA-0238-HFR3-2
NFTISRDDSKSTVYLQMNSLKTEDTAVYYCTP

SEQ ID NO: 139 - protein, 32 aa, synthetic construct
  REGION 1..32: note = TLA-0238-HFR3-3
NFTISRDNAKSSVYLQMNSLRAEDTAVYYCTP

SEQ ID NO: 140 - protein, 32 aa, synthetic construct
  REGION 1..32: note = TLA-0238-HFR3-4
RFTISRDDSKSTVYLQMNSLKTEDTAVYYCTP

SEQ ID NO: 141 - protein, 32 aa, synthetic construct
  REGION 1..32: note = TLA-0238-HFR3-5
NFAISRDDSKSTVYLQMNSLKTEDTAVYYCTP

SEQ ID NO: 142 - protein, 32 aa, synthetic construct
  REGION 1..32: note = TLA-0238-HFR3-6
RFTISRDNSKNTLYLQMNSLRAEDTAVYYCTP

SEQ ID NO: 143 - protein, 32 aa, synthetic construct
  REGION 1..32: note = TLA-0238-HFR3-7
RFTISRDNSKNTLYLQMNSLKTEDTAVYYCTP

SEQ ID NO: 144 - protein, 32 aa, synthetic construct
  REGION 1..32: note = TLA-0238-HFR3-8
RFTISRDDSKNSLYLQMNSLRDEDTAVYYCTP

SEQ ID NO: 145 - protein, 32 aa, synthetic construct
  REGION 1..32: note = TLA-0238-HFR3-9
RFTISRDDSKNTLYLQMNSLRAEDTAVYYCTP

SEQ ID NO: 146 - protein, 32 aa, synthetic construct
  REGION 1..32: note = TLA-0238-HFR3-10
RFTISRDDSKNSLYLQMNSLRAEDMAVYYCTP

SEQ ID NO: 147 - protein, 32 aa, synthetic construct
  REGION 1..32: note = TLA-0238-HFR3-11
RFTISRDDSKNTAYLQMNSLRAEDTAVYYCTP

SEQ ID NO: 148 - protein, 32 aa, synthetic construct
  REGION 1..32: note = TLA-0238-HFR3-12
RFTISRDDSKNTLYLQMNSLKTEDTAVYYCTP

SEQ ID NO: 149 - protein, 11 aa, synthetic construct
  REGION 1..11: note = TLA-0238-HFR4
  VARIANT 6: note = Xaa = Leu, Met or Thr
  VARIANT 7: note = Xaa = Leu or Val
WGQGTXXTVSS

SEQ ID NO: 150 - protein, 11 aa, synthetic construct
  REGION 1..11: note = TLA-0238-HFR4-1
WGQGTTLTVSS

SEQ ID NO: 151 - protein, 11 aa, synthetic construct
  REGION 1..11: note = TLA-0238-HFR4-3
WGQGTLLTVSS

SEQ ID NO: 152 - protein, 11 aa, synthetic construct
  REGION 1..11: note = TLA-0238-HFR4-4
WGQGTMVTVSS

SEQ ID NO: 153 - protein, 11 aa, synthetic construct
  REGION 1..11: note = TLA-0238-HFR4-5
WGQGTTVTVSS

SEQ ID NO: 154 - protein, 10 aa, synthetic construct
  REGION 1..10: note = TLA0238-LCDR1
  VARIANT 9: note = Xaa = Leu or Met
  VARIANT 10: note = Xaa = Ala, His or Thr
SASSSVSYXX

SEQ ID NO: 155 - protein, 10 aa, synthetic construct
  REGION 1..10: note = TLA0238-LCDR1-1
SASSSVSYMH

SEQ ID NO: 156 - protein, 10 aa, synthetic construct
  REGION 1..10: note = TLA0238-LCDR1-2
SASSSVSYLH

SEQ ID NO: 157 - protein, 10 aa, synthetic construct
  REGION 1..10: note = TLA0238-LCDR1-3
SASSSVSYLA

SEQ ID NO: 158 - protein, 10 aa, synthetic construct
  REGION 1..10: note = TLA0238-LCDR1-4
SASSSVSYLT

SEQ ID NO: 159 - protein, 7 aa, synthetic construct
  REGION 1..7: note = TLA0238-LCDR2
  VARIANT 5: note = Xaa = Leu or Arg
  VARIANT 7: note = Xaa = Ser or Thr
DTSNXAX

SEQ ID NO: 160 - protein, 7 aa, synthetic construct
  REGION 1..7: note = TLA0238-LCDR2-1
DTSNLAS

SEQ ID NO: 161 - protein, 7 aa, synthetic construct
  REGION 1..7: note = TLA0238-LCDR2-2
DTSNRAS

SEQ ID NO: 162 - protein, 7 aa, synthetic construct
  REGION 1..7: note = TLA0238-LCDR2-3
DTSNLAT

SEQ ID NO: 163 - protein, 9 aa, synthetic construct
  REGION 1..9: note = TLA0238-LCDR3
  VARIANT 4: note = Xaa = Glu, Asn or Ser
  VARIANT 5: note = Xaa = Ala, Glu or Gly
FQEXXHPFT

SEQ ID NO: 164 - protein, 9 aa, synthetic construct
  REGION 1..9: note = TLA0238-LCDR3-1
FQENGHPFT

SEQ ID NO: 165 - protein, 9 aa, synthetic construct
  REGION 1..9: note = TLA0238-LCDR3-2
FQESGHPFT

SEQ ID NO: 166 - protein, 9 aa, synthetic construct
  REGION 1..9: note = TLA0238-LCDR3-3
FQENEHPFT

SEQ ID NO: 167 - protein, 9 aa, synthetic construct
  REGION 1..9: note = TLA0238-LCDR3-4
FQEEGHPFT

SEQ ID NO: 168 - protein, 9 aa, synthetic construct
  REGION 1..9: note = TLA0238-LCDR3-5
FQENAHPFT

SEQ ID NO: 169 - protein, 23 aa, synthetic construct
  REGION 1..23: note = TLA0238-LFR1
  VARIANT 1: note = Xaa = Asp or Glu
  VARIANT 2: note = Xaa = Ile, Asn, Thr or Val
  VARIANT 3: note = Xaa = Gln or Val
  VARIANT 4: note = Xaa = Leu or Met
  VARIANT 9: note = Xaa = Ala or Ser
  VARIANT 10: note = Xaa = Phe, Ile, Ser or Thr
  VARIANT 11: note = Xaa = Leu, Met or Val
  VARIANT 13: note = Xaa = Ala, Leu or Val
  VARIANT 14: note = Xaa = Ser or Thr
  VARIANT 15: note = Xaa = Pro or Val
  VARIANT 17: note = Xaa = Asp or Glu
  VARIANT 18: note = Xaa = Lys or Arg
  VARIANT 19: note = Xaa = Ala or Val
  VARIANT 21: note = Xaa = Ile or Met
  VARIANT 22: note = Xaa = Ser or Thr
XXXXTQSPXXXSXXXGXXXTXXC

SEQ ID NO: 170 - protein, 23 aa, synthetic construct
  REGION 1..23: note = TLA0238-LFR1-1
ENVLTQSPAIMSASPGEKVTMTC

SEQ ID NO: 171 - protein, 23 aa, synthetic construct
  REGION 1..23: note = TLA0238-LFR1-2
ENVLTQSPATLSLSPGERATMSC

SEQ ID NO: 172 - protein, 23 aa, synthetic construct
  REGION 1..23: note = TLA0238-LFR1-3
DNQLTQSPSSLSASVGDRVTITC

SEQ ID NO: 173 - protein, 23 aa, synthetic construct
  REGION 1..23: note = TLA0238-LFR1-4/TLA0241-LFR1-2
DIQMTQSPSSVSASVGDRVTITC

SEQ ID NO: 174 - protein, 23 aa, synthetic construct
  REGION 1..23: note = TLA0238-LFR1-5
ENVLTQSPAIMSATPGEKVTITC

SEQ ID NO: 175 - protein, 23 aa, synthetic construct
  REGION 1..23: note = TLA0238-LFR1-6
EVVLTQSPAFLSVTPGEKVTITC

SEQ ID NO: 176 - protein, 23 aa, synthetic construct
  REGION 1..23: note = TLA0238-LFR1-7
ENVLTQSPAFMSATPGEKVTITC

SEQ ID NO: 177 - protein, 23 aa, synthetic construct
  REGION 1..23: note = TLA0238-LFR1-8
EIVLTQSPAFMSATPGEKVTITC

SEQ ID NO: 178 - protein, 23 aa, synthetic construct
  REGION 1..23: note = TLA0238-LFR1-9
ETVLTQSPAFMSATPGEKVTITC

SEQ ID NO: 179 - protein, 23 aa, synthetic construct
  REGION 1..23: note = TLA0238-LFR1-10
ENVLTQSPAFLSVTPGEKVTITC

SEQ ID NO: 180 - protein, 15 aa, synthetic construct
  REGION 1..15: note = TLA0238-LFR2
  VARIANT 7: note = Xaa = Asp or Gly
  VARIANT 8: note = Xaa = Lys or Gln
  VARIANT 9: note = Xaa = Ala or Ser
  VARIANT 11: note = Xaa = Lys, Gln or Arg
WYQQKPXXXPXLWIY

SEQ ID NO: 181 - protein, 15 aa, synthetic construct
  REGION 1..15: note = TLA0238-LFR2-1
WYQQKSSTSPKLWIY

SEQ ID NO: 182 - protein, 15 aa, synthetic construct
  REGION 1..15: note = TLA0238-LFR2-2
WYQQKPGQAPRLWIY

SEQ ID NO: 183 - protein, 15 aa, synthetic construct
  REGION 1..15: note = TLA0238-LFR2-3
WYQQKPGKAPKLWIY

SEQ ID NO: 184 - protein, 15 aa, synthetic construct
  REGION 1..15: note = TLA0238-LFR2-4
WYQQKPGQSPQLWIY

SEQ ID NO: 185 - protein, 15 aa, synthetic construct
  REGION 1..15: note = TLA0238-LFR2-5
WYQQKPGQSPRLWIY

SEQ ID NO: 186 - protein, 15 aa, synthetic construct
  REGION 1..15: note = TLA0238-LFR2-6
WYQQKPGKSPKLWIY

SEQ ID NO: 187 - protein, 15 aa, synthetic construct
  REGION 1..15: note = TLA0238-LFR2-7
WYQQKPDQSPKLWIY

SEQ ID NO: 188 - protein, 15 aa, synthetic construct
  REGION 1..15: note = TLA0238-LFR2-8
WYQQKPGQSPKLWIY

SEQ ID NO: 189 - protein, 32 aa, synthetic construct
  REGION 1..32: note = TLA0238-LFR3
  VARIANT 4: note = Xaa = Ala, Asp, Gly or Ser
  VARIANT 13: note = Xaa = Asn or Thr
  VARIANT 14: note = Xaa = Asp or Ser
  VARIANT 15: note = Xaa = Phe or Tyr
  VARIANT 22: note = Xaa = Leu or Met
  VARIANT 23: note = Xaa = Glu or Gln
  VARIANT 24: note = Xaa = Ala or Pro
  VARIANT 27: note = Xaa = Ala, Phe or Val
  VARIANT 29: note = Xaa = Thr or Val
GVPXRFSGSGSGXXXTLTISSXXXEDXAXYYC

SEQ ID NO: 190 - protein, 32 aa, synthetic construct
  REGION 1..32: note = TLA0238-LFR3-1
GVPGRFSGSGSGNSYSLTISSMEAEDVATYYC

SEQ ID NO: 191 - protein, 32 aa, synthetic construct
  REGION 1..32: note = TLA0238-LFR3-2
GVPARFSGSGSGNDYTLTISSMEPEDFAVYYC

SEQ ID NO: 192 - protein, 32 aa, synthetic construct
  REGION 1..32: note = TLA0238-LFR3-3
GVPGRFSGSGSGNDYTLTISSLQPEDFATYYC

SEQ ID NO: 193 - protein, 32 aa, synthetic construct
  REGION 1..32: note = TLA0238-LFR3-5
GVPDRFSGSGSGTDYTLTISSLQPEDVATYYC

SEQ ID NO: 194 - protein, 32 aa, synthetic construct
  REGION 1..32: note = TLA0238-LFR3-6
GVPDRFSGSGSGTDYTLTISSLQPEDFAVYYC

SEQ ID NO: 195 - protein, 32 aa, synthetic construct
  REGION 1..32: note = TLA0238-LFR3-7
GVPSRFSGSGSGTSYTLTISSLQPEDVATYYC

SEQ ID NO: 196 - protein, 32 aa, synthetic construct
  REGION 1..32: note = TLA0238-LFR3-8
GVPDRFSGSGSGTDYTLTISSLEAEDAATYYC

SEQ ID NO: 197 - protein, 32 aa, synthetic construct
  REGION 1..32: note = TLA0238-LFR3-9
GVPSRFSGSGSGTDYTLTISSLQPEDFATYYC

SEQ ID NO: 198 - protein, 10 aa, synthetic construct
  REGION 1..10: note = TLA0238-LFR4
  VARIANT 3: note = Xaa = Gly or Gln
  VARIANT 7: note = Xaa = Leu or Val
  VARIANT 9: note = Xaa = Ile or Met
FGXGTKXEXK

SEQ ID NO: 199 - protein, 10 aa, synthetic construct
  REGION 1..10: note = TLA0238-LFR4-1
FGGGTKLEMK

SEQ ID NO: 200 - protein, 10 aa, synthetic construct
  REGION 1..10: note = TLA0238-LFR4-2
FGGGTKVEMK

SEQ ID NO: 201 - protein, 10 aa, synthetic construct
  REGION 1..10: note = TLA0238-LFR4-4/TLA0241-LFR4-1
FGQGTKVEIK

SEQ ID NO: 202 - protein, 119 aa, synthetic construct
  REGION 1..119: note = TLA0238-VH-1
EVKVEESGGGLVQPGGSMKLSCIASGFTFSNYWMNWVRQSPEKGLEWVAQIRLKSDNYATHYAESVKGNFTISRDDSKSSVYLQMNNLRAEDTGIYYCTPLLLRYRDYWGQGTTLTVSS

SEQ ID NO: 203 - protein, 119 aa, synthetic construct
  REGION 1..119: note = TLA0238-VH-2
EVQVVESGGGLVKPGGSLRLSCAASGFTFSNYWMNWVRQAPGKGLEWVAQIRLKSDNYATHYAESVKGNFTISRDDSKSTVYLQMNSLKTEDTAVYYCTPLLLRYRDYWGQGTLVTVSS

SEQ ID NO: 204 - protein, 119 aa, synthetic construct
  REGION 1..119: note = TLA0238-VH-3
EVQVVESGGGLVQPGGSLRLSCIASGFTFSNYWMNWVRQAPGKGLEWVAQIRLKSDNYATHYAESVKGNFTISRDNAKSSVYLQMNSLRAEDTAVYYCTPLLLRYRDYWGQGTLLTVSS

SEQ ID NO: 205 - protein, 119 aa, synthetic construct
  REGION 1..119: note = TLA0238-VH-4
EVQVVESGGGLVKPGGSLRLSCAASGFTFSNYWMNWVRQAPGKGLEWVAQIRLKSDNYATHYAESVKGRFTISRDDSKSTVYLQMNSLKTEDTAVYYCTPLLLRYRDYWGQGTLVTVSS

SEQ ID NO: 206 - protein, 119 aa, synthetic construct
  REGION 1..119: note = TLA0238-VH-5
EVQVVESGGGLVKPGGSLRLSCAASGFTFSNYWMNWVRQAPGKGLEWVAQIRLKSDNYATHYAESVKGNFAISRDDSKSTVYLQMNSLKTEDTAVYYCTPLLLRYRDYWGQGTLVTVSS

SEQ ID NO: 207 - protein, 119 aa, synthetic construct
  REGION 1..119: note = TLA0238-VH-6
QVQLVESGGGVVQPGRSLRLSCAASGFTFSNYWMNWVRQAPGKGLEWVAQIRLKSDNYATHYADSVKGRFTISRDNSKNTLYLQMNSLRAEDTAVYYCTPLLLRYRDYWGQGTMVTVSS

SEQ ID NO: 208 - protein, 119 aa, synthetic construct
  REGION 1..119: note = TLA0238-VH-7
EVKVEESGGGLVQPGGSLRLSCIASGFTFSNYWMHWVRQAPGKGLEWVGQIRLKSDNYATHYAASVKGRFTISRDNSKNTLYLQMNSLKTEDTAVYYCTPLLLRYRDYWGQGTLVTVSS

SEQ ID NO: 209 - protein, 119 aa, synthetic construct
  REGION 1..119: note = TLA0238-VH-8
EVKVEESGGGLVQPGGSLKLSCIASGFTFSNYWMHWVRQAPGKGLEWVGQIRLKSDNYATHYAASVKGRFTISRDDSKNSLYLQMNSLRDEDTAVYYCTPLLLRYRDYWGQGTLVTVSS

SEQ ID NO: 210 - protein, 119 aa, synthetic construct
  REGION 1..119: note = TLA0238-VH-9
EVKVEESGGGLVQPGGALRLSCIASGFTFSNYWMHWVRQAPGKGLEWVGQIRLKSDNYATHYAASVKGRFTISRDDSKNTLYLQMNSLRAEDTAVYYCTPLLLRYRDYWGQGTLVTVSS

SEQ ID NO: 211 - protein, 119 aa, synthetic construct
  REGION 1..119: note = TLA0238-VH-10
EVKVEESGGGLVQPGGSLRLSCIASGFTFSNYWMHWVRQAPGKGLEWVGQIRLKSDNYATHYAASVKGRFTISRDDSKNSLYLQMNSLRAEDMAVYYCTPLLLRYRDYWGQGTLVTVSS

SEQ ID NO: 212 - protein, 119 aa, synthetic construct
  REGION 1..119: note = TLA0238-VH-11
EVQLVESGGGLVQPGGSLRLSCAASGFTFSNYWMNWVRQAPGKGLEWVAQIRLKSDNYATHYADSVKGRFTISRDNSKNTLYLQMNSLRAEDTAVYYCTPLLLRYRDYWGQGTMVTVSS

SEQ ID NO: 213 - protein, 119 aa, synthetic construct
  REGION 1..119: note = TLA0238-VH-12
EVKLEESGGGLVQPGGSLKLSCIASGFTFSNYWMHWVRQAPGKGLEWVGQIRLKSDNYATHYAASVKGRFTISRDDSKNTAYLQMNSLRAEDTAVYYCTPLLLRYRDYWGQGTLVTVSS

SEQ ID NO: 214 - protein, 119 aa, synthetic construct
  REGION 1..119: note = TLA0238-VH-13
EVQLVESGGGLVKPGGSLRLSCAASGFTFSNYWMNWVRQAPGKGLEWVAQIRLKSDNYATHYAAPVKGRFTISRDDSKNTLYLQMNSLKTEDTAVYYCTPLLLRYRDYWGQGTTVTVSS

SEQ ID NO: 215 - protein, 119 aa, synthetic construct
  REGION 1..119: note = TLA0238-VH-14
EVKLEESGGGLVQPGGALRLSCIASGFTFSNYWMSWVRQAPGKGLEWVGQIRLKSDNYATHYAASVKGRFTISRDDSKNTLYLQMNSLRAEDTAVYYCTPLLLRYRDYWGQGTLVTVSS

SEQ ID NO: 216 - protein, 119 aa, synthetic construct
  REGION 1..119: note = TLA0238-VH-15
EVQLVESGGGLVQPGGSLRLSCAASGFTFSNYIMNWVRQAPGKGLEWVAQIRLKSDNYATHYADSVKGRFTISRDNSKNTLYLQMNSLRAEDTAVYYCTPLLLRYRDYWGQGTLVTVSS

SEQ ID NO: 217 - protein, 119 aa, synthetic construct
  REGION 1..119: note = TLA0238-VH-16
EVQLVESGGGLVQPGGSLRLSCAASGFTFSNYLMNWVRQAPGKGLEWVAQIRLKSDNYATHYADSVKGRFTISRDNSKNTLYLQMNSLRAEDTAVYYCTPLLLRYRDYWGQGTLVTVSS

SEQ ID NO: 218 - protein, 119 aa, synthetic construct
  REGION 1..119: note = TLA0238-VH-17
EVQLVESGGGLVQPGGSLRLSCAASGFTFSNYMMNWVRQAPGKGLEWVAQIRLKSDNYATHYADSVKGRFTISRDNSKNTLYLQMNSLRAEDTAVYYCTPLLLRYRDYWGQGTLVTVSS

SEQ ID NO: 219 - protein, 119 aa, synthetic construct
  REGION 1..119: note = TLA0238-VH-18
EVQLVESGGGLVQPGGSLRLSCAASGFTFSNYWMNWVRQAPGKGLEWVAQIRLKSDNYATHYADSVKGRFTISRDNSKNTLYLQMNSLRAEDTAVYYCTPLLLRYRDYWGQGTLVTVSS

SEQ ID NO: 220 - protein, 119 aa, synthetic construct
  REGION 1..119: note = TLA0238-VH-19
EVQLVESGGGLVQPGGSLRLSCAASGFTFSNYYMNWVRQAPGKGLEWVAQIRLKSDNYATHYADSVKGRFTISRDNSKNTLYLQMNSLRAEDTAVYYCTPLLLRYRDYWGQGTLVTVSS

SEQ ID NO: 221 - protein, 106 aa, synthetic construct
  REGION 1..106: note = TLA0238-VL-1
ENVLTQSPAIMSASPGEKVTMTCSASSSVSYMHWYQQKSSTSPKLWIYDTSNLASGVPGRFSGSGSGNSYSLTISSMEAEDVATYYCFQENGHPFTFGGGTKLEMK

SEQ ID NO: 222 - protein, 106 aa, synthetic construct
  REGION 1..106: note = TLA0238-VL-2
ENVLTQSPATLSLSPGERATMSCSASSSVSYMHWYQQKPGQAPRLWIYDTSNLASGVPARFSGSGSGNDYTLTISSMEPEDFAVYYCFQENGHPFTFGGGTKVEMK

SEQ ID NO: 223 - protein, 106 aa, synthetic construct
  REGION 1..106: note = TLA0238-VL-3
DNQLTQSPSSLSASVGDRVTITCSASSSVSYMHWYQQKPGKAPKLWIYDTSNLASGVPGRFSGSGSGNDYTLTISSLQPEDFATYYCFQENGHPFTFGQGTKLEIK

SEQ ID NO: 224 - protein, 106 aa, synthetic construct
  REGION 1..106: note = TLA0238-VL-4
ENVLTQSPAIMSATPGEKVTITCSASSSVSYLHWYQQKPGQSPQLWIYDTSNRASGVPDRFSGSGSGTDYTLTISSLQPEDVATYYCFQENGHPFTFGGGTKLEMK

SEQ ID NO: 225 - protein, 106 aa, synthetic construct
  REGION 1..106: note = TLA0238-VL-5
EVVLTQSPAFLSVTPGEKVTITCSASSSVSYLAWYQQKPGQSPRLWIYDTSNRASGVPDRFSGSGSGTDYTLTISSLQPEDFAVYYCFQENGHPFTFGGGTKLEMK

SEQ ID NO: 226 - protein, 106 aa, synthetic construct
  REGION 1..106: note = TLA0238-VL-6
ENVLTQSPAFMSATPGEKVTITCSASSSVSYLAWYQQKPGKSPKLWIYDTSNLATGVPSRFSGSGSGTSYTLTISSLQPEDVATYYCFQENGHPFTFGGGTKLEMK

SEQ ID NO: 227 - protein, 106 aa, synthetic construct
  REGION 1..106: note = TLA0238-VL-7
EIVLTQSPAFMSATPGEKVTITCSASSSVSYLHWYQQKPDQSPKLWIYDTSNRASGVPDRFSGSGSGTDYTLTISSLEAEDAATYYCFQENGHPFTFGGGTKLEMK

SEQ ID NO: 228 - protein, 106 aa, synthetic construct
  REGION 1..106: note = TLA0238-VL-8
ETVLTQSPAFMSATPGEKVTITCSASSSVSYLTWYQQKPGQSPKLWIYDTSNLASGVPDRFSGSGSGTDYTLTISSLEAEDAATYYCFQENGHPFTFGGGTKLEMK

SEQ ID NO: 229 - protein, 106 aa, synthetic construct
  REGION 1..106: note = TLA0238-VL-9
DIQMTQSPSSVSASVGDRVTITCSASSSVSYMHWYQQKPGKAPKLWIYDTSNLASGVPSRFSGSGSGTDFTLTISSLQPEDFATYYCFQENGHPFTFGQGTKVEIK

SEQ ID NO: 230 - protein, 106 aa, synthetic construct
  REGION 1..106: note = TLA0238-VL-10
ENVLTQSPAFLSVTPGEKVTITCSASSSVSYLHWYQQKPDQSPKLWIYDTSNLATGVPSRFSGSGSGTDYTLTISSLQPEDFATYYCFQENGHPFTFGGGTKLEMK

SEQ ID NO: 231 - protein, 106 aa, synthetic construct
  REGION 1..106: note = TLA0238-VL-11
DIQMTQSPSSVSASVGDRVTITCSASSSVSYMHWYQQKPGKAPKLWIYDTSNLASGVPSRFSGSGSGTDFTLTISSLQPEDFATYYCFQESGHPFTFGQGTKVEIK

SEQ ID NO: 232 - protein, 106 aa, synthetic construct
  REGION 1..106: note = TLA0238-VL-12
DIQMTQSPSSVSASVGDRVTITCSASSSVSYMHWYQQKPGKAPKLWIYDTSNLASGVPSRFSGSGSGTDFTLTISSLQPEDFATYYCFQENEHPFTFGQGTKVEIK

SEQ ID NO: 233 - protein, 106 aa, synthetic construct
  REGION 1..106: note = TLA0238-VL-13
DIQMTQSPSSVSASVGDRVTITCSASSSVSYMHWYQQKPGKAPKLWIYDTSNLASGVPSRFSGSGSGTDFTLTISSLQPEDFATYYCFQEEGHPFTFGQGTKVEIK

SEQ ID NO: 234 - protein, 106 aa, synthetic construct
  REGION 1..106: note = TLA0238-VL-14
DIQMTQSPSSVSASVGDRVTITCSASSSVSYMHWYQQKPGKAPKLWIYDTSNLASGVPSRFSGSGSGTDFTLTISSLQPEDFATYYCFQENAHPFTFGQGTKVEIK

SEQ ID NO: 235 - skipped

SEQ ID NO: 236 - protein, 5 aa, synthetic construct
  REGION 1..5: note = TLA0241-HCDR1-1
DYYLN

SEQ ID NO: 237 - protein, 5 aa, synthetic construct
  REGION 1..5: note = TLA0241-HCDR1-2
DYYMH

SEQ ID NO: 238 - protein, 17 aa, synthetic construct
  REGION 1..17: note = TLA0241-HCDR2
  VARIANT 12: note = Xaa = Ala or Asn
  VARIANT 13: note = Xaa = Asp or Gln
  VARIANT 14: note = Xaa = Lys or Ser
  VARIANT 15: note = Xaa = Phe or Val
  VARIANT 16: note = Xaa = Lys, Gln or Thr
DINPNNGRTTYXXXXXG

SEQ ID NO: 239 - protein, 17 aa, synthetic construct
  REGION 1..17: note = TLA0241-HCDR2-1
DINPNNGRTTYNQKFTG

SEQ ID NO: 240 - protein, 17 aa, synthetic construct
  REGION 1..17: note = TLA0241-HCDR2-2
DINPNNGRTTYADSVKG

SEQ ID NO: 241 - protein, 17 aa, synthetic construct
  REGION 1..17: note = TLA0241-HCDR2-3
DINPNNGRTTYNQKFQG

SEQ ID NO: 242 - protein, 10 aa, synthetic construct
  REGION 1..10: note = TLA0241-HCDR3-1
EGGYGWYFDV

SEQ ID NO: 243 - protein, 30 aa, synthetic construct
  REGION 1..30: note = TLA0241-HFR1
  VARIANT 1: note = Xaa = Glu or Gln
  VARIANT 2: note = Xaa = Ile, Leu or Val
  VARIANT 5: note = Xaa = Leu, Gln or Val
  VARIANT 6: note = Xaa = Glu or Gln
  VARIANT 9: note = Xaa = Ala, Gly or Pro
  VARIANT 10: note = Xaa = Glu or Gly
  VARIANT 11: note = Xaa = Glu, Leu or Val
  VARIANT 12: note = Xaa = Lys or Val
  VARIANT 13: note = Xaa = Lys or Gln
  VARIANT 16: note = Xaa = Ala, Gly or Arg
  VARIANT 18: note = Xaa = Leu or Val
  VARIANT 20: note = Xaa = Ile or Leu
  VARIANT 23: note = Xaa = Ala, Lys or Ser
XXQLXXSGXXXXXPGXSXRXSCXASGYTFT

SEQ ID NO: 244 - protein, 30 aa, synthetic construct
  REGION 1..30: note = TLA0241-HFR1-1
EVQLVESGGGLVQPGGSLRLSCSASGYTFT

SEQ ID NO: 245 - protein, 30 aa, synthetic construct
  REGION 1..30: note = TLA0241-HFR1-2
QVQLVESGGGVVQPGRSLRLSCAASGYTFT

SEQ ID NO: 246 - protein, 30 aa, synthetic construct
  REGION 1..30: note = TLA0241-HFR1-3
EVQLQQSGPEVKKPGASVRISCKASGYTFT

SEQ ID NO: 247 - protein, 30 aa, synthetic construct
  REGION 1..30: note = TLA0241-HFR1-4
ELQLQQSGPEVKKPGASVRISCKASGYTFT

SEQ ID NO: 248 - protein, 30 aa, synthetic construct
  REGION 1..30: note = TLA0241-HFR1-5
EVQLLESGGGLVQPGGSLRLSCAASGYTFT

SEQ ID NO: 249 - protein, 30 aa, synthetic construct
  REGION 1..30: note = TLA0241-HFR1-6
EIQLQQSGAEEKKPGASVRISCKASGYTFT

SEQ ID NO: 250 - protein, 30 aa, synthetic construct
  REGION 1..30: note = TLA0241-HFR1-7
ELQLQQSGAEVKKPGASVRISCKASGYTFT

SEQ ID NO: 251 - protein, 14 aa, synthetic construct
  REGION 1..14: note = TLA0241-HFR2
  VARIANT 3: note = Xaa = Gln or Arg
  VARIANT 8: note = Xaa = Glu, Lys or Gln
WVXQAPGXGLEWIG

SEQ ID NO: 252 - protein, 14 aa, synthetic construct
  REGION 1..14: note = TLA0241-HFR2-1
WVRQAPGKGLEWIG

SEQ ID NO: 253 - protein, 14 aa, synthetic construct
  REGION 1..14: note = TLA0241-HFR2-2
WVRQAPGQGLEWIG

SEQ ID NO: 254 - protein, 14 aa, synthetic construct
  REGION 1..14: note = TLA0241-HFR2-3
WVQQAPGKGLEWIG

SEQ ID NO: 255 - protein, 14 aa, synthetic construct
  REGION 1..14: note = TLA0241-HFR2-4
WVRQAPGEGLEWIG

SEQ ID NO: 256 - protein, 32 aa, synthetic construct
  REGION 1..32: note = TLA0241-HFR3
  VARIANT 2: note = Xaa = Phe or Val
  VARIANT 5: note = Xaa = Ser or Thr
  VARIANT 6: note = Xaa = Ala or Arg
  VARIANT 8: note = Xaa = Lys, Asn or Thr
  VARIANT 10: note = Xaa = Lys, Ser or Thr
  VARIANT 11: note = Xaa = Asn or Ser
  VARIANT 13: note = Xaa = Ala or Leu
  VARIANT 15: note = Xaa = Leu or Met
  VARIANT 16: note = Xaa = Glu or Gln
  VARIANT 17: note = Xaa = Leu or Met
  VARIANT 18: note = Xaa = Asn, Arg or Ser
  VARIANT 21: note = Xaa = Arg or Thr
  VARIANT 22: note = Xaa = Ala or Ser
  VARIANT 23: note = Xaa = Asp or Glu
  VARIANT 25: note = Xaa = Met or Thr
RXTIXXDXSXXTXYXXXXSLXXXDXAVYYCAR

SEQ ID NO: 257 - protein, 32 aa, synthetic construct
  REGION 1..32: note = TLA0241-HFR3-1
RFTISRDNSKNTLYLQMSSLRAEDTAVYYCAR

SEQ ID NO: 258 - protein, 32 aa, synthetic construct
  REGION 1..32: note = TLA0241-HFR3-2
RFTISRDNSKNTLYLQMNSLRAEDTAVYYCAR

SEQ ID NO: 259 - protein, 32 aa, synthetic construct
  REGION 1..32: note = TLA0241-HFR3-3
RVTITRDTSSSTAYMELRSLRSDDTAVYYCAR

SEQ ID NO: 260 - protein, 32 aa, synthetic construct
  REGION 1..32: note = TLA0241-HFR3-4
RVTITADKSTSTAYMELRSLRSDDTAVYYCAR

SEQ ID NO: 261 - protein, 32 aa, synthetic construct
  REGION 1..32: note = TLA0241-HFR3-5
RVTITADKSTSTAYMELRSLTSDDTAVYYCAR

SEQ ID NO: 262 - protein, 32 aa, synthetic construct
  REGION 1..32: note = TLA0241-HFR3-6
RVTITADKSTSTAYMELRSLRSDDMAVYYCAR

SEQ ID NO: 263 - protein, 11 aa, synthetic construct
  REGION 1..11: note = TLA0241-HFR4
  VARIANT 3: note = Xaa = Gln, Arg or Thr
  VARIANT 6: note = Xaa = Leu, Met or Thr
WGXGTXVTVSS

SEQ ID NO: 264 - protein, 11 aa, synthetic construct
  REGION 1..11: note = TLA0241-HFR4-1
WGRGTLVTVSS

SEQ ID NO: 265 - protein, 11 aa, synthetic construct
  REGION 1..11: note = TLA0241-HFR4-2
WGQGTMVTVSS

SEQ ID NO: 266 - protein, 11 aa, synthetic construct
  REGION 1..11: note = TLA0241-HFR4-3
WGTGTLVTVSS

SEQ ID NO: 267 - protein, 11 aa, synthetic construct
  REGION 1..11: note = TLA0241-HFR4-4
WGQGTTVTVSS

SEQ ID NO: 268 - protein, 11 aa, synthetic construct
  REGION 1..11: note = TLA0241-HFR4-5
WGTGTTVTVSS

SEQ ID NO: 269 - protein, 12 aa, synthetic construct
  REGION 1..12: note = TLA0241-LCDR1
  VARIANT 2: note = Xaa = Ala or Thr
  VARIANT 11: note = Xaa = Leu or Ser
  VARIANT 12: note = Xaa = Ala or His
SXSSSIISNYXX

SEQ ID NO: 270 - protein, 12 aa, synthetic construct
  REGION 1..12: note = TLA0241-LCDR1-1
STSSSIISNYSH

SEQ ID NO: 271 - protein, 12 aa, synthetic construct
  REGION 1..12: note = TLA0241-LCDR1-2
SASSSIISNYLH

SEQ ID NO: 272 - protein, 12 aa, synthetic construct
  REGION 1..12: note = TLA0241-LCDR1-3
SASSSIISNYSA

SEQ ID NO: 273 - protein, 12 aa, synthetic construct
  REGION 1..12: note = TLA0241-LCDR1-4
SASSSIISNYLA

SEQ ID NO: 274 - protein, 7 aa, synthetic construct
  REGION 1..7: note = TLA0241-LCDR2
  VARIANT 5: note = Xaa = Leu or Arg
KTSNXPS

SEQ ID NO: 275 - protein, 7 aa, synthetic construct
  REGION 1..7: note = TLA0241-LCDR2-1
KTSNLPS

SEQ ID NO: 276 - protein, 7 aa, synthetic construct
  REGION 1..7: note = TLA0241-LCDR2-2
KTSNRPS

SEQ ID NO: 277 - protein, 9 aa, synthetic construct
  REGION 1..9: note = TLA0241-LCDR3-1
QQGSDMPIT

SEQ ID NO: 278 - protein, 23 aa, synthetic construct
  REGION 1..23: note = TLA0241-LFR1
  VARIANT 1: note = Xaa = Ala, Asp or Glu
  VARIANT 3: note = Xaa = Gln or Val
  VARIANT 4: note = Xaa = Leu or Met
  VARIANT 9: note = Xaa = Ala, Gly, Pro, Ser or Thr
  VARIANT 10: note = Xaa = Ser or Thr
  VARIANT 11: note = Xaa = Leu or Val
  VARIANT 13: note = Xaa = Ala or Leu
  VARIANT 15: note = Xaa = Pro or Val
  VARIANT 17: note = Xaa = Asp or Glu
  VARIANT 18: note = Xaa = Lys or Arg
XIXXTQSPXXXSXSXGXXVTITC

SEQ ID NO: 279 - protein, 23 aa, synthetic construct
  REGION 1..23: note = TLA0241-LFR1-3
EIVLTQSPTTLSLSPGEKVTITC

SEQ ID NO: 280 - protein, 23 aa, synthetic construct
  REGION 1..23: note = TLA0241-LFR1-4
EIVLTQSPPTLSLSPGEKVTITC

SEQ ID NO: 281 - protein, 23 aa, synthetic construct
  REGION 1..23: note = TLA0241-LFR1-5
EIVLTQSPGTLSLSPGEKVTITC

SEQ ID NO: 282 - protein, 23 aa, synthetic construct
  REGION 1..23: note = TLA0241-LFR1-6
AIQLTQSPSSLSASVGDRVTITC

SEQ ID NO: 283 - protein, 23 aa, synthetic construct
  REGION 1..23: note = TLA0241-LFR1-7
EIVLTQSPATLSLSPGEKVTITC

SEQ ID NO: 284 - protein, 15 aa, synthetic construct
  REGION 1..15: note = TLA0241-LFR2
  VARIANT 7: note = Xaa = Asp or Gly
  VARIANT 8: note = Xaa = Lys or Gln
  VARIANT 9: note = Xaa = Ala or Ser
  VARIANT 11: note = Xaa = Lys or Gln
WYQQKPXXXPXLLIY

SEQ ID NO: 285 - protein, 15 aa, synthetic construct
  REGION 1..15: note = TLA0241-LFR2-2
WYQQKPGKSPKLLIY

SEQ ID NO: 286 - protein, 15 aa, synthetic construct
  REGION 1..15: note = TLA0241-LFR2-3
WYQQKPGQSPQLLIY

SEQ ID NO: 287 - protein, 15 aa, synthetic construct
  REGION 1..15: note = TLA0241-LFR2-4
WYQQKPDQSPKLLIY

SEQ ID NO: 288 - protein, 32 aa, synthetic construct
  REGION 1..32: note = TLA0241-LFR3
  VARIANT 4: note = Xaa = Ala, Asp or Ser
  VARIANT 14: note = Xaa = Asp, Glu or Ser
  VARIANT 20: note = Xaa = Asn or Ser
  VARIANT 23: note = Xaa = Glu or Gln
  VARIANT 24: note = Xaa = Ala or Pro
  VARIANT 27: note = Xaa = Ala or Phe
GVPXRFSGSGSGTXFTLTIXSLXXEDXATYYC

SEQ ID NO: 289 - protein, 32 aa, synthetic construct
  REGION 1..32: note = TLA0241-LFR3-1
GVPSRFSGSGSGTEFTLTISSLQPEDFATYYC

SEQ ID NO: 290 - protein, 32 aa, synthetic construct
  REGION 1..32: note = TLA0241-LFR3-3
GVPSRFSGSGSGTDFTLTINSLEAEDAATYYC

SEQ ID NO: 291 - protein, 32 aa, synthetic construct
  REGION 1..32: note = TLA0241-LFR3-4
GVPDRFSGSGSGTDFTLTINSLEAEDAATYYC

SEQ ID NO: 292 - protein, 32 aa, synthetic construct
  REGION 1..32: note = TLA0241-LFR3-5
GVPARFSGSGSGTDFTLTINSLEAEDAATYYC

SEQ ID NO: 293 - protein, 32 aa, synthetic construct
  REGION 1..32: note = TLA0241-LFR3-6
GVPSRFSGSGSGTSFTLTINSLEAEDAATYYC

SEQ ID NO: 294 - protein, 10 aa, synthetic construct
  REGION 1..10: note = TLA0241-LFR4
  VARIANT 3: note = Xaa = Ala or Gln
  VARIANT 6: note = Xaa = Lys or Arg
  VARIANT 7: note = Xaa = Leu or Val
  VARIANT 9: note = Xaa = Ile or Leu
FGXGTXXEXK

SEQ ID NO: 295 - protein, 10 aa, synthetic construct
  REGION 1..10: note = TLA0241-LFR4-2
FGAGTRLELK

SEQ ID NO: 296 - protein, 10 aa, synthetic construct
  REGION 1..10: note = TLA0241-LFR4-3
FGAGTKLELK

SEQ ID NO: 297 - protein, 119 aa, synthetic construct
  REGION 1..119: note = TLA0241-VH-1
EFQLQQSGPELVKPGASVRISCKASGYTFTDYYLNWVKQSRGESLEWIGDINPNNGRTTYNQKFTGKATWTVDKSSSTVYMELRSLTSDDSAVYYCAREGGYGWYFDVWGTGTTVTVSS

SEQ ID NO: 298 - protein, 119 aa, synthetic construct
  REGION 1..119: note = TLA0241-VH-2
EVQLVESGGGLVQPGGSLRLSCSASGYTFTDYYLNWVRQAPGKGLEWIGDINPNNGRTTYADSVKGRFTISRDNSKNTLYLQMSSLRAEDTAVYYCAREGGYGWYFDVWGRGTLVTVSS

SEQ ID NO: 299 - protein, 119 aa, synthetic construct
  REGION 1..119: note = TLA0241-VH-3
QVQLVESGGGVVQPGRSLRLSCAASGYTFTDYYLNWVRQAPGKGLEWIGDINPNNGRTTYADSVKGRFTISRDNSKNTLYLQMNSLRAEDTAVYYCAREGGYGWYFDVWGQGTMVTVSS

SEQ ID NO: 300 - protein, 119 aa, synthetic construct
  REGION 1..119: note = TLA0241-VH-4
EVQLQQSGPEVKKPGASVRISCKASGYTFTDYYMHWVRQAPGQGLEWIGDINPNNGRTTYNQKFQGRVTITRDTSSSTAYMELRSLRSDDTAVYYCAREGGYGWYFDVWGTGTTVTVSS

SEQ ID NO: 301 - protein, 119 aa, synthetic construct
  REGION 1..119: note = TLA0241-VH-5
EVQLQQSGPEVKKPGASVRISCKASGYTFTDYYMHWVQQAPGKGLEWIGDINPNNGRTTYNQKFQGRVTITADKSTSTAYMELRSLRSDDTAVYYCAREGGYGWYFDVWGTGTLVTVSS

SEQ ID NO: 302 - protein, 119 aa, synthetic construct
  REGION 1..119: note = TLA0241-VH-6
ELQLQQSGPEVKKPGASVRISCKASGYTFTDYYMHWVQQAPGKGLEWIGDINPNNGRTTYNQKFQGRVTITADKSTSTAYMELRSLTSDDTAVYYCAREGGYGWYFDVWGTGTTVTVSS

SEQ ID NO: 303 - protein, 119 aa, synthetic construct
  REGION 1..119: note = TLA0241-VH-7
EVQLLESGGGLVQPGGSLRLSCAASGYTFTDYYLNWVRQAPGKGLEWIGDINPNNGRTTYADSVKGRFTISRDNSKNTLYLQMNSLRAEDTAVYYCAREGGYGWYFDVWGQGTMVTVSS

SEQ ID NO: 304 - protein, 119 aa, synthetic construct
  REGION 1..119: note = TLA0241-VH-8
EVQLQQSGPEVKKPGASVRISCKASGYTFTDYYMHWVRQAPGEGLEWIGDINPNNGRTTYNQKFQGRVTITADKSTSTAYMELRSLRSDDTAVYYCAREGGYGWYFDVWGTGTTVTVSS

SEQ ID NO: 305 - protein, 119 aa, synthetic construct
  REGION 1..119: note = TLA0241-VH-9
QVQLVESGGGVVQPGRSLRLSCAASGYTFTDYYLNWVRQAPGKGLEWIGDINPNNGRTTYADSVKGRFTISRDNSKNTLYLQMNSLRAEDTAVYYCAREGGYGWYFDVWGQGTTVTVSS

SEQ ID NO: 306 - protein, 119 aa, synthetic construct
  REGION 1..119: note = TLA0241-VH-10
EIQLQQSGAEEKKPGASVRISCKASGYTFTDYYMHWVQQAPGKGLEWIGDINPNNGRTTYNQKFQGRVTITADKSTSTAYMELRSLRSDDTAVYYCAREGGYGWYFDVWGTGTTVTVSS

SEQ ID NO: 307 - protein, 119 aa, synthetic construct
  REGION 1..119: note = TLA0241-VH-11
ELQLQQSGAEVKKPGASVRISCKASGYTFTDYYMHWVRQAPGQGLEWIGDINPNNGRTTYNQKFQGRVTITADKSTSTAYMELRSLRSDDMAVYYCAREGGYGWYFDVWGTGTLVTVSS

SEQ ID NO: 308 - protein, 108 aa, synthetic construct
  REGION 1..108: note = TLA0241-VL-1
EIVLTQSPTTMAASPGEKITITCSTSSSIISNYSHWYQQKPGFSPKLLIYKTSNLPSGVPARFSGSGSGTSYSLTIGTMEAEDAATYYCQQGSDMPITFGAGTKLELK

SEQ ID NO: 309 - protein, 108 aa, synthetic construct
  REGION 1..108: note = TLA0241-VL-2
DIQMTQSPSSLSASVGDRVTITCSTSSSIISNYSHWYQQKPGKAPKLLIYKTSNLPSGVPSRFSGSGSGTEFTLTISSLQPEDFATYYCQQGSDMPITFGQGTKVEIK

SEQ ID NO: 310 - protein, 108 aa, synthetic construct
  REGION 1..108: note = TLA0241-VL-3
EIVLTQSPTTLSLSPGEKVTITCSASSSIISNYLHWYQQKPGKSPKLLIYKTSNLPSGVPSRFSGSGSGTDFTLTINSLEAEDAATYYCQQGSDMPITFGAGTRLELK

SEQ ID NO: 311 - protein, 108 aa, synthetic construct
  REGION 1..108: note = TLA0241-VL-4
EIVLTQSPPTLSLSPGEKVTITCSASSSIISNYLHWYQQKPGQSPQLLIYKTSNRPSGVPDRFSGSGSGTDFTLTINSLEAEDAATYYCQQGSDMPITFGAGTRLELK

SEQ ID NO: 312 - protein, 108 aa, synthetic construct
  REGION 1..108: note = TLA0241-VL-5
EIVLTQSPGTLSLSPGEKVTITCSASSSIISNYLHWYQQKPDQSPKLLIYKTSNLPSGVPSRFSGSGSGTDFTLTINSLEAEDAATYYCQQGSDMPITFGAGTKLELK

SEQ ID NO: 313 - protein, 108 aa, synthetic construct
  REGION 1..108: note = TLA0241-VL-6
AIQLTQSPSSLSASVGDRVTITCSTSSSIISNYSHWYQQKPGKAPKLLIYKTSNLPSGVPSRFSGSGSGTDFTLTISSLQPEDFATYYCQQGSDMPITFGQGTKVEIK

SEQ ID NO: 314 - protein, 108 aa, synthetic construct
  REGION 1..108: note = TLA0241-VL-7
EIVLTQSPATLSLSPGEKVTITCSASSSIISNYSAWYQQKPGKSPKLLIYKTSNLPSGVPARFSGSGSGTDFTLTINSLEAEDAATYYCQQGSDMPITFGAGTRLELK

SEQ ID NO: 315 - protein, 108 aa, synthetic construct
  REGION 1..108: note = TLA0241-VL-8
DIQMTQSPSSVSASVGDRVTITCSTSSSIISNYSHWYQQKPGKAPKLLIYKTSNLPSGVPSRFSGSGSGTDFTLTISSLQPEDFATYYCQQGSDMPITFGQGTKVEIK

SEQ ID NO: 316 - protein, 108 aa, synthetic construct
  REGION 1..108: note = TLA0241-VL-9
EIVLTQSPTTLSLSPGEKVTITCSASSSIISNYLHWYQQKPDQSPKLLIYKTSNLPSGVPSRFSGSGSGTDFTLTINSLEAEDAATYYCQQGSDMPITFGAGTKLELK

SEQ ID NO: 317 - protein, 108 aa, synthetic construct
  REGION 1..108: note = TLA0241-VL-10
EIVLTQSPPTLSLSPGEKVTITCSASSSIISNYLAWYQQKPGKSPKLLIYKTSNLPSGVPSRFSGSGSGTSFTLTINSLEAEDAATYYCQQGSDMPITFGAGTKLELK

SEQ ID NO: 318 - protein, 107 aa, synthetic construct [sequence omitted]

SEQ ID NO: 319 - protein, 103 aa, synthetic construct
  REGION 1..103: note = CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 320 - protein, 227 aa, synthetic construct
  REGION 1..227: note = Fc(L234A, L235A)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 227 aa, synthetic construct
  REGION 1..227: note = Fc(L234A, L235A, M252Y, S254T, T256E)
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 124 aa, synthetic construct
  REGION 1..124: note = RVT-3101-VH
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 323 - protein, 107 aa, synthetic construct
  REGION 1..107: note = RVT-3101-VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 324 - protein, 116 aa, synthetic construct
  REGION 1..116: note = MK-7240-VH
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSS

SEQ ID NO: 325 - protein, 106 aa, synthetic construct
  REGION 1..106: note = MK-7240-VL
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIK

SEQ ID NO: 326 - protein, 251 aa, Homo sapiens
  REGION 1..251: note = Human TL1A (M1-L251)
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 327 - protein, 180 aa, Homo sapiens
  REGION 1..180: note = Human sTL1A (L72-L251)
LKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 328 - protein, 180 aa, synthetic construct
  REGION 1..180: note = sTL1A (L72-L251)
LKGQEFAPSHQQVYAPLRADGDKPRAHLTVVRQTPTQHLKNQFPALHWEHELGLAFTKNRMNYTNKFLLIPESGDYFVYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 329 - protein, 192 aa, Mus musculus
  REGION 1..192: note = Mouse sTL1A (A61-L252)
AGQLRVPGKDCMLRAITEERSEPSPQQVYSPPRGKPRAHLTIKKQTPAPHLKNQLSALHWEHDLGMAFTKNGMKYINKSLVIPESGDYFIYSQITFRGTTSVCGDISRGRRPNKPDSITVVITKVADSYPEPARLLTGSKSVCEISNNWFQSLYLGAMFSLEEGDRLMVNVSDISLVDYTKEDKTFFGAFLL

SEQ ID NO: 330 - protein, 192 aa, Rattus norvegicus
  REGION 1..192: note = Rat sTL1A (T61-I252)
TGQLRIPGKDCMFPTVTEERSAPSAQPVYTPSRDKPKAHLTIMRQTPVPHLKNELAALHWENNLGMAFTKNRMNYTNKFLVIPESGDYFIYSQITFRGTTSECGDISRVRRPKKPDSITVVITKVADSYPEPAHLLTGTKSVCEISSNWFQPIYLGAMFSLEEGDRLMVNVSDISLVDYTKEDKTFFGAFLI

SEQ ID NO: 331 - protein, 175 aa, Homo sapiens
  REGION 1..175: note = Human DR3-ECD (Q25-Q199)
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQ

SEQ ID NO: 332 - protein, 5 aa, synthetic construct
  REGION 1..5: note = Vedolizumab-HCDR1
SYWMH

SEQ ID NO: 333 - protein, 17 aa, synthetic construct
  REGION 1..17: note = Vedolizumab-HCDR2
EIDPSESNTNYNQKFKG

SEQ ID NO: 334 - protein, 12 aa, synthetic construct
  REGION 1..12: note = Vedolizumab-HCDR3
GGYDGWDYAIDY

SEQ ID NO: 335 - protein, 121 aa, synthetic construct [sequence omitted]

SEQ ID NO: 336 - protein, 16 aa, synthetic construct
  REGION 1..16: note = Vedolizumab-LCDR1
RSSQSLAKSYGNTYLS

SEQ ID NO: 337 - protein, 7 aa, synthetic construct
  REGION 1..7: note = Vedolizumab-LCDR2
GISNRFS

SEQ ID NO: 338 - protein, 9 aa, synthetic construct
  REGION 1..9: note = Vedolizumab-LCDR3
LQGTHQPYT

SEQ ID NO: 339 - protein, 112 aa, synthetic construct [sequence omitted]

SEQ ID NO: 340 - protein, 243 aa, synthetic construct
  REGION 1..243: note = scTLA0015-11 VHVL
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGQGTKLEIK

SEQ ID NO: 341 - protein, 243 aa, synthetic construct
  REGION 1..243: note = scTLA0015-11 VLVH
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGQGTKLEIKGGGGSGGGGSGGGGSGGGGSEIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 342 - protein, 243 aa, synthetic construct
  REGION 1..243: note = scTLA0015-11cc VHVL
EIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 343 - protein, 243 aa, synthetic construct
  REGION 1..243: note = scTLA0015-11cc VLVH
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSS

SEQ ID NO: 344 - protein, 245 aa, synthetic construct
  REGION 1..245: note = scTLA0238-5 VHVL
EVQLVESGGGLVQPGGSLRLSCAASGFTFSNYWMNWVRQAPGKGLEWVAQIRLKSDNYATHYADSVKGRFTISRDNSKNTLYLQMNSLRAEDTAVYYCTPLLLRYRDYWGQGTLVTVSSGGGGSGGGGSGGGGSGGGGSDIQMTQSPSSVSASVGDRVTITCSASSSVSYMHWYQQKPGKAPKLWIYDTSNLASGVPSRFSGSGSGTDFTLTISSLQPEDFATYYCFQENGHPFTFGQGTKVEIK

SEQ ID NO: 345 - protein, 245 aa, synthetic construct
  REGION 1..245: note = scTLA0238-5cc VHVL
EVQLVESGGGLVQPGGSLRLSCAASGFTFSNYWMNWVRQAPGKCLEWVAQIRLKSDNYATHYADSVKGRFTISRDNSKNTLYLQMNSLRAEDTAVYYCTPLLLRYRDYWGQGTLVTVSSGGGGSGGGGSGGGGSGGGGSDIQMTQSPSSVSASVGDRVTITCSASSSVSYMHWYQQKPGKAPKLWIYDTSNLASGVPSRFSGSGSGTDFTLTISSLQPEDFATYYCFQENGHPFTFGCGTKVEIK

SEQ ID NO: 346 - protein, 253 aa, synthetic construct
  REGION 1..253: note = scVedolizumab VHVL
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSGGGGSGGGGSGGGGSGGGGSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIK

SEQ ID NO: 347 - protein, 253 aa, synthetic construct
  REGION 1..253: note = scVedolizumab-cc VHVL
QVQLVQSGAEVKKPGASVKVSCKGSGYTFTSYWMHWVRQAPGQCLEWIGEIDPSESNTNYNQKFKGRVTLTVDISASTAYMELSSLRSEDTAVYYCARGGYDGWDYAIDYWGQGTLVTVSSGGGGSGGGGSGGGGSGGGGSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGCGTKVEIK

SEQ ID NO: 348 - protein, 219 aa, synthetic construct
  REGION 1..219: note = light chain 1
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 349 - protein, 214 aa, synthetic construct
  REGION 1..214: note = light chain 2
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 350 - protein, 714 aa, synthetic construct
  REGION 1..714: note = TLb0045 heavy chain
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 351 - protein, 714 aa, synthetic construct
  REGION 1..714: note = TLb0046 heavy chain
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 714 aa, synthetic construct
  REGION 1..714: note = TLb0065 heavy chain
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGQGTKLEIK

SEQ ID NO: 353 - protein, 714 aa, synthetic construct
  REGION 1..714: note = TLb0066 heavy chain
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGQGTKLEIKGGGGSGGGGSGGGGSGGGGSEIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSSGGGGSGGGGSGGGGSGGGGSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 354 - protein, 719 aa, synthetic construct
  REGION 1..719: note = TLb0067 heavy chain
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSGGGGSGGGGSGGGGSGGGGSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIK

SEQ ID NO: 355 - protein, 719 aa, synthetic construct
  REGION 1..719: note = TLb0068 heavy chain
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKGGGGSGGGGSGGGGSGGGGSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSGGGGSGGGGSGGGGSGGGGSEIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 356 - protein, 716 aa, synthetic construct
  REGION 1..716: note = TLb0081 heavy chain
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFTFSNYWMNWVRQAPGKGLEWVAQIRLKSDNYATHYADSVKGRFTISRDNSKNTLYLQMNSLRAEDTAVYYCTPLLLRYRDYWGQGTLVTVSSGGGGSGGGGSGGGGSGGGGSDIQMTQSPSSVSASVGDRVTITCSASSSVSYMHWYQQKPGKAPKLWIYDTSNLASGVPSRFSGSGSGTDFTLTISSLQPEDFATYYCFQENGHPFTFGQGTKVEIK

SEQ ID NO: 357 - protein, 722 aa, synthetic construct
  REGION 1..722: note = TLb0085 heavy chain
EVQLVESGGGLVQPGGSLRLSCAASGFTFSNYWMNWVRQAPGKGLEWVAQIRLKSDNYATHYADSVKGRFTISRDNSKNTLYLQMNSLRAEDTAVYYCTPLLLRYRDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSGGGGSGGGGSGGGGSGGGGSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIK

SEQ ID NO: 358 - protein, 719 aa, synthetic construct
  REGION 1..719: note = TLb0086 heavy chain
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGAEVKKPGASVKVSCKGSGYTFTSYWMHWVRQAPGQCLEWIGEIDPSESNTNYNQKFKGRVTLTVDISASTAYMELSSLRSEDTAVYYCARGGYDGWDYAIDYWGQGTLVTVSSGGGGSGGGGSGGGGSGGGGSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGCGTKVEIK

SEQ ID NO: 359 - protein, 716 aa, synthetic construct
  REGION 1..716: note = TLb0087 heavy chain
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFTFSNYWMNWVRQAPGKCLEWVAQIRLKSDNYATHYADSVKGRFTISRDNSKNTLYLQMNSLRAEDTAVYYCTPLLLRYRDYWGQGTLVTVSSGGGGSGGGGSGGGGSGGGGSDIQMTQSPSSVSASVGDRVTITCSASSSVSYMHWYQQKPGKAPKLWIYDTSNLASGVPSRFSGSGSGTDFTLTISSLQPEDFATYYCFQENGHPFTFGCGTKVEIK

SEQ ID NO: 360 - protein, 722 aa, synthetic construct
  REGION 1..722: note = TLb0088 heavy chain
EVQLVESGGGLVQPGGSLRLSCAASGFTFSNYWMNWVRQAPGKGLEWVAQIRLKSDNYATHYADSVKGRFTISRDNSKNTLYLQMNSLRAEDTAVYYCTPLLLRYRDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGAEVKKPGASVKVSCKGSGYTFTSYWMHWVRQAPGQCLEWIGEIDPSESNTNYNQKFKGRVTLTVDISASTAYMELSSLRSEDTAVYYCARGGYDGWDYAIDYWGQGTLVTVSSGGGGSGGGGSGGGGSGGGGSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGCGTKVEIK

SEQ ID NO: 361 - protein, 714 aa, synthetic construct
  REGION 1..714: note = TLb0093 heavy chain
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSS

SEQ ID NO: 362 - protein, 714 aa, synthetic construct
  REGION 1..714: note = TLb0094 heavy chain
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGQGTKLEIKGGGGSGGGGSGGGGSGGGGSEIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 363 - protein, 714 aa, synthetic construct
  REGION 1..714: note = TLb0045-1 heavy chain
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 364 - protein, 704 aa, synthetic construct
  REGION 1..704: note = TLb0045-2 heavy chain
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 365 - protein, 709 aa, synthetic construct
  REGION 1..709: note = TLb0045-3 heavy chain
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 366 - protein, 714 aa, synthetic construct
  REGION 1..714: note = TLb0065-1 heavy chain
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSEIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGQGTKLEIK

SEQ ID NO: 367 - protein, 446 aa, synthetic construct
  REGION 1..446: note = TLb0069 heavy chain-1
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - protein, 485 aa, synthetic construct
  REGION 1..485: note = TLb0069 heavy chain-2
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGCGTKVEIKGGGGSGGGGSGGGGSGGGGSQVQLVQSGAEVKKPGASVKVSCKGSGYTFTSYWMHWVRQAPGQCLEWIGEIDPSESNTNYNQKFKGRVTLTVDISASTAYMELSSLRSEDTAVYYCARGGYDGWDYAIDYWGQGTLVTVSSEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 369 - protein, 451 aa, synthetic construct
  REGION 1..451: note = TLb0070 heavy chain-1
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - protein, 475 aa, synthetic construct
  REGION 1..475: note = TLb0070 heavy chain-2
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 371 - protein, 227 aa, synthetic construct
  REGION 1..227: note = Fc(LALA+LS)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 372 - protein, 213 aa, synthetic construct
  REGION 1..213: note = light chain 3
DIQMTQSPSSVSASVGDRVTITCSASSSVSYMHWYQQKPGKAPKLWIYDTSNLASGVPSRFSGSGSGTDFTLTISSLQPEDFATYYCFQENGHPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 373 - protein, 227 aa, synthetic construct
  REGION 1..227: note = Fc-Knob
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 374 - protein, 227 aa, synthetic construct
  REGION 1..227: note = Fc-Hole
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 375 - protein, 251 aa, synthetic construct
  REGION 1..251: note = scRVT-3101 VHVL
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 376 - protein, 251 aa, synthetic construct
  REGION 1..251: note = scRVT-3101 VLVH
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKGGGGSGGGGSGGGGSGGGGSQVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 377 - protein, 251 aa, synthetic construct
  REGION 1..251: note = scRVT-3101cc
QVQLVQSGAEVKKPGASVKVSCKASGYDFTYYGISWVRQAPGQCLEWMGWISTYNGNTHYARMLQGRVTMTTDTSTRTAYMELRSLRSDDTAVYYCARENYYGSGAYRGGMDVWGQGTTVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGCGTKVEIK

SEQ ID NO: 378 - protein, 722 aa, synthetic construct
  REGION 1..722: note = TLb0111-heavy chain
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSQVQLVQSGAEVKKPGASVKVSCKASGYDFTYYGISWVRQAPGQCLEWMGWISTYNGNTHYARMLQGRVTMTTDTSTRTAYMELRSLRSDDTAVYYCARENYYGSGAYRGGMDVWGQGTTVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGCGTKVEIK

SEQ ID NO: 379 - protein, 116 aa, synthetic construct
EIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSS

SEQ ID NO: 380 - protein, 107 aa, synthetic construct
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 381 - protein, 227 aa, synthetic construct; identical to SEQ ID NO: 371

SEQ ID NO: 382 - protein, 30 aa, synthetic construct
QVQLVQSGAEVKKPGASVKVSCKGSGYTFT

SEQ ID NO: 383 - protein, 14 aa, synthetic construct
WVRQAPGQRLEWIG

SEQ ID NO: 384 - protein, 32 aa, synthetic construct
RVTLTVDISASTAYMELSSLRSEDTAVYYCAR

SEQ ID NO: 385 - protein, 11 aa, synthetic construct
WGQGTLVTVSS

SEQ ID NO: 386 - protein, 23 aa, synthetic construct
DVVMTQSPLSLPVTPGEPASISC

SEQ ID NO: 387 - protein, 15 aa, synthetic construct
WYLQKPGQSPQLLIY

SEQ ID NO: 388 - protein, 32 aa, synthetic construct
GVPDRFSGSGSGTDFTLKISRVEAEDVGVYYC

SEQ ID NO: 389 - protein, 10 aa, synthetic construct
FGQGTKVEIK

(19) United States
(12) Patent Application Publication
(10) Pub. No.: US 2026/0062471 A1
(43) Pub. Date: Mar. 5, 2026

(54) ANTIBODY AND USE THEREOF

(71) Applicant: EARENDIL LABS INC., Wilmington, DE (US)

(72) Inventors: Hao RAN, Suzhou, Jiangsu (CN); Jian HUANG, Suzhou, Jiangsu (CN); Zhenping ZHU, Suzhou, Jiangsu (CN); Jian PENG, Suzhou, Jiangsu (CN); Liang TIAN, Suzhou, Jiangsu (CN); Rui SHEN, Suzhou, Jiangsu (CN)

(21) Appl. No.: 19/312,682

(22) Filed: Aug. 28, 2025

(30) Foreign Application Priority Data
Aug. 28, 2024 (CN) 202411195305.2
Sep. 30, 2024 (CN) 202411388891.2
Jan. 27, 2025 (CN) 202510127573.9
Feb. 5, 2025 (CN) 202510130736.9
May 30, 2025 (CN) 202510717116.5

(51) Int. Cl. C07K 16/24; A61K 38/00; A61P 29/00; C07K 16/28; C12N 15/11

(52) U.S. Cl. CPC C07K 16/241; A61K 38/00; A61P 29/00; C07K 16/2839; C12N 15/11; C07K 2317/31; C07K 2317/565; C07K 2317/622; C07K 2317/76; C07K 2317/92

(57) ABSTRACT

The present application provides an isolated antigen-binding protein, comprising: a first antigen-binding domain and a second antigen-binding domain, wherein the first antigen-binding domain specifically binds to TNF-like protein A (TL1A), and the first antigen binding domain and the second antigen binding domain are different.

### RELATED APPLICATIONS

This application claims priority to Chinese Patent Application Nos. 202510717116.5, filed May 30, 2025, 202510130736.9, filed Feb. 5, 2025, 202510127573.9, filed Jan. 27, 2025, 202411388891.2, filed Sep. 30, 2024, and 202411195305.2, filed Aug. 28, 2024, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Sep. 2, 2025, is named 769255_SA9-943_ST26.xml and is 572,213 bytes in size.

### FIELD OF THE INVENTION

The present application relates to the field of biomedicine, and specifically to a TL1A antigen-binding protein.

### BACKGROUND OF THE INVENTION

TL1A is encoded by the TNFSF15 gene and is also known as vascular growth inhibitory factor (VEGI), a member of the tumor necrosis factor (TNF) ligand superfamily. TL1A is a type II transmembrane protein. TL1A is initially expressed on the surface of the cell as a membrane-bound protein, and then is sheared by extracellular matrix metalloproteinases to form a soluble form of TL1A. It has been shown that TL1A is mainly expressed in antigen-presenting cells and lymphocytes, as well as endothelial cells and fibroblasts stimulated by inflammatory factors. TL1A is mainly expressed in antigen-presenting cells and lymphocytes, as well as endothelial cells and fibroblasts stimulated by inflammatory factors. TL1A induces the activation of downstream NF-κB and MAPK cascade (e.g., ERK, p38, JNK) signaling pathways by binding to and activating its receptor DR3, which regulates the cell proliferation, survival, or apoptosis of the cells. TL1A-DR3 signaling plays an important role in the regulation of the immune system. The TL1A-DR3 signaling pathway plays an important role in the regulation of the immune system, which is mainly reflected in the following aspects: 1) synergistically promoting Th1 activation and cytokine secretion. Under the condition of TCR activation, TL1A can induce the production and secretion of proinflammatory factors, mainly including: IFN-γ, GM-CSF, TNF-α, etc. It can also synergize with IL12/IL18 factors to promote IFN-γ production by T cells, and the effect is not dependent on the presence of antigen. 2) Synergistically promoting Th2 activation, sustained overexpression of TL1A in T or DC cells in mice produces an intestinal inflammation that is dependent on the Th2 cytokine IL13. In addition, TL1A also promotes Th2 immune responses and allergen-specific antibody production in a mouse model of asthma. 3) Synergistically promotes the proliferation and activation of Th17-type cells, and inhibition of TL1A helps to alleviate the symptoms of DSS-induced intestinal inflammation in mice. 4) TL1A also promotes the activation and cell-killing capacity of NK and NKT cells. 5) TL1A also promotes the activation and cell-killing capacity of NK and NKT cells. 6) TL1A also promotes the activation of NK and NKT cells.

Antibodies targeting inhibition of TL1A have entered clinical trials and achieved positive therapeutic effects in patients with inflammatory bowel disease, and still have some efficacy in patients with resistance to or relapse of frontline biologics, but as of now, all kinds of drugs targeting inhibition of TL1A are still in the clinical stage, and there are no drugs on the market. For example, the results of phase II clinical trial of PRA023/MK7240 for Crohn's disease showed that 49 of patients achieved clinical remission and 26 of patients achieved endoscopic remission; while the results of phase II clinical trial of PRA023/MK7240 for ulcerative colitis showed that 26.5 of patients achieved clinical remission and 36.8 of patients achieved endoscopic remission. Similar therapeutic effects were also seen in the Phase II clinical trial of RVT-3101/RG6631. Overall, monoclonal antibodies targeting TL1A inhibition have shown positive therapeutic effects in re-inflammatory bowel disease, but only a fraction of patients have achieved clinical benefit, and the overall efficacy needs to be further improved.

Integrins are transmembrane glycoproteins expressed on the cell surface that have important functions in immune cell proliferation, signaling, and cell migration by mediating cell-cell interactions. Integrins are heterodimers consisting of an α-subunit and a β-subunit Integrin α4β7, a heterodimer of α4 and β7 subunits, is mainly expressed in memory T cells and memory B cells, and is important for the localization and migration of immune cells to intestinal mucosal tissues. α4β7 mediates the migration of immune cells by interacting with its ligand, MAdCAM-1, which is mainly expressed in the vascular endothelial cells of the small intestine. Currently, vedolizumab, a monoclonal antibody targeting inhibition of α4β7, is approved and marketed for the treatment of moderate-to-severe ulcerative colitis and Crohn's disease. In ulcerative colitis, vedolizumab resulted in better clinical response and disease remission in 21 of patients after 6 weeks of treatment and in 42 of patients after extending treatment to 52 weeks. In contrast, in Crohn's disease, Vedolizumab treatment for 6 weeks resulted in a better response and disease remission in 15 of patients, and after extending treatment to 52 weeks, a better clinical response and disease remission was achieved in 39% of patients.

Over the past two decades, biomolecules represented by vedolizumab, a monoclonal antibody targeting integrin α4β7, have achieved remarkable efficacy in the treatment of ulcerative colitis, driving new breakthroughs in clinical remission rates in ulcerative colitis. However, most current treatments have 1-year clinical remission rates between 30% and 50%, and more than 50 of patients still have inadequate clinical response, suggesting that the therapeutic ceiling may have been reached through the use of a single agent. In addition, there is still a huge unmet clinical need for effective yet safe therapeutic agents for patients with moderate-to-severe disease, and newer drug development is urgently needed to improve treatment outcomes.

Ulcerative colitis is the result of multiple pathways driving immune-mediated inflammatory processes, and drugs that are effective in intestinal disease may be ineffective in extraintestinal manifestations or concomitant immune-mediated inflammatory disease. With this in mind, the concept of combination therapy based on the use of two biologics may be a promising strategy for patients with refractory ulcerative colitis, patients at high risk of developing complications, or patients with concomitant uncontrolled immune-mediated inflammatory disease. This strategy is currently being applied in real-world treatment with good results. 2022 A systematic review and meta-analysis of combination therapy published in 2022 (Ahmed, W., et al. Clin Gastroenterol Hepatol. 2022) showed that in 279 patients who received combination therapy in a total of 30 included studies. Clinical remission and endoscopic remission rates reached 59% and 34%, respectively. This included 81 of patients with refractory ulcerative colitis and 12 of patients with ulcerative colitis complicated by extraintestinal manifestations or rheumatologic disease. The most common combinations in the treatment regimen included anti-integrin monoclonal antibody+anti-TNF-α monoclonal antibody (48%) and anti-integrin monoclonal antibody+Ustekinumab (19%).

### SUMMARY OF THE INVENTION

An isolated antigen-binding protein comprising: a first antigen-binding domain and a second antigen-binding domain, the first antigen-binding domain specifically binds to TNF-like protein A (TL1A), and the first antigen-binding domain is different from the second antigen-binding domain.

In some embodiments, wherein,
1) in the presence of monomeric TL1A and/or trimeric TL1A, an effect of down-regulating the expression of cell membrane α4β7 is enhanced, preferably, said antigen-binding protein or protein comprising the first antigen-binding domain forms a crosslinked structure with the monomeric TL1A and/or trimeric TL1A; or,
2) in the presence of α4β7, an effect of down-regulating the expression of monomeric TL1A and/or trimeric TL1A is enhanced, preferably, said antigen-binding protein or protein comprising the second antigen-binding domain forms a crosslinked structure with the α4β7 protein.

In some embodiments, the isolated antigen-binding protein mediates internalization of the cell membrane α4β7 or TL1A.

In some embodiments, wherein,
1) in the presence of monomeric TL1A and/or trimeric TL1A, an effect of mediating the occurrence of the internalization in the cell membrane α4β7 of the subject is enhanced, or;
2) in the presence of α4β7, an effect of mediating the occurrence of the internalization in the cell membrane α4β7 of the subject is enhanced.

In some embodiments, the isolated antigen-binding protein specifically binds to the α4β7 protein with a KD value of approximately 5E-08M or less.

In some embodiments, the isolated antigen-binding protein specifically binds to the monomeric TL1A/or trimeric TL1A with a KD value of approximately 5E-08M or less.

In some embodiments, a binding site of the first antigen-binding domain of the antigen-binding protein to the TL1A is selected from one or more of the following: R32, Q33, P35, T36, Q37, P44, E49, E51, L54, A56, S89, K102, P103, Y117, M146, S148, D161, I162, S163, L164 and Y167; and the binding sites are numbered according to an amino acid sequence of TL1A as set forth in SEQ ID NO: 327.

In some embodiments, a binding site of the first antigen-binding domain to the TL1A is selected from one or more of the following: R32, Q33, P35, T36, Q37, P44, S89, K102, P103, D161, I162, S163 and L164, including any one, two, three, four, five, six, seven, eight, nine, ten, eleven, twelve or thirteen of the above 13 sites; and the binding sites are numbered according to the amino acid sequence of TL1A as set forth in SEQ ID NO: 327.

In some embodiments, a binding site of the antigen-binding protein to the TL1A is selected from one or more of the following: R32, E49, E51, L54, A56, Y117, M146, S148, and Y167, including any one, two, three, four, five, six, seven, eight, and nine of the above 9 sites; and the binding sites are numbered according to the amino acid sequence of TL1A as set forth in SEQ ID NO: 327.

In some embodiments, the isolated antigen-binding protein downregulates the expression of the α4β7 or the monomeric TL1A and/or the trimer TL1A.

In some embodiments, the isolated antigen-binding protein downregulates the expression of the membrane α4β7 or the monomeric TL1A and/or the trimer TL1A.

In some embodiments, the isolated antigen-binding protein comprises one or more polypeptides, wherein one of the polypeptides comprises at least one first antigen-binding domain and at least one second antigen-binding domain.

In some embodiments, the isolated antigen-binding protein comprises a first polypeptide and a second polypeptide, wherein the first polypeptide comprises the first antigen-binding domain and the second antigen-binding domain, and the second polypeptide comprises a first antigen-binding domain or a second antigen-binding domain, and one or more of the following conditions are selected:
(1) the first antigen-binding domain of the first polypeptide is located at the C-terminus of the first polypeptide, and the first antigen-binding domain of the second polypeptide is located at the C-terminus of the first polypeptide;
(2) the first antigen-binding domain of the first polypeptide is located at the N-terminus of the first polypeptide, and the first antigen-binding domain of the second polypeptide is located at the N-terminus of the first polypeptide;
(3) the second antigen-binding domain of the first polypeptide is located at the N-terminus of the first polypeptide, and the second antigen-binding domain of the second polypeptide is located at the N-terminus of the second polypeptide;
(4) the second antigen-binding domain of the first polypeptide is located at the C-terminus of the second polypeptide, and the second antigen-binding domain of the second polypeptide is located at the C-terminus of the second polypeptide.

In some embodiments, the isolated antigen-binding protein comprises a first polypeptide and a second polypeptide, wherein the first polypeptide comprises the first antigen-binding domain and the second antigen-binding domain, and the second polypeptide comprises the first antigen-binding domain and the second antigen-binding domain, and one or more of the following conditions are selected:
(1) the first antigen-binding domain of the first polypeptide is located at the C-terminus of the first polypeptide, and the first antigen-binding domain of the second polypeptide is located at the C-terminus of the first polypeptide;
(2) the first antigen-binding domain of the first polypeptide is located at the N-terminus of the first polypeptide, and the first antigen-binding domain of the second polypeptide is located at the N-terminus of the first polypeptide;
(3) the second antigen-binding domain of the first polypeptide is located at the N-terminus of the first polypeptide, and the second antigen-binding domain of the second polypeptide is located at the N-terminus of the second polypeptide;
(4) the second antigen-binding domain of the first polypeptide is located at the C-terminus of the second polypeptide, and the second antigen-binding domain of the second polypeptide is located at the C-terminus of the second polypeptide.

In some embodiments, the isolated antigen-binding protein comprises a first polypeptide and a second polypeptide, wherein the first polypeptide comprises the first antigen-binding domain and does not include the second antigen-binding domain, and the second polypeptide comprises the second antigen-binding domain and does not include the first antigen-binding domain.

In some embodiments, the first antigen-binding domain or the second antigen-binding domain comprises one or any combination of the following:

Fab, Fab′, F(ab′)2, Fd and Fv fragments, disulfide bond stabilized Fv fragments (dsFv)., (dsFv)2, bispecific dsFv (dsFv-dsFv′), disulfide bond stabilized double-stranded antibody (diabody) (ds double-stranded antibody), single-chain antibody molecule (scFv), Single domain antibody (SDAB), scFv dimer (divalent double-stranded antibody), single domain antibody or nanobody, domain antibody (domain antibody (dAb), shark variable IgNAR domain, camelized VH domain, chicken heavy chain antibody domain, VHH domain, minimum identification unit composed of CDR of mimetic antibody (minimal recognition unit), alternative scaffolds for binding antigens, bivalent domain antibodies, pre-designed ankyrin repeat proteins (DARPins), multispecific proteins (including antigen-binding fragments or any other antibody fragments bound to antigens, but not intact antibody structures).

In some embodiments, the first antigen-binding domain comprises a heavy chain variable region (VH) containing an HCDR3 comprising an amino acid sequence of SEQ ID NO:14.

In some embodiments, the HCDR3 comprises an amino acid sequence of any one of SEQ ID NO:15-18.

In some embodiments, the VH comprises HCDR2 comprising an amino acid sequence of SEQ ID NO:5.

In some embodiments, the HCDR2 comprises an amino acid sequence of any one of SEQ ID NO:6-13.

In some embodiments, the VH comprises HCDR1 comprising an amino acid sequence of SEQ ID NO:1.

In some embodiments, the HCDR1 comprises an amino acid sequence of any one of the SEQ ID NO:2-4.

In some embodiments, the VH comprises the HCDR1 as shown in SEQ ID NO: 2, the HCDR2 as shown in SEQ ID NO: 6, and the HCDR3 as shown in SEQ ID NO: 15.

In some embodiments, the VH comprises the HCDR1 as shown in SEQ ID NO: 2, the HCDR2 as shown in SEQ ID NO: 7, and the HCDR3 as shown in SEQ ID NO: 15.

In some embodiments, the VH comprises the HCDR1 as shown in SEQ ID NO: 2, the HCDR2 as shown in SEQ ID NO: 8, and the HCDR3 as shown in SEQ ID NO: 15.

In some embodiments, the VH comprises the HCDR1 as shown in SEQ ID NO: 3, the HCDR2 as shown in SEQ ID NO: 6, and the HCDR3 as shown in SEQ ID NO: 15.

In some embodiments, the VH comprises the HCDR1 as shown in SEQ ID NO: 2, the HCDR2 as shown in SEQ ID NO: 6, and the HCDR3 as shown in SEQ ID NO: 16.

In some embodiments, the VH comprises the HCDR1 as shown in SEQ ID NO: 2, the HCDR2 as shown in SEQ ID NO: 6, and the HCDR3 as shown in SEQ ID NO: 17.

In some embodiments, the VH comprises the HCDR1 as shown in SEQ ID NO: 2, the HCDR2 as shown in SEQ ID NO: 6, and the HCDR3 as shown in SEQ ID NO: 18.

In some embodiments, the VH comprises the HCDR1 as shown in SEQ ID NO: 4, the HCDR2 as shown in SEQ ID NO: 6, and the HCDR3 as shown in SEQ ID NO: 15.

In some embodiments, the VH comprises the HCDR1 as shown in SEQ ID NO: 2, the HCDR2 as shown in SEQ ID NO: 9, and the HCDR3 as shown in SEQ ID NO: 15.

In some embodiments, the VH comprises the HCDR1 as shown in SEQ ID NO: 2, the HCDR2 as shown in SEQ ID NO: 10, and the HCDR3 as shown in SEQ ID NO: 15.

In some embodiments, the VH comprises the HCDR1 as shown in SEQ ID NO: 2, the HCDR2 as shown in SEQ ID NO: 11, and the HCDR3 as shown in SEQ ID NO: 15.

In some embodiments, the VH comprises the HCDR1 as shown in SEQ ID NO: 2, the HCDR2 as shown in SEQ ID NO: 12, and the HCDR3 as shown in SEQ ID NO: 15.

In some embodiments, the VH comprises the HCDR1 as shown in SEQ ID NO: 2, the HCDR2 as shown in SEQ ID NO: 13, and the HCDR3 as shown in SEQ ID NO: 15.

In some embodiments, the VH comprises the HCDR1 as shown in SEQ ID NO:3, the HCDR2 as shown in SEQ ID NO:6, and the HCDR3 as shown in SEQ ID NO: 16.

In some embodiments, the VH comprises the HCDR1 as shown in SEQ ID NO: 3, the HCDR2 as shown in SEQ ID NO: 6, and the HCDR3 as shown in SEQ ID NO: 17.

In some embodiments, the VH comprises the HCDR1 as shown in SEQ ID NO: 3, the HCDR2 as shown in SEQ ID NO: 9, and the HCDR3 as shown in SEQ ID NO: 15.

In some embodiments, the VH comprises the HCDR1 as shown in SEQ ID NO:3, the HCDR2 as shown in SEQ ID NO:10 and the HCDR3 as shown in SEQ ID NO: 15.

In some embodiments, the VH comprises the HCDR1 as shown in SEQ ID NO: 3, the HCDR2 as shown in SEQ ID NO: 11, and the HCDR3 as shown in SEQ ID NO: 15.

In some embodiments, the VH comprises the HCDR1 as shown in SEQ ID NO: 2, the HCDR2 as shown in SEQ ID NO: 9, and the HCDR3 as shown in SEQ ID NO: 16.

In some embodiments, the VH comprises the HCDR1 as shown in SEQ ID NO: 2, the HCDR2 as shown in SEQ ID NO: 10, and the HCDR3 as shown in SEQ ID NO: 16.

In some embodiments, the VH comprises the HCDR1 as shown in SEQ ID NO:2, the HCDR2 as shown in SEQ ID NO:11, and the HCDR3 as shown in SEQ ID NO: 16.

In some embodiments, the VH comprises the HCDR1 as shown in SEQ ID NO: 2, the HCDR2 as shown in SEQ ID NO: 12, and the HCDR3 as shown in SEQ ID NO: 16.

In some embodiments, the VH comprises the HCDR1 as shown in SEQ ID NO:2, the HCDR2 as shown in SEQ ID NO:10, and the HCDR3 as shown in SEQ ID NO:17.

In some embodiments, the VH comprises an HFR1 containing an amino acid sequence of SEQ ID NO:19.

In some embodiments, the HFR1 is selected from an amino acid sequence shown in any one of SEQ ID NO:20-28.

In some embodiments, the VH comprises an HFR2, and the HFR2 comprises an amino acid sequence of SEQ ID NO:29.

In some embodiments, the HFR2 is selected from an amino acid sequence shown in any one of SEQ ID NO:30-32.

In some embodiments, the VH comprises an HFR3 comprising an amino acid sequence of SEQ ID NO:33.

In some embodiments, the HFR3 is selected from an amino acid sequence shown in any one of SEQ ID NO:34-40.

In some embodiments, the VH comprises an HFR4 comprising an amino acid sequence shown in SEQ ID NO:41.

In some embodiments, the VH comprises the HFR1 as shown in SEQ ID NO: 20, the HFR2 as shown in SEQ ID NO: 30, the HFR3 as shown in SEQ ID NO: 34, and the HFR4 as shown in SEQ ID NO: 41.

In some embodiments, the VH comprises the HFR1 as shown in SEQ ID NO: 21, the HFR2 as shown in SEQ ID NO: 31, the HFR3 as shown in SEQ ID NO: 35, and the HFR4 as shown in SEQ ID NO: 41.

In some embodiments, the VH comprises the HFR1 as shown in SEQ ID NO: 22, the HFR2 as shown in SEQ ID NO: 32, the HFR3 as shown in SEQ ID NO: 35, and the HFR4 as shown in SEQ ID NO: 41.

In some embodiments, the VH comprises the HFR1 as shown in SEQ ID NO:23, the HFR2 as shown in SEQ ID NO:31, the HFR3 as shown in SEQ ID NO:36, and the HFR4 as shown in SEQ ID NO:41.

In some embodiments, the VH comprises the HFR1 as shown in SEQ ID NO: 23, the HFR2 as shown in SEQ ID NO: 31, the HFR3 as shown in SEQ ID NO: 37, and the HFR4 as shown in SEQ ID NO: 41.

In some embodiments, the VH comprises the HFR1 as shown in SEQ ID NO: 23, the HFR2 as shown in SEQ ID NO: 31, the HFR3 as shown in SEQ ID NO: 35, and the HFR4 as shown in SEQ ID NO: 41.

In some embodiments, the VH comprises the HFR1 as shown in SEQ ID NO: 24, the HFR2 as shown in SEQ ID NO: 31, the HFR3 as shown in SEQ ID NO: 38, and the HFR4 as shown in SEQ ID NO: 41.

In some embodiments, the VH comprises the HFR1 as shown in SEQ ID NO: 25, the HFR2 as shown in SEQ ID NO: 31, the HFR3 as shown in SEQ ID NO: 37, and the HFR4 as shown in SEQ ID NO: 41.

In some embodiments, the VH comprises the HFR1 as shown in SEQ ID NO: 25, the HFR2 as shown in SEQ ID NO: 31, the HFR3 as shown in SEQ ID NO: 39, and the HFR4 as shown in SEQ ID NO: 41.

In some embodiments, the VH comprises the HFR1 as shown in SEQ ID NO: 25, the HFR2 as shown in SEQ ID NO: 31, the HFR3 as shown in SEQ ID NO: 40, and the HFR4 as shown in SEQ ID NO: 41.

In some embodiments, the VH comprises the HFR1 as shown in SEQ ID NO: 26, the HFR2 as shown in SEQ ID NO: 31, the HFR3 as shown in SEQ ID NO: 35, and the HFR4 as shown in SEQ ID NO: 41.

In some embodiments, the VH comprises the HFR1 as shown in SEQ ID NO: 25, the HFR2 as shown in SEQ ID NO: 31, the HFR3 as shown in SEQ ID NO: 35, and the HFR4 as shown in SEQ ID NO: 41.

In some embodiments, the VH comprises the HFR1 as shown in SEQ ID NO: 27, the HFR2 as shown in SEQ ID NO: 31, the HFR3 as shown in SEQ ID NO: 35, and the HFR4 as shown in SEQ ID NO: 41.

In some embodiments, the VH comprises the HFR1 as shown in SEQ ID NO: 28, the HFR2 as shown in SEQ ID NO: 31, the HFR3 as shown in SEQ ID NO: 35, and the HFR4 as shown in SEQ ID NO: 41.

In some embodiments, the first antigen-binding domain of the isolated antigen-binding fragment further comprises a light chain variable region (VL) containing at least one light chain complementarity-determining region (LCDR) containing amino acid sequences selected from an amino acid sequence shown in SEQ ID NO:42, SEQ ID NO:43, and SEQ ID NO:44.

In some embodiments, the VL comprises an LCDR3 comprising an amino acid sequence of SEQ ID NO:44.

In some embodiments, the VL comprises an LCDR2 comprising an amino acid sequence of SEQ ID NO:43.

In some embodiments, the VL comprises an LCDR1 comprising an amino acid sequence of SEQ ID NO:42.

In some embodiments, the VL comprises the LFR1 comprising an amino acid sequence of SEQ ID NO:45.

In some embodiments, the LFR1 comprises an amino acid sequence shown in either of the SEQ ID NO:46-47.

In some embodiments, the VL comprises the LFR2 comprising an amino acid sequence of SEQ ID NO:48.

In some embodiments, the LFR2 comprises an amino acid sequence shown in either SEQ ID NO: 49-52.

In some embodiments, the VL comprises the LFR3 comprising an amino acid sequence of SEQ ID NO:53.

In some embodiments, the LFR3 comprises an amino acid sequence shown in either of the SEQ ID NO: 54-57.

In some embodiments, the VL comprises the LFR4 comprising an amino acid sequence of SEQ ID NO:58.

In some embodiments, the LFR1 comprise an amino acid as set forth in any one of SEQ ID NO:59-61.

In some embodiments, the VL comprises the LFR1 as shown in SEQ ID NO: 46, the LFR2 as shown in SEQ ID NO: 49, the LFR3 as shown in SEQ ID NO: 54, and the LFR4 as shown in SEQ ID NO: 59.

In some embodiments, the VL comprises the LFR1 as shown in SEQ ID NO: 47, the LFR2 as shown in SEQ ID NO: 50, the LFR3 as shown in SEQ ID NO: 55, and the LFR4 as shown in SEQ ID NO: 60.

In some embodiments, the VL comprises the LFR1 as shown in SEQ ID NO: 47, the LFR2 as shown in SEQ ID NO: 51, the LFR3 as shown in SEQ ID NO: 56, and the LFR4 as shown in SEQ ID NO: 61.

In some embodiments, the VL comprises the LFR1 as shown in SEQ ID NO: 47, the LFR2 as shown in SEQ ID NO: 52, the LFR3 as shown in SEQ ID NO: 57, and the LFR4 as shown in SEQ ID NO: 61.

In some embodiments, the VL comprises the LFR1 as shown in SEQ ID NO: 47, the LFR2 as shown in SEQ ID NO: 52, the LFR3 as shown in SEQ ID NO: 56, and the LFR4 as shown in SEQ ID NO: 61.

In some embodiments, the VH comprises a sequence as described in any one of SEQ ID NO:62-99.

In some embodiments, the VL comprises a sequence as described in any one of SEQ ID NO: 102-106.

An isolated antigen-binding protein comprising: a first antigen-binding domain and a second antigen-binding domain, the first antigen-binding domain specifically binds to TNF-like protein A (TL1A), and the first antigen-binding domain is different from the second antigen-binding domain.

In some embodiments, the first antigen-binding domain comprises a heavy chain variable region (VH) containing an amino acid sequence of an HCDR3 which comprises SEQ ID NO:120.

In some embodiments, the VH comprises an HCDR2 comprising an amino acid sequence of SEQ ID NO:115.

In some embodiments, the HCDR2 comprises an amino acid sequence of any one of SEQ ID NO: 116-119.

In some embodiments, the VH comprises an HCDR1 comprising an amino acid sequence of SEQ ID NO:107.

In some embodiments, the HCDR1 comprises an amino acid sequence of any one of SEQ ID NO: 108-114.

In some embodiments, the VH comprises the HCDR1 as shown in SEQ ID NO: 108, the HCDR2 as shown in SEQ ID NO: 116, and the HCDR3 as shown in SEQ ID NO: 120.

In some embodiments, the VH comprises the HCDR1 as shown in SEQ ID NO: 109, the HCDR2 as shown in SEQ ID NO: 118, and the HCDR3 as shown in SEQ ID NO: 120.

In some embodiments, the VH comprises the HCDR1 as shown in SEQ ID NO: 108, the HCDR2 as shown in SEQ ID NO: 119, and the HCDR3 as shown in SEQ ID NO: 120.

In some embodiments, the VH comprises the HCDR1 as shown in SEQ ID NO: 110, the HCDR2 as shown in SEQ ID NO: 118, and the HCDR3 as shown in SEQ ID NO: 120.

In some embodiments, the VH comprises the HCDR1 as shown in SEQ ID NO: 111, the HCDR2 as shown in SEQ ID NO: 117, and the HCDR3 as shown in SEQ ID NO: 120.

In some embodiments, the VH comprises the HCDR1 as shown in SEQ ID NO: 112, the HCDR2 as shown in SEQ ID NO: 117, and the HCDR3 as shown in SEQ ID NO: 120.

In some embodiments, the VH comprises the HCDR1 as shown in SEQ ID NO: 113, the HCDR2 as shown in SEQ ID NO: 117, and the HCDR3 as shown in SEQ ID NO: 120.

In some embodiments, the VH comprises the HCDR1 as shown in SEQ ID NO: 108, the HCDR2 as shown in SEQ ID NO: 117, and the HCDR3 as shown in SEQ ID NO: 120.

In some embodiments, the VH comprises the HCDR1 as shown in SEQ ID NO: 114, the HCDR2 as shown in SEQ ID NO: 117 and the HCDR3 as shown in SEQ ID NO: 120.

In some embodiments, the VH comprises an HFR1 which comprises an amino acid sequence of SEQ ID NO:121.

In some embodiments, the HFR1 is selected from an amino acid sequence shown in any one of SEQ ID NO:122-132.

In some embodiments, the VH comprises an HFR2, and the HFR2 comprises an amino acid sequence of SEQ ID NO:133.

In some embodiments, the HFR2 is selected from an amino acid sequence shown in any one of SEQ ID NO: 134-135, 30.

In some embodiments, the VH comprises an HFR3 comprising an amino acid sequence of SEQ ID NO:136.

In some embodiments, the HFR3 is selected from an amino acid sequence shown in any one of SEQ ID NO: 137-148.

In some embodiments, the VH comprises HFR4 comprising an amino acid sequence shown in SEQ ID NO:149.

In some embodiments, the HFR4 is selected from an amino acid sequence shown in ANY ONE of SEQ ID NO: 150-153, 41.

In some embodiments, the VH comprises the HFR1 as shown in SEQ ID NO: 122, the HFR2 as shown in SEQ ID NO: 134, the HFR3 as shown in SEQ ID NO: 137, and the HFR4 as shown in SEQ ID NO: 150.

In some embodiments, the VH comprises the HFR1 as shown in SEQ ID NO: 123, the HFR2 as shown in SEQ ID NO: 135, the HFR3 as shown in SEQ ID NO: 138, and the HFR4 as shown in SEQ ID NO: 41.

In some embodiments, the VH comprises the HFR1 as shown in SEQ ID NO: 124, the HFR2 as shown in SEQ ID NO: 135, the HFR3 as shown in SEQ ID NO: 139, and the HFR4 as shown in SEQ ID NO: 151.

In some embodiments, the VH comprises the HFR1 as shown in SEQ ID NO: 125, the HFR2 as shown in SEQ ID NO: 135, the HFR3 as shown in SEQ ID NO: 142, and the HFR4 as shown in SEQ ID NO: 41.

In some embodiments, the VH comprises the HFR1 as shown in SEQ ID NO: 123, the HFR2 as shown in SEQ ID NO: 135, the HFR3 as shown in SEQ ID NO: 140, and the HFR4 as shown in SEQ ID NO: 41.

In some embodiments, the VH comprises the HFR1 as shown in SEQ ID NO: 123, the HFR2 as shown in SEQ ID NO: 135, the HFR3 as shown in SEQ ID NO: 141, and the HFR4 as shown in SEQ ID NO: 41.

In some embodiments, the VH comprises the HFR1 as shown in SEQ ID NO: 126, the HFR2 as shown in SEQ ID NO: 135, the HFR3 as shown in SEQ ID NO: 142, and the HFR4 as shown in SEQ ID NO: 152.

In some embodiments, the VH comprises the HFR1 as shown in SEQ ID NO: 127, the HFR2 as shown in SEQ ID NO: 30, the HFR3 as shown in SEQ ID NO: 143, and the HFR4 as shown in SEQ ID NO: 41.

In some embodiments, the VH comprises the HFR1 as shown in SEQ ID NO: 128, the HFR2 as shown in SEQ ID NO: 30, the HFR3 as shown in SEQ ID NO: 144, and the HFR4 as shown in SEQ ID NO: 41.

In some embodiments, the VH comprises the HFR1 as shown in SEQ ID NO: 129, the HFR2 as shown in SEQ ID NO: 30, the HFR3 as shown in SEQ ID NO: 145, and the HFR4 as shown in SEQ ID NO: 41.

In some embodiments, the VH comprises the HFR1 as shown in SEQ ID NO: 127, the HFR2 as shown in SEQ ID NO: 30, the HFR3 as shown in SEQ ID NO: 146, and the HFR4 as shown in SEQ ID NO: 41.

In some embodiments, the VH comprises the HFR1 as shown in SEQ ID NO: 125, the HFR2 as shown in SEQ ID NO: 135, the HFR3 as shown in SEQ ID NO: 142, and the HFR4 as shown in SEQ ID NO: 152.

In some embodiments, the VH comprises the HFR1 as shown in SEQ ID NO: 130, the HFR2 as shown in SEQ ID NO: 30, the HFR3 as shown in SEQ ID NO: 147, and the HFR4 as shown in SEQ ID NO: 41.

In some embodiments, the VH comprises the HFR1 as shown in SEQ ID NO: 131, the HFR2 as shown in SEQ ID NO: 135, the HFR3 as shown in SEQ ID NO: 148, and the HFR4 as shown in SEQ ID NO: 153.

In some embodiments, the VH comprises the HFR1 as shown in SEQ ID NO: 132, the HFR2 as shown in SEQ ID NO: 30, the HFR3 as shown in SEQ ID NO: 145, and the HFR4 as shown in SEQ ID NO: 41.

In some embodiments, the first antigen-binding domain of the isolated antigen-binding fragment further comprises a light chain variable region (VL) containing at least one light chain complementarity-determining region (LCDR) containing amino acid sequences selected from SEQ ID NO:154, SEQ ID NO:159, and SEQ ID NO:163.

In some embodiments, the VL comprises an LCDR3 comprising an amino acid sequence of SEQ ID NO:163.

In some embodiments, the LCDR3 comprises an amino acid sequence shown in any one of SEQ ID NO: 164-168.

In some embodiments, the VL comprises an LCDR2 comprising an amino acid sequence of SEQ ID NO:159.

In some embodiments, the LCDR2 comprises an amino acid sequence shown in any one of SEQ ID NO:160-162.

In some embodiments, the VL comprises an LCDR1 comprising an amino acid sequence of SEQ ID NO:154.

In some embodiments, the LCDR1 comprises an amino acid sequence shown in any one of SEQ ID NO: 155-158.

In some embodiments, the VL comprises the LCDR1 as shown in SEQ ID NO: 157, the LCDR2 as shown in SEQ ID NO: 161 and the LCDR3 as shown in SEQ ID NO: 164.

In some embodiments, the VL comprises the LCDR1 as shown in SEQ ID NO: 157, the LCDR2 as shown in SEQ ID NO: 162 and the LCDR3 as shown in SEQ ID NO: 164.

In some embodiments, the VL comprises the LCDR1 as shown in SEQ ID NO: 156, the LCDR2 as shown in SEQ ID NO: 161 and the LCDR3 as shown in SEQ ID NO: 164.

In some embodiments, the VL comprises the LCDR1 as shown in SEQ ID NO: 158, the LCDR2 as shown in SEQ ID NO: 160 and the LCDR3 as shown in SEQ ID NO: 164.

In some embodiments, the VL comprises the LCDR1 as shown in SEQ ID NO: 155, the LCDR2 as shown in SEQ ID NO: 160 and the LCDR3 as shown in SEQ ID NO: 164.

In some embodiments, the VL comprises the LCDR1 as shown in SEQ ID NO: 156, the LCDR2 as shown in SEQ ID NO: 162 and the LCDR3 as shown in SEQ ID NO: 164.

In some embodiments, the VL comprises the LCDR1 as shown in SEQ ID NO: 155, the LCDR2 as shown in SEQ ID NO: 160 and the LCDR3 as shown in SEQ ID NO: 165.

In some embodiments, the VL comprises the LCDR1 as shown in SEQ ID NO: 155, the LCDR2 as shown in SEQ ID NO: 160 and the LCDR3 as shown in SEQ ID NO: 166.

In some embodiments, the VL comprises the LCDR1 as shown in SEQ ID NO: 155, the LCDR2 as shown in SEQ ID NO: 160 and the LCDR3 as shown in SEQ ID NO: 167.

In some embodiments, the VL comprises the LCDR1 as shown in SEQ ID NO: 155, the LCDR2 as shown in SEQ ID NO: 160 and the LCDR3 as shown in SEQ ID NO: 168.

In some embodiments, the VL comprises the LFR1 comprising an amino acid sequence of SEQ ID NO:169.

In some embodiments, the LFR1 comprises an amino acid sequence shown in any one of SEQ ID NO:170-179.

In some embodiments, the VL comprises the LFR2 comprising an amino acid sequence of SEQ ID NO:180.

In some embodiments, the LFR2 comprises an amino acid sequence shown in either of SEQ ID NO: 181-188.

In some embodiments, the VL comprises the LFR3 comprising an amino acid sequence of SEQ ID NO:189.

In some embodiments, the LFR3 comprises an amino acid sequence shown in any one of SEQ ID NO: 190-197, 57.

In some embodiments, the VL comprises the LFR4 comprising an amino acid sequence of SEQ ID NO:198.

In some embodiments, the LFR4 comprises an amino acid sequence shown in any one of the SEQ IDS NO: 199-201, 61.

In some embodiments, the VL comprises the LFR1 as shown in SEQ ID NO: 170, the LFR2 as shown in SEQ ID NO: 181, the LFR3 as shown in SEQ ID NO: 190, and the LFR4 as shown in SEQ ID NO: 199.

In some embodiments, the VL comprises the LFR1 as shown in SEQ ID NO: 171, the LFR2 as shown in SEQ ID NO: 182, the LFR3 as shown in SEQ ID NO: 191, and the LFR4 as shown in SEQ ID NO: 200.

In some embodiments, the VL comprises the LFR1 as shown in SEQ ID NO: 172, the LFR2 as shown in SEQ ID NO: 183, the LFR3 as shown in SEQ ID NO: 192, and the LFR4 as shown in SEQ ID NO: 61.

In some embodiments, the VL comprises the LFR1 as shown in SEQ ID NO: 174, the LFR2 as shown in SEQ ID NO: 184, the LFR3 as shown in SEQ ID NO: 57, and the LFR4 as shown in SEQ ID NO: 199.

In some embodiments, the VL comprises the LFR1 as shown in SEQ ID NO: 175, the LFR2 as shown in SEQ ID NO: 185, the LFR3 as shown in SEQ ID NO: 193, and the LFR4 as shown in SEQ ID NO: 199.

In some embodiments, the VL comprises the LFR1 as shown in SEQ ID NO: 176, the LFR2 as shown in SEQ ID NO: 186, the LFR3 as shown in SEQ ID NO: 194, and the LFR4 as shown in SEQ ID NO: 199.

In some embodiments, the VL comprises the LFR1 as shown in SEQ ID NO: 177, the LFR2 as shown in SEQ ID NO: 187, the LFR3 as shown in SEQ ID NO: 195, and the LFR4 as shown in SEQ ID NO: 199.

In some embodiments, the VL comprises the LFR1 as shown in SEQ ID NO: 178, the LFR2 as shown in SEQ ID NO: 188, the LFR3 as shown in SEQ ID NO: 196, and the LFR4 as shown in SEQ ID NO: 199.

In some embodiments, the VL comprises the LFR1 as shown in SEQ ID NO: 179, LFR2 as shown in SEQ ID NO: 187, LFR3 as shown in SEQ ID NO: 195, and LFR4 as shown in SEQ ID NO: 199.

In some embodiments, the VL comprises LFR1 as shown in SEQ ID NO: 173, LFR2 as shown in SEQ ID NO: 183, LFR3 as shown in SEQ ID NO: 192, and LFR4 as shown in SEQ ID NO: 201.

In some embodiments, the VL comprises LFR1 as shown in SEQ ID NO: 173, LFR2 as shown in SEQ ID NO: 183, LFR3 as shown in SEQ ID NO: 192, and LFR4 as shown in SEQ ID NO: 199.

In some embodiments, the VH comprises a sequence as described in any one of SEQ ID NO:202-220.

In some embodiments, the VL comprises a sequence as described in any one of SEQ ID NO: 221-234.

An isolated antigen-binding protein comprising: a first antigen-binding domain and a second antigen-binding domain, the first antigen-binding domain specifically binds to TNF-like protein A (TL1A), and the first antigen-binding domain is different from the second antigen-binding domain.

In some embodiments, the first antigen binding domain comprises a heavy chain variable region (VH) containing an HCDR3 which comprises an amino acid sequence of SEQ ID NO:242.

In some embodiments, the VH comprises an HCDR2 comprising an amino acid sequence of SEQ ID NO:238.

In some embodiments, the HCDR2 comprises an amino acid sequence of any one of SEQ ID NO: 239-241.

In some embodiments, the VH comprises an HCDR1 comprising an amino acid sequence of SEQ ID NO:235.

In some embodiments, the HCDR1 comprises an amino acid sequence of any one of SEQ ID NO: 236-237.

In some embodiments, the VH comprises the HCDR1 as shown in SEQ ID NO: 108, the HCDR2 as shown in SEQ ID NO: 116, and the HCDR3 as shown in SEQ ID NO: 120.

In some embodiments, the VH comprises the HCDR1 as shown in SEQ ID NO: 236, the HCDR2 as shown in SEQ ID NO: 239, and the HCDR3 as shown in SEQ ID NO: 242.

In some embodiments, the VH comprises the HCDR1 as shown in SEQ ID NO: 236, the HCDR2 as shown in SEQ ID NO: 240, and the HCDR3 as shown in SEQ ID NO: 242.

In some embodiments, the VH comprises the HCDR1 as shown in SEQ ID NO: 237, the HCDR2 as shown in SEQ ID NO: 241, and the HCDR3 as shown in SEQ ID NO: 242.

In some embodiments, the VH comprises an HFR1 comprising an amino acid sequence of SEQ ID NO:243.

In some embodiments, the HFR1 is selected from an amino acid sequence shown in any one of SEQ ID NO:244-250.

In some embodiments, the VH comprises an HFR2, and the HFR2 comprises an amino acid sequence of SEQ ID NO:251.

In some embodiments, the HFR2 is selected from an amino acid sequence shown in any one of SEQ ID NO: 252-255.

In some embodiments, the VH comprises an HFR3 comprising an amino acid sequence of SEQ ID NO:256.

In some embodiments, the HFR3 is selected from an amino acid sequence shown in any one of SEQ ID NO: 257-262.

In some embodiments, the VH comprises an HFR4 comprising an amino acid sequence shown in SEQ ID NO:263.

In some embodiments, the HFR4 is selected from an amino acid sequence shown in any one of SEQ ID NO: 264-268.

In some embodiments, the VH comprises the HFR1 as shown in SEQ ID NO: 244, the HFR2 as shown in SEQ ID NO: 252, the HFR3 as shown in SEQ ID NO: 257, and the HFR4 as shown in SEQ ID NO: 264.

In some embodiments, the VH comprises the HFR1 as shown in SEQ ID NO: 245, the HFR2 as shown in SEQ ID NO: 252, the HFR3 as shown in SEQ ID NO: 258, and the HFR4 as shown in SEQ ID NO: 265.

In some embodiments, the VH comprises the HFR1 as shown in SEQ ID NO: 246, the HFR2 as shown in SEQ ID NO: 253, the HFR3 as shown in SEQ ID NO: 259, and the HFR4 as shown in SEQ ID NO: 268.

In some embodiments, the VH comprises the HFR1 as shown in SEQ ID NO: 246, the HFR2 as shown in SEQ ID NO: 254, the HFR3 as shown in SEQ ID NO: 260, and the HFR4 as shown in SEQ ID NO: 266.

In some embodiments, the VH comprises the HFR1 as shown in SEQ ID NO: 247, the HFR2 as shown in SEQ ID NO: 254, the HFR3 as shown in SEQ ID NO: 261, and the HFR4 as shown in SEQ ID NO: 268.

In some embodiments, the VH comprises the HFR1 as shown in SEQ ID NO: 248, the HFR2 as shown in SEQ ID NO: 252, the HFR3 as shown in SEQ ID NO: 258, and the HFR4 as shown in SEQ ID NO: 265.

In some embodiments, the VH comprises the HFR1 as shown in SEQ ID NO: 246, the HFR2 as shown in SEQ ID NO: 255, the HFR3 as shown in SEQ ID NO: 260, and the HFR4 as shown in SEQ ID NO: 268.

In some embodiments, the VH comprises the HFR1 as shown in SEQ ID NO: 245, the HFR2 as shown in SEQ ID NO: 252, the HFR3 as shown in SEQ ID NO: 258, and the HFR4 as shown in SEQ ID NO: 267.

In some embodiments, the VH comprises the HFR1 as shown in SEQ ID NO: 249, the HFR2 as shown in SEQ ID NO: 254, the HFR3 as shown in SEQ ID NO: 260, and the HFR4 as shown in SEQ ID NO: 268.

In some embodiments, the VH comprises the HFR1 as shown in SEQ ID NO: 250, the HFR2 as shown in SEQ ID NO: 253, the HFR3 as shown in SEQ ID NO: 262, and the HFR4 as shown in SEQ ID NO: 266.

In some embodiments, the isolated antigen-binding fragments further include a light chain variable region (VL) containing at least one light chain complementarity-determining region (LCDR) containing amino acid sequences selected from an amino acid sequences shown in SEQ ID NO: 269, SEQ ID NO: 274, and SEQ ID NO: 277.

In some embodiments, the VL comprises an LCDR3 comprising an amino acid sequence of SEQ ID NO:277.

In some embodiments, the VL comprises an LCDR2 comprising an amino acid sequence of SEQ ID NO:274.

In some embodiments, the LCDR2 comprises an amino acid sequence shown in any one of SEQ ID NO:275-276.

In some embodiments, the VL comprises an LCDR1 comprising an amino acid sequence of SEQ ID NO:269.

In some embodiments, the LCDR1 comprises an amino acid sequence shown in any one of SEQ ID NO:270-273.

In some embodiments, the VL comprises the LCDR1 as shown in SEQ ID NO: 270, the LCDR2 as shown in SEQ ID NO: 275 and the LCDR3 as shown in SEQ ID NO: 277.

In some embodiments, the VL comprises the LCDR1 as shown in SEQ ID NO: 271, the LCDR2 as shown in SEQ ID NO: 275 and the LCDR3 as shown in SEQ ID NO: 277.

In some embodiments, the VL comprises the LCDR1 as shown in SEQ ID NO: 272, the LCDR2 as shown in SEQ ID NO: 275 and the LCDR3 as shown in SEQ ID NO: 277.

In some embodiments, the VL comprises the LCDR1 as shown in SEQ ID NO: 273, the LCDR2 as shown in SEQ ID NO: 275 and the LCDR3 as shown in SEQ ID NO: 277.

In some embodiments, the VL comprises the LCDR1 as shown in SEQ ID NO: 271, the LCDR2 as shown in SEQ ID NO: 276 and the LCDR3 as shown in SEQ ID NO: 277.

In some embodiments, the VL comprises the LFR1 comprising an amino acid sequence of SEQ ID NO:278.

In some embodiments, the LFR1 comprises an amino acid sequence as shown in any of SEQ ID NO: 47, 173, 279-283.

In some embodiments, the VL comprises the LFR2 comprising an amino acid sequence of SEQ ID NO:284.

In some embodiments, the LFR2 comprises amino acid sequences as shown in any of SEQ ID NO: 52, 285-287.

In some embodiments, the VL comprises the LFR3 comprising an amino acid sequence of SEQ ID NO:288.

In some embodiments, the LFR3 comprises an amino acid sequence shown in any one of SEQ ID NO: 289-293, 57.

In some embodiments, the VL comprises the LFR4 comprising an amino acid sequence of SEQ ID NO:294.

In some embodiments, the LFR4 comprises an amino acid sequence shown in any one of SEQ ID NO: 201, 295-296.

In some embodiments, the VL comprises the LFR1 as shown in SEQ ID NO: 170, the LFR2 as shown in SEQ ID NO: 181, the LFR3 as shown in SEQ ID NO: 190, and the LFR4 as shown in SEQ ID NO: 199.

In some embodiments, the VL comprises the LFR1 as shown in SEQ ID NO: 171, the LFR2 as shown in SEQ ID NO: 182, the LFR3 as shown in SEQ ID NO: 191, and the LFR4 as shown in SEQ ID NO: 200.

In some embodiments, the VL comprises the LFR1 as shown in SEQ ID NO: 172, the LFR2 as shown in SEQ ID NO: 183, the LFR3 as shown in SEQ ID NO: 192, and the LFR4 as shown in SEQ ID NO: 61.

In some embodiments, the VL comprises the LFR1 as shown in SEQ ID NO: 174, the LFR2 as shown in SEQ ID NO: 184, the LFR3 as shown in SEQ ID NO: 57, and the LFR4 as shown in SEQ ID NO: 199.

In some embodiments, the VL comprises the LFR1 as shown in SEQ ID NO: 175, LFR2 as shown in SEQ ID NO: 185, LFR3 as shown in SEQ ID NO: 193, and LFR4 as shown in SEQ ID NO: 199.

In some embodiments, the VL comprises LFR1 as shown in SEQ ID NO: 176, LFR2 as shown in SEQ ID NO: 186, LFR3 as shown in SEQ ID NO: 194, and LFR4 as shown in SEQ ID NO: 199.

In some embodiments, the VL comprises the LFR1 as shown in SEQ ID NO: 177, the LFR2 as shown in SEQ ID NO: 187, the LFR3 as shown in SEQ ID NO: 195, and the LFR4 as shown in SEQ ID NO: 199.

In some embodiments, the VL comprises the LFR1 as shown in SEQ ID NO: 178, the LFR2 as shown in SEQ ID NO: 188, the LFR3 as shown in SEQ ID NO: 196, and the LFR4 as shown in SEQ ID NO: 199.

In some embodiments, the VL comprises the LFR1 as shown in SEQ ID NO: 179, the LFR2 as shown in SEQ ID NO: 187, the LFR3 as shown in SEQ ID NO: 195, and the LFR4 as shown in SEQ ID NO: 199.

In some embodiments, the VL comprises the LFR1 as shown in SEQ ID NO: 173, the LFR2 as shown in SEQ ID NO: 183, the LFR3 as shown in SEQ ID NO: 192, and the LFR4 as shown in SEQ ID NO: 201.

In some embodiments, the VH comprises a sequence as described in any one of SEQ ID NO:202-220.

In some embodiments, the VL comprises a sequence as described in any one of SEQ ID NO: 221-234.

In some embodiments, it also comprises a heavy chain constant region for IgG and/or a light chain constant region for human antibodies.

In some embodiments, the isolated antigen-binding fragment comprises a heavy chain constant region of IgG1, IgG2, IgG3, or IgG4.

In some embodiments, the isolated antigen-binding fragment comprises a Kappa or Lambda light chain constant region.

In some embodiments, the isolated antigen-binding fragment is IgG, IgM, IgA, IgD, or IgE.

In some embodiments, the antigen-binding protein comprises one or more first heavy chain and one or more first light chain, and the first heavy chain comprises VH of the first antigen-binding domain and/or VH of the second antigen binding domain and/or scFv of the first antigen-binding domain and/or scFv of the second antigen binding domain; the first light chain comprises a VL of the first antigen-binding domain or a VL of the second antigen binding domain.

In some embodiments, the first heavy chain comprises VH of the first antigen-binding domain and scFv of the second antigen binding domain, or the first heavy chain comprises VH of the second antigen binding domain and scFv of the first antigen-binding domain.

In some embodiments, the N-terminus of the scFv of the first antigen-binding domain or the second antigen binding domain is either directly or by linker to the C-terminus of the first heavy chain, or the C-terminus of the scFv of the first antigen-binding domain or the second antigen binding domain is connected directly or by a linker to the N-terminus of the first heavy chain.

In some embodiments,
(1) the C-terminus of the first antigen-binding domain VH is connected directly or by linkers to the N-terminus of the first heavy chain, and the N-terminus of the second antigen binding domain of scFv is connected directly or by linkers to the C-terminus of the first heavy chain; The C-terminus of the VL of the first antigen-binding domain is attached to the N-terminus of the constant region of the light chain;
(2) the C-terminus of the second antigen binding domain VH is connected directly or by linker to the N-terminus of the first heavy chain, and the N-terminus of scFv of the first antigen-binding domain is connected directly or by linker to the C-terminus of the first heavy chain; The C-terminus of the VL of the second antigen binding domain is connected to the N-terminus of the constant region of the light chain;
(3) the C-terminus of scFv of the first antigen-binding domain is connected directly or by a linker to the N-terminus of the second antigen binding domain VH, and the C-terminus of the VL of the second antigen binding domain is connected to the N-terminus of the constant region of the light chain;
(4) the C-terminus of the scFv of the second antigen binding domain is connected directly or by a linker to the N-terminus of the first antigen-binding domain VH, and the C-terminus of the VL of the first antigen-binding domain is connected to the N-terminus of the constant region of the light chain.

In some embodiments, the first heavy chain is selected from a sequence described in any one of SEQ ID NO: 350-366 and 378.

In some embodiments, the antigen-binding fragment is selected from one of the following:
1) a light chain as shown in SEQ ID NO: 348 and heavy chain as shown in SEQ ID NO: 350;
2) a light chain as shown in SEQ ID NO: 348 and a heavy chain as shown in SEQ ID NO: 351;
3) a light chain as shown in SEQ ID NO: 348 and a heavy chain as shown in SEQ ID NO: 352;
4) a light chain as shown in SEQ ID NO: 348 and a heavy chain as shown in SEQ ID NO: 353;
5) a light chain as shown in SEQ ID NO: 349 and a heavy chain as shown in SEQ ID NO: 354;
6) a light chain as shown in SEQ ID NO: 349 and a heavy chain as shown in SEQ ID NO: 355;
7) a light chain as shown in SEQ ID NO: 348 and a heavy chain as shown in SEQ ID NO: 356;
8) a light chain as shown in SEQ ID NO: 372 and a heavy chain as shown in SEQ ID NO: 357;
9) a light chain as shown in SEQ ID NO: 349 and a heavy chain as shown in SEQ ID NO: 358;
10) a light chain as shown in SEQ ID NO: 348 and a heavy chain as shown in SEQ ID NO: 359;
11) a light chain as shown in SEQ ID NO: 372 and a heavy chain as shown in SEQ ID NO: 360;
12) a light chain as shown in SEQ ID NO: 348 and a heavy chain as shown in SEQ ID NO: 361;
13) a light chain as shown in SEQ ID NO: 348 and a heavy chain as shown in SEQ ID NO: 362;
14) a light chain as shown in SEQ ID NO: 348 and a heavy chain as shown in SEQ ID NO: 363;
15) a light chain as shown in SEQ ID NO: 348 and a heavy chain as shown in SEQ ID NO: 364;
16) a light chain as shown in SEQ ID NO: 348 and a heavy chain as shown in SEQ ID NO: 365;
17) a light chain as shown in SEQ ID NO: 348 and a heavy chain as shown in SEQ ID NO: 366;
18) a light chain as shown in SEQ ID NO: 348 and a heavy chain as shown in SEQ ID NO: 378.

In some embodiments, the isolated antigen-binding protein includes a light chain as shown in SEQ ID NO: 348 and heavy chain as shown in SEQ ID NO: 363.

In some embodiments, the antigen-binding protein comprises one or more first light chains, one or more third heavy chains, and one or more fourth heavy chains, wherein:
1) the third heavy chain comprises VH for the first antigen binding domain or the second antigen binding domain, and the fourth heavy chain comprises scFv for the first or second antigen binding domain, or;
2) the third heavy chain comprises scFv for the first antigen binding domain or the second antigen binding domain, and the fourth heavy chain comprises VH for the first or second antigen binding domain.

In some embodiments, the N-terminus of the VH of the first antigen-binding domain or the second antigen binding domain is connected directly or through a linker to the C-terminus of the third heavy chain, or the N-terminus of scFv of the first or second antigen binding domain is connected directly or through a linker to the C-terminus of the fourth heavy chain.

In some embodiments, wherein
1) the third heavy chain comprises VH for the first antigen-binding domain, and the fourth heavy comprises scFv for the second antigen binding domain, or;
2) the third heavy chain comprises scFv for the first antigen-binding domain, and the fourth heavy chain comprises VH for the second antigen binding domain, or;
3) the third heavy chain comprises VH for the second antigen binding domain, and the fourth heavy comprises scFv for the first antigen-binding domain, or
4) the third heavy chain comprises scFv for the second antigen binding domain, and the fourth heavy comprises VH for the first antigen-binding domain.

In some embodiments, the antigen-binding fragment is selected from one of the following:
1) a light chain as shown in SEQ ID NO: 349, a third heavy chain as shown in SEQ ID NO: 367 and a fourth heavy chain as shown in SEQ ID NO: 368;
2) a light chain as shown in SEQ ID NO: 348, a third heavy chain as shown in SEQ ID NO: 369 and a fourth heavy chain as shown in SEQ ID NO: 370.

In some embodiments, the first antigen-binding domain comprises HCDR1 as set forth in SEQ ID NO: 2, HCDR2 as set forth in SEQ ID NO: 6, HCDR3 as set forth in SEQ ID NO: 15, LCDR1 as set forth in SEQ ID NO: 42, LCDR2 as set forth in SEQ ID NO: 43, and LCDR3 as set forth in SEQ ID NO: 44; the second antigen-binding domain comprises HCDR1 as set forth in SEQ ID NO: 332, HCDR2 as set forth in SEQ ID NO: 333, HCDR3 as set forth in SEQ ID NO: 334, LCDR1 as set forth in SEQ ID NO: 336, LCDR2 as set forth in SEQ ID NO: 337, and LCDR3 as set forth in SEQ ID NO: 338.

In some embodiments, the first antigen-binding domain comprises the VH as set forth in SEQ ID NO: 70 and the VL as set forth in SEQ ID NO: 106, and the second antigen-binding domain comprises the VH as set forth in SEQ ID NO: 335 and the VL as set forth in SEQ ID NO: 339.

In some embodiments, (1) the first antigen-binding domain comprises an scFv as set forth in SEQ ID NO: 340 and the second antigen-binding domain comprises the VH as set forth in SEQ ID NO: 335 and the VL as set forth in SEQ ID NO: 339; or, (2) the first antigen-binding domain comprises an scFv as set forth in SEQ ID NO: 341 and the second antigen-binding domain comprises the VH as set forth in SEQ ID NO: 335 and the VL as set forth in SEQ ID NO: 339; or, (3) the first antigen-binding domain comprises an scFv as set forth in SEQ ID NO: 342; and the second antigen-binding domain comprises the VH as set forth in SEQ ID NO: 335 and the VL as set forth in SEQ ID NO: 339; or, (4) the first antigen-binding domain comprises an scFv as set forth in SEQ ID NO: 343 and the second antigen-binding domain comprises the VH as set forth in SEQ ID NO: 335 and the VL as set forth in SEQ ID NO: 339; or, (5) the first antigen binding domain comprises the VH as set forth in SEQ ID NO: 70 and the VL as set forth in SEQ ID NO: 106 and the second antigen binding domain comprises an scFv as set forth in 346; or, (6) the first antigen binding domain comprises the VH as set forth in SEQ ID NO: 70 and the VL as set forth in SEQ ID NO: 106 and the second antigen binding domain comprises an scFv as set forth in SEQ ID NO:347.

In some embodiments, the isolated antigen-binding protein includes a light chain as shown in SEQ ID NO: 348 and heavy chain as shown in SEQ ID NO: 363.

In another aspect, the present application provides an isolated nucleic acid molecule encoding the isolated antigen-binding fragment.

In another aspect, the present application provides an expression vector which comprises the aforementioned nucleic acids.

In another aspect, the present application provides a host cell containing the aforementioned isolated nucleic acids and/or the expression vector.

In another aspect, the present application provides a composition containing the aforementioned isolated antigen-binding fragment and optionally a pharmaceutically acceptable carrier.

In another aspect, the application provides a method for preparing antigen-binding fragments isolated from TL1A, comprising:
culturing the host cells claimed above, and recovering the isolated antigen-binding fragments.

In another aspect, the present application provides a method for the treatment of TL1A-mediated disease or symptoms, which comprises:
administrating of the aforementioned isolated antigen-binding fragments, aforementioned isolated nucleic acids, and/or aforementioned expression vectors, aforementioned host cells, or aforementioned combinations.

Antigen-binding fragments isolated as described above, nucleic acids isolated as described above, and/or expression vectors described above, host cells as described above, or combinations described above, which are used for the treatment of TL1A-mediated diseases or symptoms.

The use of the aforementioned isolated antigen-binding fragments, aforementioned isolated nucleic acids, and/or aforementioned expression vectors, aforementioned host cells, or aforementioned compositions in the treatment of TL1A-mediated diseases or symptoms.

The use of aforementioned isolated antigen-binding fragments, the aforementioned isolated nucleic acids, and/or 2 the aforementioned expression vectors, aforementioned host cells, or the aforementioned combinations in the preparation of drugs for the treatment of TL1A-mediated diseases or conditions.

In some embodiments, the disease or symptom mediated by TL1A is inflammatory.

In some embodiments, TL1A-mediated inflammatory disease is selected from one of the following: Non-limiting examples of inflammatory disease include, allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis (e.g., idiopathic pulmonary fibrosis), rheumatic disease, scleroderma, sinusitis, tuberculosis.

In some embodiments, inflammatory bowel disease (IBD) comprises ulcerative colitis (UC) and Crohn's disease (CD).

In some embodiments, the disease or symptom mediated by TL1A is an autoimmune disease.

In some embodiments, TL1A-mediated autoimmune disease is selected from one of the following: achalasia, Addison's disease, Adult Stil's disease, agammaglobulinemia, alopecia areata, amyloidosis, ankylosing spondylitis, anti-GBM/anti-TBM nephritis, antiphospholipid syndrome, autoimmune angioedema, autoimmune familial autonomic dysfunction, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal and neuronal neuropathy (AMAN), Barlow disease (Bal ó disease), Behcet's disease, benign mucous membrane pemphigoid, bullous pemphigoid, Castleman's disease (Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatous disease (EGPA), cicatricial pemphigoid, and Cogan's syndrome), cold agglutinin disease, congenital heart block, coxsackienew myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, primary mixed cryoglobulinemia, Evans syndrome, fibromyalgia, fibrotic alveolitis, giant cell arteritis (temporal arteritis), giant cell myocarditis, glomerulonephritis, Goodpasture's syndrome, granulomatosis with polyangiitis, Graves' disease, Guillain-Barre syndrome syndrome), Hashimoto's thyroiditis, hemolytic anemia, Henoch-Schonlein purpura (HSP), herpes gravilens or pemphigoid gestationum (PG), hidradenitis suppurativa (HS) (paradoxical acne), hypogammaglobulinemia, IgA nephropathy, IgG4-associated sclerotic disease, immune thrombocytopenic purpura (ITP), Inclusion body myositis (IBM), interstitial cystitis (IC), juvenile arthritis, juvenile diabetes mellitus (type 1 diabetes), juvenile myositis (JM), Kawasaki disease, Lambert-Eaton syndrome, leukocytoclastic vasculitis, lichen planus, lichen sclerosus, lignoconjunctivitis, linear IgA disease (LAD), lupus, chronic Lyme disease (Lymedisease chronic), Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, recurrent rheumatism (PR), PandAS, paraneoplastic cerebellar degeneration (PCD), paroxysmal nocturnal hemoglobinuria (PNH), Parry Romberg syndrome, ciliary planitis (peripheral uveitis), Parsonage-Tumer syndrome), pemphigus, peripheral neuropathy, perivenous encephalomyelitis, pernicious anemia (PA), POEMS syndrome, polyarteritis nodosa, polyglandular syndrome type I, II, type III, polymyalgia rheumatica, polymyositis, post-myocardial infarction syndrome, postpericardiotomy syndrome, primary biliary cirrhosis, primary sclerosing cholangitis, progesterone dermatitis, psoriasis, psoriatic arthritis, pure red blood cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff-person syndrome (SPS), subacute bacterial endocarditis (SBE), Susac's syndrome syndrome), sympathetic ophthalmia (SO), Takayasu's arteritis, temporal arteritis/giant cell arteritis, thrombocytopenic purpura (TTP), Tolosa-Hunt syndrome (THS), transverse myelitis, type 1 diabetes mellitus, ulcerative colitis (UC), undifferentiated connective tissue disease (UCTD), uveitis, vasculitis, Vitiligo and Vogt-Koyanagi-Harada disease.

In some embodiments, the disease or symptom mediated by TL1A is cancer.

In some embodiments, TL1A-mediated cancers are selected from one or more of the following: non-restrictive examples of cancer include adenoid cystic carcinoma, adrenal carcinoma, amyloidosis, anal cancer, ataxia-telangiectasia, atypical nevus syndrome, basal cell carcinoma, cholangiocarcinoma, Birt Hogg Dube syndrome, bladder cancer, bone cancer, brain tumor, breast cancer, male breast cancer, carcinoid tumor, cervical cancer, colorectal cancer, ductal cancer, Endometrial Cancer, Esophageal Cancer, Gastric Cancer, Gastrointestinal Stromal Tumor (GIST), HER2-Positive Breast Cancer, Pancreatic Islet Cell Tumor, Juvenile Polyposis Syndrome, Kidney Cancer, Laryngeal Cancer, Leukemia-Acute Lymphoblastic Leukemia, Acute Lymphoblastic Leukemia (ALL), Acute Myeloid Leukemia AML, Adult Leukemia, Childhood Leukemia, Chronic Lymphocytic Leukemia (CLL), Chronic Myeloid Leukemia (CML), Liver Cancer, Lobular Carcinoma, Lung Cancer, Small Cell Lung Cancer (SCLC), Non-small cell lung cancer (NSCLC), Hodgkin's lymphoma (Lymphoma-Hodgkin's), non-Hodgkin's lymphoma (Lymphoma-Non-Hodgkin's), malignant glioma, melanoma, meningioma, multiple myeloma, myelodysplastic syndrome (MDS), nasopharyngeal carcinoma, neuroendocrine tumor, oral cancer, osteosarcoma, ovarian cancer, pancreatic cancer, pancreatic neuroendocrine tumor, parathyroid carcinoma, penile cancer, peritoneal cancer, Peutz-Jeghers syndrome, pituitary tumors, polycythemia vera, prostate cancer, renal cell carcinoma, retinoblastoma, salivary gland carcinoma, sarcoma, Sarcoma-Kaposi, skin cancer, small bowel cancer, stomach cancer, testicular cancer, thymoma, thyroid cancer, uterine (endometrial) cancer, vaginal cancer, and Wilms' Tumor.

On the other hand, the present application provides a method for downregulating the expression of α4β7 and/or TL1A, comprising providing a TL1A inhibitor and an α4β7 inhibitor to a subject.

On the other hand, the present application provides a method for inducing the endocytosis of cell surface α4β7 and/or TL1A, comprising providing a TL1A inhibitor and an α4β7 inhibitor to a subject.

In some embodiments, the method further comprises: providing an antigen binding protein that can specifically bind to α4β7 or providing an antigen binding protein that can specifically bind to TL1A.

In some embodiments, the method further comprises: providing a TL1A protein or an α4β7 protein.

In some embodiments, the isolated antigen binding protein that can specifically bind to TL1A and the antigen binding protein that can specifically bind to α4β7 are the same protein.

In some embodiments, the isolated antigen binding protein comprises one or more polypeptides, wherein one polypeptide comprises at least a first antigen binding domain and at least one second antigen binding domain.

In some embodiments, the isolated antigen-binding protein comprises a first polypeptide and a second polypeptide, wherein the first polypeptide comprises a first antigen-binding domain and a second antigen-binding domain, and the second polypeptide comprises a first antigen-binding domain or a second antigen-binding domain, and one or more of the following situations are selected: (1) the first antigen-binding domain of the first polypeptide is located at the C-terminus of the first polypeptide, and the first antigen-binding domain of the second polypeptide is located at the C-terminus of the first polypeptide; (2) the first antigen-binding domain of the first polypeptide is located at the N-terminus of the first polypeptide, and the first antigen-binding domain of the second polypeptide is located at the N-terminus of the first polypeptide; (3) the second antigen-binding domain of the first polypeptide is located at the N-terminus of the first polypeptide, and the second antigen-binding domain of the second polypeptide is located at the N-terminus of the second polypeptide; (4) the second antigen-binding domain of the first polypeptide is located at the C-terminus of the second polypeptide, and the second antigen-binding domain of the second polypeptide is located at the C-terminus of the second polypeptide.

In some embodiments, the isolated antigen-binding protein comprises a first polypeptide and a second polypeptide, wherein the first polypeptide comprises a first antigen-binding domain and a second antigen-binding domain, and the second polypeptide comprises a first antigen-binding domain and a second antigen-binding domain, and one or more of the following situations are selected: (1) the first antigen-binding domain of the first polypeptide is located at the C-terminus of the first polypeptide, and the first antigen-binding domain of the second polypeptide is located at the C-terminus of the first polypeptide; (2) the first antigen-binding domain of the first polypeptide is located at the N-terminus of the first polypeptide, and the first antigen-binding domain of the second polypeptide is located at the N-terminus of the first polypeptide; (3) the second antigen-binding domain of the first polypeptide is located at the N-terminus of the first polypeptide, and the second antigen-binding domain of the second polypeptide is located at the N-terminus of the second polypeptide; (4) the second antigen-binding domain of the first polypeptide is located at the C-terminus of the second polypeptide, and the second antigen-binding domain of the second polypeptide is located at the C-terminus of the second polypeptide.

In some embodiments, the isolated antigen-binding protein comprises a first polypeptide and a second polypeptide, wherein the first polypeptide comprises the first antigen-binding domain and does not include the second antigen-binding domain, and the second polypeptide comprises the second antigen-binding domain and does not include the first antigen-binding domain.

In some embodiments, the isolated antigen binding protein specifically binds to the α4β7 protein with a KD value of about 5E-08M or less.

In some embodiments, the isolated antigen binding protein specifically binds to monomeric TL1A and/or trimeric TL1A with a KD value of about 5E-08M or less.

On the other hand, the present application provides a use of the antigen binding protein to induce the endocytosis of cell surface α4β7 and/or TL1A.

On the other hand, the present application provides a use of the antigen binding protein to downregulate the expression of α4β7 and/or TL1A.

A bispecific antibody, comprising: a first antigen-binding domain and a second antigen-binding domain, wherein the first antigen-binding domain specifically binds to TNF-like protein A (TL1A), and wherein the second antigen binding domain specifically binds to α4β7.

In some embodiments, the bispecific antibody comprises: a first antigen-binding domain and
a second antigen-binding domain, wherein the first antigen-binding domain specifically binds to TNF-like protein A (TL1A), and the second antigen binding domain specifically binds to α4β7, wherein the first antigen-binding domain comprises a heavy chain variable region (VH), and the VH comprises an HCDR3 SEQ ID NO:14.

In some embodiments, the HCDR3 of the first antigen-binding domain of the bispecific antibody comprises an amino acid sequence of any one of SEQ ID NOs: 15-18.

In some embodiments, the VH of the first antigen-binding domain of the bispecific antibody comprises an HCDR2 comprising an amino acid sequence of SEQ ID NO:5.

In some embodiments, the HCDR2 of the first antigen-binding domain of the bispecific antibody comprises an amino acid sequence of any one of SEQ ID NOs: 6-13.

In some embodiments, the VH of the first antigen-binding domain of the bispecific antibody
comprises an HCDR1 comprising an amino acid sequence of SEQ ID NO:1.

In some embodiments, the HCDR1 of the first antigen-binding domain of the bispecific antibody comprises an amino acid sequence of any one of SEQ ID NOs:2-4.

In some embodiments;
(i) the VH of the first antigen-binding domain of the bispecific antibody comprises the HCDR1 as set forth in SEQ ID NO: 2, the HCDR2 as set forth in SEQ ID NO: 6, and the HCDR3 as set forth in SEQ ID NO: 15;
(ii) the VH of the first antigen-binding domain of the bispecific antibody comprises the HCDR1 as set forth in SEQ ID NO: 2, the HCDR2 as set forth in SEQ ID NO: 7, and the HCDR3 as set forth in SEQ ID NO: 15;
(iii) the VH of the first antigen-binding domain of the bispecific antibody comprises the HCDR1 as set forth in SEQ ID NO: 2, the HCDR2 as set forth in SEQ ID NO: 8, and the HCDR3 as set forth in SEQ ID NO: 15;
(iv) the VH of the first antigen-binding domain of the bispecific antibody comprises the HCDR1 as set forth in SEQ ID NO: 3, the HCDR2 as set forth in SEQ ID NO: 6 and the HCDR3 as set forth in SEQ ID NO: 15;
(v) the VH of the first antigen-binding domain of the bispecific antibody comprises the HCDR1 as set forth in SEQ ID NO: 2, the HCDR2 as set forth in SEQ ID NO: 6, and the HCDR3 as set forth in SEQ ID NO: 16;
(vi) the VH of the first antigen-binding domain of the bispecific antibody comprises the HCDR1 as set forth in SEQ ID NO: 2, the HCDR2 as set forth in SEQ ID NO: 6, and the HCDR3 as set forth in SEQ ID NO: 17;
(vii) the VH of the first antigen-binding domain of the bispecific antibody comprises the HCDR1 as set forth in SEQ ID NO: 2, the HCDR2 as set forth in SEQ ID NO: 6, and the HCDR3 as set forth in SEQ ID NO: 18;
(viii) the VH of the first antigen-binding domain of the bispecific antibody comprises the HCDR1 as set forth in SEQ ID NO: 4, the HCDR2 as set forth in SEQ ID NO: 6, and the HCDR3 as set forth in SEQ ID NO: 15;
(ix) the VH of the first antigen-binding domain of the bispecific antibody comprises the HCDR1 as set forth in SEQ ID NO: 2, the HCDR2 as set forth in SEQ ID NO: 9, and the HCDR3 as set forth in SEQ ID NO: 15;
(x) the VH of the first antigen-binding domain of the bispecific antibody comprises the HCDR1 as set forth in SEQ ID NO: 2, the HCDR2 as set forth in SEQ ID NO: 10, and the HCDR3 as set forth in SEQ ID NO: 15;
(xi) the VH of the first antigen-binding domain of the bispecific antibody comprises the HCDR1 as set forth in SEQ ID NO: 2, the HCDR2 as set forth in SEQ ID NO: 11, and the HCDR3 as set forth in SEQ ID NO: 15;
(xii) the VH of the first antigen-binding domain of the bispecific antibody comprises the HCDR1 as set forth in SEQ ID NO: 2, the HCDR2 as set forth in SEQ ID NO: 12, and the HCDR3 as set forth in SEQ ID NO: 15;
(xiii) the VH of the first antigen-binding domain of the bispecific antibody comprises the HCDR1 as set forth in SEQ ID NO: 2, the HCDR2 as set forth in SEQ ID NO: 13, and the HCDR3 as set forth in SEQ ID NO: 15;
(xiv) the VH of the first antigen-binding domain of the bispecific antibody comprises the HCDR1 as set forth in SEQ ID NO: 3, the HCDR2 as set forth in SEQ ID NO: 6, and the HCDR3 as set forth in SEQ ID NO: 16;
(xv) the VH of the first antigen-binding domain of the bispecific antibody comprises the HCDR1 as set forth in SEQ ID NO: 3, the HCDR2 as set forth in SEQ ID NO: 6, and the HCDR3 as set forth in SEQ ID NO: 17;
(xvi) the VH of the first antigen-binding domain of the bispecific antibody comprises the HCDR1 as set forth in SEQ ID NO: 3, the HCDR2 as set forth in SEQ ID NO: 9, and the HCDR3 as set forth in SEQ ID NO: 15;
(xvii) the VH of the first antigen-binding domain of the bispecific antibody comprises the HCDR1 as set forth in SEQ ID NO: 3, the HCDR2 as set forth in SEQ ID NO: 10, and the HCDR3 as set forth in SEQ ID NO: 15;
(xviii) the VH of the first antigen-binding domain of the bispecific antibody comprises the HCDR1 as set forth in SEQ ID NO: 3, the HCDR2 as set forth in SEQ ID NO: 11, and the HCDR3 as set forth in SEQ ID NO: 15;
(xix) the VH of the first antigen-binding domain of the bispecific antibody comprises the HCDR1 as set forth in SEQ ID NO: 2, the HCDR2 as set forth in SEQ ID NO: 9, and the HCDR3 as set forth in SEQ ID NO: 16;
(xx) the VH of the first antigen-binding domain of the bispecific antibody comprises the HCDR1 as set forth in SEQ ID NO: 2, the HCDR2 as set forth in SEQ ID NO: 10, and the HCDR3 as set forth in SEQ ID NO: 16;
(xxi) the VH of the first antigen-binding domain of the bispecific antibody comprises the HCDR1 as set forth in SEQ ID NO: 2, the HCDR2 as set forth in SEQ ID NO: 11, and the HCDR3 as set forth in SEQ ID NO: 16;
(xxii) the VH of the first antigen-binding domain of the bispecific antibody comprises the HCDR1 as set forth in SEQ ID NO: 2, the HCDR2 as set forth in SEQ ID NO: 12, and the HCDR3 as set forth in SEQ ID NO: 16 or
(xxiii) the VH of the first antigen-binding domain of the bispecific antibody comprises the HCDR1 as set forth in SEQ ID NO: 2, the HCDR2 as set forth in SEQ ID NO:10, and the HCDR3 as set forth in SEQ ID NO:17.

In some embodiments, the first antigen-binding domain of the bispecific antibody further comprises a light chain variable region (VL), wherein the VL of the first antigen-binding domain comprises at least one light chain complementary determining region (LCDR) comprising an amino acid sequence selected from SEQ ID NO:42, SEQ ID NO:43 and SEQ ID NO:44.

In some embodiments, the VH of the first antigen-binding domain of the bispecific antibody
comprises a sequence as set forth in any one of SEQ ID NOs: 62-99 or 379.

In some embodiments, the VL of the first antigen-binding domain of the bispecific antibody
comprises a sequence as set forth in in any one of SEQ ID NOs: 102-106 or 380.

In some embodiments, the second antigen binding domain of the bispecific antibody comprises
a heavy chain variable region (VH), and the VH of the second antigen binding domain comprises an HCDR1 as set forth in SEQ ID NO: 332, an HCDR2 as set forth in SEQ ID NO: 333, and an HCDR3 as set forth in SEQ ID NO: 334, optionally wherein the second antigen binding domain comprises a VH as set forth in SEQ ID NO: 335.

In some embodiments, wherein the second antigen binding domain of the bispecific antibody
comprises a light chain variable region (VL), and the VL of the second antigen binding domain comprises an LCDR1 as set forth in SEQ ID NO: 336, an LCDR2 as set forth in SEQ ID NO: 337, and an LCDR3 as set forth in SEQ ID NO: 338, optionally wherein the second antigen binding domain comprises a VL as set forth in SEQ ID NO: 339.

In some embodiments, the bispecific antibody comprises a Fab, Fab′, Fv fragment, F(ab)′₂,
scFv, di-scFv and/or dAb.

In some embodiments, the bispecific antibody comprises the scFv of the first antigen binding
domain or the scFv of the second antigen binding domain.

In some embodiments, the scFv of the first antigen-binding domain of the bispecific antibody
comprises a sequence as set forth in any one of SEQ ID NOs: 340-345.

In some embodiments, the scFv of the first antigen-binding domain of the bispecific antibody
comprises a sequence as set forth in SEQ ID NO: 342.

In some embodiments, the first antigen-binding domain of the bispecific antibody is selected
from the combination of light chain and heavy chain of one of the following:
   a) a light chain of SEQ ID NO:348 and a heavy chain of SEQ ID NO:350;
   b) a light chain of SEQ ID NO:348 and a heavy chain of SEQ ID NO:351;
   c) a light chain of SEQ ID NO:348 and a heavy chain of SEQ ID NO:352;
   d) a light chain of SEQ ID NO:348 and a heavy chain of SEQ ID NO:353;
   e) a light chain of SEQ ID NO:349 and a heavy chain of SEQ ID NO:354;
   f) a light chain of SEQ ID NO:349 and a heavy chain of SEQ ID NO:355;
   g) a light chain of SEQ ID NO:348 and a heavy chain of SEQ ID NO:356;
   h) a light chain of SEQ ID NO:372 and a heavy chain of SEQ ID NO:357;
   i) a light chain of SEQ ID NO:349 and a heavy chain of SEQ ID NO:358;
   j) a light chain of SEQ ID NO:348 and a heavy chain of SEQ ID NO:359;
   k) a light chain of SEQ ID NO:372 and a heavy chain of SEQ ID NO:360;
   l) a light chain of SEQ ID NO:348 and a heavy chain of SEQ ID NO:361;
   m) a light chain of SEQ ID NO:348 and a heavy chain of SEQ ID NO:362;
   n) a light chain of SEQ ID NO:348 and a heavy chain of SEQ ID NO:363;
   o) a light chain of SEQ ID NO:348 and a heavy chain of SEQ ID NO:364;
   p) a light chain of SEQ ID NO:348 and a heavy chain of SEQ ID NO:365;
   q) a light chain of SEQ ID NO:348 and a heavy chain of SEQ ID NO:366;
   r) a light chain of SEQ ID NO:348 and a heavy chain as set forth in SEQ ID NO:378.

In some embodiments,
the first antigen-binding domain of the bispecific antibody comprises HCDR1 as set forth in SEQ ID NO: 2, HCDR2 as set forth in SEQ ID NO: 6, HCDR3 as set forth in SEQ ID NO: 15, LCDR1 as set forth in SEQ ID NO: 42, LCDR2 as set forth in SEQ ID NO: 43, and LCDR3 as set forth in SEQ ID NO: 44; and,
the second antigen-binding domain of the bispecific antibody comprises HCDR1 as set forth in SEQ ID NO: 332, HCDR2 as set forth in SEQ ID NO: 333, HCDR3 as set forth in SEQ ID NO: 334, LCDR1 as set forth in SEQ ID NO: 336, LCDR2 as set forth in SEQ ID NO: 337, and LCDR3 as set forth in SEQ ID NO: 338.

In some embodiments, the first antigen-binding domain of the bispecific antibody comprises a VH as set forth in SEQ ID NO: 379 and a VL as set forth in SEQ ID NO: 380; and the second antigen-binding domain of the bispecific antibody comprises a VH as set forth in SEQ ID NO: 335 and a VL as set forth in SEQ ID NO: 339.

In some embodiments;
(1) the first antigen-binding domain of the bispecific antibody comprises an scFv as set forth in SEQ ID NO: 340; and the second antigen-binding domain of the bispecific antibody comprises the VH as set forth in SEQ ID NO: 335 and the VL as set forth in SEQ ID NO: 339; or,
(2) the first antigen-binding domain of the bispecific antibody comprises an scFv as set forth in SEQ ID NO: 341; and the second antigen-binding domain of the bispecific antibody comprises the VH as set forth in SEQ ID NO: 335 and the VL as set forth in SEQ ID NO: 339; or,
(3) the first antigen-binding domain of the bispecific antibody comprises an scFv as set forth in SEQ ID NO: 342; and the second antigen-binding domain of the bispecific antibody comprises the VH as set forth in SEQ ID NO: 335 and the VL as set forth in SEQ ID NO: 339; or,
(4) the first antigen-binding domain of the bispecific antibody comprises an scFv as set forth in SEQ ID NO: 343; and the second antigen-binding domain of the bispecific antibody comprises the VH as set forth in SEQ ID NO: 335 and the VL as set forth in SEQ ID NO: 339; or,
(5) the first antigen binding domain of the bispecific antibody comprises the VH as set forth in SEQ ID NO: 70 and the VL as set forth in SEQ ID NO: 106; and the second antigen binding domain of the bispecific antibody comprises an scFv as set forth in 346; or,
(6) the first antigen binding domain of the bispecific antibody comprises the VH as set forth in SEQ ID NO: 70 and the VL as set forth in SEQ ID NO: 106; and the second antigen binding domain of the bispecific antibody comprises an scFv as set forth in SEQ ID NO:347.

In some embodiments, the bispecific antibody includes a light chain as shown in SEQ ID NO: 348 and a heavy chain as shown in SEQ ID NO: 363.

In another aspect, the application provides an isolated nucleic acid molecule encoding a bispecific antibody as described herein.

In another aspect, the application provides an expression vector comprising an isolated nucleic acid molecule as described herein.

In another aspect, the application provides a host cell including an isolated nucleic acid molecule and/or an expression vector as described herein.

In another aspect, the application provides a composition comprising a bispecific antibody as described herein and optionally pharmaceutical acceptable carrier.

In another aspect, the application provides a preparation method for bispecific antibodies as
described herein, comprising culturing a host cell as described herein, and recovering the bispecific antibodies.

In another aspect, the application provides a method of treating a disease or condition comprising administering a bispecific antibody, an isolated nucleic acid molecule, an expression vector, a host cell or a composition as described herein to a subject.

In another aspect, the application provides a bispecific antibody as described herein for use in the treatment of a disease or condition.

In another aspect, the application provides an isolated nucleic acid molecule, an expression
vector, a host cell or a composition as described herein for use in the treatment of a disease or condition.

In another aspect, the application provides a bispecific antibody, an isolated nucleic acid molecule, an expression vector, a host cell or a composition as described herein for the treatment of disease or condition or in the preparation of a drug for the treatment of disease or condition.

In some embodiments, the disease or the condition is a TL1A-mediated disease or condition.

In some embodiments, the TL1A-mediated disease or condition is inflammatory disease, optionally wherein the TL1A-mediated inflammatory disease is selected from one of the following: Non-limiting examples of inflammatory disease include, allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis (e.g., idiopathic pulmonary fibrosis), rheumatic disease, scleroderma, sinusitis, tuberculosis.

In some embodiments, the TL1A-mediated disease or condition is autoimmune disease, optionally wherein the TL1A-mediated autoimmune diseases are selected from one of the following: achalasia, Addison's disease, Adult Stil's disease, agammaglobulinemia, alopecia areata, amyloidosis, ankylosing spondylitis, anti-GBM/anti-TBM nephritis, antiphospholipid syndrome, autoimmune angioedema, autoimmune familial autonomic dysfunction, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal and neuronal neuropathy (AMAN), Barlow disease (Balódisease), Behcet's disease, benign mucous membrane pemphigoid, bullous pemphigoid, Castleman's disease (Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatous disease (EGPA), cicatricial pemphigoid, and Cogan's syndrome), cold agglutinin disease, congenital heart block, coxsackienew myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, primary mixed cryoglobulinemia, Evans syndrome, fibromyalgia, fibrotic alveolitis, giant cell arteritis (temporal arteritis), giant cell myocarditis, glomerulonephritis, Goodpasture's syndrome, granulomatosis with polyangiitis, Graves' disease, Guillain-Barre syndrome syndrome), Hashimoto's thyroiditis, hemolytic anemia, Henoch-Schonlein purpura (HSP), herpes gravilens or pemphigoid gestationum (PG), hidradenitis suppurativa (HS) (paradoxical acne), hypogammaglobulinemia, IgA nephropathy, IgG4-associated sclerotic disease, immune thrombocytopenic purpura (ITP), Inclusion body myositis (IBM), interstitial cystitis (IC), juvenile arthritis, juvenile diabetes mellitus (type 1 diabetes), juvenile myositis (JM), Kawasaki disease, Lambert-Eaton syndrome, leukocytoclastic vasculitis, lichen planus, lichen sclerosus, lignoconjunctivitis, linear IgA disease (LAD), lupus, chronic Lyme disease (Lyme disease chronic), Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, recurrent rheumatism (PR), PandAS, paraneoplastic cerebellar degeneration (PCD), paroxysmal nocturnal hemoglobinuria (PNH), Parry Romberg syndrome, ciliary planitis (peripheral uveitis), Parsonage-Tumer syndrome), pemphigus, peripheral neuropathy, perivenous encephalomyelitis, pernicious anemia (PA), POEMS syndrome, polyarteritis nodosa, polyglandular syndrome type I, II, type III, polymyalgia rheumatica, polymyositis, post-myocardial infarction syndrome, postpericardiotomy syndrome, primary biliary cirrhosis, primary sclerosing cholangitis, progesterone dermatitis, psoriasis, psoriatic arthritis, pure red blood cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff-person syndrome (SPS), subacute bacterial endocarditis (SBE), Susac's syndrome syndrome), sympathetic ophthalmia (SO), Takayasu's arteritis, temporal arteritis/giant cell arteritis, thrombocytopenic purpura (TTP), Tolosa-Hunt syndrome (THS), transverse myelitis, type 1 diabetes mellitus, ulcerative colitis (UC), undifferentiated connective tissue disease (UCTD), uveitis, vasculitis, Vitiligo and Vogt-Koyanagi-Harada disease.

In some embodiments, the TL1A-mediated disease or condition is cancer, optionally wherein
TL1A-mediated cancers are selected from one or more of the following: non-restrictive examples of cancer include adenoid cystic carcinoma, adrenal carcinoma, amyloidosis, anal cancer, ataxia telangiectasia, atypical nevus syndrome, basal cell carcinoma,
cholangiocarcinoma, Birt Hogg Dube syndrome, bladder cancer, bone cancer, brain tumor, breast
cancer, male breast cancer, carcinoid tumor, cervical cancer, colorectal cancer, ductal cancer, Endometrial Cancer, Esophageal Cancer, Gastric Cancer, Gastrointestinal Stromal Tumor (GIST), HER2-Positive Breast Cancer, Pancreatic Islet Cell Tumor, Juvenile Polyposis Syndrome, Kidney Cancer, Laryngeal Cancer, Leukemia-Acute Lymphoblastic Leukemia, Acute
Lymphoblastic Leukemia (ALL), Acute Myeloid Leukemia AML, Adult Leukemia, Childhood Leukemia, Chronic Lymphocytic Leukemia (CLL), Chronic Myeloid Leukemia (CML), Liver Cancer, Lobular Carcinoma, Lung Cancer, Small Cell Lung Cancer (SCLC), Non-small cell lung cancer (NSCLC), Hodgkin's lymphoma (Lymphoma-Hodgkin's), non Hodgkin's lymphoma (Lymphoma-Non-Hodgkin's), malignant glioma, melanoma, meningioma, multiple myeloma, myelodysplastic syndrome (MD), nasopharyngeal carcinoma, neuroendocrine tumor, oral cancer, osteosarcoma, ovarian cancer, pancreatic cancer, pancreatic neuroendocrine tumor, parathyroid carcinoma, penile cancer, peritoneal cancer, Peutz-Jeghers syndrome, pituitary tumors, polycythemia vera, prostate cancer, renal cell carcinoma, retinoblastoma, salivary gland carcinoma, sarcoma, Sarcoma-Kaposi, skin cancer, small bowel cancer, stomach cancer, testicular cancer, thymoma, thyroid cancer, uterine (endometrial) cancer, vaginal cancer, and Wilms' Tumor.

Other aspects and advantages of the present application will become readily apparent to those skilled in the art from the following detailed description. Only exemplary embodiments of the present application are shown and described in the following detailed description. As will be recognized by those skilled in the art, the contents of the present application enable those skilled in the art to make modifications to the specific embodiments disclosed without departing from the spirit and scope of the present application to which the present application pertains. Accordingly, the drawings and description herein are to be regarded as illustrative in nature, and not as restrictive.

### BRIEF DESCRIPTION OF THE DRAWINGS

Specific features of the present application to which the present application relates are as set forth in the appended claims. The features and advantages of the present application to which the present application relates will be better understood by reference to the exemplary embodiments and drawings described in detail below. A brief description of the drawings is as follows:

FIG. 1 shows a schematic diagram of the bispecific antibody configuration described in this application;

FIG. 2 shows the predicted structure of the TLA0015 monoclonal antibody and TL1A antigen complex in this application;

FIG. 3 shows the ELISA test results of the binding of different bispecific antibodies described in this application to human TL1A-his protein;

FIGS. 4A-4C show the binding and dissociation curves of different anti-TL1A antibodies described in this application to human TL1A-his protein;

FIGS. 5A-5C show the binding and dissociation curves of different anti-TL1A antibodies described in this application to monkey TL1A-his protein;

FIGS. 6A-6B show the binding and dissociation curves of the antibodies described in this application to human α4β7 protein.=Isolation curve;

FIG. 7A-7B shows that the bispecific antibody described in this application blocks the binding of TL1A-his protein to HEK-293-DR3 cells;

FIG. 8A-8D shows the effect of α4β7 protein on the blocking activity of the bispecific antibody described in the application;

FIG. 9 shows that the bispecific antibody described in this application inhibits the activity of Caspase3/7 activated by TL1A;

FIG. 10 shows that the bispecific antibody described in this application inhibits the NF-κB signaling pathway activated by TL1A-his protein;

FIG. 11 shows the ELISA test results of the binding of different bispecific antibodies described in this application to α4β7 protein;

FIG. 12 shows the binding of the bispecific antibody described in this application to Hut 78 cell binding curve;

FIG. 13 shows the bispecific antibody described in the present application blocking the binding of α4β7 and ligand MAdCAM-1;

FIG. 14 shows the bispecific antibody described in the present application blocking MAdCAM-1-mediated cell adhesion;

FIG. 15 shows the effect of TL1A protein on the bispecific antibody described in the present application blocking cell adhesion function;

FIGS. 16A-16B show the endocytic activity of different bispecific antibodies described in the present application on HuT 78 cells;

FIGS. 17A-17D show the effect of adding TL1A protein on the endocytic activity of the bispecific antibody described in the present application on HuT 78 cells and primary T cells;

FIGS. 18 and 19 show the endocytic activity of different bispecific antibodies described in the present application on HuT 78 cells;

FIG. 20 shows the bispecific antibody described in the present application downregulating HuT 78 cell membrane, and after adding TL1A protein, the endocytosis activity increased, which can more obviously down-regulate the expression of α4β7 on the membrane;

FIG. 21A-FIG. 21B show that the bispecific antibody described in this application down-regulates the total expression of α4β7 in HuT 78 cells, and after adding TL1A protein, the endocytosis activity increased, which can more obviously down-regulate the total expression of α4β7;

FIG. 22A shows the recovery of α4β7 on the HuT 78 cell membrane at different times after endocytosis (antibody concentration 5 nM);

FIG. 22B shows the proportion of positive cells that recover the expression of α4β7 on the HuT 78 cell membrane at different times after endocytosis (antibody concentration 5 nM);

FIG. 23A shows the recovery of α4β7 on the HuT 78 cell membrane at different times after endocytosis (antibody concentration 50 nM);

FIG. 23B shows the recovery of α4β7 on the HuT 78 cell membrane at different times after endocytosis (antibody concentration 50 nM); 78 The proportion of positive cells that restored expression of α4β7 on the cell membrane at different times (antibody concentration 50 nM);

FIG. 24A shows the restoration of expression of α4β7 on the PanT cell membrane at different times after endocytosis (antibody concentration 5 nM);

FIG. 24B shows the proportion of positive cells that restored expression of α4β7 on the PanT cell membrane at different times after endocytosis (antibody concentration 5 nM);

FIG. 25 shows the inhibitory activity of the antibody on PanT cell migration;

FIG. 26 shows the endocytosis activity of the antibody in PanT cells (Donor1);

FIGS. 27A-27B show the endocytosis of the antibody, The recovery of α4β7 expression on the membrane of PanT cells at different times (Donor1);

FIGS. 28A-28B show the migration of PanT cells at different times (Donor1);

FIG. 29 shows the endocytosis activity of antibodies in PanT cells (Donor2);

FIGS. 30A-30B show the endocytosis of antibodies and the recovery of α4β7 expression on the membrane of PanT cells at different times (Donor2);

FIGS. 31A-31B show the migration of PanT cells at different times (Donor2);

FIG. 32A shows the inhibitory activity of the antibodies disclosed in this application on CD4 T cell activation;

FIG. 32B shows the inhibitory activity of the antibodies disclosed in this application on CD4 T cell proliferation;

FIG. 32C shows the inhibitory activity of the antibodies on CD4 T cell activation and proliferation inhibitory activity;

FIG. 33 shows the inhibitory activity of the bispecific antibody described in this application on IFN-7 secretion;

FIGS. 34A-34C show the killing effect of the bispecific antibody described in this application on PBMC under different conditions;

FIGS. 35A-35F show the activation of different T cells after the bispecific antibody described in this application is co-incubated with CHO-K1 cells and PBMC antibodies;

FIGS. 36A-36F show the activation of different T cells after the bispecific antibody described in this application is co-incubated with CHO-K1-TL1A cells and PBMC antibodies;

FIGS. 37A-37F show the activation of different T cells after the bispecific antibody described in this application is co-incubated with PBMC antibodies;

FIGS. 38A-38E show the activation of different T cells after the bispecific antibody described in this application is co-incubated with CHO-K1 cells

FIG. 39A-39E shows the release of different cytokines after the bispecific antibody described in this application is co-incubated with CHO-K1-TL1A cells and PBMC antibodies;

FIG. 40 shows the PK detection of TLb0045 described in this application in rats;

FIG. 41 shows the PK detection of TLb0065 described in this application in rats;

FIG. 42 shows the PK detection of TLb0045 described in this application and TLb0065 described in this application in rats;

FIG. 43 shows the PK detection of TLb0045 described in this application in hFcRn mice;

FIG. 44 shows the PK detection of TLb0045-1 described in this application in hFcRn mice.

FIGS. 45A-45B show the weight changes of the antibody in the DSS-induced hTL1A/hα4β7 transgenic mouse enteritis efficacy experiment;

FIG. 46 shows the daily disease score of the antibody in the DSS-induced hTL1A/hα4β7 transgenic mouse enteritis efficacy experiment;

FIGS. 47A-47B show the colorectal length changes of the antibody in the DSS-induced hTL1A/hα4β7 transgenic mouse enteritis efficacy experiment;

FIGS. 48A-48B show the colorectal weight changes of the antibody in the DSS-induced hTL1A/hα4β7 transgenic mouse enteritis efficacy experiment;

FIGS. 49A-49B show the weight changes of the antibody in the TNBS-induced hTL1A/hα4β7 transgenic mouse enteritis efficacy experiment;

FIG. 50 shows the colorectal length changes of the antibody in the TNBS-induced hTL1A/Daily disease scores in the hα4β7 transgenic mouse enteritis efficacy experiment;

FIG. 51 shows the mouse survival curve results in the TNBS-induced hTL1A/hα4β7 transgenic mouse enteritis efficacy experiment;

FIGS. 52A-52B show the changes in colorectal length in the TNBS-induced hTL1A/hα4β7 transgenic mouse enteritis efficacy experiment;

FIGS. 53A-53B show the changes in colorectal weight in the TNBS-induced hTL1A/hα4β7 transgenic mouse enteritis efficacy experiment.

### DETAILED DESCRIPTION OF THE INVENTION

The embodiments of the invention of the present application are illustrated by particular examples. Other advantages and effects of the present application will become readily apparent to those skilled in the art from disclosure of the present specification.

### Definition of Terms

In the present application, the term “isolated” generally refers to a product obtained from a natural state by artificial means. If an “isolated” substance or component occurs in nature, it may be altered from its natural environment, or the substance may be isolated from its natural environment, or both. For example, an unisolated polynucleotide or polypeptide naturally occurs in a living animal and the same polynucleotide or polypeptide isolated from its natural state in high purity is the to be isolated. The term “isolated” does not exclude the admixture of artificial or synthetic substances, nor the presence of other impure substances which do not affect the activity of the substance.

In the present application, the term “antigen-binding protein” generally refers to a polypeptide molecule capable of specifically recognizing and/or neutralizing a particular antigen. In the present application, the term “antigen-binding protein” may include an “antibody” or an “antigen-binding fragment”. For example, the antibody may comprise an immunoglobulin composed of at least two heavy (H) chains and two light (L) chains interconnected by disulfide bonds, and may include any molecule comprising an antigen-binding portion thereof. The term “antibody” may include monoclonal antibodies, antibody fragments, or antibody derivatives, including, but not limited to, murine antibodies, human antibodies (fully human antibodies), humanized antibodies, chimeric antibodies, single chain antibodies (e.g., scFv), and antibody fragments that bind to an antigen (e.g., Fab, Fab′, and (Fab)2 fragments). The term “antibody” may also include all recombinant forms of antibodies, such as antibodies expressed in prokaryotic cells, non-glycosylated antibodies, and any antigen-binding antibody fragments and derivatives thereof described herein. Each heavy chain may be composed of a heavy chain variable region and a heavy chain constant region. Each light chain may be composed of a light chain variable region and a light chain constant region. The VH and VL regions can be further distinguished as hypervariable regions called complementarity determining regions (CDRs), interspersed with more conserved regions called framework regions (FRs). Each VH and VL may be composed of three CDRs and four FRs, which may be arranged from amino-terminus to carboxy-terminus in the following order: FR1, CDR1, FR2, CDR2, FR3, CDR3, and FR4. The variable regions of the heavy and light chains contain binding domains that interact with an antigen. The constant region of an antibody can mediate the binding of the immunoglobulin to host tissues or factors, including various cells of the immune system (e.g., effector cells) and the first component of the classical complement system (C1q). The exact boundaries of the CDRs have been defined differently for different systems. The system described by Kabat (Kabat et al., Sequences of Proteins of Immunological Interest (National Institutes of Health, Bethesda, Md. (1987) and (1991)) provides not only an unambiguous residue numbering system applicable to any variable region of an antigen-binding fragment, but also provides precise residue boundaries defining CDRs. These CDRs may be referred to as Kabat CDRs. Chothia and co-workers (Chothia and Lesk, J. Mol. Biol. 196: 901-917(1987) and Chothia et al., Nature 342: 877-883(1989)) found that certain sub-portions within Kabat CDRs adopt nearly identical conformations in the backbone of a peptide despite the large diversity at the amino acid sequence level. These sub-portions are designated as L1, L2, and L3 or H1, H2, and H3, where “L” and “H” refer to the light chain and heavy chain regions, respectively. These regions may be referred to as Chothia CDRs, which have boundaries that overlap with Kabat CDRs. Other boundaries that define CDRs and overlap with Kabat CDRs have been described by Padlan (FASEB J. 9: 133-139 (1995)) and MacCallum (J Mol Biol 262(5): 732-45(1996)). In addition, other CDR boundary definitions may not strictly follow one of the above systems, but will still overlap with Kabat CDRs, although they may be shortened or lengthened in light of prediction or experimental findings that a particular residue or group of residues or even the entire CDRs, do not significantly affect the antigen binding. In the present application, said CDR may be defined through the Kabat numbering system.

In the present application, the term “antigen-binding fragment” generally refers to one or more fragments of an antibody that function to specifically bind to an antigen. The antigen-binding function of an antibody can be achieved by a full-length fragment of the antibody. The antigen-binding function of an antibody may also be achieved by: a heavy chain comprising a fragment of Fv, scFv, dsFv, Fab, Fab′ or F (ab′)2, or a light chain comprising a fragment of Fv, scFv, dsFv, Fab, Fab′, or F(ab′)2. (1) An Fab fragment, i.e., a monovalent fragment consisting of the VL, VH, CL, and CH domains; (2) an F(ab′)2 fragment, a bivalent fragment comprising two Fab fragments linked by a disulfide bond at the hinge region; (3) an Fd fragment consisting of the VH and CH domains; (4) an Fv fragment consisting of the VL and VH domains of a single arm of an antibody; (5) a dAb fragment consisting of VH domains (Ward et al., (1989) Nature 341: 544-546); (6) an isolated complementarity determining region (CDR), and (7) a combination of two or more isolated CDRs which may optionally be joined by a linker. For example, monovalent single chain molecules Fv (scFv) formed by the pairing of VL and VH may also be included (see, Bird et al., (1988) Science 242: 423-426; and Huston et al., (1988) Proc. Natl. Acad. Sci. 85: 5879-5883). For example, a class of antibody VHH lacking the antibody light chain but only the heavy chain variable region can also be included (see, e.g., Kang Xiaozhen et al., Chinese Journal of Biotechnology, 2018, 34 (12): 1974-1984). The “antigen-binding portion” may also include an immunoglobulin fusion protein comprising a binding domain selected from the group consisting of: (1) a binding domain polypeptide fused to an immunoglobulin hinge region polypeptide; (2) an immunoglobulin heavy chain CH2 constant region fused to the hinge region; and (3) an immunoglobulin heavy chain CH3 constant region fused to the CH2 constant region.

The basic 4-chain antibody unit is a heterotetrameric glycoprotein composed of two identical light (L) chains and two identical heavy (H) chains. IgM antibodies consist of 5 basic heterotetrameric units and another polypeptide called the J chain and contain 10 antigen binding sites, while IgA antibodies consist of 2-5 basic 4-chain units, which can polymerize to form a multivalent assembly bound to the J chain. In the case of IgG, the 4-chain unit is usually about 150,000 Daltons. Each L chain is linked to the H chain by one covalent disulfide bond, while the two H chains are linked to each other by one or more disulfide bonds, depending on the isotype of the H chain. Each H and L chain also has regularly spaced intrachain disulfide bridges. Each H chain has a variable domain (VH) at the N-terminus, followed by three constant domains (CH) in each α and γ chain, and four CH domains in the and F isotypes. Each L chain has a variable domain (VL) at the N-terminus and then a constant domain at the other end. VL is aligned with VH, and CL is aligned with the first constant region (CH1) of the heavy chain. Specific amino acid residues are believed to form an interface between the light chain variable domain and the heavy chain variable domain. The pairing of VH and VL together forms a single antigen binding site. For the structure and properties of different classes of antibodies, see, for example, Basic and Clinical Immunology, 8th edition, Daniel P. Sties, Abba I. Terr and Tristram G. Parsolw (eds.), Appleton & Lange, Norwalk, Conn., 1994, page 71 and Chapter 6. The L chains of any vertebrate species can be divided into two clearly distinct types, called kappa and lambda, based on the amino acid sequence of their constant regions. Immunoglobulins can be divided into different classes or isotypes based on the amino acid sequence of their heavy chain constant domain (CH). There are five classes of immunoglobulins: IgA, IgD, IgE, IgG, and IgM, with heavy chains designated α, δ, ε, γ, and μ, respectively. Based on relatively minor differences in the sequence and function of CH, the gamma and alpha classes are further divided into subclasses, for example, humans express the following subclasses: IgG1, IgG2A, IgG2B, IgG3, IgG4, IgA1, and IgK1.

In the present application, the term “monoclonal antibody” generally refers to a population of substantially homologous antibodies, i.e., the individual antibodies comprising the population are identical except for possible naturally occurring mutations that may be present in minor amounts. Monoclonal antibodies are highly specific, being directed against a single antigenic site. For example, the monoclonal antibodies can be prepared by hybridoma techniques or can be produced in bacteria, eukaryotic animal or plant cells using recombinant DNA methods, or can be derived from phage antibody libraries using the techniques, for example, described in Clackson et al., Nature, 352:624-628 (1991) and Marks et al., Mol. Biol., 222:581-597 (1991).

In the present application, the term “chimeric antibody” generally refers to an antibody in which a portion of the amino acid sequence of each heavy or light chain is homologous to the corresponding amino acid sequence in an antibody from a particular species, or belongs to a particular class, while the remaining segments of the chain are homologous to the corresponding sequence in another species. For example, the variable regions of both the light and heavy chains are derived from the variable region of an antibody of one animal species (e.g., mouse, rat, etc.), while the constant portions are homologous to antibody sequences from another species (e.g., human). For example, to obtain chimeric antibodies, non-human-derived B cells or hybridoma cells can be used to generate variable regions, while which the constant regions in combination therewith are of human origin. The variable region has the advantage of being easy to prepare and its specificity is not influenced by the origin of the constant region with which it is combined. Also, since the constant region of the chimeric antibody can be derived from human, the chimeric antibodies are less likely to elicit an immune response upon injection than using antibodies whose constant regions are of non-human origin.

In the present application, the term “humanized antibody” generally refers to a chimeric antibody that contains fewer sequences from non-human immunoglobulins, thereby reducing the immunogenicity of a xenogenous antibody when introduced into humans, while maintaining the full antigen-binding affinity and specificity of the antibody. For example, non-human binding domains can be humanized using the technical means, such as CDR transplantation (Jones et al., Nature 321:522(1986)) and variants thereof, including “reshaping”, (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., “Derivation of therapeutically active humanized and veneered anti-CD18 antibodies.” In: Metcalf B W, Dalton B J, eds. Cellular adhesion: molecular definition to therapeutic potential. New York: Plenum Press, 1994: 291-312), and resurfacing (U.S. Pat. No. 5,639,641). Other regions, such as hinge and constant region domains, may also be humanized if they are also derived from non-human sources.

In the present application, the term “murine antibody” generally refers to an antibody in which the variable region framework and CDR regions are derived from mouse germline immunoglobulin sequences. In addition, if the antibody comprises constant regions, which are also derived from mouse germline immunoglobulin sequences. The murine antibodies of the present application may comprise amino acid residues not encoded by the mouse germline immunoglobulin sequences, such as mutations introduced by random or point mutations in vitro or by somatic mutation in vivo.

In the present application, the term “directly linked” is used in contrast to the term “indirect linked”, which usually means a direct connection. For example, said direct connection may be a direct connection between substances without spacers. Said spacer may be a linker. For example, said linker may be a peptide linker. The term “indirect linkage” generally refers to the case where there is no direct linkage between substances. For example, said indirect linkage may be a linkage through a spacer. For example, in the isolated antigen-binding proteins described in the present application, the C-terminal end of said L-FR1 and the N-terminal end of said LCDR1 may be directly or indirectly linked.

In this application, the term “TL1A” refers to TNF-like protein 1A. TL1A (TNFSF15) is a member of the TNF family that is expressed predominantly by endothelial cells, macrophages and dendritic cells (DCs). Its expression is induced by immune complexes (ICs) and cytokines. TL1A receptor DR3 is expressed mainly on T cells and NKT cells. In vitro, TL1A has been shown to enhance T cell proliferation and cytokine production in both humans and mice. In vivo, TL1A transgenic mice produce an IBD phenotype similar to human Crohn's disease. In addition, treatment with recombinant TL1A protein exacerbated colitis in mdr1−/− mice. The TL1A described in this application may be a monomer or trimer of TL1A. The TL1A described in this application can be a human, mouse or monkey TL1A.

The cell surface molecule “α4β7 integrin” or “α4β7” is a heterodimer formed by the α4 chain (CD49D, ITGA4) and the β7 chain (ITGB7). α4 and β7 chains also form a heterodimer α4β1 or αEβ7 with the alternative integrin chains β1 or αE, respectively. αE to form a heterodimer α4β3l or αEβ7. human α4 and β7 genes (GenBank (National Center for Biotechnology Information, Bethesda, MD) RefSeq Nos. NM_000885 and NM_000889, respectively) by B and T lymphocytes, especially memory CD4+ lymphocytes. As typical of many integrins, α4β7 can exist in either a quiescent or activated state. ligands for α4β7 include vascular cell adhesion molecule (VCAM), fibronectin, and mucosal addressin (MAdCAM (e.g., MAdCAM-1)). The α4β7 described in the present application may be human, murine or monkey α4β7.

In this application, the term “scFv” generally refers to a fusion protein comprising at least one antibody fragment comprising a light chain variable region and at least one antibody fragment comprising a heavy chain variable region, wherein said light chain and heavy chain variable regions are adjacent (e.g., via synthetic junctions, such as short flexible peptide junctions) and are capable of being expressed as a single-chain polypeptide, and said scFv retains the specificity of the intact antibody from which it is derived. Unless specifically stated, as used in this application, an scFv may have said VL and VH variable regions in any order (e.g., relative to the N-terminus and the C-terminus of the polypeptide), and an scFv may comprise a VL-linker-VH or may comprise a VH-linker-VL.

In this application, the terms “specific binding” or “specific” generally refer to measurable and reproducible interactions, such as binding between a target and an antibody, that may determine the presence of a target in the presence of a heterogeneous population of molecules, including biomolecules. For example, an antibody that specifically binds a target (which may be an epitope) may be an antibody that binds that target with greater affinity, affinity, more readily, and/or for a greater duration than it binds other targets. In some embodiments, the antibody specifically binds an epitope on a protein, said epitope being conserved across different species of proteins. In certain embodiments, specific binding may include, but is not required to exclusively bind.

In addition to the specific proteins and nucleotides mentioned herein, the present application may also include functional variants, derivatives, analogs, homologs, and fragments thereof.

The term “functional variant” refers to an amino acid sequence that is substantially identical to a naturally occurring sequence or a polypeptide encoded by a substantially identical nucleotide sequence and capable of having one or more activities of the naturally occurring sequence. In the context of the present application, a variant of any given sequence refers to a sequence in which the particular sequence of residues, whether amino acid or nucleotide residues, has been modified such that the polypeptide or polynucleotide substantially retains at least one endogenous function. Variant sequences may be obtained by the addition, deletion, substitution, modification, substitution and/or variation of at least one amino acid residue and/or nucleotide residue present in a naturally occurring protein and/or polynucleotide, so long as the original functional activity is retained.

In the present application, the term “derivative” generally refers to a polypeptide or polynucleotide of the present application comprising any substitution, variation, modification, replacement, deletion and/or addition of one (or more) amino acid residues from/on the sequence, so long as the resulting polypeptide or polynucleotide substantially retains at least one of its endogenous functions.

In the present application, the term “analog” generally refers to a polypeptide or polynucleotide including any mimetic of a polypeptide or polynucleotide, i.e., a chemical compound that possesses at least one endogenous function of the polypeptide or polynucleotide that the mimetic mimics.

Generally, amino acid substitutions, e.g., at least one (e.g., 1, 2, 3, 4, 5, 6, 7, 8, 9, 10, or 20 or more) amino acid substitution can be made, so long as the modified sequence substantially retains the desired activity or ability. Amino acid substitutions can include the use of non-naturally occurring analogs.

In the present application, the term “homologue” generally refers to an amino acid sequence or nucleotide sequence having certain homology to a naturally occurring sequence. The term “homology” can be equivalent to sequence “identity”. Homologous sequences may include amino acid sequences that may be at least 80%, 85%, 90%, 99.1%, 99.2%, 99.3%, 99.4%, 99.5%, 99.6%, 99.7%, 99.8%, or 99.9% identical to the subject sequence. Typically, the homologue will comprise the same active site or the like as the subject amino acid sequence. Homology may be considered in terms of similarity (i.e., amino acid residues having similar chemical properties/functions) or may be expressed in terms of sequence identity. In the present application, a sequence having percent identity in any one of the SEQ ID NO of a mentioned amino acid sequence or nucleotide sequence refers to a sequence having the percent identity over the entire length of the mentioned SEQ ID NO. To determine sequence identity, sequence alignments may be performed by a variety of ways known to those skilled in the art, e.g., using BLAST, BLAST-2, ALIGN, NEEDLE, or Megalign (DNASTAR) software, etc. Those skilled in the art can determine the appropriate parameters for the alignment, including any algorithm required to achieve the optimal alignment over the full length of the sequence being compared.

The proteins or polypeptides used in the present application may also have deletions, insertions, or substitutions of amino acid residues which produce silent changes and result in functionally equivalent proteins. Intentional amino acid substitutions may be made on the basis of similarity in polarity, charge, solubility, hydrophobicity, hydrophilicity, and/or amphipathic nature of the residues, so long as the endogenous function is retained. For example, negatively charged amino acids include aspartic acid and glutamic acid; positively charged amino acids include lysine and arginine; and amino acids containing uncharged polar head groups with similar hydrophilicity values include asparagine, glutamine, serine, threonine, and tyrosine.

In the present application, the term “immunoconjugate” generally refers to a conjugate formed by the conjugation of other agents (e.g., a chemotherapeutic agent, a radioactive element, a cytostatic agent, and a cytotoxic agent) to The isolated antigen-binding protein (e.g., via covalent attachment of a linking molecule), wherein the conjugate can deliver the other agents to a target cell (e.g., a tumor cell) via specific binding of The isolated antigen-binding protein to an antigen on the target cell. The immunoconjugate then undergoes such internalization and eventually enters the interior of the target cell (e.g., into vesicles such as a lysosome), at which point the linker molecule in the immunoconjugate can be cleaved to release the other agent, thereby exerting its cytotoxic effect. In addition, the antigen may also be secreted by the target cell and located in the space outside the target cell.

In the present application, the term “subject” generally refers to human or non-human animals, including, but not limited to, cats, dogs, horses, pigs, cows, caprid, rabbits, mice, rats, or monkeys.

In the present application, the term “nucleic acid molecule” generally refers to an isolated form of nucleotides, deoxyribonucleotides, or ribonucleotides or analogs thereof, of any length, isolated from their natural environment or artificially synthesized.

In the present application, the term “vector” generally refers to a nucleic acid molecule capable of self-replication in a suitable host. The vector can transfer the inserted nucleic acid molecule into and/or between cells. The vector may include a vector for primarily inserting DNA or RNA into a cell, a vector for primarily replicating DNA or RNA, and a vector for primarily expressing transcription and/or translation of DNA or RNA. The vector can be a polynucleotide capable of being transcribed and translated into a polypeptide when introduced into an appropriate cell. In general, the vector may produce the desired expression product by culturing an appropriate cell containing the vector. In the present application, the vector may include a lentiviral vector.

In the present application, the term “cell” generally refers to an individual cell, cell line or cell culture that may or may already contain a plasmid or vector comprising a nucleic acid molecule as described herein, or that is capable of expressing a polypeptide as described herein or an antigen-binding protein as described herein. The cell may include the progeny of a single cell. Due to natural, accidental, or deliberate mutations, the progeny cells may not necessarily be identical in morphology or in genome to the original parent cell, but are capable of expressing the polypeptide or antigen-binding protein as described herein. The cells can be obtained by transfecting cells in vitro with the vectors as described herein. The cells may be prokaryotic cells (e.g., E. coli) or eukaryotic cells (e.g., yeast cell, COS cells, Chinese Hamster Ovary (CHO) cells, HeLa cells, HEK293 cells, COS-1 cells, NS0 cells, or myeloma cells). In some embodiments, the cells may be immune cells. For example, the immune cell may be selected from the group consisting of T cells, B cells, natural killer cells (NK cells), macrophages, NKT cells, monocytes, dendritic cells, granulocytes, lymphocytes, leukocytes and/or peripheral blood mononuclear cells.

In the present application, the term “treatment” generally refers to: (i) the prevention of the development of a disease, disorder, and/or condition in a patient who may be susceptible to, but has not yet been diagnosed with, that disease, disorder, or condition; (ii) the suppression of the disease, disorder, or condition, i.e., the curb of the development; and (iii) remission of the disease, disorder, or condition, i.e., causing regression of the disease, disorder, and/or condition and/or symptoms associated with the disease, disorder, and/or condition.

In the present application, the terms “polypeptide”, “peptide”, and “protein” are used interchangeably and generally refer to a polymer of amino acids of any length. The polymer may be linear or branched, and may comprise modified amino acids and it may be interrupted by non-amino acids. These terms also encompass amino acid polymers that have been modified. These modifications may comprise: disulfide bond formation, glycosylation, lipidation, acetylation, phosphorylation, or any other manipulation (e.g., binding to a labeling component). The term “amino acid” includes natural and/or non-natural or synthetic amino acids, including glycine as well as the D and L optical isomers, as well as amino acid analogs and peptidomimetics.

In the present application, the terms “polynucleotide”, “nucleotide”, “nucleotide sequence”, “nucleic acid”, and “oligonucleotide” are used interchangeably and refer generally to polymeric forms of nucleotides of any length, such as deoxyribonucleotides or ribonucleotides, or analogs thereof. A polynucleotide may have any three-dimensional structure and may perform any function that is known or unknown. Non-limiting examples of polynucleotides are as follows: a coding or noncoding region of a gene or gene fragment, a plurality of loci (one locus) as defined by ligation analysis, exons, introns, messenger RNA (mRNA), transport RNA, ribosomal RNA, short interfering RNA (siRNA), short hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modification of the nucleotide structure may be performed before or after assembly of the polymer. The sequence of nucleotides may be interrupted by non-nucleotide components. The polynucleotides may be further modified after polymerization, e.g., by conjugation to labeled components.

In the present application, the term “K_{D}” (likewise, “K_{D}” or “KD”) generally refers to an “affinity constant” or an “equilibrium dissociation constant” and refers to a value obtained at equilibrium in a titration measurement, or by dividing the dissociation rate constant (k_{d}) by the binding rate constant (kₐ). The binding affinity of a binding protein (e.g., an isolated antigen-binding protein as described herein) for an antigen is expressed using an association rate constant (kₐ), a dissociation rate constant (k_{d}), and an equilibrium dissociation constant (K_{D}). Methods for determining association and dissociation rate constants are well known in the art. The use of fluorescence-based techniques provides high sensitivity and the ability to examine samples at equilibrium in physiological buffers. For example, the K_{D} value can be determined by Octet assay, and other experimental approaches and instruments such as BIAcore (Biomolecular Interaction Analysis) can be used (e.g., instruments available from BIAcoreInternationalAB, aGEHealthcarecompany, Uppsala, Sweden). Alternatively, the K_{D} value can be determined using KinExA (dynamic exclusion assay (KineticExclusionAssay)) available from SapidyneInstruments (Boise, Idaho) or using a surface plasmon resonance (SPR) instrument.

In the present application, the term “IC50 value” or the term “half-maximal inhibitory concentration” (IC50) indicates the concentration of a particular compound that is required to achieve 50% inhibition of a biological process in vitro. The IC50 value can be logarithmically converted to a pIC50 value (−log IC50), where higher values indicate exponentially greater potency. The IC50 value is not an absolute value, but rather depends on experimental conditions, such as the concentration used. The IC50 value can be converted to an absolute inhibitory constant (Ki) using the Cheng-Prusoff equation (Biochem. Pharmacol. (1973) 22:3099).

The term “EC50”, in the context of in vitro or in vivo analyses using isolated antigen-binding fragments, refers to the concentration of the antibody or antigen-binding portion thereof when the induced response is 50 of the maximal response, i.e., halfway between the maximal response and the baseline.

The terms “α4β7 inhibitor” and “α4β7 antagonist” are used interchangeably. Each is a molecule that detectably inhibits at least one function of α4β7. Conversely, an “α4β7 agonist” is a molecule that detectably enhances at least one function of α4β7. The inhibition caused by the α4β7 inhibitor need not be complete, as long as it is detectable by, for example, employing an assay. Any assay of α4β7 function may be employed, examples of which are provided herein. Examples of α4β7 functions that can be inhibited by α4β7 inhibitors (or enhanced by α4β7 agonists) include ligand binding (i.e., binding to MAdCAM-1), adhesion to cells expressing the ligand, transport to specific compartments (e.g., intestines), release of cytokines, chemokines, and other mediators, exacerbation or worsening of inflammatory responses and tissue damage, and the like. α4β7 inhibitors and α4β7 agonists of the Examples of types include, but are not limited to, α4β7-binding polypeptides, such as antigen-binding proteins (e.g., α4β7 antigen-binding proteins), antibodies, antibody fragments, and antibody derivatives.

The terms “TL1A inhibitor” and “TL1A antagonist” are used interchangeably. Each is a molecule that detectably inhibits at least one function of TL1A. Conversely, a “TL1A agonist” is a molecule that detectably enhances at least one function of TL1A. The inhibition caused by the TL1A inhibitor need not be complete, as long as it is detectable by, for example, using an assay. Any assay for α4β7 function may be employed, examples of which are provided herein. Examples of functions of TL1A that can be inhibited by TL1A inhibitors (or enhanced by TL1A agonists) include ligand binding (i.e., binding to MAdCAM-1), adhesion to cells expressing the ligand, transport to specific compartments (e.g., intestines), release of cytokines, chemokines, and other mediators, exacerbation or worsening of inflammatory responses and tissue damage, and the like. The TL1A inhibitors and the TL1A agonists of the Examples of types include, but are not limited to, TL1A-binding polypeptides, such as antigen-binding proteins (e.g., TL1A antigen-binding proteins), antibodies, antibody fragments, and antibody derivatives.

The term “endocytosis” or “internalization” usually refers to the process by which the cell membrane transfers substances from outside the cell into the cell through deformation movements. Said endocytosis may involve both phagocytosis and cytotoxicity. For example, the cell may be depressed to form “vesicles” that encapsulates said macromolecules (e.g., proteins), but then detach from the cell membrane and enter the cell interior.

The degree of degradation by endocytosis can be evaluated by means of an “endocytosis degradation rate”. For a cell-derived macromolecule (e.g., a soluble protein) interacting with a cell, the “endocytosis degradation rate” of said macromolecule may refer to the amount of said macromolecule that enters the cell via said endocytosis as a percentage of the total amount of said macromolecule interacting with said cell (which may generally be the sum of the amount endocytosed and the amount bound to the surface of said cell and the amount of said macromolecule bound to the surface of said cell). (generally the total amount can be the sum of the amount endocytosed and the amount bound to the surface of said cell) as a proportion (usually expressed as a percentage) of the total amount of said macromolecule interacting with said cell. For a macromolecule on the surface of a cell membrane (e.g. a membrane protein on said cell), said “endocytosis degradation rate” of a macromolecule may refer to the ratio of the amount of said macromolecule which enters the cell via said endocytosis and is degraded to the total amount of said macromolecule on said cell (which generally may be the sum of the amount endocytosed and the amount of the macromolecule which is endocytosed and degraded). (generally the total amount can be the sum of the amount degraded by endocytosis and the amount remaining on the surface of the cell membrane after endocytosis; or it can also be expressed as the total amount on the surface of the cell membrane in the absence of endocytotic degradation), which is usually expressed as a percentage.)

As used herein, the term “epitope” or “antigenic determinant cluster” generally refers to a site on an antigen to which an antibody binds. Epitopes may be formed from contiguous amino acids (linear epitopes) or discontinuous amino acids (conformational epitopes) adjacent to each other by tertiary folding of the protein. Epitopes formed from contiguous amino acids are typically retained upon exposure to denaturing solvents, whereas epitopes formed by tertiary folding are typically lost upon treatment with denaturing solvents. Epitopes typically include at least 3, and more typically at least 5 or 8-10 amino acids in a unique spatial conformation. Methods for determining the spatial conformation of epitopes include, for example, x-ray crystallography and two-dimensional nuclear magnetic resonance. See, e.g., Epitope Mapping Protocols in Methods in Molecular Biology, vol. 66, edited by Glenn E. Morris (1996).

As used herein, the term “conformational epitope” generally refers to non-contiguous amino acid residues of an antigen (such as the PD-L1 antigen) that are adjacent to the protein by tertiary folding. These non-contiguous amino acid residues may converge on the surface when the polypeptide chain folds to form a natural protein. Conformational epitopes include, but are not limited to, functional epitopes.

As used herein, the term “cross-linking” refers to the process by which an antigen-binding protein binds to more than one antigen at the same time, or an antigen binds to more than one antigen-binding protein at the same time. For example, RVT-3101 (Analogue) monoclonal antibody binds to huTL1A-his antigen, which can be seen as three monoclonal antibodies bound to a single TL1A trimer, i.e., a 1Ag (Ag represents the antigen)+3Ab (Ab represents the antibody) complex. tla0015-11 monoclonal antibody binds to the huTL1A-his antigen, which results in the formation of a multiple-antigen binding protein that binds to a number of antigen-binding proteins cross-linked complexes.

As used herein, the term “functional epitope” generally refers to the amino acid residues of an antigen that actively contributes to the binding of an antibody, i.e., form an “active epitope.” Mutation of any active promoting residue of the antigen to alanine will destroy the binding of the antibody, so that the relative KD ratio of the antibody (KD mutant/KD wild type) can be, for example, greater than 2 times, such as greater than 3 times, greater than 4 times, greater than 6 times, greater than 10 times, greater than 20 times, greater than 30 times, greater than 40 times, greater than 50 times, greater than 60 times, greater than 70 times, greater than 80 times, greater than 90 times, greater than 100 times, greater than 150 times, greater than 200 times or more.

In the present application, the term “and/or” should be understood as meaning any one of the alternatives or both of the alternatives.

In the present application, the term “comprising” or “containing” generally refers to the inclusion of explicitly specified features, but not excluding other elements. In certain instances, “comprising” or “contain” also encompasses the inclusion of only the specified components.

In the present application, the term “about” generally refers to a range from 0.5% to 10% above or below the specified value, for example, a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below the specified value.

In the present application, the term “including” generally means comprising, summing up, encompassing or covering. In certain instances, the meaning of “being”, “consisting of . . . ” is also indicated.

### DETAILED DESCRIPTION

### First Antigen Binding Domain

In one aspect, the present application provides an isolated antigen-binding protein comprising a first antigen-binding domain which specifically binds to TL1A. The first binding domain includes a heavy chain variable region (VH). The VH includes at least one heavy chain complementarity determining regions (HCDR). The VH may include at least one, two or three of HCDR1, HCDR2 or HCDR3. In some embodiments, HCDR 3, HCDR2 and HCDR1 are sequences as set forth in SEQ ID NO:14, 5 and 1 respectively.

An HCDR3 of the antigen-binding protein may include amino acid sequence as set forth in SEQ ID NO:14. For example, the sequence of HCDR3 of the antigen-binding protein may be defined by Kabat.

In some embodiments, the HCDR3 may be GX1X2DAMDY(SEQ ID NO:14). X1 may be D or L or N, X2 may be F or Y.

In some embodiments, the HCDR3 may include an amino acid sequence as set forth in any one of SEQ ID NOs:15-18.

An HCDR2 of the antigen-binding protein may include amino acid sequence as set forth in SEQ ID NO:5. For example, the sequence of the HCDR2 of the antigen-binding protein may be defined by Kabat.

In some embodiments, the HCDR2 may be X3IWGFGX4TX5YX6X7ALKS(SEQ ID NO:5). X3 may be R or V, and X4 may be G or K, and X5 may be D or H or N, and X6 may be N or Q, and X7 may be A or P or S.

In some embodiments, the HCDR2 may include an amino acid sequence as set forth in any one of SEQ ID NOs:6-13.

An HCDR1 of the antigen-binding protein may include amino acid sequence as set forth in SEQ ID NO:1. For example, the sequence of the HCDR1 of the antigen-binding protein may be defined by Kabat.

In some embodiments, the HCDR1 may be X8YGVD (SEQ ID NO:1). X8 may be S or T or Y.

In some embodiments, the HCDR1 may include an amino acid as set forth in any one of SEQ ID NO:2-4.

In some embodiments, the VH comprises the HFR1, and C-terminus of the HFR1 is directly or indirectly linked to the N-terminus of the HCDR1. For example, the sequence of HFR1 of the antigen-binding protein may be defined by Kabat. HFR1 comprises an amino acid as set forth in SEQ ID NO:19.

In some embodiments, the HFR1 is EX9QLX10ESGGGLX11QPGGSLRX12SCAVSSGFXx1LXx2 (SEQ ID NO: 19). X9 may be I or V, X10 may be L or V, X11 may be I or V, X12 may be I or L. Xx1 is D or S, Xx2 is I or K or S.

In some embodiments, the HFR1 is selected from an amino acid sequence as set forth in any one of SEQ ID NO:20-28.

In some embodiments, the VH comprises the HFR2, and the HFR2 is located between the HCDR1 and the HCDR2. For example, the sequence of the HFR2 of the antigen-binding protein may be defined by Kabat. the HFR2 comprises an amino acid sequence as set forth in SEQ ID NO:29.

In some embodiments, the HFR2 is WVRQX13PGKGLEWX14G(SEQ ID NO:29). X13 may be A or S, and X14 may be L or V.

In some embodiments, the HFR2 is selected from an amino acid sequence as set forth in any one of SEQ ID NO:30-32.

In some embodiments, the VH comprises the HFR3, and the HFR3 is located between the HCDR2 and the HCDR3. For example, the sequence of the HFR3 of the antigen-binding protein may be defined by Kabat. the HFR3 comprises an amino acid as set forth in SEQ ID NO:33.

In some embodiments, the HFR3 is RX15TISX16DNSKNTX17YLQMNSLRAEDTAVYYCAX18(SEQ ID NO:33. X15 may be F or L, X16 may be A or K or R or V, X17 may be L or V, X18 may be R or S.

In some embodiments, the HFR3 is selected from an amino acid sequence as set forth in any one of SEQ ID NO:34-40.

In some embodiments, VH comprises the HFR4, and N-terminus of the HFR4 is directly or indirectly linked to C-terminus of the HCDR3. For example, the sequence of the HFR4 of the antigen-binding protein may be defined by Kabat. the HFR4 comprises an amino acid sequence as set forth in SEQ ID NO:41.

In some embodiments, the isolated antigen-binding protein further comprises a light chain variable region (VL). The VL comprises at least one light chain complementarity determining regions (LCDR). VL comprises an LCDR1, an LCDR2 and an LCDR3, which is selected from an amino acid sequence as set forth in any one of SEQ ID NO:42, SEQ ID NO:43 and SEQ ID NO:44 accordingly.

In some embodiments, the VL comprises an LCDR3, including an amino acid sequence as set forth in SEQ ID NO:44. For example, the sequence of the LCDR3 of the antigen-binding protein may be defined by Kabat.

In some embodiments, the VL comprises an LCDR2, including an amino acid sequence as set forth in SEQ ID NO:43. For example, the sequence of the LCDR2 of the antigen-binding protein may be defined by Kabat.

In some embodiments, the VL comprises an LCDR1, including an amino acid sequence as set forth in SEQ ID NO:42. For example, the sequence of the LCDR1 of the antigen-binding protein may be defined by Kabat.

In some embodiments, VL comprises the LFR1, and C-terminus of the LFR1 is directly or indirectly linked to N-terminus of the LCDR1. For example, the sequence of the LFR1 of the antigen-binding protein may be defined by Kabat. The LFR1 comprises an amino acid sequence as set forth in SEQ ID NO:45.

In some embodiments, the LFR1 is DIQMTQX19X20SSLSASVGDRVTIX21C(SEQ ID NO:45). X19 may be S or T, X20 may be P or T, X21 may be T or S.

In some embodiments, the LFR1 comprises an amino acid sequence as set forth in any one of SEQ ID NO:46-47.

In some embodiments, the VL comprises the LFR2, and the LFR2 is located between the LCDR1 and the LCDR2. For example, the sequence of the LFR2 of the antigen-binding protein may be defined by Kabat, the LFR2 comprises an amino acid sequence as set forth in SEQ ID NO:48.

In some embodiments, the LFR2 is WYQQKPGKX22X23KLLIY(SEQ ID NO:48). X22 may be A or V, and X23 may be P or V.

In some embodiments, the LFR2 comprises an amino acid sequence as set forth in any one of SEQ ID NO:49-52.

In some embodiments, the VL comprises the LFR3, the LFR3 is located between the LCDR2 and the LCDR3. For example, the sequence of the LFR3 of the antigen-binding protein may be defined by Kabat. The LFR3 comprises an amino acid sequence as set forth in SEQ ID NO:53.

In some embodiments, the LFR3 is GVPSRFSGSGSGTDX24TLTISSLQPEDX25ATYX26C(SEQ ID NO:53). X24 may be F or Y, X25 may be F or V, X26 may be F or Y.

In some embodiments, the LFR3 comprises an amino acid sequence as set forth in any one of SEQ ID NO:54-57.

In some embodiments, the isolated antigen-binding fragment comprises the LFR4, and N-terminus of the LFR4 is directly or indirectly linked to C terminus of the LCDR3. For example, the sequence of the LFR4 of the antigen-binding protein may be defined by Kabat. The LFR4 comprises an amino acid sequence as set forth in SEQ ID NO:58.

In some embodiments, the LFR4 is FGX27GTKLEIK(SEQ ID NO:58). X27 may be P or Q.

In some embodiments, the LFR4 comprises an amino acid sequence as set forth in any one of SEQ ID NO:59-61.

In some embodiments, the VH comprises sequences as set forth in SEQ ID NO:62-99.

In some embodiments, the VL comprises sequences as set forth in SEQ ID NO:102-106.

In some embodiments, the isolated antigen-binding protein comprises a VH as shown in SEQ ID NO: 70 and a VL as shown in SEQ ID NO: 106.

In some embodiments, the VH of the isolated antigen-binding protein comprises a HCDR1 as shown in SEQ ID NO: 2, a HCDR2 as shown in SEQ ID NO: 6, and a HCDR3 as shown in SEQ ID NO: 15, and further comprises a light chain variable region (VL), wherein the VL comprises at least one light chain complementary determining region (LCDR), which comprises a LCDR1 as shown in SEQ ID NO: 42, a LCDR2 as shown in SEQ ID NO: 43, and a LCDR3 as shown in SEQ ID NO: 44.

In some embodiments, the antigen-binding proteins described above are described in table A and table B.

In some embodiments, the distribution of antibodies for the TLA-0015 line is shown in the following table, with Table A showing the distribution of heavy chain components and Table B showing the distribution of light chain components.

TABLE A

| | VH | | | | | | | | | | | | | |
|---|---|---|---|---|---|---|---|---|---|---|---|---|---|---|
| | | SEQ | | SEQ | | SEQ | | SEQ | | SEQ | | SEQ | | SEQ |
| antibody | HFR1 | ID | HCDR1 | ID | HFR2 | ID | HCDR2 | ID | HFR3 | ID | HCDR3 | ID | HFR4 | ID |
| TLA0015 | | #N/A | TLA0015- | 2 | TLA0015- | 32 | TLA0015- | 6 | | | TLA0015- | 15 | | #N/A |
| (mouse) | | | HCDR1-1 | | HFR2-3 | | HCDR2-1 | | | | HCDR3-1 | | | |
| TLA0015-1 | TLA0015- | 20 | TLA0015- | 2 | TLA0015- | 30 | TLA0015- | 6 | TLA0015- | 34 | TLA0015- | 15 | TLA0015- | 41 |
| | HFR1-1 | | HCDR1-1 | | HFR2-1/ | | HCDR2-1 | | HFR3-1 | | HCDR3-1 | | HFR4-1/ | |
| | | | | | | | | | | | | | TLA-0238- | |
| | | | | | | | | | | | | | HFR4-2 | |
| TLA0015-2 | TLA0015- | 20 | TLA0015- | 2 | TLA0015- | 30 | TLA0015- | 6 | TLA0015- | 34 | TLA0015- | 15 | TLA0015- | 41 |
| | HFR1-1 | | HCDR1-1 | | HFR2-1/ | | HCDR2-1 | | HFR3-1 | | HCDR3-1 | | HFR4-1/ | |
| | | | | | | | | | | | | | TLA-0238- | |
| | | | | | | | | | | | | | HFR4-2 | |
| TLA0015-3 | TLA0015- | 21 | TLA0015- | 2 | TLA0015- | 31 | TLA0015- | 6 | TLA0015- | 35 | TLA0015- | 15 | TLA0015- | 41 |
| | HFR1-2 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-4 | TLA0015- | 21 | TLA0015- | 2 | TLA0015- | 31 | TLA0015- | 6 | TLA0015- | 35 | TLA0015- | 15 | TLA0015- | 41 |
| | HFR1-2 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-5 | TLA0015- | 22 | TLA0015- | 2 | TLA0015- | 32 | TLA0015- | 6 | TLA0015- | 35 | TLA0015- | 15 | TLA0015- | 41 |
| | HFR1-3 | | HCDR1-1 | | HFR2-3 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-6 | TLA0015- | 22 | TLA0015- | 2 | TLA0015- | 32 | TLA0015- | 6 | TLA0015- | 35 | TLA0015- | 15 | TLA0015- | 41 |
| | HFR1-3 | | HCDR1-1 | | HFR2-3 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-7 | TLA0015- | 23 | TLA0015- | 2 | TLA0015- | 31 | TLA0015- | 6 | TLA0015- | 36 | TLA0015- | 15 | TLA0015- | 41 |
| | HFR1-4 | | HCDR1-1 | | HFR2-2 | | HCDR2-1 | | HFR3-3 | | HCDR3-1 | | HFR4-1/ | |
| | | | | | | | | | | | | | TLA-0238- | |
| | | | | | | | | | | | | | HFR4-2 | |
| TLA0015-8 | TLA0015- | 23 | TLA0015- | 2 | TLA0015- | 31 | TLA0015- | 6 | TLA0015- | 37 | TLA0015- | 15 | TLA0015- | 41 |
| | HFR1-4 | | HCDR1-1 | | HFR2-2 | | HCDR2-1 | | HFR3-4 | | HCDR3-1 | | HFR4-1/ | |
| | | | | | | | | | | | | | TLA-0238- | |
| | | | | | | | | | | | | | HFR4-2 | |
| TLA0015-9 | TLA0015- | 23 | TLA0015- | 2 | TLA0015- | 31 | TLA0015- | 6 | TLA0015- | 35 | TLA0015- | 15 | TLA0015- | 41 |
| | HFR1-4 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-10 | TLA0015- | 24 | TLA0015- | 2 | TLA0015- | 31 | TLA0015- | 6 | TLA0015- | 38 | TLA0015- | 15 | TLA0015- | 41 |
| | HFR1-5 | | HCDR1-1 | | HFR2-2 | | HCDR2-1 | | HFR3-5 | | HCDR3-1 | | HFR4-1/ | |
| | | | | | | | | | | | | | TLA-0238- | |
| | | | | | | | | | | | | | HFR4-2 | |
| TLA0015-11 | TLA0015- | 25 | TLA0015- | 2 | TLA0015- | 31 | TLA0015- | 6 | TLA0015- | 35 | TLA0015- | 15 | TLA0015- | 41 |
| | HFR1-6 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-12 | TLA0015- | 25 | TLA0015- | 2 | TLA0015- | 31 | TLA0015- | 6 | TLA0015- | 35 | TLA0015- | 15 | TLA0015- | 41 |
| | HFR1-6 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-13 | TLA0015- | 25 | TLA0015- | 2 | TLA0015- | 31 | TLA0015- | 6 | TLA0015- | 35 | TLA0015- | 15 | TLA0015- | 41 |
| | HFR1-6 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-14 | TLA0015- | 23 | TLA0015- | 2 | TLA0015- | 31 | TLA0015- | 6 | TLA0015- | 36 | TLA0015- | 15 | TLA0015- | 41 |
| | HFR1-4 | | HCDR1-1 | | HFR2-2 | | HCDR2-1 | | HFR3-3 | | HCDR3-1 | | HFR4-1/ | |
| | | | | | | | | | | | | | TLA-0238- | |
| | | | | | | | | | | | | | HFR4-2 | |
| TLA0015-15 | TLA0015- | 23 | TLA0015- | 2 | TLA0015- | 31 | TLA0015- | 6 | TLA0015- | 37 | TLA0015- | 15 | TLA0015- | 41 |
| | HFR1-4 | | HCDR1-1 | | HFR2-2 | | HCDR2-1 | | HFR3-4 | | HCDR3-1 | | HFR4-1/ | |
| | | | | | | | | | | | | | TLA-0238- | |
| | | | | | | | | | | | | | HFR4-2 | |
| TLA0015-16 | TLA0015- | 23 | TLA0015- | 2 | TLA0015- | 31 | TLA0015- | 6 | TLA0015- | 36 | TLA0015- | 15 | TLA0015- | 41 |
| | HFR1-4 | | HCDR1-1 | | HFR2-2 | | HCDR2-1 | | HFR3-3 | | HCDR3-1 | | HFR4-1/ | |
| | | | | | | | | | | | | | TLA-0238- | |
| | | | | | | | | | | | | | HFR4-2 | |
| TLa0015-17 | TLA0015- | 25 | TLA0015- | 2 | TLA0015- | 31 | TLA0015- | 6 | TLA0015- | 37 | TLA0015- | 15 | TLA0015- | 41 |
| | HFR1-6 | | HCDR1-1 | | HFR2-2 | | HCDR2-1 | | HFR3-4 | | HCDR3-1 | | HFR4-1 | |
| TLa0015-18 | TLA0015- | 25 | TLA0015- | 2 | TLA0015- | 31 | TLA0015- | 6 | TLA0015- | 39 | TLA0015- | 15 | TLA0015- | 41 |
| | HFR1-6 | | HCDR1-1 | | HFR2-2 | | HCDR2-1 | | HFR3-6 | | HCDR3-1 | | HFR4-1 | |
| TLa0015-19 | TLA0015- | 25 | TLA0015- | 2 | TLA0015- | 31 | TLA0015- | 7 | TLA0015- | 35 | TLA0015- | 15 | TLA0015- | 41 |
| | HFR1-6 | | HCDR1-1 | | HFR2-2 | | HCDR2-2 | | HFR3-2 | | HCDR3-1 | | HFR4-1 | |
| TLa0015-20 | TLA0015- | 25 | TLA0015- | 2 | TLA0015- | 31 | TLA0015- | 8 | TLA0015- | 35 | TLA0015- | 15 | TLA0015- | 41 |
| | HFR1-6 | | HCDR1-1 | | HFR2-2 | | HCDR2-3 | | HFR3-2 | | HCDR3-1 | | HFR4-1/ | |
| | | | | | | | | | | | | | TLA-0238- | |
| | | | | | | | | | | | | | HFR4-2 | |
| TLa0015-21 | TLA0015- | 25 | TLA0015- | 2 | TLA0015- | 31 | TLA0015- | 6 | TLA0015- | 40 | TLA0015- | 15 | TLA0015- | 41 |
| | HFR1-6 | | HCDR1-1 | | HFR2-2 | | HCDR2-1 | | HFR3-7 | | HCDR3-1 | | HFR4-1 | |
| TLA015-22 | TLA0015- | 25 | TLA0015- | 3 | TLA0015- | 31 | TLA0015- | 6 | TLA0015- | 35 | TLA0015- | 15 | TLA0015- | 41 |
| | HFR1-6 | | HCDR1-2 | | HFR2-2 | | HCDR2-1 | | HFR3-2 | | HCDR3-1 | | HFR4-1 | |
| TLA015-23 | TLA0015- | 25 | TLA0015- | 2 | TLA0015- | 31 | TLA0015- | 6 | TLA0015- | 35 | TLA0015- | 16 | TLA0015- | 41 |
| | HFR1-6 | | HCDR1-1 | | HFR2-2 | | HCDR2-1 | | HFR3-2 | | HCDR3-2 | | HFR4-1 | |
| TLA015-24 | TLA0015- | 25 | TLA0015- | 2 | TLA0015- | 31 | TLA0015- | 6 | TLA0015- | 35 | TLA0015- | 17 | TLA0015- | 41 |
| | HFR1-6 | | HCDR1-1 | | HFR2-2 | | HCDR2-1 | | HFR3-2 | | HCDR3-3 | | HFR4-1 | |
| TLA015-25 | TLA0015- | 25 | TLA0015- | 2 | TLA0015- | 31 | TLA0015- | 6 | TLA0015- | 35 | TLA0015- | 18 | TLA0015- | 41 |
| | HFR1-6 | | HCDR1-1 | | HFR2-2 | | HCDR2-1 | | HFR3-2 | | HCDR3-4 | | HFR4-1 | |
| TLA015-26 | TLA0015- | 25 | TLA0015- | 4 | TLA0015- | 31 | TLA0015- | 6 | TLA0015- | 35 | TLA0015- | 15 | TLA0015- | 41 |
| | HFR1-6 | | HCDR1-3 | | HFR2-2 | | HCDR2-1 | | HFR3-2 | | HCDR3-1 | | HFR4-1 | |
| TLA015-27 | TLA0015- | 26 | TLA0015- | 2 | TLA0015- | 31 | TLA0015- | 6 | TLA0015- | 35 | TLA0015- | 15 | TLA0015- | 41 |
| | HFR1-7 | | HCDR1-1 | | HFR2-2 | | HCDR2-1 | | HFR3-2 | | HCDR3-1 | | HFR4-1 | |
| TLA015-28 | TLA0015- | 25 | TLA0015- | 2 | TLA0015- | 31 | TLA0015- | 9 | TLA0015- | 35 | TLA0015- | 15 | TLA0015- | 41 |
| | HFR1-6 | | HCDR1-1 | | HFR2-2 | | HCDR2-4 | | HFR3-2 | | HCDR3-1 | | HFR4-1 | |
| TLA015-29 | TLA0015- | 27 | TLA0015- | 2 | TLA0015- | 31 | TLA0015- | 6 | TLA0015- | 35 | TLA0015- | 15 | TLA0015- | 41 |
| | HFR1-8 | | HCDR1-1 | | HFR2-2 | | HCDR2-1 | | HFR3-2 | | HCDR3-1 | | HFR4-1 | |
| TLA015-30 | TLA0015- | 25 | TLA0015- | 2 | TLA0015- | 31 | TLA0015- | 10 | TLA0015- | 35 | TLA0015- | 15 | TLA0015- | 41 |
| | HFR1-6 | | HCDR1-1 | | HFR2-2 | | HCDR2-5 | | HFR3-2 | | HCDR3-1 | | HFR4-1/ | |
| TLA015-31 | TLA0015- | 25 | TLA0015- | 2 | TLA0015- | 31 | TLA0015- | 11 | TLA0015- | 35 | TLA0015- | 15 | TLA015- | 41 |
| | HFR1-6 | | HCDR1-1 | | HFR2-2 | | HCDR2-6 | | HFR3-2 | | HCDR3-1 | | HFR4-1/ | |
| | | | | | | | | | | | | | TLA-0238- | |
| | | | | | | | | | | | | | HFR4-2 | |
| TLA015-32 | TLA0015- | 28 | TLA0015- | 2 | TLA0015- | 31 | TLA0015- | 6 | TLA0015- | 35 | TLA0015- | 15 | TLA0015- | 41 |
| | HFR1-9 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA015-33 | TLA0015- | 25 | TLA0015- | 2 | TLA0015- | 31 | TLA0015- | 12 | TLA0015- | 35 | TLA0015- | 15 | TLA0015- | 41 |
| | HFR1-6 | | HCDR1-1 | | HFR2-2 | | HCDR2-7 | | HFR3-2 | | HCDR3-1 | | HFR4-1/ | |
| | | | | | | | | | | | | | TLA-0238- | |
| | | | | | | | | | | | | | HFR4-2 | |
| TLA015-34 | TLA0015- | 25 | TLA0015- | 2 | TLA0015- | 31 | TLA0015- | 13 | TLA0015- | 35 | TLA0015- | 15 | TLA0015- | 41 |
| | HFR1-6 | | HCDR1-1 | | HFR2-2 | | HCDR2-8 | | HFR3-2 | | HCDR3-1 | | HFR4-1/ | |
| | | | | | | | | | | | | | TLA-0238- | |
| | | | | | | | | | | | | | HFR4-2 | |
| TLA015-35 | TLA0015- | 25 | TLA0015- | 3 | TLA0015- | 31 | TLA0015- | 6 | TLA0015- | 35 | TLA0015- | 16 | TLA0015- | 41 |
| | HFR1-6 | | HCDR1-2 | | HFR2-2 | | HCDR2-1 | | HFR3-2 | | HCDR3-2 | | HFR4-1/ | |
| TLA015-36 | TLA0015- | 25 | TLA0015- | 3 | TLA0015- | 31 | TLA0015- | 6 | TLA0015- | 35 | TLA0015- | 17 | TLA0015- | 41 |
| | HFR1-6 | | HCDR1-2 | | HFR2-2 | | HCDR2-1 | | HFR3-2 | | HCDR3-3 | | HFR4-1 | |
| TLA015-37 | TLA0015- | 25 | TLA0015- | 3 | TLA0015- | 31 | TLA0015- | 9 | TLA0015- | 35 | TLA0015- | 15 | TLA0015- | 41 |
| | HFR1-6 | | HCDR1-2 | | HFR2-2 | | HCDR2-4 | | HFR3-2 | | HCDR3-1 | | HFR4-1 | |
| TLA015-38 | TLA0015- | 25 | TLA0015- | 3 | TLA0015- | 31 | TLA0015- | 10 | TLA0015- | 35 | TLA0015- | 15 | TLA0015- | 41 |
| | HFR1-6 | | HCDR1-2 | | HFR2-2 | | HCDR2-5 | | HFR3-2 | | HCDR3-1 | | HFR4-1 | |
| TLA015-39 | TLA0015- | 25 | TLA0015- | 3 | TLA0015- | 31 | TLA0015- | 11 | TLA0015- | 35 | TLA0015- | 15 | TLA0015- | 41 |
| | HFR1-6 | | HCDR1-2 | | HFR2-2 | | HCDR2-6 | | HFR3-2 | | HCDR3-1 | | HFR4-1/ | |
| | | | | | | | | | | | | | TLA-0238- | |
| TLA015-40 | TLA0015- | 28 | TLA0015- | 3 | TLA0015- | 31 | TLA0015- | 6 | TLA0015- | 35 | TLA0015- | 15 | TLA0015- | 41 |
| | HFR1-9 | | HCDR1-2 | | HFR2-2 | | HCDR2-1 | | HFR3-2 | | HCDR3-1 | | HFR4-1 | |
| TLA015-41 | TLA0015- | 26 | TLA0015- | 2 | TLA0015- | 31 | TLA0015- | 6 | TLA0015- | 35 | TLA0015- | 16 | TLA0015- | 41 |
| | HFR1-7 | | HCDR1-1 | | HFR2-2 | | HCDR2-1 | | HFR3-2 | | HCDR3-2 | | HFR4-1 | |
| TLA015-42 | TLA0015- | 25 | TLA0015- | 2 | TLA0015- | 31 | TLA0015- | 9 | TLA0015- | 35 | TLA0015- | 16 | TLA0015- | 41 |
| | HFR1-6 | | HCDR1-1 | | HFR2-2 | | HCDR2-4 | | HFR3-2 | | HCDR3-2 | | HFR4-1 | |
| TLA015-43 | TLA0015- | 25 | TLA0015- | 2 | TLA0015- | 31 | TLA0015- | 10 | TLA0015- | 35 | TLA0015- | 16 | TLA0015- | 41 |
| | HFR1-6 | | HCDR1-1 | | HFR2-2 | | HCDR2-5 | | HFR3-2 | | HCDR3-2 | | HFR4-1 | |
| TLA015-44 | TLA0015- | 25 | TLA0015- | 2 | TLA0015- | 31 | TLA0015- | 11 | TLA0015- | 35 | TLA0015- | 16 | TLA0015- | 41 |
| | HFR1-6 | | HCDR1-1 | | HFR2-2 | | HCDR2-6 | | HFR3-2 | | HCDR3-2 | | HFR4-1 | |
| TLA015-45 | TLA0015- | 25 | TLA0015- | 2 | TLA0015- | 31 | TLA0015- | 12 | TLA0015- | 35 | TLA0015- | 16 | TLA0015- | 41 |
| | HFR1-6 | | HCDR1-1 | | HFR2-2 | | HCDR2-7 | | HFR3-2 | | HCDR3-2 | | HFR4-1 | |
| TLA015-46 | TLA0015- | 26 | TLA0015- | 2 | TLA0015- | 31 | TLA0015- | 6 | TLA0015- | 35 | TLA0015- | 17 | TLA0015- | 41 |
| | HFR1-7 | | HCDR1-1 | | HFR2-2 | | HCDR2-1 | | HFR3-2 | | HCDR3-3 | | HFR4-1 | |
| TLA015-47 | TLA0015- | 25 | TLA0015- | 2 | TLA0015- | 31 | TLA0015- | 10 | TLA0015- | 35 | TLA0015- | 17 | TLA0015- | 41 |
| | HFR1-6 | | HCDR1-1 | | HFR2-2 | | HCDR2-5 | | HFR3-2 | | HCDR3-3 | | HFR4-1/ | |
| | | | | | | | | | | | | | TLA-0238- | |
| | | | | | | | | | | | | | HFR4-2 | |

TABLE B

| | VL | | | | | | | | | | | | | |
|---|---|---|---|---|---|---|---|---|---|---|---|---|---|---|
| | | SEQ | | SEQ | | SEQ | | SEQ | | SEQ | | SEQ | | SEQ |
| antibody | LFR1 | ID | CDR1 | ID | LFR2 | ID | LCDR2 | ID | LFR3 | ID | LCDR3 | ID | LFR4 | ID |
| TLA0015 | TL0015- | 46 | TL0015- | 42 | TL0015- | 49 | TL0015- | 43 | TL0015- | 54 | TL0015- | 44 | TL0015- | 59 |
| (mouse) | LFR1-1 | | LCDR1-1 | | LFR2-1 | | LCDR2-1 | | LFR3-1 | | LCDR3-1 | | LFR4-1 | |
| TLA0015-1 | TL0015- | 47 | TL0015- | 42 | TL0015- | 50 | TL0015- | 43 | TL0015- | 55 | TL0015- | 44 | TL0015- | 60 |
| | LFR1-2/ | | LCDR1-1 | | LFR2-2 | | LCDR2-1 | | LFR3-2 | | LCDR3-1 | | LFR4-2 | |
| | TLA0241- | | | | | | | | | | | | | |
| | LFR1-1 | | | | | | | | | | | | | |
| TLA0015-2 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-3 | TL0015- | 47 | TL0015- | 42 | TL0015- | 50 | TL0015- | 43 | TL0015- | 55 | TL0015- | 44 | TL0015- | 60 |
| | LFR1-2/ | | LCDR1-1 | | LFR2-2 | | LCDR2-1 | | LFR3-2 | | LCDR3-1 | | LFR4-2 | |
| | TLA0241- | | | | | | | | | | | | | |
| | LFR1-1 | | | | | | | | | | | | | |
| TLA0015-4 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-5 | TL0015- | 47 | TL0015- | 42 | TL0015- | 50 | TL0015- | 43 | TL0015- | 55 | TL0015- | 44 | TL0015- | 60 |
| | LFR1-2/ | | LCDR1-1 | | LFR2-2 | | LCDR2-1 | | LFR3-2 | | LCDR3-1 | | LFR4-2 | |
| | TLA0241- | | | | | | | | | | | | | |
| | LFR1-1 | | | | | | | | | | | | | |
| TLA0015-6 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-7 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-8 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-9 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-10 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-11 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-12 | TL0015- | 47 | TL0015- | 42 | TL0015- | 52 | TL0015- | 43 | TL0015- | 57 | TL0015- | 44 | TL0015- | 61 |
| | LFR1-2/ | | LCDR1-1 | | LFR2-4/ | | LCDR2-1 | | LFR3-4/ | | LCDR3-1 | | LFR4-3/ | |
| | TLA0241- | | | | TLA0241- | | | | TLA0238- | | | | TLA0238- | |
| | LFR1-1 | | | | LFR2-1 | | | | LFR3-4/ | | | | LFR4-3 | |
| | | | | | | | | | TLA0241- | | | | | |
| | | | | | | | | | LFR3-2 | | | | | |
| TLA0015-13 | TL0015- | 47 | TL0015- | 42 | TL0015- | 52 | TL0015- | 43 | TL0015- | 56 | TL0015- | 44 | TL0015- | 61 |
| | LFR1-2/ | | LCDR1-1 | | LFR2-4/ | | LCDR2-1 | | LFR3-3 | | LCDR3-1 | | LFR4-3/ | |
| | TLA0241- | | | | TLA0241- | | | | | | | | TLA0238- | |
| | LFR1-1 | | | | LFR2-1 | | | | | | | | LFR4-3 | |
| TLA0015-14 | TL0015- | 47 | TL0015- | 42 | TL0015- | 52 | TL0015- | 43 | TL0015- | 57 | TL0015- | 44 | TL0015- | 61 |
| | LFR1-2/ | | LCDR1-1 | | LFR2-4/ | | LCDR2-1 | | LFR3-4/ | | LCDR3-1 | | LFR4-3/ | |
| | TLA0241- | | | | TLA0241- | | | | TLA0238- | | | | TLA0238- | |
| | LFR1-1 | | | | LFR2-1 | | | | LFR3-4/ | | | | LFR4-3 | |
| | | | | | | | | | TLA0241- | | | | | |
| | | | | | | | | | LFR3-2 | | | | | |
| TLA0015-15 | TL0015- | 47 | TL0015- | 42 | TL0015- | 52 | TL0015- | 43 | TL0015- | 57 | TL0015- | 44 | TL0015- | 61 |
| | LFR1-2/ | | LCDR1-1 | | LFR2-4/ | | LCDR2-1 | | LFR3-4/ | | LCDR3-1 | | LFR4-3/ | |
| | TLA0241- | | | | TLA0241- | | | | TLA0238- | | | | TLA0238- | |
| | LFR1-1 | | | | LFR2-1 | | | | LFR3-4/ | | | | LFR4-3 | |
| | | | | | | | | | TLA0241- | | | | | |
| | | | | | | | | | LFR3-2 | | | | | |
| TLA0015-16 | TL0015- | 47 | TL0015- | 42 | TL0015- | 52 | TL0015- | 43 | TL0015- | 56 | TL0015- | 44 | TL0015- | 61 |
| | LFR1-2/ | | LCDR1-1 | | LFR2-4/ | | LCDR2-1 | | LFR3-3 | | LCDR3-1 | | LFR4-3/ | |
| | TLA0241- | | | | TLA0241- | | | | | | | | TLA0238- | |
| | LFR1-1 | | | | LFR2-1 | | | | | | | | LFR4-3 | |
| TLa0015-17 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLa0015-18 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLa0015-19 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLa0015-20 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLa0015-21 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-22 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-23 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-24 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-25 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-26 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-27 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-28 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-29 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-30 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-31 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-32 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-33 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-34 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-35 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-36 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-37 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-38 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-39 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-40 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-41 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-42 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-43 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-44 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-45 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-46 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-47 | TL0015- | 47 | TL0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |

In one aspect, the present application provides an isolated antigen-binding protein comprising a first antigen-binding domain which specifically binds to TL1A. The first binding domain includes a heavy chain variable region (VH). The VH comprises at least one heavy chain complementarity determining regions (HCDR). The VH may include at least one, two or three of HCDR1, HCDR2 or HCDR3. In some embodiments, the HCDR 1, the HCDR3 and the HCDR3 are sequences as set forth in SEQ TD NO: 107, 115 and 120 respectively.

The HCDR3 of the antigen-binding protein may include amino acid sequence as set forth in SEQ TD NO: 120. For example, the sequence of the HCDR3 of the antigen-binding protein may be defined by Kabat.

The HCDR2 of the antigen-binding protein may include amino acid sequence as set forth in SEQ ID NO:115. For example, the sequence of the HCDR2 of the antigen-binding protein may be defined by Kabat.

In some embodiments, the HCDR2 may be QIRLKSDNYATHYAX28X29VKG(SEQ ID NO:115). X28 may be A or D or E. X29 may be P or S.

In some embodiments, the HCDR2 may include an amino acid sequence as set forth in any one of SEQ ID NOs:116-119.
the HCDR1 of the antigen-binding protein may include amino acid sequence as set forth in SEQ ID NO:107. For example, the sequence of the HCDR1 of the antigen-binding protein may be defined by Kabat.

In some embodiments, the HCDR1 may be NYX30MX31 (SEQ ID NO:107). X30 may be I or L or M or Y or W. X31 may be N or H or S.

In some embodiments, the HCDR1 may include an amino acid as set forth in any one of SEQ ID NO:108-114.

In some embodiments, the VH comprises the HFR1, and C-terminus of the HFR1 is directly or indirectly linked to the N-terminus of the HCDR1. For example, the sequence of the HFR1 of the antigen-binding protein may be defined by Kabat. the HFR1 comprises an amino acid as set forth in SEQ ID NO:121.

In some embodiments, the HFR1 is X32VX33X34X35ESGGGX36VX37PGX38X39LX40LSCX41ASGFTFS (SEQ ID NO:121). X32 may be E or Q. X33 may be K or Q.X34 may be L or VX35 may be E or V.X36 may be L or VX37 may be K or Q. X38 may be G or R.X39 may be A or S.X40 may be K or R.X41 may be A or I.

In some embodiments, the HFR1 is selected from an amino acid sequence as set forth in any one of SEQ ID NO:122-132.

In some embodiments, VH comprises the HFR2, and the HFR2 is located between the HCDR1 and the HCDR2. For example, the sequence of the HFR2 of the antigen-binding protein may be defined by Kabat. the HFR2 comprises an amino acid sequence as set forth in SEQ ID NO:133.

In some embodiments, the HFR2 is WVRQX42PX43KGLEWVX44(SEQ ID NO:133). X42 may be A or S. X43 may be E or G.X44 may be A or G.

In some embodiments, the HFR2 is selected from an amino acid sequence as set forth in any one of SEQ ID NO:134-135 and 30.

In some embodiments, VH comprises the HFR3, and the HFR3 is located between the HCDR2 and the HCDR3. For example, the sequence of the HFR3 of the antigen-binding protein may be defined by Kabat. the HFR3 comprises an amino acid as set forth in SEQ ID NO:136.

In some embodiments, the HFR3 is X45FX46ISRDX47X48KX49X50X51YLQMNSLX52X53EDX54AVYYCTP(SEQ ID NO:136). X45 may be N or R.X46 may be A or T.X47 may be D or N.X48 may be A or S.X49 may be N or S.X50 may be S or T.X51 may be A or L or V.X52 may be K or R.X53 may be A or D or T.X54 may be M or T.

In some embodiments, the HFR3 is selected from an amino acid sequence as set forth in any one of SEQ ID NO:137-148.

In some embodiments, VH comprises the HFR4, and N-terminus of the HFR4 is directly or indirectly linked to C-terminus of the HCDR3. For example, the sequence of the HFR4 of the antigen-binding protein may be defined by Kabat. The HFR4 comprises an amino acid sequence as set forth in SEQ ID NO:149.

In some embodiments, the HFR4 is WGQGT X55X56TVSS (SEQ ID NO:149). X55 may be L or M OR H or T.X56 may be L or V.

In some embodiments, the isolated antigen-binding fragment further comprises a light chain variable region(VL), and the VL comprises at least one light chain complementarity determining regions (LCDR). VL comprises an LCDR1, an LCDR2 and an LCDR3, which is selected from an amino acid sequence as set forth in any one of SEQ ID NO: 154, SEQ ID NO:159 and SEQ ID NO: 163 accordingly.

In some embodiments, the VL comprises the LCDR3, including an amino acid sequence as set forth in SEQ ID NO:163. For example, the sequence of the LCDR3 of the antigen-binding protein may be defined by Kabat.

In some embodiments, the LCDR3 is FQEX57X58HPFT(SEQ ID NO:163). X57 may be N or S or E.X58 may be G or E or A.

In some embodiments, the LCDR3 is selected from an amino acid sequence as set forth in any one of SEQ ID NO:164-168.

In some embodiments, the VL comprises the LCDR2, including an amino acid sequence as set forth in SEQ ID NO:159. For example, the sequence of the LCDR2 of the antigen-binding protein may be defined by Kabat.

In some embodiments, the LCDR2 is DTSNX59AX60 (SEQ ID NO:159). X59 may be L or R. X60 may be S or T.

In some embodiments, the LCDR2 is selected from an amino acid sequence as set forth in any one of SEQ ID NO:160-162.

In some embodiments, the VL comprises the LCDR1, including an amino acid sequence as set forth in SEQ ID NO:42. For example, the sequence of the LCDR1 of the antigen-binding protein may be defined by Kabat.

In some embodiments, the LCDR1 is SASSSVSYX61X62 (SEQ ID NO:154). X61 may be L or M. X62 may be A or H or T.

In some embodiments, the LCDR1 is selected from an amino acid sequence as set forth in any one of SEQ ID NO:155-158.

In some embodiments, the VL comprises the LFR1, and C-terminus of the LFR1 is directly or indirectly linked to N-terminus of the LCDR1. For example, the sequence of the LFR1 of the antigen-binding protein may be defined by Kabat. The LFR1 comprises an amino acid sequence as set forth in SEQ ID NO:169.

In some embodiments, the LFR1 is X63X64X65X66TQSPX67X68X69SX70X71X72GX73X74X75TX76X77C(SEQ ID NO:169). X63 may be E or D.X64 may be I or N or T or V.X65 may be Q or V.X66 may be L or M.X67 may be A or S. X68 may be F or I or S or T.X69 may be L or M or V. X70 may be A or L or V. X71 may be S or T.X72 may be P or V.X73 may be D or E. X74 may be K or R.X75 may be A or V.X76 may be I or M.X77 may be S or T.

In some embodiments, the LFR1 comprises an amino acid sequence as set forth in any one of SEQ ID NO:170-179.

In some embodiments, the VL comprises the LFR2, and the LFR2 is located between the LCDR1 and the LCDR2. For example, the sequence of the LFR2 of the antigen-binding protein may be defined by Kabat, the LFR2 comprises an amino acid sequence as set forth in SEQ ID NO:180.

In some embodiments, the LFR2 is WYQQKP X78 X79 X80P X81LWIY(SEQ ID NO:180). X78 may be D or G.X79 may be K or Q.X80 may be A or S.X81 may be K or Q or R.

In some embodiments, the LFR2 comprises an amino acid sequence as set forth in any one of SEQ ID NO:181-188.

In some embodiments, the VL comprises the LFR3, and the LFR3 is located between the LCDR2 and the LCDR3. For example, the sequence of the LFR3 of the antigen-binding protein may be defined by Kabat. The LFR3 comprises an amino acid sequence as set forth in SEQ ID NO:189.

In some embodiments, the LFR3 is GVP X82RFSGSGSGX83X84X85TLTISSX86X87X88EDX89AX90YYC(SEQ ID NO:189). X82 may be A or D or G or S. X83 may be N or T.X84 may be D or S. X85 may be F or Y X86 may be L or M.X87 may be E or Q. X88 may be A or P.X89 may be A or F or V. X90 may be T or V.

In some embodiments, the LFR3 comprises an amino acid sequence as set forth in any one of SEQ ID NO:190-197 and 57.

In some embodiments, the isolated antigen-binding fragment comprises the LFR4, and N-terminus of the LFR4 is directly or indirectly linked to C terminus of the LCDR3. For example, the sequence of the LFR4 of the antigen-binding protein may be defined by Kabat. The LFR4 comprises an amino acid sequence as set forth in SEQ ID NO: 198.

In some embodiments, the LFR4 is FG X91GTK X92EX93K(SEQ TD NO: 198). X91 may be G or Q. X92 may be L or V X93 may be I or M.

In some embodiments, the LFR4 comprises an amino acid sequence as set forth in any one of SEQ TD NO: 199-201 and 61.

In some embodiments, VH comprises sequences as set forth in SEQ TD NO:202-220.

In some embodiments, VL comprises sequences as set forth in SEQ ID NO:221-234.

In some embodiments, the antigen-binding proteins described above are as described in Tables Cand D.

In some embodiments, the distribution of antibodies for the TLA-0238 line is shown in the following table, with Table C showing the distribution of heavy chain components and Table D showing the distribution of light chain components.

TABLE C

| | VH | | | | | | | | | | | | | |
|---|---|---|---|---|---|---|---|---|---|---|---|---|---|---|
| | | SEQ | | SEQ | | SEQ | | SEQ | | SEQ | | SEQ | | SEQ |
| antibody | HFR1 | ID | HCDR1 | ID | HFR2 | ID | HCDR2 | ID | HFR3 | ID | HCDR3 | ID | HFR4 | ID |
| TLA0238 | | | TLA-0238- | 108 | | 134 | TLA-0238- | 116 | | | TLA-0238- | 120 | | |
| (mouse) | | | HCDR1-1 | | | | HCDR2-1 | | | | HCDR3-1 | | | |
| TLA0238-1 | TLA-0238- | 123 | TLA-0238- | 108 | TLA-0238- | 135 | TLA-0238- | 116 | TLA-0238- | 138 | TLA-0238- | 120 | TLA0015- | 41 |
| | HFR1-2 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0238-2 | TLA-0238- | 123 | TLA-0238- | 108 | TLA-0238- | 135 | TLA-0238- | 116 | TLA-0238- | 138 | TLA-0238- | 120 | TLA0015- | 41 |
| | HFR1-2 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0238-3 | TLA-0238- | 124 | TLA-0238- | 108 | TLA-0238- | 135 | TLA-0238- | 116 | TLA-0238- | 139 | TLA-0238- | 120 | TLA-0238- | 151 |
| | HFR1-3 | | HCDR1-1 | | HFR2-2 | | HCDR2-1 | | HFR3-3 | | HCDR3-1 | | HFR4-3 | |
| TLA0238-4 | TLA-0238- | 124 | TLA-0238- | 108 | TLA-0238- | 135 | TLA-0238- | 116 | TLA-0238- | 139 | TLA-0238- | 120 | TLA-0238- | 151 |
| | HFR1-3 | | HCDR1-1 | | HFR2-2 | | HCDR2-1 | | HFR3-3 | | HCDR3-1 | | HFR4-3 | |
| TLA0238-2-1 | TLA-0238- | 123 | TLA-0238- | 108 | TLA-0238- | 135 | TLA-0238- | 116 | TLA-0238- | 140 | TLA-0238- | 120 | TLA0015- | 41 |
| | HFR1-2 | | HCDR1-1 | | HFR2-2 | | HCDR2-1 | | HFR3-4 | | HCDR3-1 | | HFR4-1/ | |
| | | | | | | | | | | | | | TLA-0238- | |
| | | | | | | | | | | | | | HFR4-2 | |
| TLA0238-2-2 | TLA-0238- | 123 | TLA-0238- | 108 | TLA-0238- | 135 | TLA-0238- | 116 | TLA-0238- | 141 | TLA-0238- | 120 | TLA0015- | 41 |
| | HFR1-2 | | HCDR1-1 | | HFR2-2 | | HCDR2-1 | | HFR3-5 | | HCDR3-1 | | HFR4-1/ | |
| | | | | | | | | | | | | | TLA-0238- | |
| | | | | | | | | | | | | | HFR4-2 | |
| TLA0238-5 | TLA-0238- | 125 | TLA-0238- | 108 | TLA-0238- | 135 | TLA-0238- | 117 | TLA-0238- | 142 | TLA-0238- | 120 | TLA0015- | 41 |
| | HFR1-4 | | HCDR1-1 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-6 | TLA-0238- | 126 | TLA-0238- | 108 | TLA-0238- | 135 | TLA-0238- | 117 | TLA-0238- | 142 | TLA-0238- | 120 | TLA-0238- | 152 |
| | HFR1-5 | | HCDR1-1 | | HFR2-2 | | HCDR2-2 | | HFR3-6 | | HCDR3-1 | | HFR4-4 | |
| TLA0238-7 | TLA-0238- | 127 | TLA-0238- | 109 | TLA0015- | 30 | TLA-0238- | 118 | TLA-0238- | 143 | TLA-0238- | 120 | TLA0015- | 41 |
| | HFR1-6 | | HCDR1-2 | | HFR2-1/ | | HCDR2-3 | | HFR3-7 | | HCDR3-1 | | HFR4-1/ | |
| | | | | | TLA-0238- | | | | | | | | TLA-0238- | |
| | | | | | HFR2-3 | | | | | | | | HFR4-2 | |
| TLA0238-8 | TLA-0238- | 128 | TLA-0238- | 109 | TLA0015- | 30 | TLA-0238- | 118 | TLA-0238- | 144 | TLA-0238- | 120 | TLA0015- | 41 |
| | HFR1-7 | | HCDR1-2 | | HFR2-1/ | | HCDR2-3 | | HFR3-8 | | HCDR3-1 | | HFR4-1/ | |
| | | | | | TLA-0238- | | | | | | | | TLA-0238- | |
| | | | | | HFR2-3 | | | | | | | | HFR4-2 | |
| TLA0238-9 | TLA-0238- | 129 | TLA-0238- | 109 | TLA0015- | 30 | TLA-0238- | 118 | TLA-0238- | 145 | TLA-0238- | 120 | TLA0015- | 41 |
| | HFR1-8 | | HCDR1-2 | | HFR2-1/ | | HCDR2-3 | | HFR3-9 | | HCDR3-1 | | HFR4-1/ | |
| | | | | | TLA-0238- | | | | | | | | TLA-0238- | |
| | | | | | HFR2-3 | | | | | | | | HFR4-2 | |
| TLA0238-10 | TLA-0238- | 127 | TLA-0238- | 109 | TLA0015- | 30 | TLA-0238- | 118 | TLA-0238- | 146 | TLA-0238- | 120 | TLA0015- | 41 |
| | HFR1-6 | | HCDR1-2 | | HFR2-1/ | | HCDR2-3 | | HFR3-10 | | HCDR3-1 | | HFR4-1/ | |
| | | | | | TLA-0238- | | | | | | | | TLA-0238- | |
| | | | | | HFR2-3 | | | | | | | | HFR4-2 | |
| TLA0238-11 | TLA-0238- | 125 | TLA-0238- | 108 | TLA-0238- | 135 | TLA-0238- | 117 | TLA-0238- | 142 | TLA-0238- | 120 | TLA-0238- | 152 |
| | HFR1-4 | | HCDR1-1 | | HFR2-2 | | HCDR2-2 | | HFR3-6 | | HCDR3-1 | | HFR4-4 | |
| TLA0238-12 | TLA-0238- | 130 | TLA-0238- | 109 | TLA0015- | 30 | TLA-0238- | 118 | TLA-0238- | 147 | TLA-0238- | 120 | TLA0015- | 41 |
| | HFR1-9 | | HCDR1-2 | | HFR2-1/ | | HCDR2-3 | | HFR3-11 | | HCDR3-1 | | HFR4-1/ | |
| | | | | | TLA-0238- | | | | | | | | TLA-0238- | |
| | | | | | HFR2-3 | | | | | | | | HFR4-2 | |
| TLA0238-13 | TLA-0238- | 131 | TLA-0238- | 108 | TLA-0238- | 135 | TLA-0238- | 119 | TLA-0238- | 148 | TLA-0238- | 120 | TLA-0238- | 153 |
| | HFR1-10 | | HCDR1-1 | | HFR2-2 | | HCDR2-4 | | HFR3-12 | | HCDR3-1 | | HFR4-5 | |
| TLA0238-14 | TLA-0238- | 132 | TLA-0238- | 110 | TLA0015- | 30 | TLA-0238- | 118 | TLA-0238- | 145 | TLA-0238- | 120 | TLA0015- | 41 |
| | HFR1-11 | | HCDR1-3 | | HFR2-1/ | | HCDR2-3 | | HFR3-9 | | HCDR3-1 | | HFR4-1/ | |
| | | | | | TLA-0238- | | | | | | | | TLA-0238- | |
| | | | | | HFR2-3 | | | | | | | | HFR4-2 | |
| TLA0238-15 | TLA-0238- | 125 | TLA-0238- | 111 | TLA-0238- | 135 | TLA-0238- | 117 | TLA-0238- | 142 | TLA-0238- | 120 | TLA0015- | 41 |
| | HFR1-4 | | HCDR1-4 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-16 | TLA-0238- | 125 | TLA-0238- | 111 | TLA-0238- | 135 | TLA-0238- | 117 | TLA-0238- | 142 | TLA-0238- | 120 | TLA0015- | 41 |
| | HFR1-4 | | HCDR1-4 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-17 | TLA-0238- | 125 | TLA-0238- | 112 | TLA-0238- | 135 | TLA-0238- | 117 | TLA-0238- | 142 | TLA-0238- | 120 | TLA0015- | 41 |
| | HFR1-4 | | HCDR1-5 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-18 | TLA-0238- | 125 | TLA-0238- | 112 | TLA-0238- | 135 | TLA-0238- | 117 | TLA-0238- | 142 | TLA-0238- | 120 | TLA0015- | 41 |
| | HFR1-4 | | HCDR1-5 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-19 | TLA-0238- | 125 | TLA-0238- | 113 | TLA-0238- | 135 | TLA-0238- | 117 | TLA-0238- | 142 | TLA-0238- | 120 | TLA0015- | 41 |
| | HFR1-4 | | HCDR1-6 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-20 | TLA-0238- | 125 | TLA-0238- | 113 | TLA-0238- | 135 | TLA-0238- | 117 | TLA-0238- | 142 | TLA-0238- | 120 | TLA0015- | 41 |
| | HFR1-4 | | HCDR1-6 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-21 | TLA-0238- | 125 | TLA-0238- | 111 | TLA-0238- | 135 | TLA-0238- | 117 | TLA-0238- | 142 | TLA-0238- | 120 | TLA0015- | 41 |
| | HFR1-4 | | HCDR1-4 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-22 | TLA-0238- | 125 | TLA-0238- | 111 | TLA-0238- | 135 | TLA-0238- | 117 | TLA-0238- | 142 | TLA-0238- | 120 | TLA0015- | 41 |
| | HFR1-4 | | HCDR1-4 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-23 | TLA-0238- | 125 | TLA-0238- | 112 | TLA-0238- | 135 | TLA-0238- | 117 | TLA-0238- | 142 | TLA-0238- | 120 | TLA0015- | 41 |
| | HFR1-4 | | HCDR1-5 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-24 | TLA-0238- | 125 | TLA-0238- | 112 | TLA-0238- | 135 | TLA-0238- | 117 | TLA-0238- | 142 | TLA-0238- | 120 | TLA0015- | 41 |
| | HFR1-4 | | HCDR1-5 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-25 | TLA-0238- | 125 | TLA-0238- | 113 | TLA-0238- | 135 | TLA-0238- | 117 | TLA-0238- | 142 | TLA-0238- | 120 | TLA0015- | 41 |
| | HFR1-4 | | HCDR1-6 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-26 | TLA-0238- | 125 | TLA-0238- | 113 | TLA-0238- | 135 | TLA-0238- | 117 | TLA-0238- | 142 | TLA-0238- | 120 | TLA0015- | 41 |
| | HFR1-4 | | HCDR1-6 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-27 | TLA-0238- | 125 | TLA-0238- | 108 | TLA-0238- | 135 | TLA-0238- | 117 | TLA-0238- | 142 | TLA-0238- | 120 | TLA0015- | 41 |
| | HFR1-4 | | HCDR1-1 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-28 | TLA-0238- | 125 | TLA-0238- | 108 | TLA-0238- | 135 | TLA-0238- | 117 | TLA-0238- | 142 | TLA-0238- | 120 | TLA0015- | 41 |
| | HFR1-4 | | HCDR1-1 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-29 | TLA-0238- | 125 | TLA-0238- | 114 | TLA-0238- | 135 | TLA-0238- | 117 | TLA-0238- | 142 | TLA-0238- | 120 | TLA0015- | 41 |
| | HFR1-4 | | HCDR1-7 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |
| TLA0238-30 | TLA-0238- | 125 | TLA-0238- | 114 | TLA-0238- | 135 | TLA-0238- | 117 | TLA-0238- | 142 | TLA-0238- | 120 | TLA0015- | 41 |
| | HFR1-4 | | HCDR1-7 | | HFR2-2 | | HCDR2-2 | | HFR3-6 | | HCDR3-1 | | HFR4-1/ | |
| | | | | | | | | | | | | | TLA-0238- | |
| | | | | | | | | | | | | | HFR4-2 | |

TABLE D

| | VL | | | | | | | | | | | | | |
|---|---|---|---|---|---|---|---|---|---|---|---|---|---|---|
| | | SEQ | | SEQ | | SEQ | | SEQ | | SEQ | | SEQ | | SEQ |
| antibody | the LFR1 | ID | CDR1 | ID | the LFR2 | ID | LCDR2 | ID | LFR3 | ID | LCDR3 | ID | LFR4 | ID |
| TLA0238 | | | TLA0238- | 155 | | | TLA0238- | 160 | | | TLA0238- | 164 | | |
| (mouse) | | | LCDR1-1 | | | | LCDR2-1 | | | | LCDR3-1 | | | |
| TLA0238-1 | TLA0238- | 171 | TLA0238- | 155 | TLA0238- | 182 | TLA0238- | 160 | TLA0238- | 191 | TLA0238- | 164 | TLA0238- | 200 |
| | LFR1-2 | | LCDR1-1 | | LFR2-2 | | LCDR2-1 | | LFR3-2 | | LCDR3-1 | | LFR4-2 | |
| TLA0238-2 | TLA0238- | 172 | TLA0238- | 155 | TLA0238- | 183 | TLA0238- | 160 | TLA0238- | 192 | TLA0238- | 164 | TL0015- | 61 |
| | LFR1-3 | | LCDR1-1 | | LFR2-3 | | LCDR2-1 | | LFR3-3 | | LCDR3-1 | | LFR4-3/ | |
| | | | | | | | | | | | | | TLA0238- | |
| | | | | | | | | | | | | | LFR4-3 | |
| TLA0238-3 | TLA0238- | 171 | TLA0238- | 155 | TLA0238- | 182 | TLA0238- | 160 | TLA0238- | 191 | TLA0238- | 164 | TLA0238- | 200 |
| | LFR1-2 | | LCDR1-1 | | LFR2-2 | | LCDR2-1 | | LFR3-2 | | LCDR3-1 | | LFR4-2 | |
| TLA0238-4 | TLA0238- | 172 | TLA0238- | 155 | TLA0238- | 183 | TLA0238- | 160 | TLA0238- | 192 | TLA0238- | 164 | TL0015- | 61 |
| | LFR1-3 | | LCDR1-1 | | LFR2-3 | | LCDR2-1 | | LFR3-3 | | LCDR3-1 | | LFR4-3/ | |
| | | | | | | | | | | | | | TLA0238- | |
| | | | | | | | | | | | | | LFR4-3 | |
| TLA0238-2-1 | TLA0238- | 172 | TLA0238- | 155 | TLA0238- | 183 | TLA0238- | 160 | TLA0238- | 192 | TLA0238- | 164 | TL0015- | 61 |
| | LFR1-3 | | LCDR1-1 | | LFR2-3 | | LCDR2-1 | | LFR3-3 | | LCDR3-1 | | LFR4-3/ | |
| | | | | | | | | | | | | | TLA0238- | |
| | | | | | | | | | | | | | LFR4-3 | |
| TLA0238-2-2 | TLA0238- | 172 | TLA0238- | 155 | TLA0238- | 183 | TLA0238- | 160 | TLA0238- | 192 | TLA0238- | 164 | TL0015- | 61 |
| | LFR1-3 | | LCDR1-1 | | LFR2-3 | | LCDR2-1 | | LFR3-3 | | LCDR3-1 | | LFR4-3/ | |
| | | | | | | | | | | | | | TLA0238- | |
| | | | | | | | | | | | | | LFR4-3 | |
| TLA0238-5 | TLA0238- | 173 | TLA0238- | 155 | TLA0238- | 183 | TLA0238- | 160 | TL0015- | 57 | TLA0238- | 164 | TLA0238- | 201 |
| | LFR1-4/ | | LCDR1-1 | | LFR2-3 | | LCDR2-1 | | LFR3-4/ | | LCDR3-1 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-6 | TLA0238- | 173 | TLA0238- | 155 | TLA0238- | 183 | TLA0238- | 160 | TL0015- | 57 | TLA0238- | 164 | TLA0238- | 201 |
| | LFR1-4/ | | LCDR1-1 | | LFR2-3 | | LCDR2-1 | | LFR3-4/ | | LCDR3-1 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-7 | TLA0238- | 174 | TLA0238- | 156 | TLA0238- | 184 | TLA0238- | 161 | TLA0238- | 193 | TLA0238- | 164 | TLA0238- | 199 |
| | LFR1-5 | | LCDR1-2 | | LFR2-4 | | LCDR2-2 | | LFR3-5 | | LCDR3-1 | | LFR4-1 | |
| TLA0238-8 | TLA0238- | 175 | TLA0238- | 157 | TLA0238- | 185 | TLA0238- | 161 | TLA0238- | 194 | TLA0238- | 164 | TLA0238- | 199 |
| | LFR1-6 | | LCDR1-3 | | LFR2-5 | | LCDR2-2 | | LFR3-6 | | LCDR3-1 | | LFR4-1 | |
| TLA0238-9 | TLA0238- | 176 | TLA0238- | 157 | TLA0238- | 186 | TLA0238- | 162 | TLA0238- | 195 | TLA0238- | 164 | TLA0238- | 199 |
| | LFR1-7 | | LCDR1-3 | | LFR2-6 | | LCDR2-3 | | LFR3-7 | | LCDR3-1 | | LFR4-1 | |
| TLA0238-10 | TLA0238- | 177 | TLA0238- | 156 | TLA0238- | 187 | TLA0238- | 161 | TLA0238- | 196 | TLA0238- | 164 | TLA0238- | 199 |
| | LFR1-8 | | LCDR1-2 | | LFR2-7 | | LCDR2-2 | | LFR3-8 | | LCDR3-1 | | LFR4-1 | |
| TLA0238-11 | TLA0238- | 173 | TLA0238- | 155 | TLA0238- | 183 | TLA0238- | 160 | TL0015- | 57 | TLA0238- | 164 | TLA0238- | 201 |
| | LFR1-4/ | | LCDR1-1 | | LFR2-3 | | LCDR2-1 | | LFR3-4/ | | LCDR3-1 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-12 | TLA0238- | 178 | TLA0238- | 158 | TLA0238- | 188 | TLA0238- | 160 | TLA0238- | 196 | TLA0238- | 164 | TLA0238- | 199 |
| | LFR1-9 | | LCDR1-4 | | LFR2-8 | | LCDR2-1 | | LFR3-8 | | LCDR3-1 | | LFR4-1 | |
| TLA0238-13 | TLA0238- | 173 | TLA0238- | 155 | TLA0238- | 183 | TLA0238- | 160 | TL0015- | 57 | TLA0238- | 164 | TLA0238- | 201 |
| | LFR1-4/ | | LCDR1-1 | | LFR2-3 | | LCDR2-1 | | LFR3-4/ | | LCDR3-1 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-14 | TLA0238- | 179 | TLA0238- | 156 | TLA0238- | 187 | TLA0238- | 162 | TLA0238- | 197 | TLA0238- | 164 | TLA0238- | 199 |
| | LFR1-10 | | LCDR1-2 | | LFR2-7 | | LCDR2-3 | | LFR3-9 | | LCDR3-1 | | LFR4-1 | |
| TLA0238-15 | TLA0238- | 173 | TLA0238- | 155 | TLA0238- | 183 | TLA0238- | 160 | TL0015- | 57 | TLA0238- | 165 | TLA0238- | 201 |
| | LFR1-4/ | | LCDR1-1 | | LFR2-3 | | LCDR2-1 | | LFR3-4/ | | LCDR3-2 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-16 | TLA0238- | 173 | TLA0238- | 155 | TLA0238- | 183 | TLA0238- | 160 | TL0015- | 57 | TLA0238- | 166 | TLA0238- | 201 |
| | LFR1-4/ | | LCDR1-1 | | LFR2-3 | | LCDR2-1 | | LFR3-4/ | | LCDR3-3 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-17 | TLA0238- | 173 | TLA0238- | 155 | TLA0238- | 183 | TLA0238- | 160 | TL0015- | 57 | TLA0238- | 165 | TLA0238- | 201 |
| | LFR1-4/ | | LCDR1-1 | | LFR2-3 | | LCDR2-1 | | LFR3-4/ | | LCDR3-2 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-18 | TLA0238- | 173 | TLA0238- | 155 | TLA0238- | 183 | TLA0238- | 160 | TL0015- | 57 | TLA0238- | 166 | TLA0238- | 201 |
| | LFR1-4/ | | LCDR1-1 | | LFR2-3 | | LCDR2-1 | | LFR3-4/ | | LCDR3-3 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-19 | TLA0238- | 173 | TLA0238- | 155 | TLA0238- | 183 | TLA0238- | 160 | TL0015- | 57 | TLA0238- | 165 | TLA0238- | 201 |
| | LFR1-4/ | | LCDR1-1 | | LFR2-3 | | LCDR2-1 | | LFR3-4/ | | LCDR3-2 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-20 | TLA0238- | 173 | TLA0238- | 155 | TLA0238- | 183 | TLA0238- | 160 | TL0015- | 57 | TLA0238- | 166 | TLA0238- | 201 |
| | LFR1-4/ | | LCDR1-1 | | LFR2-3 | | LCDR2-1 | | LFR3-4/ | | LCDR3-3 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-21 | TLA0238- | 173 | TLA0238- | 155 | TLA0238- | 183 | TLA0238- | 160 | TL0015- | 57 | TLA0238- | 167 | TLA0238- | 201 |
| | LFR1-4/ | | LCDR1-1 | | LFR2-3 | | LCDR2-1 | | LFR3-4/ | | LCDR3-4 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-22 | TLA0238- | 173 | TLA0238- | 155 | TLA0238- | 183 | TLA0238- | 160 | TL0015- | 57 | TLA0238- | 168 | TLA0238- | 201 |
| | LFR1-4/ | | LCDR1-1 | | LFR2-3 | | LCDR2-1 | | LFR3-4/ | | LCDR3-5 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-23 | TLA0238- | 173 | TLA0238- | 155 | TLA0238- | 183 | TLA0238- | 160 | TL0015- | 57 | TLA0238- | 167 | TLA0238- | 201 |
| | LFR1-4/ | | LCDR1-1 | | LFR2-3 | | LCDR2-1 | | LFR3-4/ | | LCDR3-4 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-24 | TLA0238- | 173 | TLA0238- | 155 | TLA0238- | 183 | TLA0238- | 160 | TL0015- | 57 | TLA0238- | 168 | TLA0238- | 201 |
| | LFR1-4/ | | LCDR1-1 | | LFR2-3 | | LCDR2-1 | | LFR3-4/ | | LCDR3-5 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-25 | TLA0238- | 173 | TLA0238- | 155 | TLA0238- | 183 | TLA0238- | 160 | TL0015- | 57 | TLA0238- | 167 | TLA0238- | 201 |
| | LFR1-4/ | | LCDR1-1 | | LFR2-3 | | LCDR2-1 | | LFR3-4/ | | LCDR3-4 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-26 | TLA0238- | 173 | TLA0238- | 155 | TLA0238- | 183 | TLA0238- | 160 | TL0015- | 57 | TLA0238- | 168 | TLA0238- | 201 |
| | LFR1-4/ | | LCDR1-1 | | LFR2-3 | | LCDR2-1 | | LFR3-4/ | | LCDR3-5 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-27 | TLA0238- | 173 | TLA0238- | 155 | TLA0238- | 183 | TLA0238- | 160 | TL0015- | 57 | TLA0238- | 167 | TLA0238- | 201 |
| | LFR1-4/ | | LCDR1-1 | | LFR2-3 | | LCDR2-1 | | LFR3-4/ | | LCDR3-4 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-28 | TLA0238- | 173 | TLA0238- | 155 | TLA0238- | 183 | TLA0238- | 160 | TL0015- | 57 | TLA0238- | 168 | TLA0238- | 201 |
| | LFR1-4/ | | LCDR1-1 | | LFR2-3 | | LCDR2-1 | | LFR3-4/ | | LCDR3-5 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-29 | TLA0238- | 173 | TLA0238- | 155 | TLA0238- | 183 | TLA0238- | 160 | TL0015- | 57 | TLA0238- | 167 | TLA0238- | 201 |
| | LFR1-4/ | | LCDR1-1 | | LFR2-3 | | LCDR2-1 | | LFR3-4/ | | LCDR3-4 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |
| TLA0238-30 | TLA0238- | 173 | TLA0238- | 155 | TLA0238- | 183 | TLA0238- | 160 | TL0015- | 57 | TLA0238- | 168 | TLA0238- | 201 |
| | LFR1-4/ | | LCDR1-1 | | LFR2-3 | | LCDR2-1 | | LFR3-4/ | | LCDR3-5 | | LFR4-4/ | |
| | TLA0241- | | | | | | | | TLA0238- | | | | TLA0241- | |
| | LFR1-2 | | | | | | | | LFR3-4/ | | | | LFR4-1 | |
| | | | | | | | | | TLA0241- | | | | | |
| | | | | | | | | | LFR3-2 | | | | | |

In one aspect, the present application provides an isolated antigen-binding protein comprising a first antigen-binding domain which specifically binds to TL1A. The first binding domain includes a heavy chain variable region (VHI). The VHI comprises at least one heavy chain complementarity determining regions (HCDR). The VHI may include at least one, two or three of the HCDR1, the HCDR2 or the HCDR3. In some embodiments, the HCDR 1, the HCDR3 and the HCDR3 are sequences as set forth in SEQ TD NO: 235, 238 and 242 respectively.

The HCDR3 of the antigen-binding protein may include amino acid sequence as set forth in SEQ TD NO: 242. For example, the sequence of the HCDR3 of the antigen-binding protein may be defined by Kabat.

The HCDR2 of the antigen-binding protein may include amino acid sequence as set forth in SEQ ID NO:238. For example, the sequence of the HCDR2 of the antigen-binding protein may be defined by Kabat.

In some embodiments, the HCDR2 may be DINPNNGRTTYX94X95X96X97X98G(SEQ ID NO:238). X94 may be A or N. X95 may be D or Q. X96 may be K or S. X97 may be F or V. X98 may be K or Q or T.

In some embodiments, the HCDR2 may include an amino acid sequence as set forth in any one of SEQ ID NOs:239-241.
the HCDR1 of the antigen-binding protein may include amino acid sequence as set forth in SEQ ID NO:235. For example, the sequence of the HCDR1 of the antigen-binding protein may be defined by Kabat.

In some embodiments, the HCDR1 may be DYY X99 X100(SEQ ID NO:235). X99 may be L or M. X100 may be N or H.

In some embodiments, the HCDR1 may include an amino acid as set forth in any one of SEQ ID NO:236-237.

In some embodiments, the VH comprises HFR1, and C-terminus of HFR1 is directly or indirectly linked to the N-terminus of the HCDR1. For example, the sequence of HFR1 of the antigen-binding protein may be defined by Kabat. HFR1 comprises an amino acid as set forth in SEQ ID NO:243.

In some embodiments, HFR1 is X101IX102 X103TQSPX104 X105 X106S X107S X108G X109 X110VTITC(SEQ ID NO: 243). X101 may be A or D or E. X102 may be V or Q. X103 may be L or M. X104 may be A or G or P or S or T. X105 may be S or T. X106 may be L or V X107 may be A or L. X108 may be P or V. X109 may be D or E. X110 may be K or R.

In some embodiments, HFR1 is selected from an amino acid sequence as set forth in any one of SEQ ID NO:244-250.

In some embodiments, VH comprises the HFR2, and the HFR2 is located between the HCDR1 and the HCDR2. For example, the sequence of the HFR2 of the antigen-binding protein may be defined by Kabat. HFR2 comprises an amino acid sequence as set forth in SEQ ID NO:251.

In some embodiments, the HFR2 is WYQQKP X111 X112 X113P X114LLIY(SEQ ID NO:251). X111 may be D or G. X112 may be K or Q. X113 may be A or S. X114 may be K or Q.

In some embodiments, the HFR2 is selected from an amino acid sequence as set forth in any one of SEQ ID NO: 252-255.

In some embodiments, VH comprises the HFR3, and the HFR3 is located between the HCDR2 and the HCDR3. For example, the sequence of the HFR3 of the antigen-binding protein may be defined by Kabat. the HFR3 comprises an amino acid as set forth in SEQ ID NO:256.

In some embodiments, the HFR3 is GVPX115RFSGSGSGTX116FTLTIX117SLX118X119ED X120ATYYC(SEQ ID NO:256). X115 may be A or D or S. X116 may be D or E or S. X117 may be S or N. X118 may be E or Q. X119 may be A or P. X120 may be A or F.

In some embodiments, the HFR3 is selected from an amino acid sequence as set forth in any one of SEQ ID NO:257-262.

In some embodiments, VH comprises the HFR4, and N-terminus of the HFR4 is directly or indirectly linked to C-terminus of the HCDR3. For example, the sequence of the HFR4 of the antigen-binding protein may be defined by Kabat. the HFR4 comprises an amino acid sequence as set forth in SEQ ID NO:263.

In some embodiments, the HFR4 is FGX121GTX122X123EX124K(SEQ ID NO:263). X121 may be A or Q. X122 may be K or R. X123 may be L or V. X124 may be I or L.

In some embodiments, the HFR4 is selected from an amino acid sequence as set forth in any one of SEQ ID NO:264-268.

In some embodiments, the isolated antigen-binding fragment further comprises a light chain variable region(VL), an VL comprises at least one light chain complementarity determining regions (LCDR). VL comprises an LCDR1, an LCDR2 and an LCDR3, which is selected from an amino acid sequence as set forth in any one of SEQ ID NO: 269, SEQ ID NO:274 and SEQ ID NO:277 accordingly.

In some embodiments, VL comprises the LCDR3, including an amino acid sequence as set forth in SEQ ID NO:277. For example, the sequence of the LCDR3 of the antigen-binding protein may be defined by Kabat.

In some embodiments, VL comprises the LCDR2, including an amino acid sequence as set forth in SEQ ID NO:274. For example, the sequence of the LCDR2 of the antigen-binding protein may be defined by Kabat.

In some embodiments, the LCDR2 is KTSN X125PS(SEQ ID NO:274). X125 may be L or R.

In some embodiments, the LCDR2 is selected from an amino acid sequence as set forth in any one of SEQ ID NO:275-276.

In some embodiments, VL comprises the LCDR1, including an amino acid sequence as set forth in SEQ ID NO:269. For example, the sequence of the LCDR1 of the antigen-binding protein may be defined by Kabat.

In some embodiments, the LCDR1 is SX126SSSIISNYX127X128(SEQ ID NO:269). X126 may be A or T. X127 may be L or S. X128 may be A or H.

In some embodiments, the LCDR1 is selected from an amino acid sequence as set forth in any one of SEQ ID NO:270-273.

In some embodiments, VL comprises the LFR1, and C-terminus of the LFR1 is directly or indirectly linked to N-terminus of the LCDR1. For example, the sequence of the LFR1 of the antigen-binding protein may be defined by Kabat. the LFR1 comprises an amino acid sequence as set forth in SEQ ID NO:278.

In some embodiments, the LFR1 is

(SEQ ID NO: 278)
X129IX130X131TQSPX132X133X134SX135SX136GX137X138
VTITC.

X129 may be A or E or D. X130 may be Q or V X131 may be L or M. X132 may be A or G or P or S or T. X133 may be S or T. X134 may be L or V. X135 may be L or A. X136 may be Por V. X137 may be D or E. X138 may be K or R.

In some embodiments, the LFR1 comprises an amino acid sequence as set forth in any one of SEQ ID NO: 47, 173, 279-284.

In some embodiments, VL comprises the LFR2, and the LFR2 is located between the LCDR1 and the LCDR2. For example, the sequence of the LFR2 of the antigen-binding protein may be defined by Kabat, the LFR2 comprises an amino acid sequence as set forth in SEQ ID NO:284.

In some embodiments, the LFR2 is WYQQKP X139 X140 X141P X142LLIY(SEQ ID NO:284). X139 may be D or G. X140 may be K or Q. X141 may be A or S. X142 may be K or Q.

In some embodiments, the LFR2 comprises an amino acid sequence as set forth in any one of SEQ ID NO:52, 285-287.

In some embodiments, VL comprises the LFR3, the LFR3 is located between the LCDR2 and the LCDR3. For example, the sequence of the LFR3 of the antigen-binding protein may be defined by Kabat. The LFR3 comprises an amino acid sequence as set forth in SEQ ID NO: 288.

In some embodiments, the LFR3 is

(SEQ ID NO: 288)
GVPX143RFSGSGSGTX144FTLTIX145
SLX146X147EDX148ATYYC.

### X143 may be A or D or S. X144 may be D or E or S. X145 may be N or S. X146 may be E or Q. X147 may be A or P. X148 may be A or F.

In some embodiments, the LFR3 comprises an amino acid sequence as set forth in any one of SEQ ID NO:289-293 and 57.

In some embodiments, the isolated antigen-binding fragment comprises the LFR4, and N-terminus of the LFR4 is directly or indirectly linked to C terminus of the LCDR3. For example, the sequence of the LFR4 of the antigen-binding protein may be defined by Kabat. The LFR4 comprises an amino acid sequence as set forth in SEQ ID NO:294.

In some embodiments, the LFR4 is FG FGX148GTX149X150EX151K(SEQ ID NO:294). X148 may be A or Q. X149 may be K or R. X150 may be L or V. X151 may be I or L.

In some embodiments, the LFR4 comprises an amino acid sequence as set forth in any one of SEQ TD NO:295-296 and 201.

In some embodiments, VH comprises sequences as set forth in SEQ TD NO:297-230.

In some embodiments, VL comprises sequences as set forth in SEQ TD NO:308-317.

In some embodiments, the antigen-binding proteins described above are as described in Tables E and F.

In some embodiments, the distribution of antibodies for the TLA-0238 series is shown in the following table, with Table E showing the distribution of heavy chain components and Table F showing the distribution of light chain components.

TABLE E

| | VH | | | | | | | | | | | | | |
|---|---|---|---|---|---|---|---|---|---|---|---|---|---|---|
| | | SEQ | | SEQ | | SEQ | | SEQ | | SEQ | | SEQ | | SEQ |
| Antibody | HFR1 | ID | HCDR1 | ID | HFR2 | ID | HCDR2 | ID | HFR3 | ID | HCDR3 | ID | HFR4 | ID |
| TLA0241 | #N/A | #N/A | TLA0241- | 236 | #N/A | #N/A | TLA0241- | 239 | #N/A | #N/A | TLA0241- | 242 | TLA0241- | 268 |
| | | | HCDR1-1 | | | | HCDR2-1 | | | | HCDR3-1 | | HFR4-5 | |
| TLA0241-1 | TLA0241- | 244 | TLA0241- | 236 | TLA0241- | 252 | TLA0241- | 240 | TLA0241- | 257 | TLA0241- | 242 | TLA0241- | 264 |
| | HFR1-1 | | HCDR1-1 | | HFR2-1 | | HCDR2-2 | | HFR3-1 | | HCDR3-1 | | HFR4-1 | |
| TLA0241-2 | TLA0241- | 245 | TLA0241- | 236 | TLA0241- | 252 | TLA0241- | 240 | TLA0241- | 258 | TLA0241- | 242 | TLA-0238- | 152 |
| | HFR1-2 | | HCDR1-1 | | HFR2-1 | | HCDR2-2 | | HFR3-2 | | HCDR3-1 | | HFR4-4 | |
| TLA0241-3 | TLA0241- | 246 | TLA0241- | 237 | TLA0241- | 253 | TLA0241- | 241 | TLA0241- | 259 | TLA0241- | 242 | TLA0241- | 268 |
| | HFR1-3 | | HCDR1-2 | | HFR2-2 | | HCDR2-3 | | HFR3-3 | | HCDR3-1 | | HFR4-5 | |
| TLA0241-4 | TLA0241- | 246 | TLA0241- | 237 | TLA0241- | 254 | TLA0241- | 241 | TLA0241- | 260 | TLA0241- | 242 | TLA0241- | 266 |
| | HFR1-3 | | HCDR1-2 | | HFR2-3 | | HCDR2-3 | | HFR3-4 | | HCDR3-1 | | HFR4-3 | |
| TLA0241-5 | TLA0241- | 247 | TLA0241- | 237 | TLA0241- | 254 | TLA0241- | 241 | TLA0241- | 261 | TLA0241- | 242 | TLA0241- | 268 |
| | HFR1-4 | | HCDR1-2 | | HFR2-3 | | HCDR2-3 | | HFR3-5 | | HCDR3-1 | | HFR4-5 | |
| TLA0241-6 | TLA0241- | 248 | TLA0241- | 236 | TLA0241- | 252 | TLA0241- | 240 | TLA0241- | 258 | TLA0241- | 242 | TLA-0238- | 152 |
| | HFR1-5 | | HCDR1-1 | | HFR2-1 | | HCDR2-2 | | HFR3-2 | | HCDR3-1 | | HFR4-4 | |
| TLA0241-7 | TLA0241- | 246 | TLA0241- | 237 | TLA0241- | 255 | TLA0241- | 241 | TLA0241- | 260 | TLA0241- | 242 | TLA0241- | 268 |
| | HFR1-3 | | HCDR1-2 | | HFR2-4 | | HCDR2-3 | | HFR3-4 | | HCDR3-1 | | HFR4-5 | |
| TLA0241-8 | TLA0241- | 245 | TLA0241- | 236 | TLA0241- | 252 | TLA0241- | 240 | TLA0241- | 258 | TLA0241- | 242 | TLA-0238- | 153 |
| | HFR1-2 | | HCDR1-1 | | HFR2-1 | | HCDR2-2 | | HFR3-2 | | HCDR3-1 | | HFR4-5 | |
| TLA0241-9 | TLA0241- | 249 | TLA0241- | 237 | TLA0241- | 254 | TLA0241- | 241 | TLA0241- | 260 | TLA0241- | 242 | TLA0241- | 268 |
| | HFR1-6 | | HCDR1-2 | | HFR2-3 | | HCDR2-3 | | HFR3-4 | | HCDR3-1 | | HFR4-5 | |
| TLA0241-10 | TLA0241- | 250 | TLA0241- | 237 | TLA0241- | 253 | TLA0241- | 241 | TLA0241- | 262 | TLA0241- | 242 | TLA0241- | 266 |
| | HFR1-7 | | HCDR1-2 | | HFR2-2 | | HCDR2-3 | | HFR3-6 | | HCDR3-1 | | HFR4-3 | |

TABLE F

| | VL | | | | | | | | | | | | | |
|---|---|---|---|---|---|---|---|---|---|---|---|---|---|---|
| | | SEQ | | SEQ | | SEQ | | SEQ | | SEQ | | SEQ | | SEQ |
| Antibody | LFR1 | ID | CDR1 | ID | LFR2 | ID | LCDR2 | ID | LFR3 | ID | LCDR3 | ID | LFR4 | ID |
| TLA0241 | #N/A | #N/A | TLA0241- | 270 | #N/A | #N/A | TLA0241- | 275 | #N/A | #N/A | TLA0241- | 277 | TLA0241- | 296 |
| | | | LCDR1-1 | | | | LCDR2-1 | | | | LCDR3-1 | | LFR4-3 | |
| TLA0241-1 | TL0015- | 47 | TLA0241- | 270 | TL0015- | 52 | TLA0241- | 275 | TLA0241- | 289 | TLA0241- | 277 | TLA0238- | 201 |
| | LFR1-2/ | | LCDR1-1 | | LFR2-4/ | | LCDR2-1 | | LFR3-1 | | LCDR3-1 | | LFR4-4/ | |
| | TLA0241- | | | | TLA0241- | | | | | | | | TLA0241- | |
| | LFR1-1 | | | | LFR2-1 | | | | | | | | LFR4-1 | |
| TLA0241-2 | TLA0238- | 173 | TLA0241- | 270 | TL0015- | 52 | TLA0241- | 275 | TL0015- | 57 | TLA0241- | 277 | TLA0238- | 201 |
| | LFR1-4/ | | LCDR1-1 | | LFR2-4/ | | LCDR2-1 | | LFR3-4/ | | LCDR3-1 | | LFR4-4/ | |
| | TLA0241- | | | | TLA0241- | | | | TLA0238- | | | | TLA0241- | |
| | LFR1-2 | | | | LFR2-1 | | | | LFR3-4/ | | | | LFR4-1 | |
| | | | | | | | | | TLA0241- | | | | | |
| | | | | | | | | | LFR3-2 | | | | | |
| TLA0241-3 | TLA0241- | 279 | TLA0241- | 271 | TLA0241- | 285 | TLA0241- | 275 | TLA0241- | 290 | TLA0241- | 277 | TLA0241- | 295 |
| | LFR1-3 | | LCDR1-2 | | LFR2-2 | | LCDR2-1 | | LFR3-3 | | LCDR3-1 | | LFR4-2 | |
| TLA0241-4 | TLA0241- | 280 | TLA0241- | 271 | TLA0241- | 286 | TLA0241- | 276 | TLA0241- | 291 | TLA0241- | 277 | TLA0241- | 295 |
| | LFR1-4 | | LCDR1-2 | | LFR2-3 | | LCDR2-2 | | LFR3-4 | | LCDR3-1 | | LFR4-2 | |
| TLA0241-5 | TLA0241- | 281 | TLA0241- | 271 | TLA0241- | 287 | TLA0241- | 275 | TLA0241- | 290 | TLA0241- | 277 | TLA0241- | 296 |
| | LFR1-5 | | LCDR1-2 | | LFR2-4 | | LCDR2-1 | | LFR3-3 | | LCDR3-1 | | LFR4-3 | |
| TLA0241-6 | TLA0241- | 282 | TLA0241- | 270 | TL0015- | 52 | TLA0241- | 275 | TL0015- | 57 | TLA0241- | 277 | TLA0238- | 201 |
| | LFR1-6 | | LCDR1-1 | | LFR2-4/ | | LCDR2-1 | | LFR3-4/ | | LCDR3-1 | | LFR4-4/ | |
| | | | | | TLA0241- | | | | TLA0238- | | | | TLA0241- | |
| | | | | | LFR2-1 | | | | LFR3-4/ | | | | LFR4-1 | |
| | | | | | | | | | TLA0241- | | | | | |
| | | | | | | | | | LFR3-2 | | | | | |
| TLA0241-7 | TLA0241- | 283 | TLA0241- | 272 | TLA0241- | 285 | TLA0241- | 275 | TLA0241- | 292 | TLA0241- | 277 | TLA0241- | 295 |
| | LFR1-7 | | LCDR1-3 | | LFR2-2 | | LCDR2-1 | | LFR3-5 | | LCDR3-1 | | LFR4-2 | |
| TLA0241-8 | TLA0238- | 173 | TLA0241- | 270 | TL0015- | 52 | TLA0241- | 275 | TL0015- | 57 | TLA0241- | 277 | TLA0238- | 201 |
| | LFR1-4/ | | LCDR1-1 | | LFR2-4/ | | LCDR2-1 | | LFR3-4/ | | LCDR3-1 | | LFR4-4/ | |
| | TLA0241- | | | | TLA0241- | | | | TLA0238- | | | | TLA0241- | |
| | LFR1-2 | | | | LFR2-1 | | | | LFR3-4/ | | | | LFR4-1 | |
| | | | | | | | | | TLA0241- | | | | | |
| | | | | | | | | | LFR3-2 | | | | | |
| TLA0241-9 | TLA0241- | 279 | TLA0241- | 271 | TLA0241- | 287 | TLA0241- | 275 | TLA0241- | 290 | TLA0241- | 277 | TLA0241- | 296 |
| | LFR1-3 | | LCDR1-2 | | LFR2-4 | | LCDR2-1 | | LFR3-3 | | LCDR3-1 | | LFR4-3 | |
| TLA0241-10 | TLA0241- | 280 | TLA0241- | 273 | TLA0241- | 285 | TLA0241- | 275 | TLA0241- | 293 | TLA0241- | 277 | TLA0241- | 296 |
| | LFR1-4 | | LCDR1-4 | | LFR2-2 | | LCDR2-1 | | LFR3-6 | | LCDR3-1 | | LFR4-3 | |

In some embodiments, said first antigen-binding domain may be a binding domain of an RVT3101 antibody, e.g., comprising VH as shown in SEQ ID NO:322 and VL as shown in SEQ TD NO:323.

### Second Antigen Binding Domain

In the present application, said isolated antigen-binding protein may comprise a second antigen-binding domain.

The second antigen-binding domain does not specifically bind to TL1A.

The second antigen-binding domain specifically binds to α4β7.

In the present application, the second antigen-binding domain of said isolated antigen-binding protein may comprise scFv.

In the present application, in said isolated antigen-binding protein wherein said second antigen binding domain VH comprises an HCDR1 as shown in SEQ ID NO: 332, an HCDR2 as shown in SEQ ID NO: 333, and an HCDR3 as shown in SEQ ID NO: 334.

In the present application, in said isolated antigen-binding protein, said second antigen-binding domain may comprise a heavy chain variable region VH, and said VH may comprise the amino acid sequence shown in SEQ ID NO: 335.

In the present application, in said isolated antigen-binding protein, said second antigen-binding domain may comprise the LCDR3, and said the LCDR3 may comprise the amino acid sequence shown in SEQ ID NO: 34.

The second antigen binding domain VL comprises the LCDR1 as shown in SEQ ID NO: 336, the LCDR2 as shown in SEQ ID NO: 337, and the LCDR3 as shown in SEQ ID NO: 338.

In some embodiments, the second antigen binding domain VH comprises the HFR1 as shown in SEQ ID NO: 382, the HFR2 as shown in SEQ ID NO: 383, the HFR3 as shown in SEQ ID NO: 384, the HFR4 as shown in SEQ ID NO: 385 and/or the second antigen binding domain VL comprises the LFR1 as shown in SEQ ID NO: 386, the LFR2 as shown in SEQ ID NO: 387, the LFR3 as shown in SEQ ID NO: 388, the LFR4 as shown in SEQ ID NO: 389.

In the present application, said second antigen binding domain comprises VL as shown in SEQ ID NO: 339.

In some embodiments, the first antigen-binding domain comprises HCDR1 as set forth in SEQ ID NO: 2, HCDR2 as set forth in SEQ ID NO: 6, HCDR3 as set forth in SEQ ID NO: 15, LCDR1 as set forth in SEQ ID NO: 42, LCDR2 as set forth in SEQ ID NO: 43, and LCDR3 as set forth in SEQ ID NO: 44; and, the second antigen-binding domain comprises HCDR1 as set forth in SEQ ID NO: 332, HCDR2 as set forth in SEQ ID NO: 333, HCDR3 as set forth in SEQ ID NO: 334, LCDR1 as set forth in SEQ ID NO: 336, LCDR2 as set forth in SEQ ID NO: 337, and LCDR3 as set forth in SEQ ID NO: 338.

In some embodiments, the first antigen-binding domain comprises a VH as set forth in SEQ ID NO: 70 and a VL as set forth in SEQ ID NO: 106; and the second antigen-binding domain comprises a VH as set forth in SEQ ID NO: 335 and a VL as set forth in SEQ ID NO: 339.

In some embodiments, (1) the first antigen-binding domain comprises an scFv as set forth in SEQ ID NO: 340; and the second antigen-binding domain comprises a VH as set forth in SEQ ID NO: 335 and a VL as set forth in SEQ ID NO: 339; or, (2) the first antigen-binding domain comprises an scFv as set forth in SEQ ID NO: 341; and the second antigen-binding domain comprises a VH as set forth in SEQ ID NO: 335 and a VL as set forth in SEQ ID NO: 339; or, (3) the first antigen-binding domain comprises an scFv as set forth in SEQ ID NO: 342; and the second antigen-binding domain comprises a VH as set forth in SEQ ID NO: 335 and a VL as set forth in SEQ ID NO: 339; or, (4) the first antigen-binding domain comprises an scFv as set forth in SEQ ID NO: 343; and the second antigen-binding domain comprises a VH as set forth in SEQ ID NO: 335 and a VL as set forth in SEQ ID NO: 339; or, (5) the first antigen binding domain comprises the VH as set forth in SEQ ID NO: 70 and the VL as set forth in SEQ ID NO: 106; and the second antigen binding domain comprises an scFv as set forth in SEQ ID NO:346; or, (6) the first antigen binding domain comprises the VH as set forth in SEQ ID NO: 70 and the VL as set forth in SEQ ID NO: 106; and the second antigen binding domain comprises an scFv as set forth in SEQ ID NO:347.

### Isolated Antigen-Binding Protein

In one aspect, the present application provides an isolated antigen-binding protein that can bind to a monomer TL1A and/or trimer TL1A with a K_{D} value of about 1.0E-8M or less (For example, said KD is no greater than about 5E-08M, no greater than about 4.5E-08M, no greater than about 4E-08M, no greater than about 3.5E-08M, no greater than about 3E-08M, no greater than about 2E-08M, no greater than about 2E-08M, no greater than about 1.5E-08M, no greater than about 1E-08M, no greater than about 5E-09M, no greater than about 4.5E-09M, no greater than about 4E-09M, no greater than about 3.5E-09M, no greater than about 3E-09M, no greater than about 2.5E-09M, no greater than about 2E-09M, no greater than about 1.5E-09M, no greater than about 1E-09M, no greater than about 9E-10M, no greater than about 5E-10M, no greater than about 1E-10M, no greater than about 5E-11M, no greater than about 1E-11M, or no greater than 5E-12M or less.)

In the present application, the isolated antigen-binding protein of the present disclosure is an antagonist of the TL1A receptor, including, but not limited to, DR3 and TR6/DcR3. In the present application, the isolated antigen-binding protein of the present disclosure blocks the interaction of TL1A with death receptor 3 (“DR3”). In the present application, said isolated antigen-binding protein blocks monomeric TL1A-protein and/or trimeric TL1A in a flow-through assay at an IC50 value of about 50 nM or less (e.g., said IC50 value is no higher than about 50 nM, no higher than about 45 nM, no higher than about 40 nM, no higher than about 35 nM, no higher than about 30 nM, no higher than about 25 nM or less) binding to DR3 (DR3: UniprotKB NO. Q93038).

In one aspect, the present application provides an isolated antigen-binding protein that can bind to a α4β7 protein with a K_{D} value of about 1.0E-8M or less (For example, said KD is no greater than about 5E-08M, no greater than about 4.5E-08M, no greater than about 4E-08M, no greater than about 3.5E-08M, no greater than about 3E-08M, no greater than about 2E-08M, no greater than about 2E-08M, no greater than about 1.5E-08M, no greater than about 1E-08M, no greater than about 5E-09M, no greater than about 4.5E-09M, no greater than about 4E-09M, no greater than about 3.5E-09M, no greater than about 3E-09M, no greater than about 2.5E-09M, no greater than about 2E-09M, no greater than about 1.5E-09M, no greater than about 1E-09M, no greater than about 9E-10M, no greater than about 5E-10M, no greater than about 1E-10M, no greater than about 5E-11M, no greater than about 1E-11M, or no greater than 5E-12M or less.).

In the present application, the isolated antigen-binding protein is an antagonist of α4β7 protein receptors, including, but not limited to, MAdCAM-1. In the present application, said isolated antigen-binding protein blocks the interaction of TL1A with mucosal addressin cell adhesion molecule-1 (“MAdCAM-1”). In the present application, the isolated antigen-binding protein blocks binding of α4β7 protein and MAdCAM-1 in a flow-through assay at an IC50 value of about 50 nM or less (e.g., said IC50 value is no higher than about 50 nM, no higher than about 45 nM, no higher than about 40 nM, no higher than about 35 nM, no higher than about 30 nM, no higher than about 25 nM or less).

In the present application, the α4β7 protein does not affect the blocking activity of isolated antigen-binding proteins against TL1A proteins.

In the present application, the isolated antigen-binding proteins can inhibit the activity of the soluble TL1A-activated NF-κB signaling pathway. For example, the soluble TL1A-activated NF-κB signaling pathway can be tested for inhibition by a luciferase reporter gene system.

In the present application, said isolated antigen-binding protein can inhibit the activity of the membrane-bound TL1A-activated NF-κB signaling pathway. For example, whether the membrane-bound TL1A-activated NF-κB signaling pathway is inhibited can be detected by a luciferase reporter gene system.

In the present application, said isolated antigen-binding protein may function to inhibit Caspase3/7 activity. For example, the function of the Caspase3/7 activity can be tested for inhibition by a luciferase reporter gene system.

In the present application, said isolated antigen-binding protein blocks MAdCAM-1 mediated cell adhesion. The blocking ability can be determined, for example, by comparing curves of different antibody concentrations blocking MAdCAM-1 mediated cell adhesion to Hut 78.

In the present application, said isolated antigen-binding protein down-regulates the expression of α4β7. Said expression of α4β7 can be total expression or membrane expression.

In the present application, said isolated antigen-binding protein can inhibit the activity of CD4T cells.

In the present application, said isolated antigen-binding protein inhibits the activity of IFN-γ.

In another aspect, the present application provides an antigen-binding protein which competitively binds α4β7 protein with the antigen-binding protein described in the present application.

In another aspect, the present application provides an antigen-binding protein which competitively binds TL1A with the antigen-binding protein described in the present application.

A binding site of the first antigen-binding domain binding to the TL1A is selected from one or more of the following: R32, Q33, P35, T36, Q37, P44, S89, K102, P103, D161, I162, S163 and L164, including any one, two, three, four, five, six, seven, eight, nine, ten, eleven, twelve or thirteen of the above mentioned sites; and the binding sites are numbered according to the amino acid sequence of TL1A as set forth in SEQ ID NO: 327.

A binding site of the antigen-binding protein binding to the TL1A is selected from one or more of the following: R32, E49, E51, L54, A56, Y117, M146, S148, and Y167, including any one, two, three, four, five, six, seven, eight, and nine of the above mentioned site; and the binding sites are numbered according to the amino acid sequence of TL1A as set forth in SEQ ID NO: 327.

In the present application, the first antigen-binding domain or the second antigen-binding domain comprises one or any combination of the following: Fab, Fab′, F(ab′)2, Fd and Fv fragments, disulfide bond stabilized Fv fragments (dsFv)., (dsFv)2, bispecific dsFv (dsFv-dsFv′), disulfide bond stabilized double-stranded antibody (diabody) (ds double-stranded antibody), single-chain antibody molecule (scFv), Single domain antibody (SDAB), scFv dimer (divalent double-stranded antibody), single domain antibody or nanobody, domain antibody (domain antibody (dAb), shark variable IgNAR domain, camelized VH domain, chicken heavy chain antibody domain, VHH domain, minimum identification unit composed of CDR of mimetic antibody (minimal recognition unit), alternative scaffolds for binding antigens, bivalent domain antibodies, pre-designed ankyrin repeat proteins (DARPins), multispecific proteins (including antigen-binding fragments or any other antibody fragments bound to antigens, but not intact antibody structures).

In the present application, the antigen-binding protein comprises the scFv of the first antigen binding domain or the scFv of the second antigen binding domain.

In the present application, the scFv of the first antigen-binding domain comprises VL and VH of the first antigen-binding domain.

In the present application, in the first antigen-binding domain, the C-terminus of the VL of the first antigen-binding domain is linked directly or via a linker to the N-terminus of the VH of the first antigen-binding domain, or the N-terminus of the VL of the first antigen-binding domain is linked directly or via a linker to the C-terminus of the VH of the first antigen-binding domain.

In the present application, scFv of the first antigen-binding domains comprises sequences as set forth in any one of SEQ ID NO: 340-345.

In the present application, the scFv of the second antigen-binding domain comprises VL and VH of the second antigen-binding domain.

In the present application, in the second antigen binding domain, the C-terminus of the VL of the second antigen binding domain is linked directly or via a linker to the N-terminus of the VH of the second antigen binding domain, or the N-terminus of the VL of the second antigen binding domain is linked directly or via a linker to the C-terminus of the VH of the second antigen binding domain.

In the present application, scFv of the second antigen-binding domains comprises sequences as set forth in any one of SEQ ID NO: 346-347.

In the present application, the isolated antigen-binding protein comprises a heavy chain constant region of IgG and/or the light chain constant region of a human antibody

In the present application, the isolated antigen-binding protein comprises heavy chain constant region of IgG1, IgG2, IgG3 or IgG4

In the present application, the isolated antigen-binding protein comprises a Kappa or Lambda light chain constant region.

In the present application, the isolated antigen-binding protein is derived from IgG, IgM, IgA, IgD or IgE.

In the present application, the isolated antigen-binding protein comprises one or more first heavy chains and one or more first light chains.

The first heavy chain comprises VH of the first antigen-binding domain and/or VH of the second antigen binding domain and/or scFv of the first antigen-binding domain and/or scFv of the second antigen binding domain;
the first light chain comprises a VL of the first antigen-binding domain or a VL of the second antigen binding domain.

In the present application, the first heavy chain comprises VH of the first antigen-binding domain and scFv of the second antigen binding domain, or the first heavy chain comprises VH of the second antigen binding domain and scFv of the first antigen-binding domain.

In the present application, the N-terminus of the scFv of the first antigen-binding domain or the second antigen binding domain is linked directly or via a linker to the C-terminus of the first heavy chain, or the C-terminus of the scFv of the first antigen-binding domain or the second antigen binding domain is linked directly or via a linker to the N-terminus of the first heavy chain.

In the present application, the isolated antigen-binding protein is selected from one of the following:
the C-terminus of the first antigen-binding domain VH is linked to the N-terminus of said first heavy chain directly or via a linker, and the N-terminus of the scFv of the second antigen binding domain is linked to the C-terminus of said first heavy chain directly or via a linker; the C-terminus of the VL of the first antigen-binding domain is located at the N-terminus of the constant region of the light chain;
(2) the C-terminus of the second antigen binding domain VH is linked to the N-terminus of said first heavy chain directly or via a linker, and the N-terminus of the scFv of the first antigen-binding domain is linked to the C-terminus of said first heavy chain directly or via a linker; the C-terminus of the VL of the second antigen binding domain is located at the N-terminus of the light-chain constant region;
(3) the C-terminus of the scFv of the first antigen-binding domain is linked, directly or via a linker, to the N-terminus of said second antigen binding domain VH, and the C-terminus of the VL of the second antigen binding domain is linked to the N-terminus of the light chain constant region;
(4) the C-terminus of the scFv of the second antigen binding domain is connected, directly or via a linker, to the N-terminus of said first antigen-binding domain VH, and the C-terminus of the VL of the first antigen-binding domain is linked to the N-terminus of the light chain constant region.

The first heavy chain is selected from the sequence as set forth in any one of the SEQ ID NO: 350-366 and 378.

The antigen-binding fragment is selected from one of the following:
1) light chain of SEQ ID NO:348 and heavy chain of SEQ ID NO: 350;
2) light chain of SEQ ID NO:348 and heavy chain of SEQ ID NO:351;
3) light chain of SEQ ID NO:348 and heavy chain of SEQ ID NO:352;
4) light chain of SEQ ID NO:348 and heavy chain of SEQ ID NO:353;
5) light chain of SEQ ID NO:349 and heavy chain of SEQ ID NO:354;
6) light chain of SEQ ID NO:349 and heavy chain of SEQ ID NO:355;
7) light chain of SEQ ID NO:348 and heavy chain of SEQ ID NO:356;
8) light chain of SEQ ID NO:372 and heavy chain of SEQ ID NO:357;
9) light chain of SEQ ID NO:349 and heavy chain of SEQ ID NO:358;
10) light chain of SEQ ID NO:348 and heavy chain of SEQ ID NO:359;
11) light chain of SEQ ID NO:372 and heavy chain of SEQ ID NO:360;
12) light chain of SEQ ID NO:348 and heavy chain of SEQ ID NO:361;
13) light chain of SEQ ID NO:348 and heavy chain of SEQ ID NO:362;
14) light chain of SEQ ID NO:348 and heavy chain of SEQ ID NO:363;
15) light chain of SEQ ID NO:348 and heavy chain of SEQ ID NO:364;
16) light chain of SEQ ID NO:348 and heavy chain of SEQ ID NO:365;
17) light chain of SEQ ID NO:348 and heavy chain of SEQ ID NO:366;
18) light chain of SEQ ID NO:348 and heavy chain of SEQ ID NO:378.

In some embodiments, the isolated antigen-binding protein is selected from one of the following: a light chain as shown in SEQ ID NO: 348 and heavy chain as shown in SEQ ID NO: 363.

In some embodiments, the antigen-binding proteins described in the present application have one, two, three, or four combinations of the light and heavy chains described above.

In the present application, antigen-binding proteins comprises one or more first light chains, one or more third heavy chains, and one or more fourth heavy chains, wherein:
1) the third heavy chain comprises VH for the first antigen binding domain or the second antigen binding domain, and the fourth heavy chain comprises the ScFv of the first or second antigen binding domain;
2) the third heavy chain comprises ScFv of the first or second antigen binding domain, the fourth heavy chain comprises the VH of the first or second antigen binding domain.

In the present application, the N-terminus of the first antigen-binding domain or the VH of the second antigen binding domain is linked directly or via a linker to the C-terminus of the third heavy chain, or the N-terminus of the scFv of the first or second antigen binding domains is linked directly or via a linker to the C-terminus of the fourth heavy chain.

In the present application,
1) the third heavy chain comprises the VH of the first antigen-binding domain, and the fourth heavy chain comprises the scFv of the second antigen binding domain; or,
2) the third heavy chain comprises the scFv of the first antigen binding domain, and the fourth chain comprises the VH of the second antigen binding domain, or;
3) The third heavy chain comprises VH of the second antigen binding domain, and the fourth heavy chain comprises the scFv of the first antigen binding domain; or
4) The third chain comprises the scFv of the second antigen binding domain, and the fourth heavy chain comprises VH of the first antigen-binding domain.

In the present application, antigen-binding protein is selected from one of the following:
1) light chain of SEQ ID NO:349, third heavy chain of SEQ ID NO:367 and fourth heavy chain of SEQ ID NO:368;
2) light chain of SEQ ID NO:348, third heavy chain of SEQ ID NO:369 and fourth heavy chain of SEQ ID NO:370.

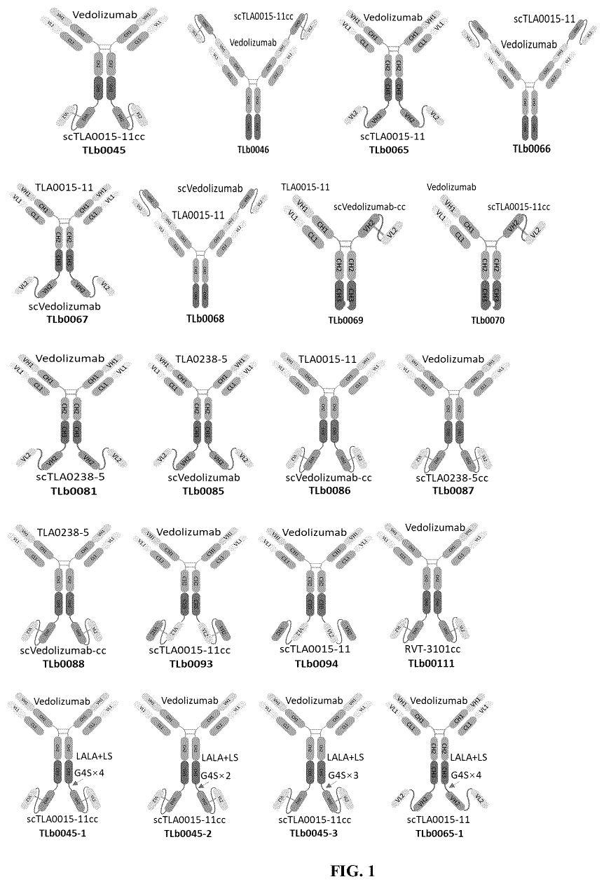

As shown in FIG. 1, the antigen-binding proteins of the present application may have the following structures (not including linkers):
(1) Heavy chain from N-terminal to C-terminal: VH of the second antigen binding domain, CH1, CH2, CH3, VH of the first antigen binding domain scFv, VL of the first antigen binding domain scFv; light chain from N-terminal to C-terminal VL of the second antigen binding domain, light chain constant region;
(2) Heavy chain from N-terminal to C-terminal: VH of the second antigen binding domain, CH1, CH2, CH3, VL of the first antigen binding domain scFv, VH of the first antigen binding domain scFv; light chain from N-terminal to C-terminal VL of the second antigen binding domain, light chain constant region;
(3) Heavy chain from N-terminal to C-terminal: VL of the first antigen binding domain scFv, VH of the first antigen binding domain scFv, VH of the second antigen binding domain, CH1, CH2, CH3; light chain from N-terminal to C-terminal: VL of the second antigen binding domain, light chain constant region.
(4) Heavy chain from N-terminal to C-terminal: VH of the first antigen binding domain, CH1, CH2, CH3, VH of the second antigen binding domain scFv, VL of the second antigen binding domain scFv; light chain from N-terminal to C-terminal: VL of the first antigen binding domain, light chain constant region;
(5) Heavy chain from N-terminal to C-terminal: VH of the first antigen binding domain, CH1, CH2, CH3, VL of the second antigen binding domain scFv, VH of the second antigen binding domain scFv; light chain from N-terminal to C-terminal: VL of the first antigen binding domain, light chain constant region;
(6) Heavy chain from N-terminal to C-terminal: VL of the second antigen binding domain scFv, VH of the second antigen binding domain scFv, VH of the first antigen binding domain, CH1, CH2, CH3; light chain from N-terminal to C-terminal: VL of the first antigen binding domain, light chain constant region.

Specifically, the antigen-binding protein of the present application as shown in FIG. 1 may have the following structure (not counting linkers). Wherein specific first antigen binding domains and or second antigen binding domains of the species are shown in the Figure.

TLb0045, heavy chain from N-terminal to C-terminal: VH of the second antigen binding domain, CH1, CH2, CH3, VH of the first antigen binding domain scFv, VL of the first antigen binding domain scFv; light chain from N-terminal to C-terminal VL of the second antigen binding domain, light chain constant region.

TLb0046, heavy chain from N-terminal to C-terminal: VL of first antigen binding domain scFv, VH of first antigen binding domain scFv, VH of second antigen binding domain, CH1, CH2, CH3; light chain from N-terminal to C-terminal: VL of second antigen binding domain, light chain constant region.

TLb0065, heavy chain from N-terminal to C-terminal: VH of second antigen binding domain, CH1, CH2, CH3, VH of first antigen binding domain scFv, VL of first antigen binding domain scFv; light chain from N-terminal to C-terminal VL of second antigen binding domain, light chain constant region.

TLb0066, heavy chain from N-terminal to C-terminal: VL of first antigen binding domain scFv, VH of first antigen binding domain scFv, VH of second antigen binding domain, CH1, CH2, CH3; light chain from N-terminal to C-terminal: VL of second antigen binding domain, light chain constant region.

TLb0067, Heavy chain from N-terminal to C-terminal: VH of the first antigen binding domain, CH1, CH2, CH3, VL of the second antigen binding domain scFv, VH of the second antigen binding domain scFv; Light chain from N-terminal to C-terminal: VL of the first antigen binding domain, light chain constant region.

TLb0068 Heavy chain from N-terminal to C-terminal: VL of the second antigen binding domain scFv, VH of the second antigen binding domain scFv, VH of the first antigen binding domain, CH1, CH2, CH3; Light chain from N-terminal to C-terminal: VL of the first antigen binding domain, light chain constant region.

TLb0081, heavy chain from N-terminal to C-terminal: VH of second antigen binding domain, CH1, CH2, CH3, VH of first antigen binding domain scFv, VL of first antigen binding domain scFv; light chain from N-terminal to C-terminal VL of second antigen binding domain, light chain constant region.

TLb0085, heavy chain from N-terminal to C-terminal: VH of first antigen binding domain, CH1, CH2, CH3, VL of second antigen binding domain scFv, VH of second antigen binding domain scFv; light chain from N-terminal to C-terminal: VL of first antigen binding domain, light chain constant region.

TLb0086, heavy chain from N-terminal to C-terminal: VH of first antigen binding domain, CH1, CH2, CH3, VL of second antigen binding domain scFv, VH of second antigen binding 15 domain scFv,; light chain from N-terminal to C-terminal: VL of first antigen binding domain, light chain constant region.

TLb0087, heavy chain from N-terminal to C-terminal: VH of second antigen binding domain, CH1, CH2, CH3, VH of first antigen binding domain scFv, VL of first antigen binding domain scFv; light chain from N-terminal to C-terminal VL of second antigen binding domain, light chain constant region.

TLb0088, heavy chain from N-terminal to C-terminal: VH of first antigen binding domain, CH1, CH2, CH3, VL of second antigen binding domain scFv, VH of second antigen binding domain scFv; light chain from N-terminal to C-terminal: VL of first antigen binding domain, light chain constant region.

TLb0093, heavy chain from N-terminal to C-terminal: VH of second antigen binding domain, CH1, CH2, CH3, VL of first antigen binding domain scFv, VH of first antigen binding domain scFv; light chain from N-terminal to C-terminal VL of second antigen binding domain, light chain constant region.

TLb0094, heavy chain from N-terminal to C-terminal: VH of second antigen binding domain, CH1, CH2, CH3, VL of first antigen binding domain scFv, VH of first antigen binding domain scFv; light chain from N-terminal to VL of second antigen binding domain at C-terminal, light chain constant region.

TLb00111, VH of second antigen binding domain, CH1, CH2, CH3, VH of first antigen binding domain scFv, VL of first antigen binding domain scFv; light chain from N-terminal to C-terminal VL of second antigen binding domain, light chain constant region.

TLb0045-1, heavy chain from N-terminal to C-terminal: VH of second antigen binding domain, CH1, CH2, CH3, VH of first antigen binding domain scFv, VL of first antigen binding domain scFv; light chain from N-terminal to C-terminal VL of second antigen binding domain, light chain constant region.

TLb0045-2, heavy chain from N-terminal to C-terminal: VH of second antigen binding domain, CH1, CH2, CH3, VH of first antigen binding domain scFv, VL of first antigen binding domain scFv; light chain from N-terminal to C-terminal second antigen binding domain, VL of light chain constant region.

TLb0045-3, heavy chain from N-terminal to C-terminal: VH of second antigen binding domain, CH1, CH2, CH3, VH of first antigen binding domain scFv, VL of first antigen binding domain scFv; light chain from N-terminal to C-terminal second antigen binding domain, light chain constant region.

TLb0061-1, heavy chain from N-terminal to C-terminal: VH of second antigen binding domain, CH1, CH2, CH3, VH of first antigen binding domain scFv, VL of first antigen binding domain scFv; light chain from N-terminal to C-terminal second antigen binding domain, light chain constant region.

### Recombinant Antibodies

In the present application, said antigen binding protein may include a heavy chain constant region, said heavy chain constant region may include a constant region derived from IgG or a constant region derived from IgY.

For example, said antigen-binding protein may comprise a constant region derived from IgG. For example, said antigen-binding protein may comprise a heavy chain. The heavy chain may comprise a heavy chain variable region and a heavy chain constant region. The heavy chain variable region and said heavy chain constant region may be directly or indirectly linked.

For example, said heavy chain variable region may be directly linked to said heavy chain constant region. For example, said heavy chain constant region may comprise a constant region derived from a protein selected from the following group: IgG1, IgG2, IgG3, and IgG4. In some embodiments, said heavy chain constant region may comprise a constant region derived from a protein selected from the following group: IgG1. Fc constant region of an antigen-binding protein may comprise an amino acid sequence as shown in SEQ ID NO:320 or SEQ ID NO:321 or SEQ ID NO:371, 373, 374, and CH1 may be an amino acid sequence as shown in SEQ ID NO:319.

In the present application, said antigen binding protein may include a light chain constant region, said light chain constant region may include a constant region derived from Igκ or a constant region derived from Igλ. For example, said light chain constant region may include a Lambda light chain constant region, such as SEQ ID NO:318.

For example, the light chain constant region of said antigen-binding protein comprises the amino acid sequence shown in SEQ ID NO:318.

### Peptides and Immunoconjugates

In another aspect, the present application provides one or more polypeptides, which may comprise an isolated antigen-binding protein of the present application.

In another aspect, the present application provides one or more immunoconjugates, which may comprise an isolated antigen-binding protein of the present application. In some embodiments, the immunoconjugate also comprises a pharmaceutically acceptable therapeutic agent.

### Nucleic Acid, Vector, and Cell

In another aspect, the present application also provides one or more isolated nucleic acid molecules that may encode the isolated antigen-binding proteins described herein. For example, each of the one or more nucleic acid molecules may encode the entire antigen-binding protein or a portion thereof (e.g., one or more of the HCDR1-3 and the heavy chain variable regions).

The nucleic acid molecules described herein may be isolated. For example, it may be produced or synthesized by the following methods: (i) in vitro amplification, e.g., by polymerase chain reaction (PCR) amplification, (ii) clonal recombination, (iii) purification, e.g., by digestion and gel electrophoresis fractionation, or (iv) synthesis, e.g., by chemical synthesis. For example, the isolated nucleic acid may be a nucleic acid molecule prepared by recombinant DNA technology.

In the present application, the nucleic acids encoding the isolated antigen-binding proteins may be prepared by a variety of methods known in the art including, but not limited to, using reverse transcription PCR and PCR to obtain nucleic acid molecules of the isolated antigen-binding proteins described herein.

In another aspect, the present application provides one or more vectors comprising one or more nucleic acid molecules described herein. Each vector may comprise one or more of the nucleic acid molecules. In addition, the vector may also comprise other genes, such as marker genes that allow for selection of the vector in an appropriate host cell and under appropriate conditions. In addition, the vector may also comprise expression control elements that allow for the proper expression of the coding region in an appropriate host. Such control elements are well known to those skilled in the art and may include, for example, promoters, ribosome binding sites, enhancers, and other control elements that regulate gene transcription or mRNA translation, etc. In some embodiments, the expression control sequence is a regulatable element. The specific structure of the expression control sequences may vary depending on the function of the species or cell type, but typically comprise 5′ non-transcribed sequences and 5′ and 3′ non-translated sequences involved in transcription and translation initiation, respectively, such as TATA cassettes, capping sequences, CAAT sequences, etc. For example, the 5′ non-transcribed expression control sequence may comprise a promoter region, which may comprise a promoter sequence for transcription control of a functionally linked nucleic acid. The expression control sequences may also include enhancer sequences or upstream activator sequences. In the present application, suitable promoters may include, for example, promoters for SP6, T3, and T7 polymerases, human U6RNA promoters, CMV promoters, and artificial hybrid promoters thereof (e.g., CMV), wherein some portion of the promoter may be fused to some portion of the promoter of a gene for another cellular proteins (e.g., human GAPDH, glyceraldehyde-3-phosphate dehydrogenase), which may or may not comprise an additional intron. One or more nucleic acid molecules described herein can be operably linked to the expression control elements.

Such vectors may include, for example, plasmids, cosmids, viruses, phages, or other vectors commonly used in, for example, genetic engineering. For example, the vector may be an expression vector. For example, the vector may be a viral vector. The patient may be administered directly (in vivo) with the viral vector or may be administered indirectly, e.g., the patient may be administered with the cell treated with the virus in vitro (ex vivo). Viral vector technology is well known in the art and is described, for example, in Sambrook et al., (2001, Molecular Cloning: A Laboratory Manual, Cold Spring Harbor Laboratory, New York) and other virology and molecular biology manuals. Conventional virus-based systems may include retroviral vectors, lentiviral vectors, adenoviral vectors, adeno-associated viral vectors, and herpes simplex viral vectors for gene transfer. In some cases, retroviral, lentiviral, and adeno-associated viral methods can be used to transfer and integrate gene into the host genome for long term expression of the inserted gene. Lentiviral vectors are retroviral vectors capable of transducing or infecting non-dividing cells and typically producing higher viral titers. Lentiviral vectors may comprise a long terminal repeat 5′ LTR and a truncated 3′ LTR, a RRE, a rev response element (cPPT), a central termination sequence (CTS), and/or a post-translational regulatory element (WPRE). The vectors described herein can be introduced into cells.

According to another aspect, the present application provides a cell. The cell may comprise an isolated antigen-binding protein as described herein, a polypeptide as described herein, an immunoconjugate as described herein, one or more nucleic acid molecules and/or one or more vectors as described herein. For example, each or every cell may comprise a nucleic acid molecule or vector described herein. For example, each or every cell may comprise many (e.g., 2 or more) or multiple (e.g., 2 or more) kinds of nucleic acid molecules or vectors described herein. For example, the vectors described herein can be introduced into said host cells, such as prokaryotic cells (e.g., bacterial cells), CHO cells, NS/0 cells, HEK293T cells, 293F cells, or HEK293A cells, or other eukaryotic cells, such as cells from plants, fungal or yeast cells, etc. The vectors described herein can be introduced into the host cells by methods known in the art, such as electroporation, lipofectine transfection, lipofectamin transfection, etc. For example, the cells may include yeast cells. For example, the cells may include E. coli cells. For example, the cells may include mammalian cells. For example, the cells may include immune cells.

The cells may include immune cells. In some cases, the cells may include immune cells. For example, the cells may include T cells, B cells, natural killer cells (NK cells), macrophages, NKT cells, monocytes, dendritic cells, granulocytes, lymphocytes, leukocytes and/or peripheral blood mononuclear cells.

### Pharmaceutical Composition and Pharmaceutical Combination

In another aspect, the present application provides a pharmaceutical composition. The pharmaceutical composition may comprise the isolated antigen-binding protein, the polypeptide, the immunoconjugate, the isolated nucleic acid molecule, the vector, the cell, and/or the pharmaceutically acceptable adjuvant and/or excipient as described herein. In the present application, the pharmaceutically acceptable adjuvants may include buffers, antioxidants, preservatives, low molecular weight polypeptides, proteins, hydrophilic polymers, amino acids, sugars, chelating agents, counter ions, metal complexes and/or nonionic surfactants. Any conventional media or agent is contemplated for the pharmaceutical compositions of the present application, unless they are incompatible with the cells described herein. In the present application, the pharmaceutically acceptable excipients may include an additive other than the main drug in the pharmaceutical preparation, which may also be referred to as pharmaceutical necessities. For example, the excipients may include binders, fillers, disintegrants, and lubricants in tablets. For example, the excipients may include alcohol, vinegar, medicinal juice, etc. in traditional Chinese medicine pills. For example, the excipients may include the base portion of semi-solid formulation ointments and creams. For example, the excipients may include preservatives, antioxidants, flavoring agents, perfuming agents, solubilizing assistant, emulsifiers, solubilizers, osmotic pressure regulators, and colorants in liquid formulations.

### Kit, Use and Method

In one aspect, the present application provides a method for detecting or assaying TL1A, said method may comprise using said isolated antigen-binding protein or said peptide.

In the present application, said method may comprise an in vitro method, an ex vivo method, a method for non-diagnostic or non-therapeutic purposes.

For example, said methods may comprise methods for detecting the presence and/or amount of TL1A for non-diagnostic purposes, which may comprise the following steps:
1) contacting a sample with an antigen-binding protein of the present application; and
2) detecting the presence and/or amount of said antigen-binding protein bound by the sample to determine the presence and/or expression level of TL1A in a sample obtained from a subject.

In another aspect, the present application provides a kit for TL1A, which may comprise the use of said isolated antigen-binding protein or said peptide.

In the present application, said kit may also include instructions for use, said instructions for use documenting a method for detecting the presence and/or level of TL1A. For example, said methods may include in vitro methods, ex vivo methods, methods for non-diagnostic or non-therapeutic purposes.

In another aspect, the present application provides a use of said isolated antigen-binding protein or said polypeptide in the preparation of a kit, said kit being usable for methods for detecting the presence and/or the amount of TL1A. For example, said methods may include in vitro methods, ex vivo methods, methods for non-diagnostic or non-therapeutic purposes.

In another aspect, the present application provides a method of inhibiting the binding of TL1A to DR3/TR6/DcR3 comprising administering to a subject in need thereof an effective amount of said isolated antigen-binding protein, said peptide, said immuno-fix, said isolated nucleic acid molecule, said vector, and/or said cell. Said methods may be in vitro or ex vivo methods.

In another aspect, the present application provides a method for detecting or assaying α4β7, said method may comprise using said isolated antigen-binding protein or said peptide.

In the present application, said method may comprise an in vitro method, an ex vivo method, a method for non-diagnostic or non-therapeutic purposes.

For example, said methods may comprise methods for detecting the presence and/or amount of α4β7 for non-diagnostic purposes, which may comprise the following steps:
1) contacting a sample with an antigen-binding protein of the present application; and
2) detecting the presence and/or amount of said antigen-binding protein bound by the sample to determine the presence and/or expression level of α4β7 in a sample obtained from a subject.

In another aspect, the present application provides a kit for α4β7, which may comprise the use of said isolated antigen-binding protein or said peptide.

In the present application, said kit may also include instructions for use, said instructions for use documenting a method for detecting the presence and/or level of α4β7. For example, said methods may include in vitro methods, ex vivo methods, methods for non-diagnostic or non-therapeutic purposes.

In another aspect, the present application provides a use of said isolated antigen-binding protein or said polypeptide in the preparation of a kit, said kit being usable for methods for detecting the presence and/or the amount of α4β7. For example, said methods may include in vitro methods, ex vivo methods, methods for non-diagnostic or non-therapeutic purposes.

In another aspect, the present application provides a method of inhibiting the binding of α4β7 to MAdCAM-1 comprising administering to a subject in need thereof an effective amount of said isolated antigen-binding protein, said peptide, said immuno-fix, said isolated nucleic acid molecule, said vector, and/or said cell. Said methods may be in vitro or ex vivo methods.

In another aspect, in the present application, said kits and/or said pharmaceutical compositions are used for the prevention, mitigation and/or treatment of a disease or condition.

In another aspect, the present application provides the use of a pharmaceutical composition in the preparation of a drug, said drug being used for the prevention, alleviation and/or treatment of a disease or condition.

In another aspect, the present application provides a method of preventing and/or treating a disease or condition comprising administering to a subject in need thereof said isolated antigen-binding protein, said isolated nucleic acid molecule, said vector, said cell, said pharmaceutical composition.

The disease or condition disclosed herein may be an inflammatory disease, a fibrostenotic disease, or a fibrotic disease. In some instances, the disease or the condition is a TL1A-mediated disease or condition. The term, “TL1A-mediated disease or condition” refers to a disease or a condition pathology or pathogenesis that is driven, at least in part, by TL1A signaling. In some instances, the disease or the condition is immune-mediated disease or condition, such as those mediated by TL1A.

In some embodiments, the disease or the condition is an inflammatory disease or disorder that is mediated, at least in part, by TL1A signaling. Non-limiting examples of inflammatory disease include, allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis (e.g., idiopathic pulmonary fibrosis), rheumatic disease, scleroderma, sinusitis, tuberculosis.

In some embodiments, the disease or the condition is an autoimmune disease that is mediated, at least in part, by TL1A signaling. Non-limiting examples of autoimmune disease or disorder include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal MotorNeuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

In some embodiments, the disease or the condition is a cancer that is mediated, at least in part, by TL1A signaling. Non-limiting examples of cancers include Adenoid Cystic Carcinoma, Adrenal Gland Cancer, Amyloidosis, Anal Cancer, Ataxia-Telangiectasia, Atypical Mole Syndrome, Basal Cell Carcinoma, Bile Duct Cancer, Birt Hogg Dube Syndrome, Bladder Cancer, Bone Cancer, Brain Tumor, Breast Cancer, Breast Cancer in Men, Carcinoid Tumor, Cervical Cancer, Colorectal Cancer, Ductal Carcinoma, Endometrial Cancer, Esophageal Cancer, Gastric Cancer, Gastrointestinal Stromal Tumor (GIST), HER2-Positive Breast Cancer, Islet Cell Tumor, Juvenile Polyposis Syndrome, Kidney Cancer, Laryngeal Cancer, Leukemia—Acute Lymphoblastic Leukemia, Leukemia—Acute Lymphocytic (ALL), Leukemia—Acute Myeloid AML, Leukemia—Adult, Leukemia—Childhood, Leukemia Chronic Lymphocytic (CLL), Leukemia—Chronic Myeloid (CIVIL), Liver Cancer, Lobular Carcinoma, Lung Cancer, Lung Cancer—Small Cell (SCLC), Lung Cancer—Non-small Cell (NSCLC), Lymphoma—Hodgkin's, Lymphoma—Non-Hodgkin's, Malignant Glioma, Melanoma, Meningioma, Multiple Myeloma, Myelodysplastic Syndrome (MDS), Nasopharyngeal Cancer, Neuroendocrine Tumor, Oral Cancer, Osteosarcoma, Ovarian Cancer, Pancreatic Cancer, Pancreatic Neuroendocrine Tumors, Parathyroid Cancer, Penile Cancer, Peritoneal Cancer, Peutz-Jeghers Syndrome, Pituitary Gland Tumor, Polycythemia Vera, Prostate Cancer, Renal Cell Carcinoma, Retinoblastoma, Salivary Gland Cancer, Sarcoma, Sarcoma—Kaposi, Skin Cancer, Small Intestine Cancer, Stomach Cancer, Testicular Cancer, Thymoma, Thyroid Cancer, Uterine (Endometrial) Cancer, Vaginal Cancer, and Wilms' Tumor.

In some embodiments, the disease or the condition is an inflammatory bowel disease, such as Crohn's disease (CD) or ulcerative colitis (UC). A subject may suffer from fibrosis, fibrostenosis, or a fibrotic disease, either isolated or in combination with an inflammatory disease. In some cases, the CD is severe CD. The severe CD may result from inflammation that has led to the formation of scar tissue in the intestinal wall (fibrostenosis) and/or swelling. In some cases, the severe CD is characterized by the presence of fibrotic and/or inflammatory strictures. The strictures may be determined by computed tomography enterography (CTE), and magnetic resonance imaging enterography (MRE). The disease or condition may be characterized as refractory, which in some cases, means the disease is resistant to a standard treatment (e.g., anti-TNFα therapy). Non-limiting examples of standard treatment include glucocorticosteroids, anti-TNF therapy, anti-a4-b7 therapy (vedolizumab), anti-IL12p40 therapy (ustekinumab), Thalidomide, and Cytoxin.

In the present application, the subject may include a human or non-human animal. For example, the non-human animal may be selected from the group consisting of: a monkey, a chicken, a goose, a cat, a dog, a mouse, and a rat. In addition, a non-human animal may also include any animal species other than human, such as livestock animals, or rodents, or primates, or domestic animals, or poultry animals. The human can be Caucasian, African, Asian, Sumerian, or other ethnicity, or a hybrid of various ethnicities. As another example, the human may be the elderly, adults, adolescents, children or infants.

Effective amounts in human can be extrapolated from the effective amounts in experimental animals. For example, Freireich et al. described the interrelationship of dosages for animals and humans (based on milligrams per meter squared of body surface) (Freireich et al., Cancer Chemother. Rep. 50, 219 (1966)). Body surface area can be approximately determined from the height and weight of the patient. See, e.g., Scientific Tables, Geigy Pharmaceuticals, Ardsley, N.Y., 537 (1970).

Without intending to be bound by any theory, the following examples are intended merely to illustrate the fusion proteins, methods of preparation, uses, etc., of the present application and are not intended to limit the scope of the present application.

On the other hand, the present application provides a method for downregulating the expression of α4β7/TL1A, comprising: providing an isolated antigen-binding protein that can specifically bind to TL1A. In some embodiments, the method further comprises: providing an antigen-binding protein that can specifically bind to α4β7. In some embodiments, the method further comprises: providing a TL1A protein. In some embodiments, the isolated antigen-binding protein that can specifically bind to TL1A and the antigen-binding protein that can specifically bind to α4β7 are the same protein. In some embodiments, the same protein is the aforementioned antigen-binding protein.

On the other hand, the present application provides a method for inducing endocytosis of cell surface α4β7/TL1A, comprising: providing an isolated antigen-binding protein that can specifically bind to TL1A. In some embodiments, the method further comprises: providing an antigen-binding protein that can specifically bind to α4β7. In some embodiments, the method further comprises: providing a TL1A protein. In some embodiments, the isolated antigen-binding protein that can specifically bind to TL1A and the antigen-binding protein that can specifically bind to α4β7 are the same protein. In some embodiments, the same protein is the aforementioned antigen-binding protein.

In the present application, the isolated antigen binding protein can induce the endocytosis of cell surface α4β7/TL1A. In some embodiments, the endocytosis of the protein can be determined by a fluorescent labeling method. For example, the efficiency of the antigen binding protein in inducing the endocytosis of cell surface α4P7/TL1A can be obtained by detecting the fluorescence intensity of the cell using flow cytometry. In some cases, the isolated antigen binding protein can induce the endocytosis of α4P7/TL1A with an efficiency of about 50 or more.

On the other hand, the present application provides the use of the antigen binding protein to induce the endocytosis of cell surface α4β7/TL1A.

On the other hand, the present application provides the use of the antigen binding protein to downregulate the expression of α4β7/TL1A.

Without being limited by any theory, the following examples are only for illustrating the fusion protein, preparation method and use of the present application, and are not intended to limit the scope of the invention of the present application.

### EXAMPLE

### Example 1 Anti-TL1A and Antibody α4P7 Monoclonal Antibody Sequence

Anti-TL1A monoclonal antibodies: Anti-TL1A monoclonal antibodies were screened using TLA0015-11 and TLA0238-5 monoclonal antibodies, and their VH and VL amino acid sequences, scFv configuration sequences, and scFv sequences after the addition of disulfide bonds are shown in Table 1. Anti-α4β7 monoclonal antibody: Anti-Anti-α4β7 monoclonal antibody uses Vedolizumab, whose VH and VL amino acid sequences, scFv configuration sequences, and scFv sequences after the addition of disulfide bonds are shown in Table 1. In addition, the amino acid sequences of human IgG1 antibody Cκ, CH1 and Fc, and the sequences of Fc mutants are shown in Table 2.

TABLE 1
Amino acid sequence of TLA0015-11,TLA0238-5 and Vedolizumab

| No | Amino acid sequences |
|---|---|
| TLA0015-11 | EIQLVESGGGLIQPGGSLRISCAVSGFSLSSYGVDWVRQAP |
| VH | GKGLEWLGVIWGFGGTNYNSALKSRLTISKDNSKNTVYLQMN |
| | SLRAEDTAVYYCASGNFDAMDYWGQGTLVTVSS |
| TLA0015-11 | DIQMTQSPSSLSASVGDRVTITCRASQDISNYLNWYQQKP |
| VL | GKAVKLLIYYTSRLHSGVPSRFSGSGSGTDYTLTISSLQPEDFA |
| | TYFCQQGNTLPFTFGQGTKLEIK |
| scTLA0015- | EIQLVESGGGLIQPGGSLRISCAVSGFSLSSYGVDWVRQAP |
| 11 VHVL | GKGLEWLGVIWGFGGTNYNSALKSRLTISKDNSKNTVYLQMN |
| | SLRAEDTAVYYCASGNFDAMDYWGQGTLVTVSSGGGGSGGG |
| | GSGGGGSGGGGSDIQMTQSPSSLSASVGDRVTITCRASQDISN |
| | YLNWYQQKPGKAVKLLIYYTSRLHSGVPSRFSGSGSGTDYTLT |
| | ISSLQPEDFATYFCQQGNTLPFTFGQGTKLEIK |
| scTLA0015- | DIQMTQSPSSLSASVGDRVTITCRASQDISNYLNWYQQKP |
| 11 VLVH | GKAVKLLIYYTSRLHSGVPSRFSGSGSGTDYTLTISSLQPEDFA |
| | TYFCQQGNTLPFTFGQGTKLEIKGGGGSGGGGSGGGGSGGGG |
| | SEIQLVESGGGLIQPGGSLRISCAVSGFSLSSYGVDWVRQAPGK |
| | GLEWLGVIWGFGGTNYNSALKSRLTISKDNSKNTVYLQMNSL |
| | RAEDTAVYYCASGNFDAMDYWGQGTLVTVSS |
| scTLA0015- | EIQLVESGGGLIQPGGSLRISCAVSGFSLSSYGVDWVRQAP |
| 11cc VHVL | GKCLEWLGVIWGFGGTNYNSALKSRLTISKDNSKNTVYLQMN |
| | SLRAEDTAVYYCASGNFDAMDYWGQGTLVTVSSGGGGSGGG |
| | GSGGGGSGGGGSDIQMTQSPSSLSASVGDRVTITCRASQDISN |
| | YLNWYQQKPGKAVKLLIYYTSRLHSGVPSRFSGSGSGTDYTLT |
| | ISSLQPEDFATYFCQQGNTLPFTFGCGTKLEIK |
| scTLA0015- | DIQMTQSPSSLSASVGDRVTITCRASQDISNYLNWYQQKP |
| 11cc VLVH | GKAVKLLIYYTSRLHSGVPSRFSGSGSGTDYTLTISSLQPEDFA |
| | TYFCQQGNTLPFTFGCGTKLEIKGGGGSGGGGSGGGGSGGGG |
| | SEIQLVESGGGLIQPGGSLRISCAVSGFSLSSYGVDWVRQAPGK |
| | CLEWLGVIWGFGGTNYNSALKSRLTISKDNSKNTVYLQMNSL |
| | RAEDTAVYYCASGNFDAMDYWGQGTLVTVSS |
| TLA0238-5 | EVQLVESGGGLVQPGGSLRLSCAASGFTFSNYWMNWVR |
| VH | QAPGKGLEWVAQIRLKSDNYATHYADSVKGRFTISRDNSKNT |
| | LYLQMNSLRAEDTAVYYCTPLLLRYRDYWGQGTLVTVSS |
| TLA0238-5 | DIQMTQSPSSVSASVGDRVTITCSASSSVSYMHWYQQKPG |
| VL | KAPKLWIYDTSNLASGVPSRFSGSGSGTDFTLTISSLQPEDFAT |
| | YYCFQENGHPFTFGQGTKVEIK |
| scTLA0238- | EVQLVESGGGLVQPGGSLRLSCAASGFTFSNYWMNWVR |
| 5 VHVL | QAPGKGLEWVAQIRLKSDNYATHYADSVKGRFTISRDNSKNT |
| | LYLQMNSLRAEDTAVYYCTPLLLRYRDYWGQGTLVTVSSGG |
| | GGSGGGGSGGGGSGGGGSDIQMTQSPSSVSASVGDRVTITCSA |
| | SSSVSYMHWYQQKPGKAPKLWIYDTSNLASGVPSRFSGSGSG |
| | TDFTLTISSLQPEDFATYYCFQENGHPFTFGQGTKVEIK |
| scTLA0238- | EVQLVESGGGLVQPGGSLRLSCAASGFTFSNYWMNWVR |
| 5cc VHVL | QAPGKCLEWVAQIRLKSDNYATHYADSVKGRFTISRDNSKNT |
| | LYLQMNSLRAEDTAVYYCTPLLLRYRDYWGQGTLVTVSSGG |
| | GGSGGGGSGGGGSGGGGSDIQMTQSPSSVSASVGDRVTITCSA |
| | SSSVSYMHWYQQKPGKAPKLWIYDTSNLASGVPSRFSGSGSG |
| | TDFTLTISSLQPEDFATYYCFQENGHPFTFGCGTKVEIK |
| Vedolizumab | QVQLVQSGAEVKKPGASVKVSCKGSGYTFTSYWMHWV |
| VH | RQAPGQRLEWIGEIDPSESNTNYNQKFKGRVTLTVDISASTAY |
| | MELSSLRSEDTAVYYCARGGYDGWDYAIDYWGQGTLVTVSS |
| Vedolizumab | DVVMTQSPLSLPVTPGEPASISCRSSQSLAKSYGNTYLSW |
| VL | YLQKPGQSPQLLIYGISNRFSGVPDRESGSGSGTDFTLKISRVEA |
| | EDVGVYYCLQGTHQPYTFGQGTKVEIK |
| sc Vedolizumab | QVQLVQSGAEVKKPGASVKVSCKGSGYTFTSYWMHWV |
| VHVL | RQAPGQRLEWIGEIDPSESNTNYNQKFKGRVTLTVDISASTAY |
| | MELSSLRSEDTAVYYCARGGYDGWDYAIDYWGQGTLVTVSS |
| | GGGGSGGGGSGGGGSGGGGSDVVMTQSPLSLPVTPGEPASISC |
| | RSSQSLAKSYGNTYLSWYLQKPGQSPQLLIYGISNRFSGVPDRF |
| | SGSGSGTDFTLKISRVEAEDVGVYYCLQGTHQPYTFGQGTKVE |
| | IK |
| sc Vedolizumab- | QVQLVQSGAEVKKPGASVKVSCKGSGYTFTSYWMHWV |
| cc VHVL | RQAPGQCLEWIGEIDPSESNTNYNQKFKGRVTLTVDISASTAY |
| | MELSSLRSEDTAVYYCARGGYDGWDYAIDYWGQGTLVTVSS |
| | GGGGSGGGGSGGGGSGGGGSDVVMTQSPLSLPVTPGEPASISC |
| | RSSQSLAKSYGNTYLSWYLQKPGQSPQLLIYGISNRFSGVPDRF |
| | SGSGSGTDFTLKISRVEAEDVGVYYCLQGTHQPYTFGCGTKVE |
| | IK |
| RVT-3101 | QVQLVQSGAEVKKPGASVKVSCKASGYDFTYYGISWVR |
| VH | QAPGQGLEWMGWISTYNGNTHYARMLQGRVTMTTDTSTRTA |
| | YMELRSLRSDDTAVYYCARENYYGSGAYRGGMDVWGQGTT |
| | VTVSS |
| RVT-3101 | EIVLTQSPATLSLSPGERATLSCRASQSVSSYLAWYQQKP |
| VL | GQAPRLLIYDASNRATGIPARFSGSGSGTDFTLTISSLEPEDFAV |
| | YYCQQRSNWPWTFGQGTKVEIK |
| scRVT-3101 | QVQLVQSGAEVKKPGASVKVSCKASGYDFTYYGISWVR |
| VHVL | QAPGQGLEWMGWISTYNGNTHYARMLQGRVTMTTDTSTRTA |
| | YMELRSLRSDDTAVYYCARENYYGSGAYRGGMDVWGQGTT |
| | VTVSS |
| | GGGGSGGGGSGGGGSGGGGS |
| | EIVLTQSPATLSLSPGERATLSCRASQSVSSYLAWYQQKP |
| | GQAPRLLIYDASNRATGIPARFSGSGSGTDFTLTISSLEPEDFAV |
| | YYCQQRSNWPWTFGQGTKVEIK |
| scRVT-3101 | EIVLTQSPATLSLSPGERATLSCRASQSVSSYLAWYQQKP |
| VLVH | GQAPRLLIYDASNRATGIPARFSGSGSGTDFTLTISSLEPEDFAV |
| | YYCQQRSNWPWTFGQGTKVEIK |
| | GGGGSGGGGSGGGGSGGGGS |
| | QVQLVQSGAEVKKPGASVKVSCKASGYDFTYYGISWVR |
| | QAPGQGLEWMGWISTYNGNTHYARMLQGRVTMTTDTSTRTA |
| | YMELRSLRSDDTAVYYCARENYYGSGAYRGGMDVWGQGTT |
| | VTVSS |
| scRVT- | QVQLVQSGAEVKKPGASVKVSCKASGYDFTYYGISWVR |
| 3101cc | QAPGQCLEWMGWISTYNGNTHYARMLQGRVTMTTDTSTRTA |
| | YMELRSLRSDDTAVYYCARENYYGSGAYRGGMDVWGQGTT |
| | VTVSS |
| | GGGGSGGGGSGGGGSGGGGS |
| | EIVLTQSPATLSLSPGERATLSCRASQSVSSYLAWYQQKP |
| | GQAPRLLIYDASNRATGIPARFSGSGSGTDFTLTISSLEPEDFAV |
| | YYCQQRSNWPWTFGCGTKVEIK |

TABLE 2
Amino acid sequence of the constant region
of Human IgG1

| NO | Amino acid sequence |
|---|---|
| Cκ | RTVAAPSVFIFPPSDEQLKSGTASVVCLLNNFYPREAKVQ |
| | WKVDNALQSGNSQESVTEQDSKDSTYSLSSTLTLSKADYE |
| | KHKVYACEVTHQGLSSPVTKSFNRGEC |
| CH1 | ASTKGPSVFPLAPSSKSTSGGTAALGCLVKDYFPEPVTVS |
| | WNSGALTSGVHTFPAVLQSSGLYSLSSVVTVPSSSLGTQT |
| | YICNVNHKPSNTKVDKKVEPKSC |
| Fc(LALA + | DKTHTCPPCPAPEAAGGPSVFLFPPKPKDTLYITREPEVTC |
| YTE) | VVVDVSHEDPEVKFNWYVDGVEVHNAKTKPREEQYNSTYRV |
| | VSVLTVLHQDWLNGKEYKCKVSNKALPAPIEKTISKAKGQP |
| | REPQVYTLPPSRDELTKNQVSLTCLVKGFYPSDIAVEWESN |
| | GQPENNYKTTPPVLDSDGSFFLYSKLTVDKSRWQQGNVFSC |
| | SVMHEALHNHYTQKSLSLSPGK |
| Fc(LALA + | DKTHTCPPCPAPEAAGGPSVFLFPPKPKDTLMISRTPEVTC |
| LS) | VVVDVSHEDPEVKFNWYVDGVEVHNAKTKPREEQYNSTYRV |
| | VSVLTVLHQDWLNGKEYKCKVSNKALPAPIEKTISKAKGQP |
| | REPQVYTLPPSRDELTKNQVSLTCLVKGFYPSDIAVEWESN |
| | GQPENNYKTTPPVLDSDGSFFLYSKLTVDKSRWQQGNVFSC |
| | SVLHEALHSHYTQKSLSLSPGK |
| Fc-Knob | DKTHTCPPCPAPEAAGGPSVFLFPPKPKDTLMISRTPEVTC |
| | VVVDVSHEDPEVKFNWYVDGVEVHNAKTKPREEQYNSTYRV |
| | VSVLTVLHQDWLNGKEYKCKVSNKALPAPIEKTISKAKGQP |
| | REPQVYTLPPCRDELTKNQVSLWCLVKGFYPSDIAVEWESN |
| | GQPENNYKTTPPVLDSDGSFFLYSKLTVDKSRWQQGNVFSC |
| | SVMHEALHNHYTQKSLSLSPGK |
| Fc-Hole | DKTHTCPPCPAPEAAGGPSVFLFPPKPKDTLMISRTPEVTC |
| | VVVDVSHEDPEVKFNWYVDGVEVHNAKTKPREEQYNSTYRV |
| | VSVLTVLHQDWLNGKEYKCKVSNKALPAPIEKTISKAKGQP |
| | REPQVCTLPPSRDELTKNQVSLSCAVKGFYPSDIAVEWESN |
| | GQPENNYKTTPPVLDSDGSFFLVSKLTVDKSRWQQGNVFSC |
| | SVMHEALHNRFTQKSLSLSPGK |

### Example 2 Design of Anti-TL1A×α4β7 Bispecific Antibody

Anti-TL1A antibodies TLA0015-11 and TLA0238-5 were assembled with vedolizumab monoclonal antibodies into bispecific antibody molecules of different configurations, the amino acid sequences of each bispecific antibody are shown in Table 3A and 3B, and the schematic configuration of each bispecific antibody is shown in FIG. 1.

TABLE 3A
Bispecific antibody amino acid sequence with format 2 + 2

| No. | Light Chain sequence | Heavy chain sequence |
|---|---|---|
| TLb0045 | DVVMTQSPLSLPVTPGEPASISCRS | QVQLVQSGAEVKKPGASVKVSCKGS |
| | SQSLAKSYGNTYLSWYLQKPGQSPQ | GYTFTSYWMHWVRQAPGQRLEWIGE |
| | LLIYGISNRFSGVPDRFSGSGSGTD | IDPSESNTNYNQKFKGRVTLTVDIS |
| | FTLKISRVEAEDVGVYYCLQGTHQP | ASTAYMELSSLRSEDTAVYYCARGG |
| | YTFGQGTKVEIKRTVAAPSVFIFPP | YDGWDYAIDYWGQGTLVTVSSASTK |
| | SDEQLKSGTASVVCLLNNFYPREAK | GPSVFPLAPSSKSTSGGTAALGCLV |
| | VQWKVDNALQSGNSQESVTEQDSKD | KDYFPEPVTVSWNSGALTSGVHTFP |
| | STYSLSSTLTLSKADYEKHKVYACE | AVLQSSGLYSLSSVVTVPSSSLGTQ |
| | VTHQGLSSPVTKSFNRGEC | TYICNVNHKPSNTKVDKKVEPKSCD |
| | | KTHTCPPCPAPEAAGGPSVFLFPPK |
| | | PKDTLYITREPEVTCVVVDVSHEDP |
| | | EVKFNWYVDGVEVHNAKTKPREEQY |
| | | NSTYRVVSVLTVLHQDWLNGKEYKC |
| | | KVSNKALPAPIEKTISKAKGQPREP |
| | | QVYTLPPSRDELTKNQVSLTCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSPG |
| | | KGGGGSGGGGSGGGGSGGGGSEIQL |
| | | VESGGGLIQPGGSLRISCAVSGFSL |
| | | SSYGVDWVRQAPGKCLEWLGVIWGF |
| | | GGTNYNSALKSRLTISKDNSKNTVY |
| | | LQMNSLRAEDTAVYYCASGNFDAMD |
| | | YWGQGTLVTVSSGGGGSGGGGSGGG |
| | | GSGGGGSDIQMTQSPSSLSASVGDR |
| | | VTITCRASQDISNYLNWYQQKPGKA |
| | | VKLLIYYTSRLHSGVPSRFSGSGSG |
| | | TDYTLTISSLQPEDFATYFCQQGNT |
| | | LPFTFGCGTKLEIK |
| TLb0046 | DVVMTQSPLSLPVTPGEPASISCRS | DIQMTQSPSSLSASVGDRVTITCRA |
| | SQSLAKSYGNTYLSWYLQKPGQSPQ | SQDISNYLNWYQQKPGKAVKLLIYY |
| | LLIYGISNRFSGVPDRFSGSGSGTD | TSRLHSGVPSRFSGSGSGTDYTLTI |
| | FTLKISRVEAEDVGVYYCLQGTHQP | SSLQPEDFATYFCQQGNTLPFTFGC |
| | YTFGQGTKVEIKRTVAAPSVFIFPP | GTKLEIKGGGGSGGGGSGGGGSGGG |
| | SDEQLKSGTASVVCLLNNFYPREAK | GSEIQLVESGGGLIQPGGSLRISCA |
| | VQWKVDNALQSGNSQESVTEQDSKD | VSGFSLSSYGVDWVRQAPGKCLEWL |
| | STYSLSSTLTLSKADYEKHKVYACE | GVIWGFGGTNYNSALKSRLTISKDN |
| | VTHQGLSSPVTKSFNRGEC | SKNTVYLQMNSLRAEDTAVYYCASG |
| | | NFDAMDYWGQGTLVTVSSGGGGSGG |
| | | GGSGGGGSGGGGSQVQLVQSGAEVK |
| | | KPGASVKVSCKGSGYTFTSYWMHWV |
| | | RQAPGQRLEWIGEIDPSESNTNYNQ |
| | | KFKGRVTLTVDISASTAYMELSSLR |
| | | SEDTAVYYCARGGYDGWDYAIDYWG |
| | | QGTLVTVSSASTKGPSVFPLAPSSK |
| | | STSGGTAALGCLVKDYFPEPVTVSW |
| | | NSGALTSGVHTFPAVLQSSGLYSLS |
| | | SVVTVPSSSLGTQTYICNVNHKPSN |
| | | TKVDKKVEPKSCDKTHTCPPCPAPE |
| | | AAGGPSVFLFPPKPKDTLYITREPE |
| | | VTCVVVDVSHEDPEVKFNWYVDGVE |
| | | VHNAKTKPREEQYNSTYRVVSVLTV |
| | | LHQDWLNGKEYKCKVSNKALPAPIE |
| | | KTISKAKGQPREPQVYTLPPSRDEL |
| | | TKNQVSLTCLVKGFYPSDIAVEWES |
| | | NGQPENNYKTTPPVLDSDGSFFLYS |
| | | KLTVDKSRWQQGNVFSCSVMHEALH |
| | | NHYTQKSLSLSPGK |
| TLb0065 | DVVMTQSPLSLPVTPGEPASISCRS | QVQLVQSGAEVKKPGASVKVSCKGS |
| | SQSLAKSYGNTYLSWYLQKPGQSPQ | GYTFTSYWMHWVRQAPGQRLEWIGE |
| | LLIYGISNRFSGVPDRFSGSGSGTD | IDPSESNTNYNQKFKGRVTLTVDIS |
| | FTLKISRVEAEDVGVYYCLQGTHQP | ASTAYMELSSLRSEDTAVYYCARGG |
| | YTFGQGTKVEIKRTVAAPSVFIFPP | YDGWDYAIDYWGQGTLVTVSSASTK |
| | SDEQLKSGTASVVCLLNNFYPREAK | GPSVFPLAPSSKSTSGGTAALGCLV |
| | VQWKVDNALQSGNSQESVTEQDSKD | KDYFPEPVTVSWNSGALTSGVHTFP |
| | STYSLSSTLTLSKADYEKHKVYACE | AVLQSSGLYSLSSVVTVPSSSLGTQ |
| | VTHQGLSSPVTKSFNRGEC | TYICNVNHKPSNTKVDKKVEPKSCD |
| | | KTHTCPPCPAPEAAGGPSVFLFPPK |
| | | PKDTLYITREPEVTCVVVDVSHEDP |
| | | EVKFNWYVDGVEVHNAKTKPREEQY |
| | | NSTYRVVSVLTVLHQDWLNGKEYKC |
| | | KVSNKALPAPIEKTISKAKGQPREP |
| | | QVYTLPPSRDELTKNQVSLTCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSPG |
| | | KGGGGSGGGGSGGGGSGGGGSEIQL |
| | | VESGGGLIQPGGSLRISCAVSGFSL |
| | | SSYGVDWVRQAPGKGLEWLGVIWGF |
| | | GGTNYNSALKSRLTISKDNSKNTVY |
| | | LQMNSLRAEDTAVYYCASGNFDAMD |
| | | YWGQGTLVTVSSGGGGSGGGGSGGG |
| | | GSGGGGSDIQMTQSPSSLSASVGDR |
| | | VTITCRASQDISNYLNWYQQKPGKA |
| | | VKLLIYYTSRLHSGVPSRFSGSGSG |
| | | TDYTLTISSLQPEDFATYFCQQGNT |
| | | LPFTFGQGTKLEIK |
| TLb0066 | DVVMTQSPLSLPVTPGEPASISCRS | DIQMTQSPSSLSASVGDRVTITCRA |
| | SQSLAKSYGNTYLSWYLQKPGQSPQ | SQDISNYLNWYQQKPGKAVKLLIYY |
| | LLIYGISNRFSGVPDRFSGSGSGTD | TSRLHSGVPSRFSGSGSGTDYTLTI |
| | FTLKISRVEAEDVGVYYCLQGTHQP | SSLQPEDFATYFCQQGNTLPFTFGQ |
| | YTFGQGTKVEIKRTVAAPSVFIFPP | GTKLEIKGGGGSGGGGSGGGGSGGG |
| | SDEQLKSGTASVVCLLNNFYPREAK | GSEIQLVESGGGLIQPGGSLRISCA |
| | VQWKVDNALQSGNSQESVTEQDSKD | VSGFSLSSYGVDWVRQAPGKGLEWL |
| | STYSLSSTLTLSKADYEKHKVYACE | GVIWGFGGTNYNSALKSRLTISKDN |
| | VTHQGLSSPVTKSFNRGEC | SKNTVYLQMNSLRAEDTAVYYCASG |
| | | NFDAMDYWGQGTLVTVSSGGGGSGG |
| | | GGSGGGGSGGGGSQVQLVQSGAEVK |
| | | KPGASVKVSCKGSGYTFTSYWMHWV |
| | | RQAPGQRLEWIGEIDPSESNTNYNQ |
| | | KFKGRVTLTVDISASTAYMELSSLR |
| | | SEDTAVYYCARGGYDGWDYAIDYWG |
| | | QGTLVTVSSASTKGPSVFPLAPSSK |
| | | STSGGTAALGCLVKDYFPEPVTVSW |
| | | NSGALTSGVHTFPAVLQSSGLYSLS |
| | | SVVTVPSSSLGTQTYICNVNHKPSN |
| | | TKVDKKVEPKSCDKTHTCPPCPAPE |
| | | AAGGPSVFLFPPKPKDTLYITREPE |
| | | VTCVVVDVSHEDPEVKFNWYVDGVE |
| | | VHNAKTKPREEQYNSTYRVVSVLTV |
| | | LHQDWLNGKEYKCKVSNKALPAPIE |
| | | KTISKAKGQPREPQVYTLPPSRDEL |
| | | TKNQVSLTCLVKGFYPSDIAVEWES |
| | | NGQPENNYKTTPPVLDSDGSFFLYS |
| | | KLTVDKSRWQQGNVFSCSVMHEALH |
| | | NHYTQKSLSLSPGK |
| TLb0067 | DIQMTQSPSSLSASVGDRVTITCRA | EIQLVESGGGLIQPGGSLRISCAVS |
| | SQDISNYLNWYQQKPGKAVKLLIYY | GFSLSSYGVDWVRQAPGKGLEWLGV |
| | TSRLHSGVPSRFSGSGSGTDYTLTI | IWGFGGTNYNSALKSRLTISKDNSK |
| | SSLQPEDFATYFCQQGNTLPFTFGQ | NTVYLQMNSLRAEDTAVYYCASGNF |
| | GTKLEIKRTVAAPSVFIFPPSDEQL | DAMDYWGQGTLVTVSSASTKGPSVF |
| | KSGTASVVCLLNNFYPREAKVQWKV | PLAPSSKSTSGGTAALGCLVKDYFP |
| | DNALQSGNSQESVTEQDSKDSTYSL | EPVTVSWNSGALTSGVHTFPAVLQS |
| | SSTLTLSKADYEKHKVYACEVTHQG | SGLYSLSSVVTVPSSSLGTQTYICN |
| | LSSPVTKSFNRGEC | VNHKPSNTKVDKKVEPKSCDKTHTC |
| | | PPCPAPEAAGGPSVFLFPPKPKDTL |
| | | YITREPEVTCVVVDVSHEDPEVKFN |
| | | WYVDGVEVHNAKTKPREEQYNSTYR |
| | | VVSVLTVLHQDWLNGKEYKCKVSNK |
| | | ALPAPIEKTISKAKGQPREPQVYTL |
| | | PPSRDELTKNQVSLTCLVKGFYPSD |
| | | IAVEWESNGQPENNYKTTPPVLDSD |
| | | GSFFLYSKLTVDKSRWQQGNVFSCS |
| | | VMHEALHNHYTQKSLSLSPGKGGGG |
| | | SGGGGSGGGGSGGGGSQVQLVQSGA |
| | | EVKKPGASVKVSCKGSGYTFTSYWM |
| | | HWVRQAPGQRLEWIGEIDPSESNTN |
| | | YNQKFKGRVTLTVDISASTAYMELS |
| | | SLRSEDTAVYYCARGGYDGWDYAID |
| | | YWGQGTLVTVSSGGGGSGGGGSGGG |
| | | GSGGGGSDVVMTQSPLSLPVTPGEP |
| | | ASISCRSSQSLAKSYGNTYLSWYLQ |
| | | KPGQSPQLLIYGISNRFSGVPDRFS |
| | | GSGSGTDFTLKISRVEAEDVGVYYC |
| | | LQGTHQPYTFGQGTKVEIK |
| TLb0068 | DIQMTQSPSSLSASVGDRVTITCRA | DVVMTQSPLSLPVTPGEPASISCRS |
| | SQDISNYLNWYQQKPGKAVKLLIYY | SQSLAKSYGNTYLSWYLQKPGQSPQ |
| | TSRLHSGVPSRFSGSGSGTDYTLTI | LLIYGISNRFSGVPDRFSGSGSGTD |
| | SSLQPEDFATYFCQQGNTLPFTFGQ | FTLKISRVEAEDVGVYYCLQGTHQP |
| | GTKLEIKRTVAAPSVFIFPPSDEQL | YTFGQGTKVEIKGGGGSGGGGSGGG |
| | KSGTASVVCLLNNFYPREAKVQWKV | GSGGGGSQVQLVQSGAEVKKPGASV |
| | DNALQSGNSQESVTEQDSKDSTYSL | KVSCKGSGYTFTSYWMHWVRQAPGQ |
| | SSTLTLSKADYEKHKVYACEVTHQG | RLEWIGEIDPSESNTNYNQKFKGRV |
| | LSSPVTKSFNRGEC | TLTVDISASTAYMELSSLRSEDTAV |
| | | YYCARGGYDGWDYAIDYWGQGTLVT |
| | | VSSGGGGSGGGGSGGGGSGGGGSEI |
| | | QLVESGGGLIQPGGSLRISCAVSGF |
| | | SLSSYGVDWVRQAPGKGLEWLGVIW |
| | | GFGGTNYNSALKSRLTISKDNSKNT |
| | | VYLQMNSLRAEDTAVYYCASGNFDA |
| | | MDYWGQGTLVTVSSASTKGPSVFPL |
| | | APSSKSTSGGTAALGCLVKDYFPEP |
| | | VTVSWNSGALTSGVHTFPAVLQSSG |
| | | LYSLSSVVTVPSSSLGTQTYICNVN |
| | | HKPSNTKVDKKVEPKSCDKTHTCPP |
| | | CPAPEAAGGPSVFLFPPKPKDTLYI |
| | | TREPEVTCVVVDVSHEDPEVKFNWY |
| | | VDGVEVHNAKTKPREEQYNSTYRVV |
| | | SVLTVLHQDWLNGKEYKCKVSNKAL |
| | | PAPIEKTISKAKGQPREPQVYTLPP |
| | | SRDELTKNQVSLTCLVKGFYPSDIA |
| | | VEWESNGQPENNYKTTPPVLDSDGS |
| | | FFLYSKLTVDKSRWQQGNVFSCSVM |
| | | HEALHNHYTQKSLSLSPGK |
| TLb0081 | DVVMTQSPLSLPVTPGEPASISCRS | QVQLVQSGAEVKKPGASVKVSCKGS |
| | SQSLAKSYGNTYLSWYLQKPGQSPQ | GYTFTSYWMHWVRQAPGQRLEWIGE |
| | LLIYGISNRFSGVPDRFSGSGSGTD | IDPSESNTNYNQKFKGRVTLTVDIS |
| | FTLKISRVEAEDVGVYYCLQGTHQP | ASTAYMELSSLRSEDTAVYYCARGG |
| | YTFGQGTKVEIKRTVAAPSVFIFPP | YDGWDYAIDYWGQGTLVTVSSASTK |
| | SDEQLKSGTASVVCLLNNFYPREAK | GPSVFPLAPSSKSTSGGTAALGCLV |
| | VQWKVDNALQSGNSQESVTEQDSKD | KDYFPEPVTVSWNSGALTSGVHTFP |
| | STYSLSSTLTLSKADYEKHKVYACE | AVLQSSGLYSLSSVVTVPSSSLGTQ |
| | VTHQGLSSPVTKSFNRGEC | TYICNVNHKPSNTKVDKKVEPKSCD |
| | | KTHTCPPCPAPEAAGGPSVFLFPPK |
| | | PKDTLYITREPEVTCVVVDVSHEDP |
| | | EVKFNWYVDGVEVHNAKTKPREEQY |
| | | NSTYRVVSVLTVLHQDWLNGKEYKC |
| | | KVSNKALPAPIEKTISKAKGQPREP |
| | | QVYTLPPSRDELTKNQVSLTCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSPG |
| | | KGGGGSGGGGSGGGGSGGGGSEVQL |
| | | VESGGGLVQPGGSLRLSCAASGFTF |
| | | SNYWMNWVRQAPGKGLEWVAQIRLK |
| | | SDNYATHYADSVKGRFTISRDNSKN |
| | | TLYLQMNSLRAEDTAVYYCTPLLLR |
| | | YRDYWGQGTLVTVSSGGGGSGGGGS |
| | | GGGGSGGGGSDIQMTQSPSSVSASV |
| | | GDRVTITCSASSSVSYMHWYQQKPG |
| | | KAPKLWIYDTSNLASGVPSRFSGSG |
| | | SGTDFTLTISSLQPEDFATYYCFQE |
| | | NGHPFTFGQGTKVEIK |
| TLb0085 | DIQMTQSPSSVSASVGDRVTITCSA | EVQLVESGGGLVQPGGSLRLSCAAS |
| | SSSVSYMHWYQQKPGKAPKLWIYDT | GFTFSNYWMNWVRQAPGKGLEWVAQ |
| | SNLASGVPSRFSGSGSGTDFTLTIS | IRLKSDNYATHYADSVKGRFTISRD |
| | SLQPEDFATYYCFQENGHPFTFGQG | NSKNTLYLQMNSLRAEDTAVYYCTP |
| | TKVEIKRTVAAPSVFIFPPSDEQLK | LLLRYRDYWGQGTLVTVSSASTKGP |
| | SGTASVVCLLNNFYPREAKVQWKVD | SVFPLAPSSKSTSGGTAALGCLVKD |
| | NALQSGNSQESVTEQDSKDSTYSLS | YFPEPVTVSWNSGALTSGVHTFPAV |
| | STLTLSKADYEKHKVYACEVTHQGL | LQSSGLYSLSSVVTVPSSSLGTQTY |
| | SSPVTKSFNRGEC | ICNVNHKPSNTKVDKKVEPKSCDKT |
| | | HTCPPCPAPEAAGGPSVFLFPPKPK |
| | | DTLYITREPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQV |
| | | YTLPPSRDELTKNQVSLTCLVKGFY |
| | | PSDIAVEWESNGQPENNYKTTPPVL |
| | | DSDGSFFLYSKLTVDKSRWQQGNVF |
| | | SCSVMHEALHNHYTQKSLSLSPGKG |
| | | GGGSGGGGSGGGGSGGGGSQVQLVQ |
| | | SGAEVKKPGASVKVSCKGSGYTFTS |
| | | YWMHWVRQAPGQRLEWIGEIDPSES |
| | | NTNYNQKFKGRVTLTVDISASTAYM |
| | | ELSSLRSEDTAVYYCARGGYDGWDY |
| | | AIDYWGQGTLVTVSSGGGGSGGGGS |
| | | GGGGSGGGGSDVVMTQSPLSLPVTP |
| | | GEPASISCRSSQSLAKSYGNTYLSW |
| | | YLQKPGQSPQLLIYGISNRFSGVPD |
| | | RFSGSGSGTDFTLKISRVEAEDVGV |
| | | YYCLQGTHQPYTFGQGTKVEIK |
| TLb0086 | DIQMTQSPSSLSASVGDRVTITCRA | EIQLVESGGGLIQPGGSLRISCAVS |
| | SQDISNYLNWYQQKPGKAVKLLIYY | GFSLSSYGVDWVRQAPGKGLEWLGV |
| | TSRLHSGVPSRFSGSGSGTDYTLTI | IWGFGGTNYNSALKSRLTISKDNSK |
| | SSLQPEDFATYFCQQGNTLPFTFGQ | NTVYLQMNSLRAEDTAVYYCASGNF |
| | GTKLEIKRTVAAPSVFIFPPSDEQL | DAMDYWGQGTLVTVSSASTKGPSVF |
| | KSGTASVVCLLNNFYPREAKVQWKV | PLAPSSKSTSGGTAALGCLVKDYFP |
| | DNALQSGNSQESVTEQDSKDSTYSL | EPVTVSWNSGALTSGVHTFPAVLQS |
| | SSTLTLSKADYEKHKVYACEVTHQG | SGLYSLSSVVTVPSSSLGTQTYICN |
| | LSSPVTKSFNRGEC | VNHKPSNTKVDKKVEPKSCDKTHTC |
| | | PPCPAPEAAGGPSVFLFPPKPKDTL |
| | | YITREPEVTCVVVDVSHEDPEVKFN |
| | | WYVDGVEVHNAKTKPREEQYNSTYR |
| | | VVSVLTVLHQDWLNGKEYKCKVSNK |
| | | ALPAPIEKTISKAKGQPREPQVYTL |
| | | PPSRDELTKNQVSLTCLVKGFYPSD |
| | | IAVEWESNGQPENNYKTTPPVLDSD |
| | | GSFFLYSKLTVDKSRWQQGNVFSCS |
| | | VMHEALHNHYTQKSLSLSPGKGGGG |
| | | SGGGGSGGGGSGGGGSQVQLVQSGA |
| | | EVKKPGASVKVSCKGSGYTFTSYWM |
| | | HWVRQAPGQCLEWIGEIDPSESNTN |
| | | YNQKFKGRVTLTVDISASTAYMELS |
| | | SLRSEDTAVYYCARGGYDGWDYAID |
| | | YWGQGTLVTVSSGGGGSGGGGSGGG |
| | | GSGGGGSDVVMTQSPLSLPVTPGEP |
| | | ASISCRSSQSLAKSYGNTYLSWYLQ |
| | | KPGQSPQLLIYGISNRFSGVPDRFS |
| | | GSGSGTDFTLKISRVEAEDVGVYYC |
| | | LQGTHQPYTFGCGTKVEIK |
| TLb0087 | DVVMTQSPLSLPVTPGEPASISCRS | QVQLVQSGAEVKKPGASVKVSCKGS |
| | SQSLAKSYGNTYLSWYLQKPGQSPQ | GYTFTSYWMHWVRQAPGQRLEWIGE |
| | LLIYGISNRFSGVPDRFSGSGSGTD | IDPSESNTNYNQKFKGRVTLTVDIS |
| | FTLKISRVEAEDVGVYYCLQGTHQP | ASTAYMELSSLRSEDTAVYYCARGG |
| | YTFGQGTKVEIKRTVAAPSVFIFPP | YDGWDYAIDYWGQGTLVTVSSASTK |
| | SDEQLKSGTASVVCLLNNFYPREAK | GPSVFPLAPSSKSTSGGTAALGCLV |
| | VQWKVDNALQSGNSQESVTEQDSKD | KDYFPEPVTVSWNSGALTSGVHTFP |
| | STYSLSSTLTLSKADYEKHKVYACE | AVLQSSGLYSLSSVVTVPSSSLGTQ |
| | VTHQGLSSPVTKSFNRGEC | TYICNVNHKPSNTKVDKKVEPKSCD |
| | | KTHTCPPCPAPEAAGGPSVFLFPPK |
| | | PKDTLYITREPEVTCVVVDVSHEDP |
| | | EVKFNWYVDGVEVHNAKTKPREEQY |
| | | NSTYRVVSVLTVLHQDWLNGKEYKC |
| | | KVSNKALPAPIEKTISKAKGQPREP |
| | | QVYTLPPSRDELTKNQVSLTCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSPG |
| | | KGGGGSGGGGSGGGGSGGGGSEVQL |
| | | VESGGGLVQPGGSLRLSCAASGFTF |
| | | SNYWMNWVRQAPGKCLEWVAQIRLK |
| | | SDNYATHYADSVKGRFTISRDNSKN |
| | | TLYLQMNSLRAEDTAVYYCTPLLLR |
| | | YRDYWGQGTLVTVSSGGGGSGGGGS |
| | | GGGGSGGGGSDIQMTQSPSSVSASV |
| | | GDRVTITCSASSSVSYMHWYQQKPG |
| | | KAPKLWIYDTSNLASGVPSRFSGSG |
| | | SGTDFTLTISSLQPEDFATYYCFQE |
| | | NGHPFTFGCGTKVEIK |
| TLb0088 | DIQMTQSPSSVSASVGDRVTITCSA | EVQLVESGGGLVQPGGSLRLSCAAS |
| | SSSVSYMHWYQQKPGKAPKLWIYDT | GFTFSNYWMNWVRQAPGKGLEWVAQ |
| | SNLASGVPSRFSGSGSGTDFTLTIS | IRLKSDNYATHYADSVKGRFTISRD |
| | SLQPEDFATYYCFQENGHPFTFGQG | NSKNTLYLQMNSLRAEDTAVYYCTP |
| | TKVEIKRTVAAPSVFIFPPSDEQLK | LLLRYRDYWGQGTLVTVSSASTKGP |
| | SGTASVVCLLNNFYPREAKVQWKVD | SVFPLAPSSKSTSGGTAALGCLVKD |
| | NALQSGNSQESVTEQDSKDSTYSLS | YFPEPVTVSWNSGALTSGVHTFPAV |
| | STLTLSKADYEKHKVYACEVTHQGL | LQSSGLYSLSSVVTVPSSSLGTQTY |
| | SSPVTKSFNRGEC | ICNVNHKPSNTKVDKKVEPKSCDKT |
| | | HTCPPCPAPEAAGGPSVFLFPPKPK |
| | | DTLYITREPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQV |
| | | YTLPPSRDELTKNQVSLTCLVKGFY |
| | | PSDIAVEWESNGQPENNYKTTPPVL |
| | | DSDGSFFLYSKLTVDKSRWQQGNVF |
| | | SCSVMHEALHNHYTQKSLSLSPGKG |
| | | GGGSGGGGSGGGGSGGGGSQVQLVQ |
| | | SGAEVKKPGASVKVSCKGSGYTFTS |
| | | YWMHWVRQAPGQCLEWIGEIDPSES |
| | | NTNYNQKFKGRVTLTVDISASTAYM |
| | | ELSSLRSEDTAVYYCARGGYDGWDY |
| | | AIDYWGQGTLVTVSSGGGGSGGGGS |
| | | GGGGSGGGGSDVVMTQSPLSLPVTP |
| | | GEPASISCRSSQSLAKSYGNTYLSW |
| | | YLQKPGQSPQLLIYGISNRFSGVPD |
| | | RFSGSGSGTDFTLKISRVEAEDVGV |
| | | YYCLQGTHQPYTFGCGTKVEIK |
| TLb0093 | DVVMTQSPLSLPVTPGEPASISCRS | QVQLVQSGAEVKKPGASVKVSCKGS |
| | SQSLAKSYGNTYLSWYLQKPGQSPQ | GYTFTSYWMHWVRQAPGQRLEWIGE |
| | LLIYGISNRFSGVPDRFSGSGSGTD | IDPSESNTNYNQKFKGRVTLTVDIS |
| | FTLKISRVEAEDVGVYYCLQGTHQP | ASTAYMELSSLRSEDTAVYYCARGG |
| | YTFGQGTKVEIKRTVAAPSVFIFPP | YDGWDYAIDYWGQGTLVTVSSASTK |
| | SDEQLKSGTASVVCLLNNFYPREAK | GPSVFPLAPSSKSTSGGTAALGCLV |
| | VQWKVDNALQSGNSQESVTEQDSKD | KDYFPEPVTVSWNSGALTSGVHTFP |
| | STYSLSSTLTLSKADYEKHKVYACE | AVLQSSGLYSLSSVVTVPSSSLGTQ |
| | VTHQGLSSPVTKSFNRGEC | TYICNVNHKPSNTKVDKKVEPKSCD |
| | | KTHTCPPCPAPEAAGGPSVFLFPPK |
| | | PKDTLYITREPEVTCVVVDVSHEDP |
| | | EVKFNWYVDGVEVHNAKTKPREEQY |
| | | NSTYRVVSVLTVLHQDWLNGKEYKC |
| | | KVSNKALPAPIEKTISKAKGQPREP |
| | | QVYTLPPSRDELTKNQVSLTCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSPG |
| | | KGGGGSGGGGSGGGGSGGGGSDIQM |
| | | TQSPSSLSASVGDRVTITCRASQDI |
| | | SNYLNWYQQKPGKAVKLLIYYTSRL |
| | | HSGVPSRFSGSGSGTDYTLTISSLQ |
| | | PEDFATYFCQQGNTLPFTFGCGTKL |
| | | EIKGGGGSGGGGSGGGGSGGGGSEI |
| | | QLVESGGGLIQPGGSLRISCAVSGF |
| | | SLSSYGVDWVRQAPGKCLEWLGVIW |
| | | GFGGTNYNSALKSRLTISKDNSKNT |
| | | VYLQMNSLRAEDTAVYYCASGNFDA |
| | | MDYWGQGTLVTVSS |
| TLb0094 | DVVMTQSPLSLPVTPGEPASISCRS | QVQLVQSGAEVKKPGASVKVSCKGS |
| | SQSLAKSYGNTYLSWYLQKPGQSPQ | GYTFTSYWMHWVRQAPGQRLEWIGE |
| | LLIYGISNRFSGVPDRFSGSGSGTD | IDPSESNTNYNQKFKGRVTLTVDIS |
| | FTLKISRVEAEDVGVYYCLQGTHQP | ASTAYMELSSLRSEDTAVYYCARGG |
| | YTFGQGTKVEIKRTVAAPSVFIFPP | YDGWDYAIDYWGQGTLVTVSSASTK |
| | SDEQLKSGTASVVCLLNNFYPREAK | GPSVFPLAPSSKSTSGGTAALGCLV |
| | VQWKVDNALQSGNSQESVTEQDSKD | KDYFPEPVTVSWNSGALTSGVHTFP |
| | STYSLSSTLTLSKADYEKHKVYACE | AVLQSSGLYSLSSVVTVPSSSLGTQ |
| | VTHQGLSSPVTKSFNRGEC | TYICNVNHKPSNTKVDKKVEPKSCD |
| | | KTHTCPPCPAPEAAGGPSVFLFPPK |
| | | PKDTLYITREPEVTCVVVDVSHEDP |
| | | EVKFNWYVDGVEVHNAKTKPREEQY |
| | | NSTYRVVSVLTVLHQDWLNGKEYKC |
| | | KVSNKALPAPIEKTISKAKGQPREP |
| | | QVYTLPPSRDELTKNQVSLTCLVKG |
| | | FYPSDIAVEWESNGQPENNYKTTPP |
| | | VLDSDGSFFLYSKLTVDKSRWQQGN |
| | | VFSCSVMHEALHNHYTQKSLSLSPG |
| | | KGGGGSGGGGSGGGGSGGGGSDIQM |
| | | TQSPSSLSASVGDRVTITCRASQDI |
| | | SNYLNWYQQKPGKAVKLLIYYTSRL |
| | | HSGVPSRFSGSGSGTDYTLTISSLQ |
| | | PEDFATYFCQQGNTLPFTFGQGTKL |
| | | EIKGGGGSGGGGSGGGGSGGGGSEI |
| | | QLVESGGGLIQPGGSLRISCAVSGF |
| | | SLSSYGVDWVRQAPGKGLEWLGVIW |
| | | GFGGTNYNSALKSRLTISKDNSKNT |
| | | VYLQMNSLRAEDTAVYYCASGNFDA |
| | | MDYWGQGTLVTVSS |
| TLb0045-1 | DVVMTQSPLSLPVTPGEPASISCRS | QVQLVQSGAEVKKPGASVKVSCKGS |
| | SQSLAKSYGNTYLSWYLQKPGQSPQ | GYTFTSYWMHWVRQAPGQRLEWIGE |
| | LLIYGISNRFSGVPDRFSGSGSGTD | IDPSESNTNYNQKFKGRVTLTVDIS |
| | FTLKISRVEAEDVGVYYCLQGTHQP | ASTAYMELSSLRSEDTAVYYCARGG |
| | YTFGQGTKVEIKRTVAAPSVFIFPP | YDGWDYAIDYWGQGTLVTVSSASTK |
| | SDEQLKSGTASVVCLLNNFYPREAK | GPSVFPLAPSSKSTSGGTAALGCLV |
| | VQWKVDNALQSGNSQESVTEQDSKD | KDYFPEPVTVSWNSGALTSGVHTFP |
| | STYSLSSTLTLSKADYEKHKVYACE | AVLQSSGLYSLSSVVTVPSSSLGTQ |
| | VTHQGLSSPVTKSFNRGEC | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALPAPIEKTISKAKGQPREP |
| | | QVYTLPPSRDELTKNQVSLTCLVKG |
| | | FYPSDIAVEWESNGQPENNYKTTPP |
| | | VLDSDGSFFLYSKLTVDKSRWQQGN |
| | | VFSCSVLHEALHSHYTQKSLSLSPG |
| | | KGGGGSGGGGSGGGGSGGGGSEIQL |
| | | VESGGGLIQPGGSLRISCAVSGFSL |
| | | SSYGVDWVRQAPGKCLEWLGVIWGF |
| | | GGTNYNSALKSRLTISKDNSKNTVY |
| | | LQMNSLRAEDTAVYYCASGNFDAMD |
| | | YWGQGTLVTVSSGGGGSGGGGSGGG |
| | | GSGGGGSDIQMTQSPSSLSASVGDR |
| | | VTITCRASQDISNYLNWYQQKPGKA |
| | | VKLLIYYTSRLHSGVPSRFSGSGSG |
| | | TDYTLTISSLQPEDFATYFCQQGNT |
| | | LPFTFGCGTKLEIK |
| TLb0045-2 | DVVMTQSPLSLPVTPGEPASISCRS | QVQLVQSGAEVKKPGASVKVSCKGS |
| | SQSLAKSYGNTYLSWYLQKPGQSPQ | GYTFTSYWMHWVRQAPGQRLEWIGE |
| | LLIYGISNRFSGVPDRFSGSGSGTD | IDPSESNTNYNQKFKGRVTLTVDIS |
| | FTLKISRVEAEDVGVYYCLQGTHQP | ASTAYMELSSLRSEDTAVYYCARGG |
| | YTFGQGTKVEIKRTVAAPSVFIFPP | YDGWDYAIDYWGQGTLVTVSSASTK |
| | SDEQLKSGTASVVCLLNNFYPREAK | GPSVFPLAPSSKSTSGGTAALGCLV |
| | VQWKVDNALQSGNSQESVTEQDSKD | KDYFPEPVTVSWNSGALTSGVHTFP |
| | STYSLSSTLTLSKADYEKHKVYACE | AVLQSSGLYSLSSVVTVPSSSLGTQ |
| | VTHQGLSSPVTKSFNRGEC | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALPAPIEKTISKAKGQPREP |
| | | QVYTLPPSRDELTKNQVSLTCLVKG |
| | | FYPSDIAVEWESNGQPENNYKTTPP |
| | | VLDSDGSFFLYSKLTVDKSRWQQGN |
| | | VFSCSVLHEALHSHYTQKSLSLSPG |
| | | KGGGGSGGGGSEIQLVESGGGLIQP |
| | | GGSLRISCAVSGFSLSSYGVDWVRQ |
| | | APGKCLEWLGVIWGFGGTNYNSALK |
| | | SRLTISKDNSKNTVYLQMNSLRAED |
| | | TAVYYCASGNFDAMDYWGQGTLVTV |
| | | SSGGGGSGGGGSGGGGSGGGGSDIQ |
| | | MTQSPSSLSASVGDRVTITCRASQD |
| | | ISNYLNWYQQKPGKAVKLLIYYTSR |
| | | LHSGVPSRFSGSGSGTDYTLTISSL |
| | | QPEDFATYFCQQGNTLPFTFGCGTK |
| | | LEIK |
| TLb0045-3 | DVVMTQSPLSLPVTPGEPASISCRS | QVQLVQSGAEVKKPGASVKVSCKGS |
| | SQSLAKSYGNTYLSWYLQKPGQSPQ | GYTFTSYWMHWVRQAPGQRLEWIGE |
| | LLIYGISNRFSGVPDRFSGSGSGTD | IDPSESNTNYNQKFKGRVTLTVDIS |
| | FTLKISRVEAEDVGVYYCLQGTHQP | ASTAYMELSSLRSEDTAVYYCARGG |
| | YTFGQGTKVEIKRTVAAPSVFIFPP | YDGWDYAIDYWGQGTLVTVSSASTK |
| | SDEQLKSGTASVVCLLNNFYPREAK | GPSVFPLAPSSKSTSGGTAALGCLV |
| | VQWKVDNALQSGNSQESVTEQDSKD | KDYFPEPVTVSWNSGALTSGVHTFP |
| | STYSLSSTLTLSKADYEKHKVYACE | AVLQSSGLYSLSSVVTVPSSSLGTQ |
| | VTHQGLSSPVTKSFNRGEC | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALPAPIEKTISKAKGQPREP |
| | | QVYTLPPSRDELTKNQVSLTCLVKG |
| | | FYPSDIAVEWESNGQPENNYKTTPP |
| | | VLDSDGSFFLYSKLTVDKSRWQQGN |
| | | VFSCSVLHEALHSHYTQKSLSLSPG |
| | | KGGGGSGGGGSGGGGSEIQLVESGG |
| | | GLIQPGGSLRISCAVSGFSLSSYGV |
| | | DWVRQAPGKCLEWLGVIWGFGGTNY |
| | | NSALKSRLTISKDNSKNTVYLQMNS |
| | | LRAEDTAVYYCASGNFDAMDYWGQG |
| | | TLVTVSSGGGGSGGGGSGGGGSGGG |
| | | GSDIQMTQSPSSLSASVGDRVTITC |
| | | RASQDISNYLNWYQQKPGKAVKLLI |
| | | YYTSRLHSGVPSRFSGSGSGTDYTL |
| | | TISSLQPEDFATYFCQQGNTLPFTF |
| | | GCGTKLEIK |
| TLb0065-1 | DVVMTQSPLSLPVTPGEPASISCRS | QVQLVQSGAEVKKPGASVKVSCKGS |
| | SQSLAKSYGNTYLSWYLQKPGQSPQ | GYTFTSYWMHWVRQAPGQRLEWIGE |
| | LLIYGISNRFSGVPDRFSGSGSGTD | IDPSESNTNYNQKFKGRVTLTVDIS |
| | FTLKISRVEAEDVGVYYCLQGTHQP | ASTAYMELSSLRSEDTAVYYCARGG |
| | YTFGQGTKVEIKRTVAAPSVFIFPP | YDGWDYAIDYWGQGTLVTVSSASTK |
| | SDEQLKSGTASVVCLLNNFYPREAK | GPSVFPLAPSSKSTSGGTAALGCLV |
| | VQWKVDNALQSGNSQESVTEQDSKD | KDYFPEPVTVSWNSGALTSGVHTFP |
| | STYSLSSTLTLSKADYEKHKVYACE | AVLQSSGLYSLSSVVTVPSSSLGTQ |
| | VTHQGLSSPVTKSFNRGEC | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALPAPIEKTISKAKGQPREP |
| | | QVYTLPPSRDELTKNQVSLTCLVKG |
| | | FYPSDIAVEWESNGQPENNYKTTPP |
| | | VLDSDGSFFLYSKLTVDKSRWQQGN |
| | | VFSCSVLHEALHSHYTQKSLSLSPG |
| | | KGGGGSGGGGSGGGGSGGGGSEIQL |
| | | VESGGGLIQPGGSLRISCAVSGFSL |
| | | SSYGVDWVRQAPGKGLEWLGVIWGF |
| | | GGTNYNSALKSRLTISKDNSKNTVY |
| | | LQMNSLRAEDTAVYYCASGNFDAMD |
| | | YWGQGTLVTVSSGGGGSGGGGSGGG |
| | | GSGGGGSDIQMTQSPSSLSASVGDR |
| | | VTITCRASQDISNYLNWYQQKPGKA |
| | | VKLLIYYTSRLHSGVPSRFSGSGSG |
| | | TDYTLTISSLQPEDFATYFCQQGNT |
| | | LPFTFGQGTKLEIK |
| TLb0111 | DVVMTQSPLSLPVTPGEPASISCRS | QVQLVQSGAEVKKPGASVKVSCKGS |
| | SQSLAKSYGNTYLSWYLQKPGQSPQ | GYTFTSYWMHWVRQAPGQRLEWIGE |
| | LLIYGISNRFSGVPDRFSGSGSGTD | IDPSESNTNYNQKFKGRVTLTVDIS |
| | FTLKISRVEAEDVGVYYCLQGTHQP | ASTAYMELSSLRSEDTAVYYCARGG |
| | YTFGQGTKVEIKRTVAAPSVFIFPP | YDGWDYAIDYWGQGTLVTVSSASTK |
| | SDEQLKSGTASVVCLLNNFYPREAK | GPSVFPLAPSSKSTSGGTAALGCLV |
| | VQWKVDNALQSGNSQESVTEQDSKD | KDYFPEPVTVSWNSGALTSGVHTFP |
| | STYSLSSTLTLSKADYEKHKVYACE | AVLQSSGLYSLSSVVTVPSSSLGTQ |
| | VTHQGLSSPVTKSFNRGEC | TYICNVNHKPSNTKVDKKVEPKSCD |
| | | KTHTCPPCPAPEAAGGPSVFLFPPK |
| | | PKDTLMISRTPEVTCVVVDVSHEDP |
| | | EVKFNWYVDGVEVHNAKTKPREEQY |
| | | NSTYRVVSVLTVLHQDWLNGKEYKC |
| | | KVSNKALPAPIEKTISKAKGQPREP |
| | | QVYTLPPSRDELTKNQVSLTCLVKG |
| | | FYPSDIAVEWESNGQPENNYKTTPP |
| | | VLDSDGSFFLYSKLTVDKSRWQQGN |
| | | VFSCSVLHEALHSHYTQKSLSLSPG |
| | | KGGGGSGGGGSGGGGSGGGGSQVQL |
| | | VQSGAEVKKPGASVKVSCKASGYDF |
| | | TYYGISWVRQAPGQCLEWMGWISTY |
| | | NGNTHYARMLQGRVTMTTDTSTRTA |
| | | YMELRSLRSDDTAVYYCARENYYGS |
| | | GAYRGGMDVWGQGTTVTVSSGGGGS |
| | | GGGGSGGGGSGGGGSEIVLTQSPAT |
| | | LSLSPGERATLSCRASQSVSSYLAW |
| | | YQQKPGQAPRLLIYDASNRATGIPA |
| | | RFSGSGSGTDFTLTISSLEPEDFAV |
| | | YYCQQRSNWPWTFGCGTKVEIK |

TABLE 3B
Bispecific antibody amino acid sequence with format 1+1

| | Light chain | Heavy chain 1 | Heavy chain 2 |
|---|---|---|---|
| No | sequence | sequence | sequence |
| TLb0069 | DIQMTQSPSSLSASV | EIQLVESGGGLIQPG | DVVMTQSPLSLPVTP |
| | GDRVTITCRASQDIS | GSLRISCAVSGFSLS | GEPASISCRSSQSLA |
| | NYLNWYQQKPGKAVK | SYGVDWVRQAPGKGL | KSYGNTYLSWYLQKP |
| | LLIYYTSRLHSGVPS | EWLGVIWGFGGTNYN | GQSPQLLIYGISNRF |
| | RFSGSGSGTDYTLTI | SALKSRLTISKDNSK | SGVPDRFSGSGSGTD |
| | SSLQPEDFATYFCQQ | NTVYLQMNSLRAEDT | FTLKISRVEAEDVGV |
| | GNTLPFTFGQGTKLE | AVYYCASGNFDAMDY | YYCLQGTHQPYTFGC |
| | IKRTVAAPSVFIFPP | WGQGTLVTVSSASTK | GTKVEIKGGGGSGGG |
| | SDEQLKSGTASVVCL | GPSVFPLAPSSKSTS | GSGGGGSGGGGSQVQ |
| | LNNFYPREAKVQWKV | GGTAALGCLVKDYFP | LVQSGAEVKKPGASV |
| | DNALQSGNSQESVTE | EPVTVSWNSGALTSG | KVSCKGSGYTFTSYW |
| | QDSKDSTYSLSSTLT | VHTFPAVLOSSGLYS | MHWVRQAPGQCLEWI |
| | LSKADYEKHKVYACE | LSSVVTVPSSSLGTQ | GEIDPSESNTNYNQK |
| | VTHQGLSSPVTKSFN | TYICNVNHKPSNTKV | FKGRVTLTVDISAST |
| | RGEC | DKKVEPKSCDKTHTC | AYMELSSLRSEDTAV |
| | | PPCPAPEAAGGPSVF | YYCARGGYDGWDYAI |
| | | LFPPKPKDTLMISRT | DYWGQGTLVTVSSEP |
| | | PEVTCVVVDVSHEDP | KSADKTHTCPPCPAP |
| | | EVKFNWYVDGVEVHN | EAAGGPSVFLFPPKP |
| | | AKTKPREEQYNSTYR | KDTLMISRTPEVTCV |
| | | VVSVLTVLHQDWLNG | VVDVSHEDPEVKENW |
| | | KEYKCKVSNKALPAP | YVDGVEVHNAKTKPR |
| | | IEKTISKAKGQPREP | EEQYNSTYRVVSVLT |
| | | QVYTLPPCRDELTKN | VLHQDWLNGKEYKCK |
| | | QVSLWCLVKGFYPSD | VSNKALPAPIEKTIS |
| | | IAVEWESNGQPENNY | KAKGQPREPQVCTLP |
| | | KTTPPVLDSDGSFFL | PSRDELTKNQVSLSC |
| | | YSKLTVDKSRWQQGN | AVKGFYPSDIAVEWE |
| | | VFSCSVMHEALHNHY | SNGQPENNYKTTPPV |
| | | TQKSLSLSPGK | LDSDGSFFLVSKLTV |
| | | | DKSRWQQGNVFSCSV |
| | | | MHEALHNRFTQKSLS |
| | | | LSPGK |
| TLb0070 | DVVMTQSPLSLPVTP | QVQLVQSGAEVKKPG | DIQMTQSPSSLSASV |
| | GEPASISCRSSQSLA | ASVKVSCKGSGYTFT | GDRVTITCRASQDIS |
| | KSYGNTYLSWYLQKP | SYWMHWVRQAPGQRL | NYLNWYQQKPGKAVK |
| | GQSPQLLIYGISNRF | EWIGEIDPSESNTNY | LLIYYTSRLHSGVPS |
| | SGVPDRFSGSGSGTD | NQKFKGRVTLTVDIS | RFSGSGSGTDYTLTI |
| | FTLKISRVEAEDVGV | ASTAYMELSSLRSED | SSLQPEDFATYFCQQ |
| | YYCLQGTHQPYTFGQ | TAVYYCARGGYDGWD | GNTLPFTFGCGTKLE |
| | GTKVEIKRTVAAPSV | YAIDYWGQGTLVTVS | IKGGGGSGGGGSGGG |
| | FIFPPSDEQLKSGTA | SASTKGPSVFPLAPS | GSGGGGSEIQLVESG |
| | SVVCLLNNFYPREAK | SKSTSGGTAALGCLV | GGLIQPGGSLRISCA |
| | VQWKVDNALQSGNSQ | KDYFPEPVTVSWNSG | VSGFSLSSYGVDWVR |
| | ESVTEQDSKDSTYSL | ALTSGVHTFPAVLQS | QAPGKCLEWLGVIWG |
| | SSTLTLSKADYEKHK | SGLYSLSSVVTVPSS | FGGTNYNSALKSRLT |
| | VYACEVTHQGLSSPV | SLGTQTYICNVNHKP | ISKDNSKNTVYLQMN |
| | TKSFNRGEC | SNTKVDKKVEPKSCD | SLRAEDTAVYYCASG |
| | | KTHTCPPCPAPEAAG | NFDAMDYWGQGTLVT |
| | | GPSVFLFPPKPKDTL | VSSEPKSADKTHTCP |
| | | MISRTPEVTCVVVDV | PCPAPEAAGGPSVFL |
| | | SHEDPEVKFNWYVDG | FPPKPKDTLMISRTP |
| | | VEVHNAKTKPREEQY | EVTCVVVDVSHEDPE |
| | | NSTYRVVSVLTVLHQ | VKFNWYVDGVEVHNA |
| | | DWLNGKEYKCKVSNK | KTKPREEQYNSTYRV |
| | | ALPAPIEKTISKAKG | VSVLTVLHQDWLNGK |
| | | QPREPQVYTLPPCRD | EYKCKVSNKALPAPI |
| | | ELTKNQVSLWCLVKG | EKTISKAKGQPREPQ |
| | | FYPSDIAVEWESNGQ | VCTLPPSRDELTKNQ |
| | | PENNYKTTPPVLDSD | VSLSCAVKGFYPSDI |
| | | GSFFLYSKLTVDKSR | AVEWESNGQPENNYK |
| | | WQQGNVFSCSVMHEA | TTPPVLDSDGSFFLV |
| | | LHNHYTQKSLSLSPG | SKLTVDKSRWQQGNV |
| | | K | FSCSVMHEALHNRFT |
| | | | QKSLSLSPGK |

### Example 3 Expression of Anti-TL1A×α4β7 Bispecific Antibody

The designed bispecific antibody was expressed, and the purity of the bispecific antibody with a purity of less than 9500 after one-step purification was purified in two steps, and the expression yield and purity of the bispecific antibody were counted in Table 4. Most of the bispecific antibodies express a volume of 100 ml, and about 20 mg of purity can be obtained to meet the requirements of the bispecific antibody.

TABLE 4
Bispecific antibody expression details

| | | | Purity | Purity | | |
|---|---|---|---|---|---|---|
| | Conc. | Amount | (SDS- | (SEC- | Expression | |
| Abs | (mg/ml) | (mg) | PAGE) | HPLC) | Volume | Host cell |
| TLb0045 | 1.73 | 25 | 95% | 99% | 100 ml | CHO—S |
| TLb0046 | 0.91 | 18 | 95% | 99% | | |
| TLb0065 | 1.13 | 24 | 95% | 99% | | |
| TLb0066 | 1.06 | 31 | 95% | 98% | | |
| TLb0067 | 0.63 | 20 | 95% | 98% | | |
| TLb0068 | 1.33 | 20 | 95% | 99% | | |
| TLb0069 | 1.26 | 13 | 95% | 97% | | |
| TLb0070 | 1.36 | 5 | 95% | 100% | | |
| TLb0081 | 1.38 | 44 | 95% | 97% | | |
| TLb0085 | 3.47 | 66 | 95% | 94% | | |
| TLb0086 | 0.97 | 35 | 95% | 99% | | |
| TLb0087 | 0.47 | 12 | 95% | 99% | | |
| TLb0088 | 1.35 | 54 | 95% | 99% | | |
| TLb0093 | 1.31 | 11 | 95% | 100% | | |
| TLb0094 | 1.76 | 18 | 95% | 99% | | |
| TLb0045-1 | 0.73 | 16 | 95% | 99% | | |
| TLb0065-1 | 1.71 | 25 | 95% | 99% | | |

### Example 4 Prediction of Sites where TLA0015 Binds to TL1A Antigen

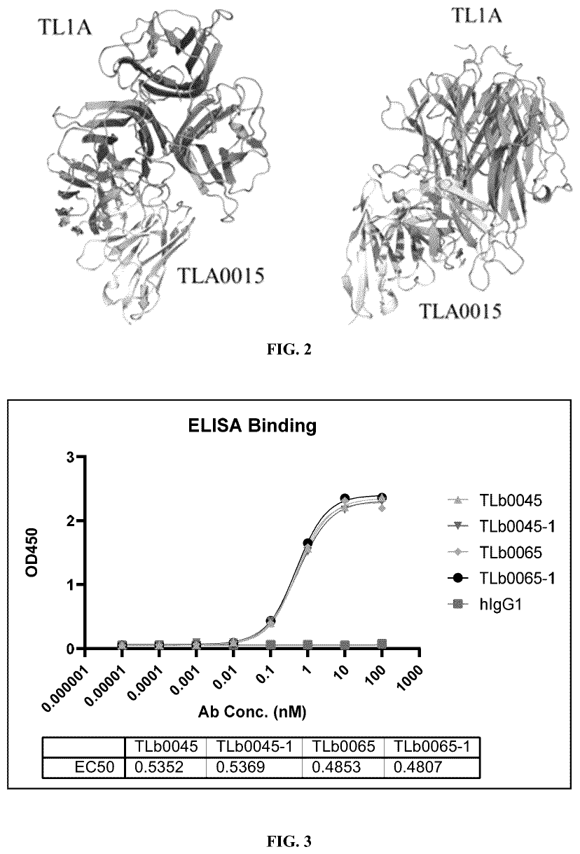

The AI-based antigen-antibody complex structure prediction model predicts the complex structure of the TL1A monomer crystal structure and the monoclonal antibody TLA0015, and obtains a high-quality complex structure. Its binding schematic diagram in different directions is shown in FIG. 2. TLA0015 binds to the TL1A monomer, but it will shift toward the gap between the two monomers in the TL1A trimer, so it has good activity in blocking the binding of TL1A to the receptor DR3. In addition, based on the results of the complex structure, the key sites for TLA0015 monoclonal antibody to bind to the TL1A antigen were obtained. These key sites on the TL1A antigen include: R32, Q33, P35, T36, Q37, P44, S89, K102, P103, D161, I162, S163, and L164.

### Example 5 ELISA Detects the Binding Ability of Bispecific Antibody to TL1A Protein

The binding ability of a plurality of bispecific antibodies prepared by example 1-3 to human TL1A (purchased from Acrobiosystems, Cat. No. TLA-H5243) was detected based on an ELISA assay. The binding capacity of different concentrations of bispecific antibodies is determined by comparing the binding curves of different concentrations to TL1A-his protein.

Human TL1A-his protein was diluted to 2 μg/ml in PBS at 100 μl/well and added to a 96-well plate and coated overnight at 4° C. The next day, remove the coating solution, add 5% BSA blocking solution at 200 μl/well, and incubate for 2 hours at room temperature. Remove the blocking solution, wash three times at 250 μl/well with 0.5‰ PBST, and dilute the bispecific antibody to 100 nM with the blocking solution, 10-fold dilution to form 8 concentration gradients (maximum concentration 100 nM) at 100 μl/The wells were added to the closed plate and incubated for 60 minutes at room temperature. After washing the plate 3 times with PBST, pat it dry and incubate at 100 μl/well with 10,000-fold dilution of HRP-labeled goat anti-human IgG secondary antibody for 60 min at room temperature. Wash the plate 5 times with 0.5‰ PBST at 100 μl/well, add TMB solution, incubate at room temperature in the dark for 10 min, at 100 μl/well, add stop solution, stop the substrate chromogenic reaction, read the OD value at 450 nm with a microplate reader, analyze and plot the data with a GraphPad and calculate EC50. As shown in FIG. 3, the bispecific antibody has good binding activity to the TL1A protein.

### Example 6 Biacore Detects the Affinity of the Bispecific Antibody with the TL1A and α 4β7 Protein

The affinity of the bispecific antibodies prepared by the present invention through example 1-3 with human TL1A-his protein, monkey TL1A-his and human α4β7 protein is detected based on the Biacore biomolecular interaction instrument.

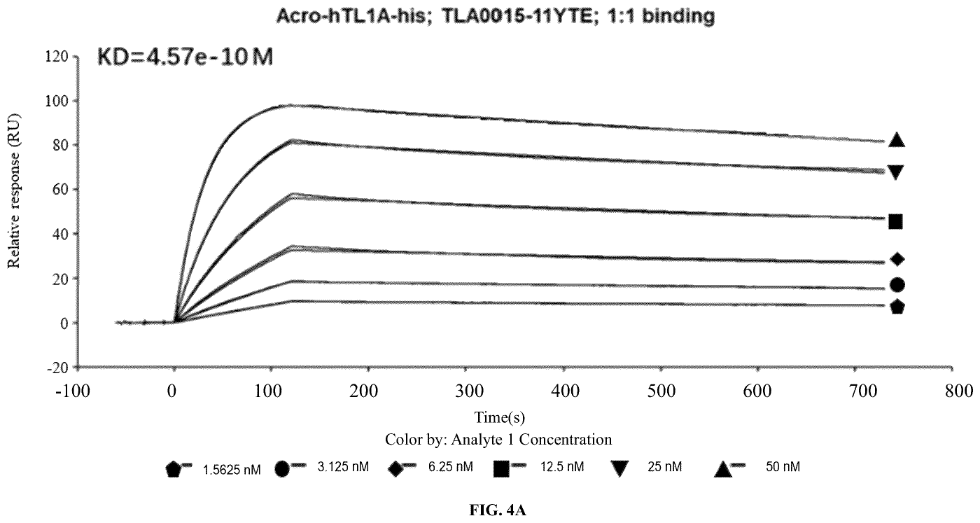

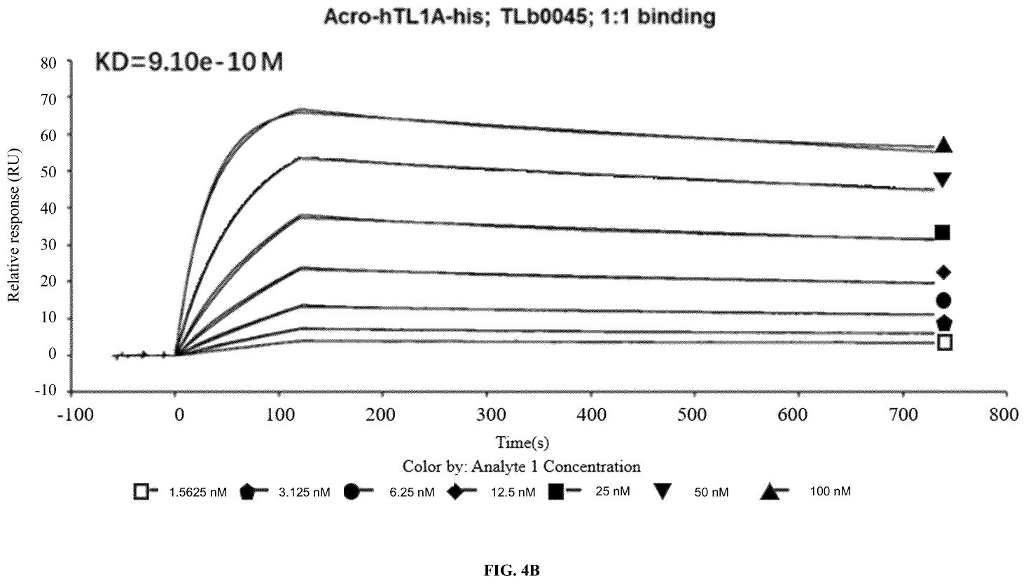

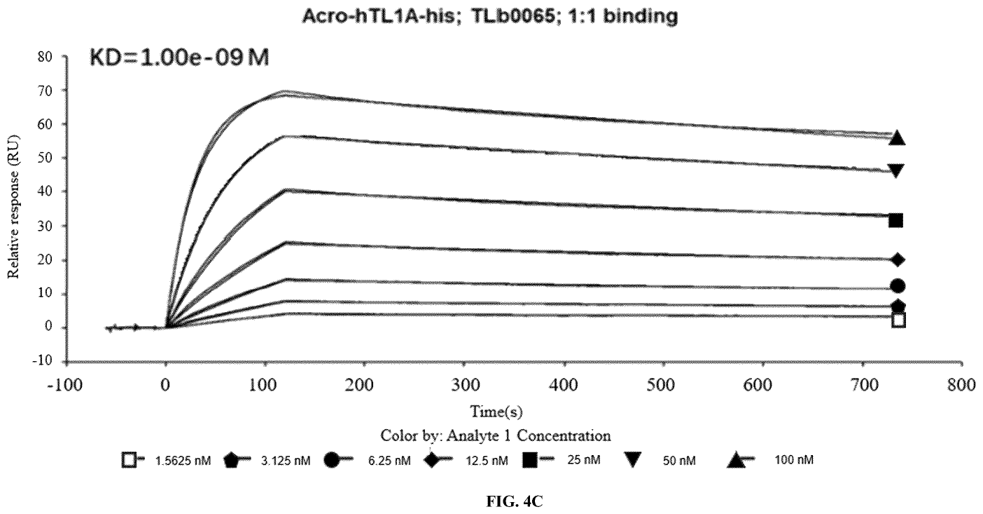

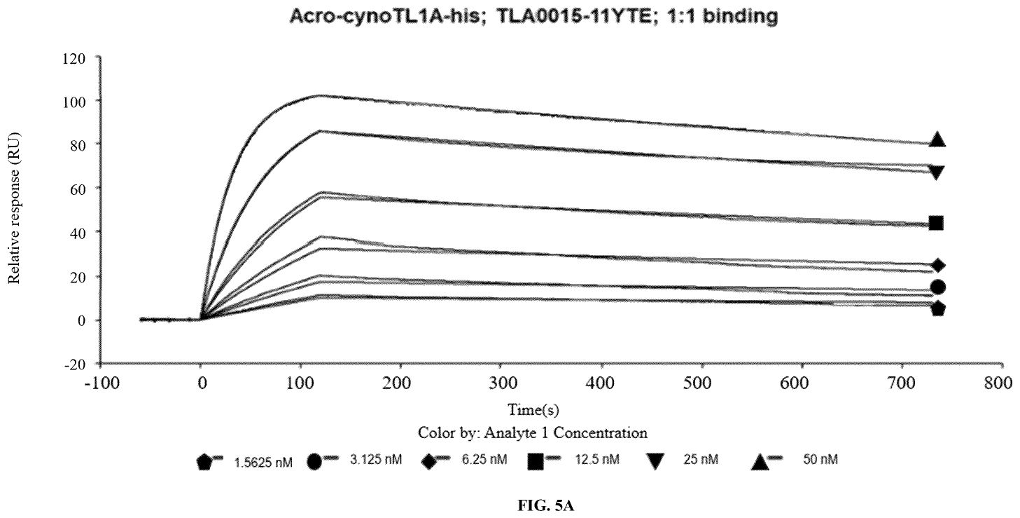

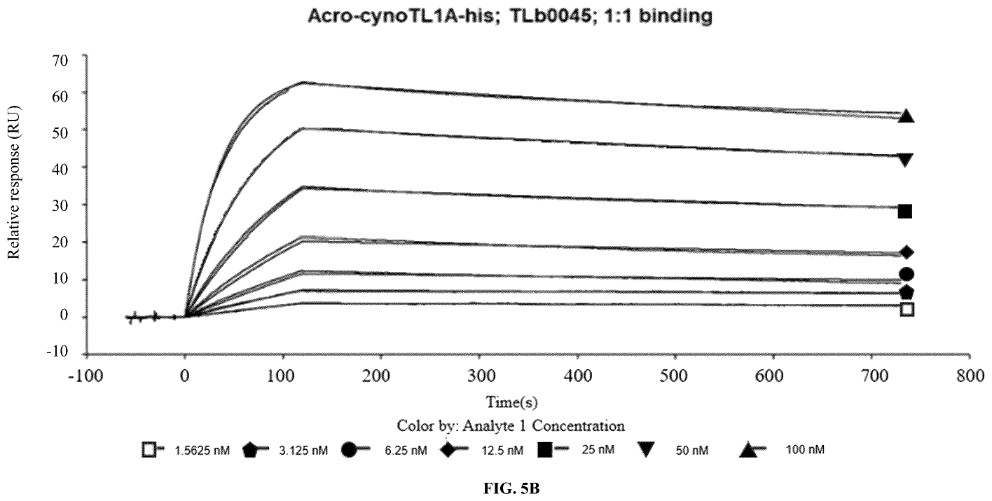

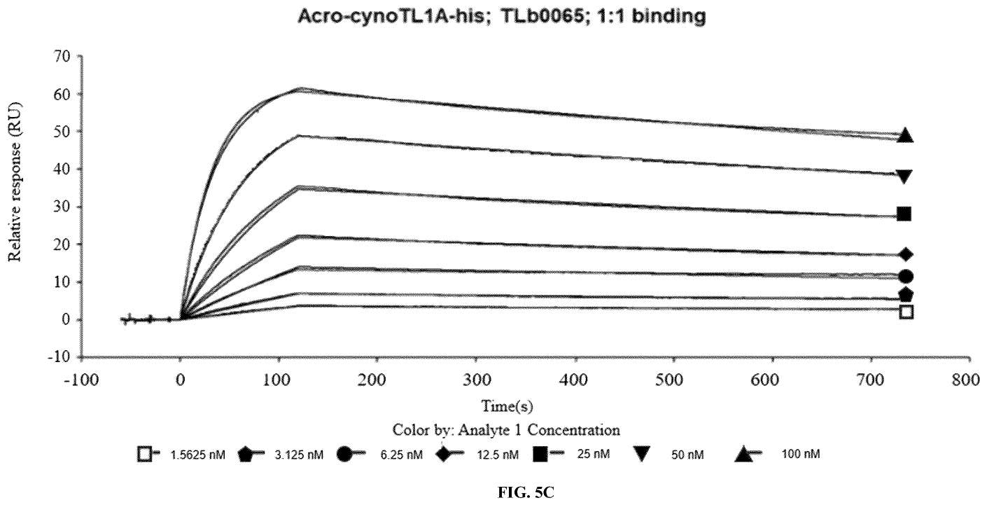

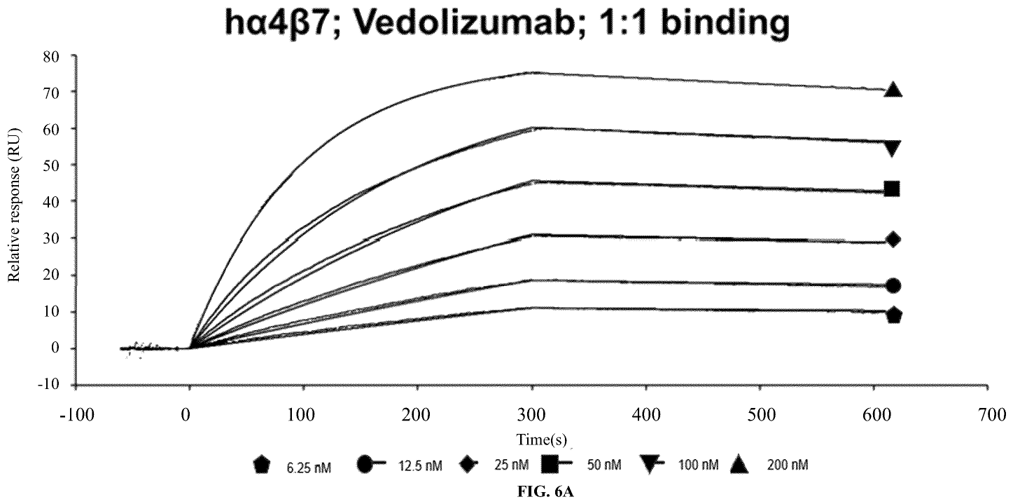

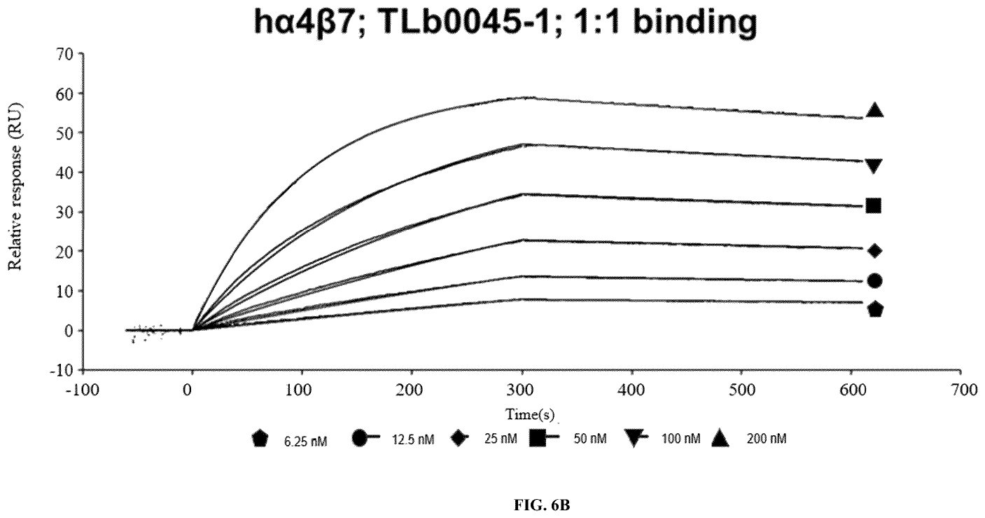

A certain amount of antibody to be tested was captured affinity using the biosensing chip Protein A (Cytiva, 29127556), and then a series of different concentrations of antigen (50 nM, 25 nM, 12.5 nM, 6.25 nM, 3.125 nM, 1.5625 nM) were passed on the surface of the chip to obtain binding and dissociation curves by detecting the reaction signal in real time with Biacore (Cytiva, 1K). After the completion of each cycle of dissociation, the biochips are washed and regenerated with 10 mM glycine-hydrochloric acid solution pH 1.5 (Cytiva, BR-1003-54). The experimental data were fitted to a 1:1 model using Biacore Insight Evaluation Software 5.0 to calculate the value of the affinity. The results are shown in FIGS. 4 and 5 and Table 5. TLb0045 and TLb0065 bispecific antibodies have high affinity for both human TL1A and monkey TL1A proteins. The affinity results of Vedolizumab (analog) and bispecific antibody TLb0045-1 to human α4β7 are shown in FIGS. 6A-6B and Table 6. The affinity of TLb0045-1 to human α4β7 is comparable to that of Vedolizumab (analog).

TABLE 5
Affinity of antibodies to TL1A proteins of different species

| Abs | Ag | ka (1/Ms) | kd (1/s) | KD (M) |
|---|---|---|---|---|
| TLA0015-11 | hTL1A-his | 6.57e+05 | 3.00e−04 | 4.57e−10 |
| TLb0045 | (Trimer) | 3.16e+05 | 2.87e−04 | 9.10e−10 |
| TLb0065 | | 3.35e+05 | 3.36e−04 | 1.00e−09 |
| TLA0015-11 | cynoTL1A-his | 6.32e+05 | 4.04e−04 | 6.39e−10 |
| TLb0045 | | 2.69e+05 | 2.68e−04 | 9.93e−10 |
| TLb0065 | | 3.09e+05 | 3.93e−04 | 1.27e−09 |

TABLE 6
Affinity of antibodies to human α4β protein

| Abs | Ag | ka (1/Ms) | kd (1/s) | KD (M) |
|---|---|---|---|---|
| Vedolizumab | Human α4β7 | 5.07e+04 | 2.05e−04 | 4.04e−09 |
| TLb0045-1 | | 4.85e+04 | 3.02e−04 | 6.24e−09 |

### Example 7 FACS Detects the Ability of Bispecific Antibody to Block the Binding of TL1A-his to 293-DR3 Cells

The ability of multiple bispecific antibodies prepared by example 1-3 to block the binding of hu TL1A-his protein to human DR3 (DR3: UniprotKB NO. Q93038) expressed on the surface of Expi293 cells was detected based on flow cytometry assay. The blocking ability of huTL1A-his protein is determined by comparing the curves of different antibody concentrations blocking huTL1A-his protein to 293-huDR3 cells.

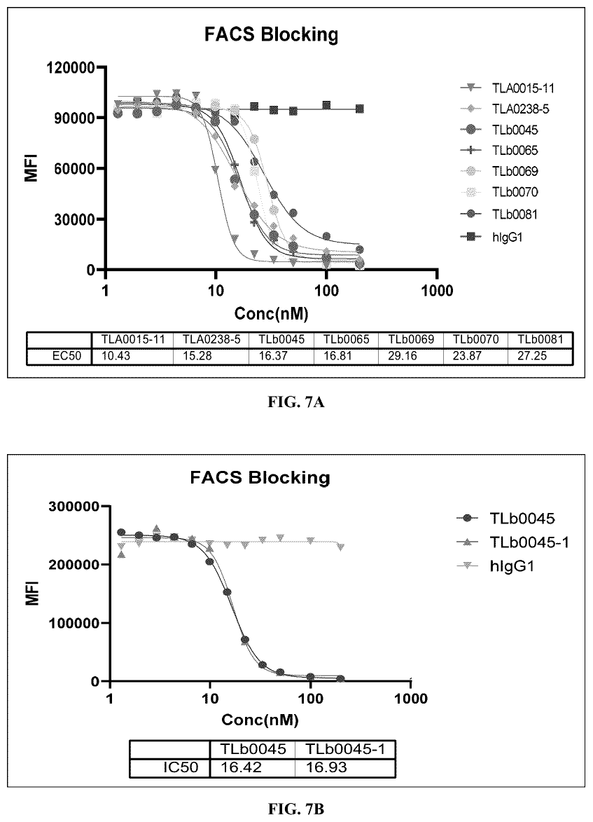

The DNA sequence of the receptor DR3 extracellular domain protein encoding TL1A was cloned into the mDeZ-TM (with transmembrane sequence and Zeocin resistance gene) vector, transiently transfected into Expi293 cells, and 100 μg/mL Zeocin (final concentration) was added 24 h later for 7 days to obtain the Expi293 cell line (293-huDR3) expressing human DR3 on the cell surface. The huTL1A-his-Biotin protein was obtained by Biotin conjugation using a biotin labeling kit (ThermoFisher, A35389). FACS Binding was used to determine the EC80 concentration of protein binding between huTL1A-his-Biotin protein and 293-huDR3 cells. Wash 293-huDR3 cells three times with staining buffer (PBS+2% FBS (Gibco, Cat #10091148)+5 mM EDTA (Gibco, Cat #15575020)) at 300 g each for 5 min and discard the supernatant. Resuspend cells in PBS, adjust the cell density to 5×105 cells/mL, 100 μl/well, add to a 96-well plate, centrifuge to remove the supernatant, and add 2*EC80 concentration of huTL1A-his-Biotin protein, 50 μl/well. Dilute the bispecific antibody and the positive control antibody to 400 nM in 2-fold increments over 12 gradients (up to a final concentration of 200 nM), 50 μl/well, add to a 96-well plate and mix well with huTL1A-his-Biotin protein as well as 293-huDR3 cells. Incubate at 4° C. for 30 min. Cells are washed twice in PBS to remove unbound antibodies to be tested. Add another 100 μl/well of Streptavidin-PE secondary antibody and incubate at 4° C. for 30 min. Centrifuge at 300 g for 5 min and wash the cells twice in PBS to remove unbound secondary antibody. Finally, the cells are resuspended in 200 μl PBS, and the ability of multiple antibodies to block the binding of TL1A protein to 293-huDR3 cells is determined by a Beckman Coulter CytoFLEX flow cytometer. The resulting data were fitted and analyzed by GraphPad Prism software. As shown in FIG. 7A, the antibodies all have the ability to block the binding of TL1A-his protein to 293-DR3 cells. In addition, there was no difference in the blocking activity between TLb0045 and TLb0045-1 (FIG. 7B).

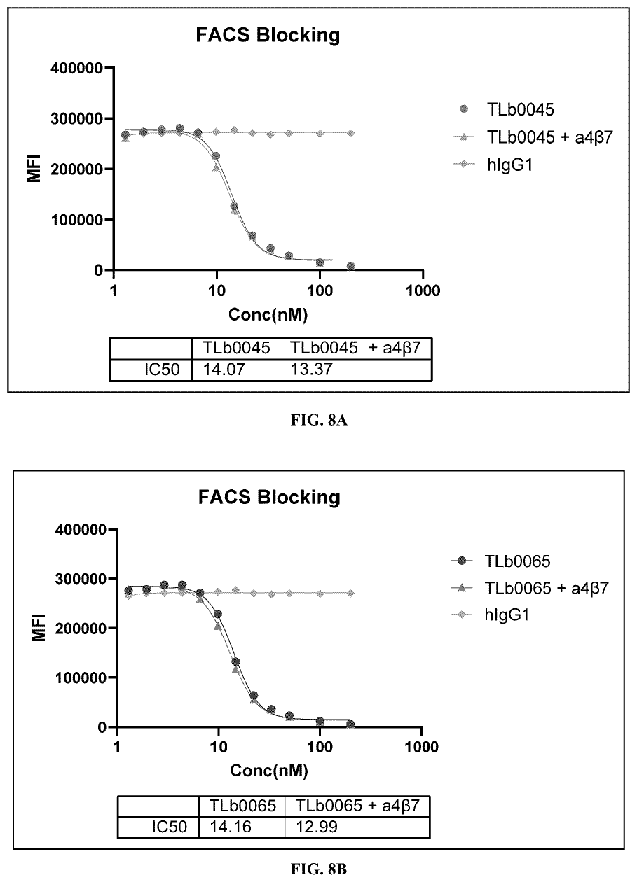

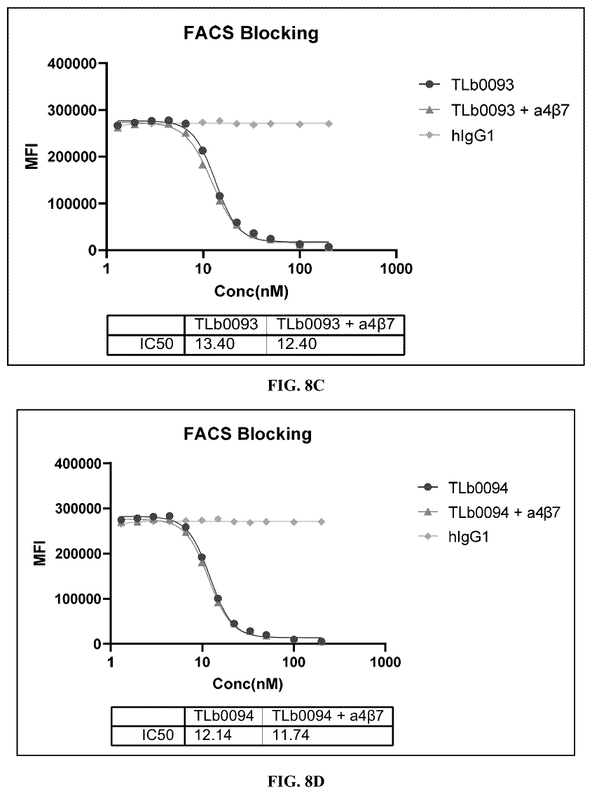

In addition, 10 nM of α4β7 protein (purchased from Acrobiosystems, item number MAM-H52H4) was added at the same time as incubation of the bispecific antibody with huTL1A-his-biotin protein and 293-DR3 cells, and was used to verify that binding of α4β7 protein to the bispecific antibody interfered with the binding of the bispecific antibody to huTL1A-his-biotin. protein, the results are shown in FIG. 8, α4β7 protein binding to the bispecific antibodies does not interfere with the blocking activity of the bispecific antibodies on the TL1A protein.

### Example 8 Detects the Ability of Bispecific Antibody to Inhibit TL1A-DR3-Activated caspase3/7

A method based on the Caspase-Glo® 3/7 Assay System Activity Assay Kit (Promega, Cat #G8092) was used to assay the ability of the plurality of bispecific antibodies of the present invention, prepared by means of Examples 1-3, to inhibit the TL1A-induced apoptosis signaling pathway in TF-1 (endogenously expressing DR3) cells. The inhibitory capacity was determined by comparing the curves of inhibition of caspase3/7 activity in TF-1 cells with different antibody concentrations.

The huTL1A-his protein was configured to a 4× working concentration of 16 nM; the Cycloheximide small molecule drug was configured to a 4× working concentration of 10 μg/ml. 25 μl of the mixture was added to each well in a white-bottomed 96-well plate. The starting concentration of the antibody was configured as 200 nM (final concentration was 50 nM), 4× dilution of four concentration points, then 2× dilution for a total of 10 concentrations, and 25 μl of each well was added to the white bottom 96-well plate. Pre-incubate the above mixture in a 37° C. incubator for 1 h. At the same time, centrifuge the TF-1 cells (300 g for 5 min) and resuspend them with buffer (1640 medium+10% FBS) and count them, and adjust the density of cells to 1E6 cells/ml. When the incubation time of the antigen-antibody is over, take out the white 96-well flat-bottomed plate, and inoculate TF-1 cells according to the adjusted density of 50 μl/well, and then add 25 μl of each well into the white bottom 96-well plate. After incubation, take out the white 96-well flat-bottom plate, inoculate with the adjusted density of TF-1 cells at 50 μl/well, and continue to incubate for 6 h in a carbon dioxide incubator. After incubation, take out the plate from the incubator and equilibrate the plate at room temperature for about 30 min, and take out the Caspase-Glo® 3/7 Assay System reagent beforehand and equilibrate the plate at room temperature. Add Caspase-Glo® 3/7 Assay System reagent, 50 μl per well, shake the bed at 300 rpm for 30 s, and then leave it for 15-30 min to make the cells fully lysed, and then carry out the reading of the RLU value on a multifunctional enzyme labeling instrument, and then carry out a four-parameter fit plot of the logarithm of the sample concentration to the measured RLU value with Graphpad. The results are shown in FIG. 9 and Table 7, the bispecific antibodies such as TLb0045, TLb0065 and TLb0081 all functioned well to inhibit Caspase3/7 activity.

TABLE 7
IC50 of the inhibitory effect on TL1A-
activated Caspase3/7 activity

| Abs | IC50 (nM) |
|---|---|
| TLb0045 | 0.821 |
| TLb0065 | 1.029 |
| TLb0069 | 3.184 |
| TLb0070 | 2.519 |
| TLb0081 | 1.300 |
| TLA0015-11 | 1.101 |
| TLA0238-5 | 0.884 |

### Example 9 Fluorescent Reporter Method to Detect the Ability of Bispecific Antibodies to Inhibit the NF-κB Signaling Pathway Activated by TL1A

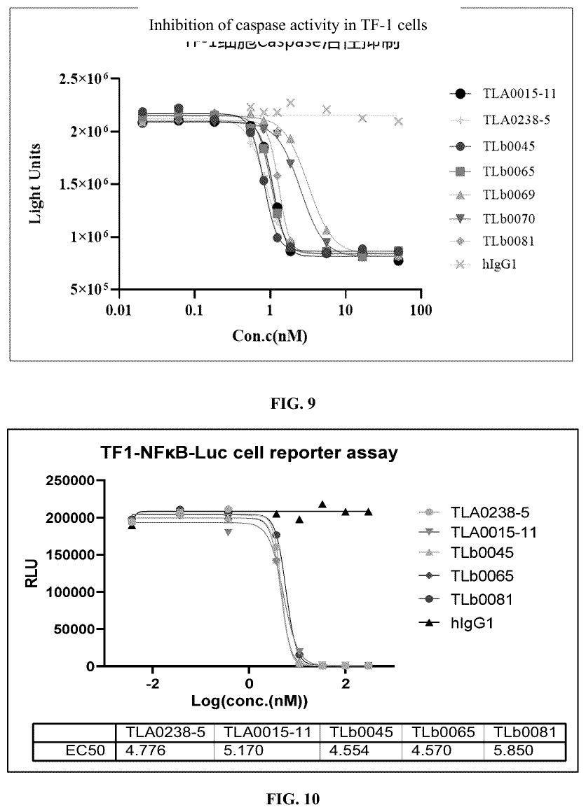

A luciferase reporter gene-based method was used to test the ability of multiple bispecific antibodies obtained by the present invention prepared by Examples 1-3 to inhibit the NF-κB signaling pathway in TF-1 (endogenously expressing DR3) cells activated by the TL1A-his protein. The TF-1-NFκB-Luc cell line was purchased from Yoshimitsu Biotechnology Ltd (GM-C30289, H_TNFSF15(TL1A) Reporter Cell Line) and screened using NF-κB-Luciferase lentiviral infection of TF-1 cells. In this study, TF1-NFκB-luc cells were used to detect the biological activity of the DR3-NF κB signaling pathway activated by TL1A-his protein by bispecific antibody. In this experiment, 3× of the analytical concentration of huTL1A-his antigen solution was prepared with analytical buffer (RPMI 1640 medium+1% FBS) at 0.9 μg/ml (final concentration of 0.3 μg/ml) and added to a white 96-well flat-bottom plate at 40 μl/well. Prepare bispecific antibodies at 3× analytical concentrations with a maximum concentration of 300 nM (final concentration of 100 nM) and a 5-fold serial dilution for a total of 8 concentration points. After the antibody is diluted, add 40 μl/well to the white 96-well flat-bottom plate described above, then place the white 96-well flat-bottom plate in a carbon dioxide incubator, and pre-incubate the bispecific antibody and antigen for 1 h. During bispecific antibody and antigen pre-incubation, TF1-NFκB-luc cells were centrifuged (300 g, 5 min) and then resuspended with analysis buffer and counted, adjusting the cell density to 5E5 cells/ml. After the antigen and antibody incubation time was over, the white 96-well flat-bottom plate was removed, and TF1-NFκB-luc cells with adjusted density were inoculated at 40 μl/well, and placed in a carbon dioxide incubator for 6 h. At the end of the incubation, remove the white 96-well flat-bottom plate from the incubator, add 60 μl of Bio-Lite Luciferase Assay reagent (Novozan, DD1201-02) to each well, shake slightly for 5 minutes, and read the RLU values on a multimode microplate reader. The four-parameter fitting plot of the logarithm of the sample concentration with Graphpad was performed with the measured signal value, and the results are shown in FIG. 10: TLb0045, TLb0065, and TLb0081 bispecific antibodies all have good activity in inhibiting the NF-κB signaling pathway activated by TL1A-his protein.

### Example 10 ELISA Detects the Binding Ability of Bispecific Antibody to α4P7 Protein

Based on the ELISA assay method to detect the binding ability of a plurality of bispecific antibodies prepared by example 1-3 of the present invention and human α4β7 protein (purchased from Acrobiosystems, Cat. No. IT7-H52W4). The binding capacity of different bispecific antibodies is determined by comparing the binding curves of different bispecific antibodies to α4β7 protein.

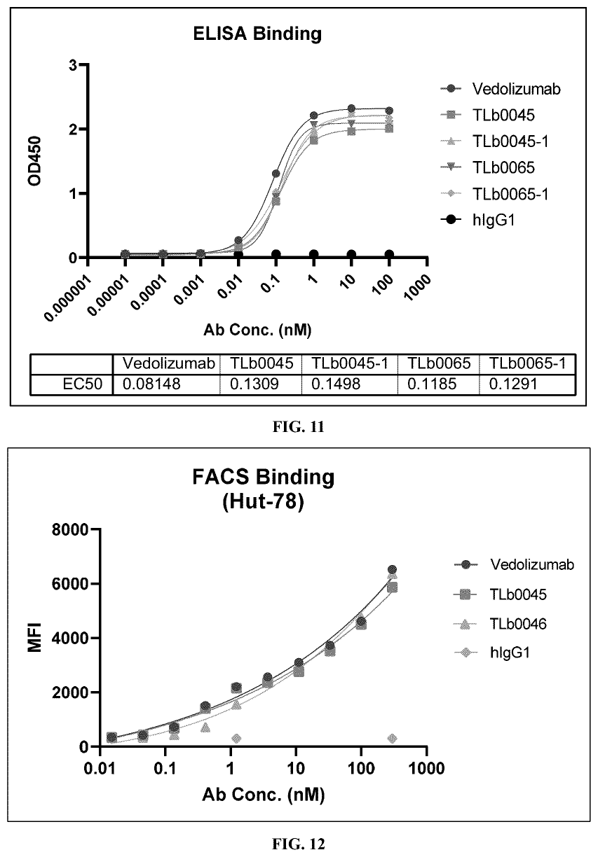

Dilute α4β7 protein to 2 μg/ml with coating solution (PBS+1 mM CaCl₂)+1 mM MgCl2) at 100 μl/well, add to the microplate and incubate overnight at 4° C. Remove the coating solution and incubate at 200 μl/well with 5% BSA blocking solution (containing 1 mM MnCl2) for 2 hours. Remove the blocking solution, wash three times at 250 μl/well with 0.5‰ PBST (containing 1 mM MnCl2), and dilute the bispecific antibody to 100 nM with the blocking solution, 10-fold dilution to form 8 concentration gradients (maximum concentration 100 nM) at 100 μl/The wells were added to the closed plate and incubated for 60 minutes at room temperature. Wash the plate 3 times with 0.5‰ PBST (containing 1 mM MnCl2) and pat dry, add HRP-labeled goat anti-human IgG antibody at 100 μl/well, and incubate for 60 min at room temperature. Wash the plate 5 times with 0.5‰ PBST (containing 1 mM MnCl2) at 100 μl/well, add TMB solution, incubate for 10 min at room temperature in the dark, at 100 μl/well, add stop solution, stop the substrate chromogenic reaction, read the OD value at 450 nm with a microplate reader, analyze and plot the data with a GraphPad and calculate EC50. As shown in FIG. 11, the bispecific antibodies have good binding activity to the α4β7 protein, as with the vedolizumab monoclonal antibody.

### Example 11 FACS was Used to Detect the Binding Ability of Bispecific Antibody to Cells Endogenously Expressing α4P7

The binding ability of a plurality of bispecific antibodies prepared by the present invention through example 1-3 to cells endogenously expressing α4β7 is detected based on flow cytometry assay. Hut 78 cells (purchased from the Cell Bank of the Chinese Academy of Sciences, Cat #TCHu206) are a T lymphocyte line with α4β7 molecules endogenously expressed on the cell membrane surface. The binding capacity of different concentrations of antibodies is determined by comparing their binding curves with those of the Hut78 cell line.

In this experiment, Hut 78 cells endogenously expressing α4β7 were used as target cells. Wash cells three times with staining buffer (PBS+2% FBS (Gibco, Cat #10091148)+5 mM EDTA (Gibco, Cat #15575020)) at 450 g each time for 5 min and discard the supernatant. Resuspend cells in staining buffer, adjust cell density to 1×106 cells/mL, 100 μl/well, and add to a 96-well plate. Bispecific antibody and positive control antibody were diluted to 200 nM, 10-fold stepwise dilution of 8 gradients (maximum concentration 200 nM), 100 μl/well, added to a 96-well plate, and mixed well with Hut 78 cells. After 30 min of incubation at 4° C., the cells are washed twice in PBS to remove unbound antibodies to be tested. Add another 100 μl/well of goat anti-human IgG-PE and incubate at 4° C. for 30 min. Centrifuge at 300 g for 5 min and wash the cells twice in PBS to remove unbound secondary antibody. Finally, the cells were resuspended in 200 μl PBS, and the binding capacity of the bispecific antibody to Hut 78 cells was determined by Beckman Coulter CytoFLEX flow cytometry. The resulting data were fitted and analyzed by GraphPad Prism software. As shown in FIG. 12, both TLb0045 and TLb0065 bispecific antibodies bind well to Hut 78 cells, as with the vedolizumab monoclonal antibody.

### Example 12 ELISA Detection of the Binding of Bispecific Antibody Blocking α4P7 to the Ligand MAdCAM-1

The ability of multiple bispecific antibodies prepared by example 1-3 to block the binding of α4β7 protein and the ligand MAdCAM-1 protein (purchased from Acrobiosystems, Cat. No. MAM-H5253) is detected by ELISA assay by comparing different concentrations of bispecific antibodies. The curve of α4β7 protein binding to the ligand MAdCAM-1 was used to determine its blocking ability.

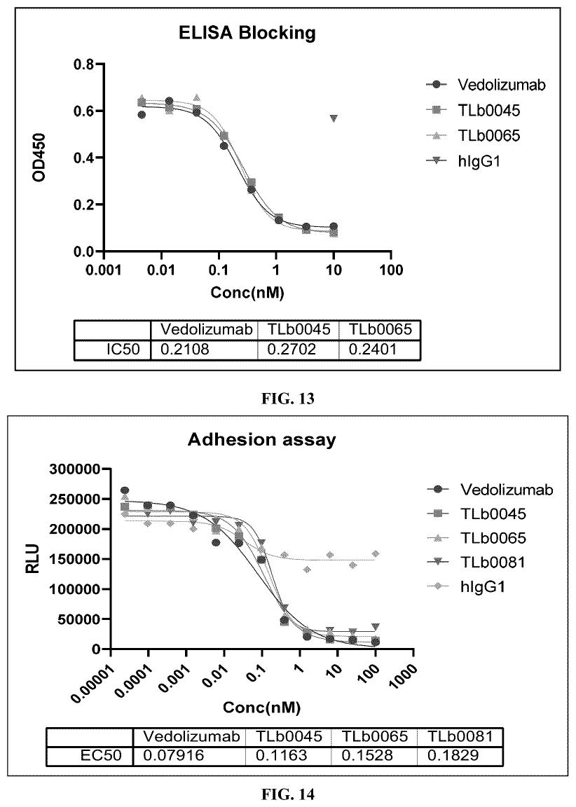

Biotin conjugation of MAdCAM-1-Fc protein was performed using a biotin labeling kit (ThermoFisher, A35389) to obtain MAdCAM-1-Fc-Biotin protein backup. Dilute α4β7 protein to 2 μg/ml with PBS (containing 1 mM CaCl₂) and 1 mM MgCl2) at 100 μl/well, add to the microplate and incubate overnight at 4° C. Remove the coating solution and incubate at 200 μl/well with 5% BSA blocking solution for 2 h at room temperature. Remove the blocking solution and wash three times at 250 μl/well by adding PBST wash (PBST containing 0.5‰ Tween-20 and 1 mM MnCl2) and pat dry for later use. Dilute the bispecific antibody to 20 nM with blocking solution, form 8 concentration gradients (maximum concentration of 20 nM) with 3-fold dilution, and add sequentially to the blocked plate at 50 μl/well, while dilute the MAdCAM-1-Fc-Biotin protein to 1 μg/ml using blocking solution, add sequentially to the plate at 50 μl/well, and incubate for 1 hour at room temperature. Wash the plate 3 times with PBST wash containing MnCl2 (remove residual droplets with absorbent paper) at 100 μl/wells, add SA-HRP secondary antibody containing 5,000-fold dilution, and incubate for 60 min at room temperature. Wash the plate 3 times with PBST wash containing MnCl2 at 100 μl/well, add TMB solution, incubate for 10 min at room temperature in the dark, at 100 μl/well, add stop solution, stop the substrate chromogenic reaction, read the OD value at 450 nm with a microplate reader, analyze the data with GraphPad and calculate EC50. As shown in FIG. 13, both TLb0045 and TLb0065 bispecific antibodies have a good ability to block the binding of α4β7 to MAdCAM-1, as with vedolizumab monoclonal antibodies.

### Example 13 Detection of Bispecific Antibody Blockade of MAdCAM-1-Mediated Cell Adhesion

The blocking ability of MAdCAM-1-mediated cell adhesion function mediated by multiple bispecific antibodies prepared by example 1-3 was detected based on CTG bioluminescence detection technology, and its blocking ability was determined by comparing the curves of different antibody concentrations blocking MAdCAM-1-mediated adhesion of Hut 78 cells.

In this experiment, Hut 78 cells endogenously expressing α4β7 were used as target cells. MAdCAM-1-Fc protein was diluted to 2 μg/ml in PBS at 100 μl/well and added to a 96-well plate and coated overnight at 4° C. Remove the coating solution and incubate at 200 μl/well with 5% BSA blocking solution for 2 h at room temperature. At the end of the incubation, remove the blocking solution and wash three times with PBST wash at 250 μl/well and pat dry for later use. Dilute cultured Hut 78 cells to 3E5/ml, 100 μl/ml with FACS buffer (DPBS+1% BSA+1 mM MnCl2). The wells were plated into a 96-well cell plate, and the supernatant was discarded by centrifugation at 450 g for 5 min; At the same time, dilute the antibody to 100 nM with FACS buffer, dilute 4-fold to form 12 concentration gradients, add to a 96-well cell plate, 100 μl/well, resuspend the cells, and then transfer to a coated microplate plate, incubate in a cell culture incubator for 40 min, centrifuge at 450 g for 5 min, discard the supernatant, wash the cells five times with FACS buffer, and then add CTG (Vazyme, Cat #DD1101-02), 50 μl/wells, microplate reader for reading. Analyze the data with GraphPad, graph it, and calculate the EC50. As shown in FIG. 14, TLb0045, TLb0065, and TLb0081 bispecific antibodies all have a good blockade of MAdCAM-1-mediated cell adhesion function, as shown in FIG. 12.

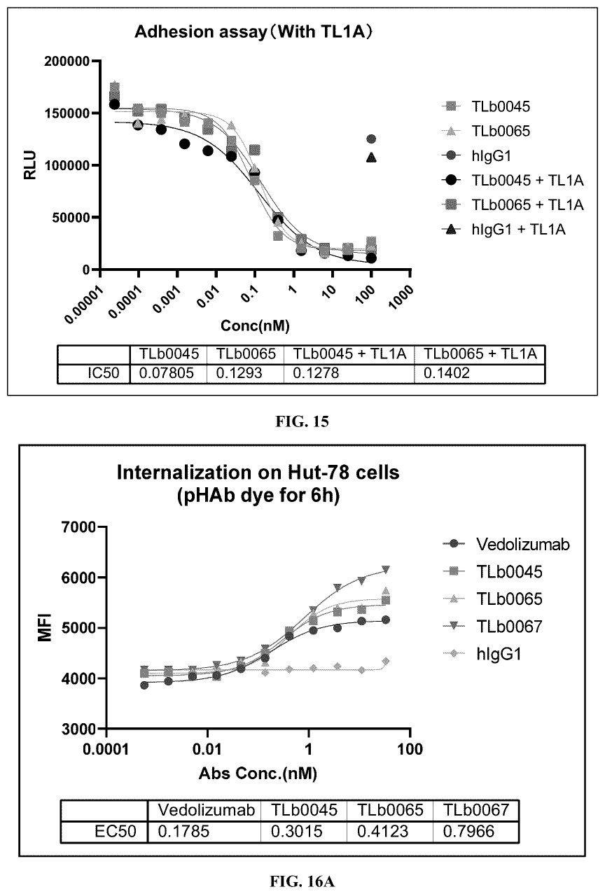

At the same time as the cell adhesion experiment, TL1A protein was added and incubated with bispecific antibody and Hut 78 cells to verify the effect of TL1A protein on the cell adhesion function of bispecific antibody blockade. As shown in FIG. 15, the addition of TL1A protein had little effect on the blockade of MAdCAM-1-mediated cell adhesion by bispecific antibodies.

### Example 14 Detection of the Internalization Activity of the Bispecific Antibody

The method based on pH Dye is used to detect the internalization activity of a plurality of bispecific antibodies prepared by example 1-3 of the present invention. The internalization capacity of bispecific antibodies is determined by comparing the internalization signal curves of different concentrations.

Antibody pH Dye labeling: antibodies were labeled using pHAb Amine Reactive Dye (Promega, Cat #G9845) reagent. Take 120 g of the antibody to be labeled and add it to a 30 kDa protein concentrate tube, then add 400 μl of 10 mM sodium bicarbonate buffer (pH 8.5), centrifuge at 7500 rpm for 5 min, repeat the above steps twice (total 3 changes), centrifuge to a volume of about 100 μl; transfer the antibody to a 1.5 mL centrifuge tube. Add 1 μl of pHAb Amine Reactive Dye solution to each tube, mix thoroughly, and shake the reaction at low speed for 60 minutes at room temperature and away from light; transfer the antibody to a 30 kDa protein concentrate tube, add 400 μl of PBS, centrifuge at 7500 rpm for 5 minutes, and repeat the above steps for 4 times (totaling 5 changes of solution); centrifuge until the volume of the solution is about 100 μl, and then transfer the antibody to a 30 kDa protein concentrate tube. Transfer the antibody to a 1.5 mL centrifuge tube, keep it away from light, test the absorbance of A280 and A532 of the labeled antibody, calculate the concentration of the labeled antibody, and set aside.

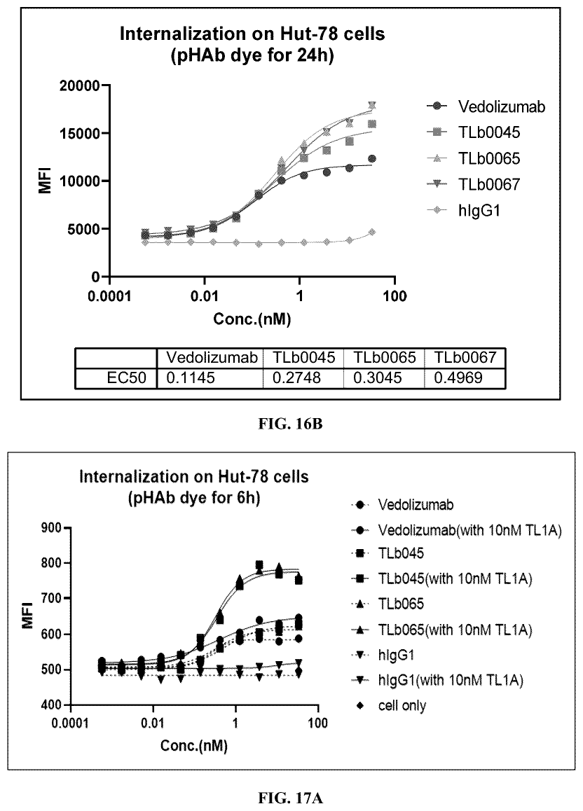

The cultured Hut 78 cells were centrifuged at 300 g for 5 min, the supernatant was discarded, resuspended in freshly prepared RPMI1640 medium, the cell density was adjusted to 2E6/mL, and the cell density was adjusted to a 96-well low-adsorption U bottom plate, a volume of 50 μl was added to each well, and the bispecific antibody and the positive control antibody were diluted to 66 nM with RPMI1640 medium. Dilute 11 gradients (maximum final concentration 33 nM) in 3-fold steps, 50 μl/well, add to a 96-well plate, mix well with Hut 78 cells, and place in a 37° C. incubator for incubation. The cell plates incubated for 6 h and 24 h were removed, centrifuged at 450 g for 5 min, the supernatant was discarded, 200 μl of FACS buffer was added to each well, 450 g was centrifuged for 5 min, the supernatant was discarded, and the step was repeated. Prepare Live/Dead dye (Invitrogen, CAT #L34976) at a ratio of 1:1,000, add 100 μl per well, and incubate at 4° C. for 30 min. After the end of the incubation, centrifuge at 450 g for 5 min, discard the supernatant, repeat this step twice, resuspend the cells with 100 μl FACS buffer, and detect the PE channel by flow cytometry. Analyze the data with GraphPad, graph it, and calculate the EC50. The 6-hour internalization is shown in FIG. 16A, and the 24-hour internalization result is shown in FIG. 16B, and the bispecific antibodies have good internalization activity.

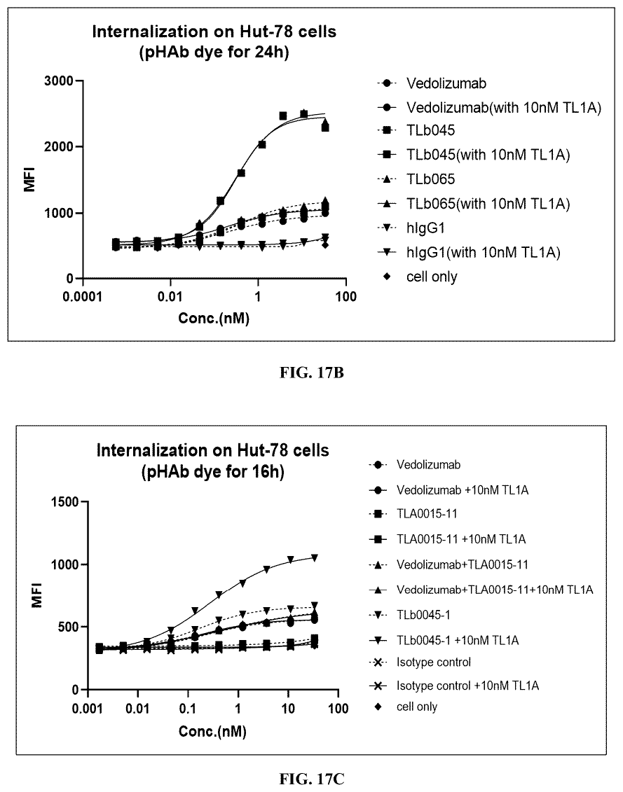

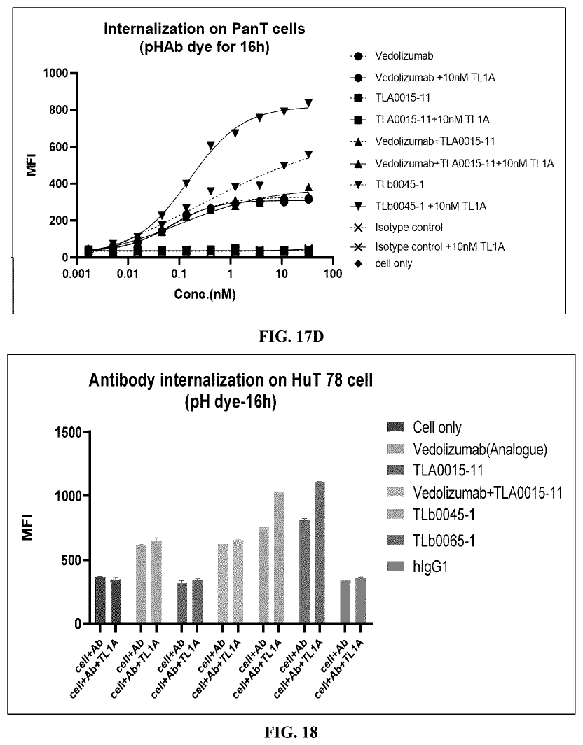

In addition, during the incubation of the internalization experimental antibody with Hut 78 cells, 10 nM TL1A protein was added to verify the effect of TL1A protein on the internalization activity of the bispecific antibody, the internalization results of 6 hours are shown in FIG. 17A, and the results of internalization in 24 hours are shown in FIG. 17B, and the internalization activity of the bispecific antibody TLb0045 and TLb0065 can be significantly improved after the addition of TL1A protein. The results of the endocytic activity of the bispecific antibody TLb0045-1 on Hut 78 cells are shown in FIG. 17C. The addition of TL1A protein significantly enhanced the endocytic activity of TLb0045-1. Using healthy human peripheral blood mononuclear cells, Pan T cells were sorted and tested for the endocytic activity of the bispecific antibody on T cells. The results of the endocytic activity of the bispecific antibody TLb0045-1 on primary T cells are shown in FIG. 17D. Consistent with the endocytic results on Hut 78 cells, the addition of TL1A protein significantly enhanced the endocytic activity of TLb0045-1.

### Example 15 Detection of Bispecific Antibody Endocytosis Downregulation of α4P7 Expression

Based on flow cytometry assay, the effect of internalization of a plurality of bispecific antibodies prepared by example 1-3 of the present invention on the cell membrane and the total α4β7 expression of cells is detected.

Cultured Hut 78 cells were centrifuged at 450 g for 5 min, the supernatant was discarded, resuspended in freshly prepared RPMI1640 medium, the cell density was adjusted to 4E6/mL, and added to a 96-well low-adsorption U bottom plate, with a volume of 25 μl per well. Dilute pH Dye-labeled bispecific antibody and positive control antibody to 10 nM in RPMI1640 medium, 50 μl/well and add to a 96-well plate. TL1A protein was diluted to 40 nM with RPMI1640 medium and 25 μl/well was added to the 96-well plate described above; For wells that do not require TL1A protein, add 25 μl of RPMI1640 medium, mix well with Hut 78 cells, and incubate in a 37° C. incubator for 16 hours (three 96-well plates, one for internalization activity, another for α4β7 expression on the membrane, and the last for total α4β7 expression).

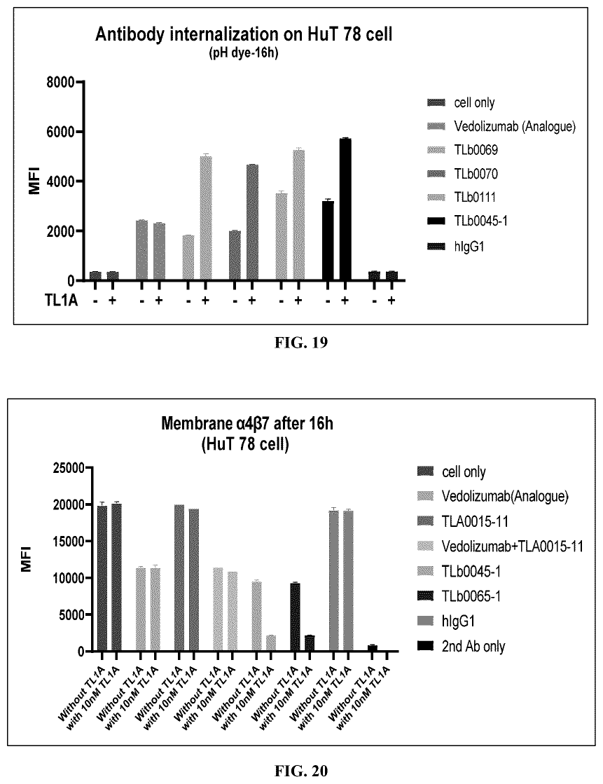

Internalization activity assay: After the end of incubation, the first cell plate was removed, 450 g centrifuged for 5 min, the supernatant was discarded, 200 μl FACS Buffer was added to each well, 450 g centrifuged for 5 min, the supernatant was discarded, and the step was repeated. Configure the Live/Dead dye at a ratio of 1:1,000, add 100 μl per well, and incubate at 4° C. for 30 min. After the end of incubation, centrifuge at 450 g for 5 min, discard the supernatant, repeat this step twice, resuspend the cells using 100 μl FACS buffer, and FACS to detect antibody internalization activity. The data were analyzed and graphed with GraphPad, and the results are shown in FIG. 18, where the vedolizumab monoclonal antibody has internalization activity and is not affected by the TL1A protein; TLA0015-11 monoclonal antibody has no internalization activity; Internalization in the Vedolizumab+TLA0015-11 combo group is mediated by Vedolizumab monoclonal antibody and is therefore also not affected by the TL1A protein; TLb0045-1 and TLb0065-1 bispecific antibodies have good internalization activity in the absence of TL1A protein, and the increase of internalization activity is significantly enhanced after the addition of TL1A protein. The reason is that after the bispecific antibodies TLb0045-1 or TLb0065-1 bind to the TL1A antigen, they will promote endocytosis activity better through cross-linking. As shown in FIG. 19, the endocytosis activity of the bispecific antibodies increased significantly after the addition of TL1A protein. The reason is that when the TL1A monoclonal antibodies in these four bispecific antibodies bind to the TL1A protein, they will form a complex antigen-antibody complex through cross-linking. Therefore, complex antigen-antibody complexes will also be formed in the bispecific antibodies through cross-linking, thereby promoting endocytosis.

Detection of α4β7 expression on the membrane: after the end of incubation, the second cell plate was removed, 450 g centrifuged for 5 min, the supernatant was discarded, 200 μl FACS Buffer was added to each well, 450 g was centrifuged for 5 min, the supernatant was discarded, and the step was repeated; Configure the Live/Dead dye at a ratio of 1:1,000 while adding 9.87 μl of Vedolizumab (at a concentration of 4.56 mg/mL) for a final concentration of 100 nM, adding 100 μl per well, and incubating at 4° C. for 60 min; Centrifuge at 450 g for 5 min, discard the supernatant, add 200 μl of FACS Buffer per well, and repeat this step three times; Dilute AF488 Goat anti-human Fc secondary antibody at a ratio of 1:200, add 100 μl per well, and incubate at 4° C. for 30 min; Centrifuge at 450 g for 5 min, discard the supernatant, add 200 μl of FACS Buffer per well, repeat this step three times, resuspend the cells with 100 μl FACS Buffer, and detect the fluorescence signal by flow cytometry. The data were analyzed and graphed with GraphPad, and the results are shown in FIG. 20, which shows that Vedolizumab monoclonal antibody has internalization activity and can down-regulate the expression of α4β7 on Hut 78 membrane, and is not affected by TL1A protein. Internalization in the Vedolizumab+TLA0015-11 combo group is mediated by Vedolizumab monoclonal antibody and is therefore also not affected by the TL1A protein; TLb0045-1 and TLb0065-1 bispecific antibodies have good internalization activity, so in the absence of TL1A protein, the expression of α4β7 on the membrane can be down-regulated, and after the addition of TL1A protein, the internalization activity increases, and the expression of α4β7 on the membrane can be more significantly down-regulated.

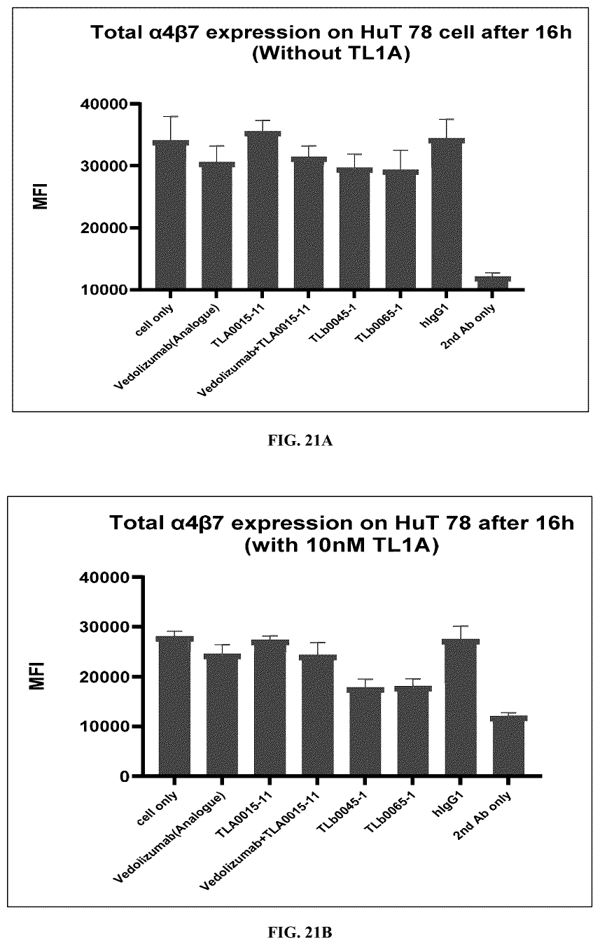

Detection of total α4β7: the second cell plate was removed at the end of incubation, centrifuged at 450 g for 5 min, discarded the supernatant, 200 μl of FACS Buffer was added to each well, centrifuged at 450 g for 5 min, discarded the supernatant, and repeated the step once; Live/Dead dye was configured at a ratio of 1:1000, and at the same time, 9.87 μl of Vedolizumab (concentration of 4.56 mg/mL), the final concentration of 100 nM, 100 μl of each well, incubation at 4° C. for 60 min; 450 g centrifugation for 5 min, discard the supernatant, 200 μl of FACS Buffer per well, repeat the step three times; 100 μl of 2% PFA Fix Solution per well, incubation at room temperature Add 100 μl 2% PFA Fix Solution per well, incubate at room temperature for 10 min, centrifuge at 600 g for 5 min, discard the supernatant, add 200 μl FACS Buffer per well, repeat the step twice; add 100 μl 0.1% Triton X-100 per well, incubate at ice for 7 min, centrifuge at 600 g for 5 min, discard the supernatant, add 200 μl FACS Buffer per well, repeat the step twice; add 100 μl 0.1% Triton X-100 per well, incubate at a final concentration of 100 nM Add Vedolizumab at a final concentration of 100 nM to each well and incubate at 4° C. for 60 min; after incubation, centrifuge at 600 g for 5 min, discard the supernatant, and add 200 μl of FACS Buffer to each well and repeat the procedure three times; Dilute the AF488 Goat anti-human Fc secondary antibody at a ratio of 1:200 and add 100 μl to each well and incubate for 30 min at 4° C., and then add the AF488 Goat anti-human Fc secondary antibody to each well. After incubation for 30 min at ° C., centrifuge at 600 g for 5 min, discard the supernatant, add 200 μl of FACS Buffer to each well, repeat the procedure three times, resuspend the cells with 100 μl of FACS Buffer, and detect the fluorescence signal by flow-through. The data were analyzed and plotted using GraphPad, and the results without TL1A protein are shown in FIG. 21A. TLb0045-1 and TLb0065-1 bispecific antibodies downregulated the expression of total α4β7 as well as Vedolizumab monoclonal antibody. With the addition of TL1A protein, the results are shown in FIG. 21B, the bispecific antibodies TLb0045-1 and TLb0065-1 could significantly down-regulate the expression of total α4β7 and were superior to Vedolizumab monoclonal antibody.

### Example 16 the Reverted Expression of α4P7 on the Membrane after Internalization of Bispecific Antibody was Detected

Based on flow cytometry assay, the relationship between the amount of α4β7 reverting to expression and time on the cell membrane after internalization of a plurality of bispecific antibodies prepared by example 1-3 of the present invention is detected.

Cultured Hut 78 cells were centrifuged at 450 g for 5 min, the supernatant was discarded, resuspended in freshly prepared RPMI1640 medium, the cell density was adjusted to 4E6/mL, and added to a 96-well low-adsorption U bottom plate, with a volume of 25 μl per well. pH Dye-labeled bispecific antibody and positive control antibody were diluted to 10 nM and 100 nM in RPMI1640 medium, respectively, and 50 μl/well were added to a 96-well plate. TL1A protein was diluted to 40 nM with RPMI1640 medium and 25 μl/well was added to the 96-well plate described above; For wells that do not require TL1A protein, add 25 μl of RPMI1640 medium, mix well with Hut 78 cells, and incubate in a 37° C. incubator for 24 hours. After the end of incubation, 450 g was centrifuged for 5 min, the supernatant was discarded, and the cells were washed twice using freshly prepared RPMI1640 medium. The first group is incubated with Vedolizumab-488 fluorescently labeled antibody at 4° C. for 60 min. After the end of incubation, centrifuge at 450 g for 5 min, discard the supernatant, wash the cells twice with flow cytometry, add an appropriate amount of flow cytometry buffer to resuspend the cells, and detect the fluorescence signal by flow cytometry. The second, third, and fourth groups were supplemented with 200 μl of RPMI1640 medium per well, and on the second, third, and fourth days, the newly expressed α4β7 molecule on the membrane was detected using Vedolizumab-488 fluorescently labeled antibody, respectively.

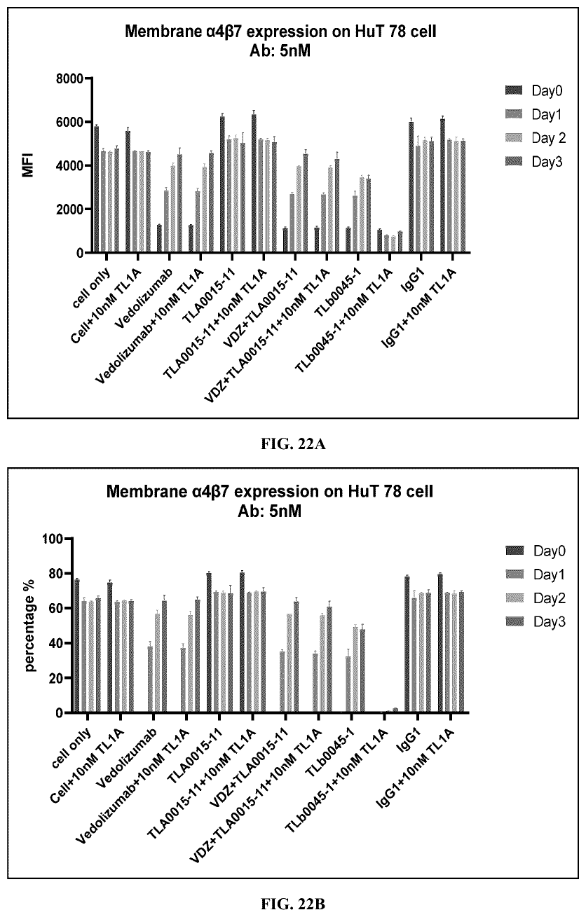

The results of antibody incubation concentration of 5 nM are shown in FIG. 22A (fluorescent signal), after 3 days of washing off the incubated antibody, the α4β7 molecule on the cell membrane surface of the cell membrane basically returned to the original level, and the addition of TL1A protein had no effect on the expression of α4β7 molecule on the membrane surface; Vedolizumab+TLA0015-11 in combination with vedolizumab monoclonal antibody results; The expression of α4β7 molecule on the membrane of TLb0045-1 bispecific antibody did not return to the original level after 3 days of washing off the incubated antibody The results indicated that TLb0045-1 bispecific antibody had a stronger inhibitory effect on α4β7 expression on the membrane surface than that of Vedolizumab monoclonal antibody group and Vedolizumab+TLA0015-11 group. TLb0045-1 had a strong inhibitory effect on the expression of α4β7 molecules on the upper surface of the membrane when TL1A protein was added, and the expression of α4β7 molecules on the membrane was still at a very low level after 3 days of washing off the incubated antibody and TL1A protein. The results of the proportion of positive cells are shown in FIG. 22B, and the trend of the results is consistent with the results of the fluorescence signal in FIG. 22A, TLb0045-1 has a good inhibition effect on the expression of α4β7 molecule, and the inhibition effect will be more obvious when TL1A protein is added.

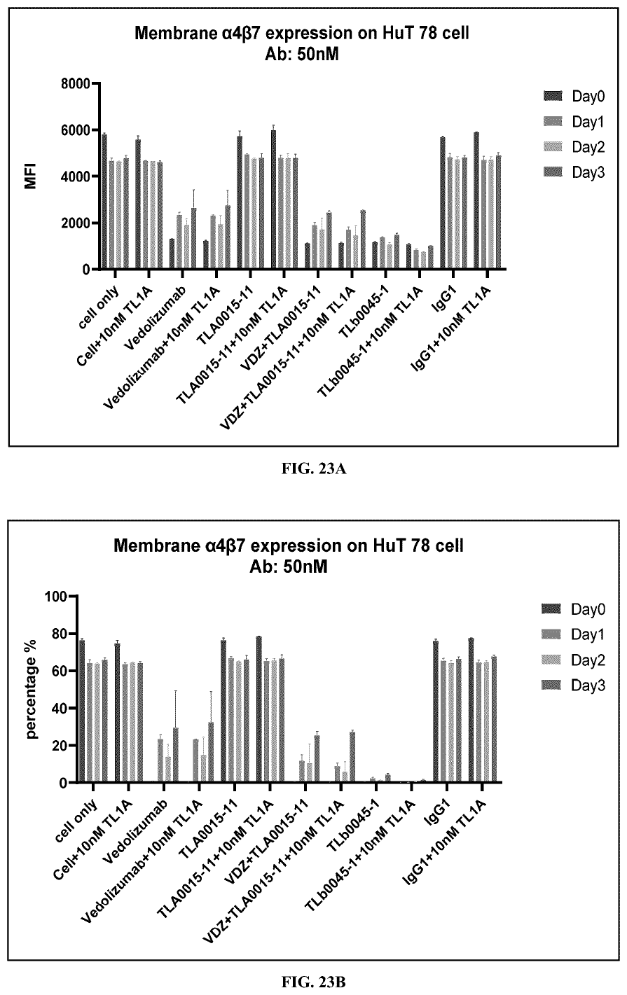

The results of the antibody incubation at 50 nM are shown in FIG. 23A (fluorescent signal) and FIG. 23B (proportion of positive cells), and the trend is consistent with the results of the 5 nM incubation, but due to the increase in antibody concentration, the expression of α4β7 molecule on the membrane surface has not returned to the original level after 3 days of washing off the incubation antibody. Among them, TLb0045-1 bispecific antibody had a stronger inhibitory effect on the expression of α4β7 molecule on the membrane than that of Vedolizumab monoclonal antibody group and Vedolizumab+TLA0015-11 group, and the inhibitory effect was more obvious after the addition of TL1A protein.

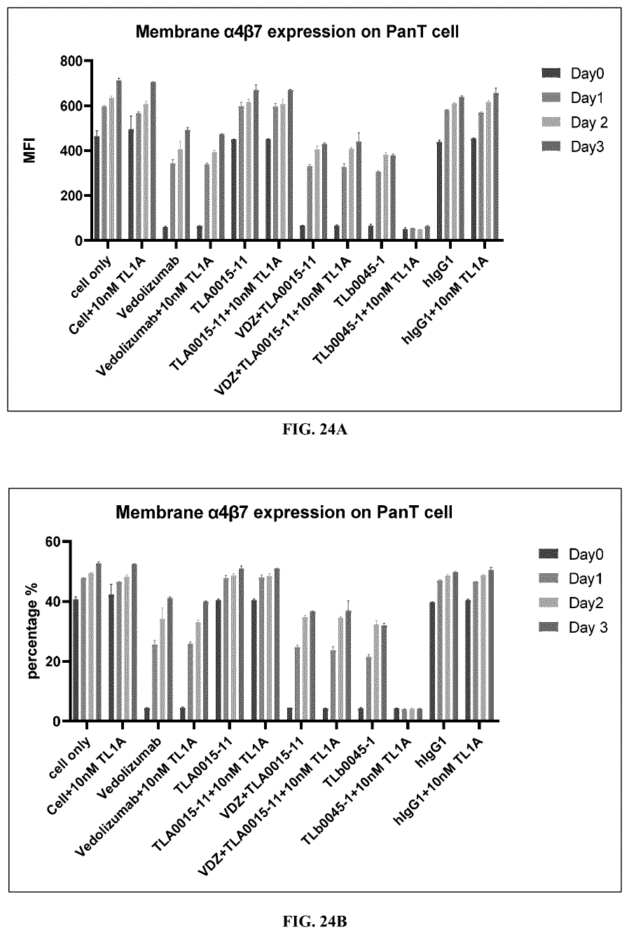

At the same time, PanT cells isolated from PBMC cells were used to detect the recovery of α4β7 expression on the membrane. The results were consistent with the results on HuT 78. As shown in FIGS. 24A and 24B, after the addition of TL1A protein, the inhibitory effect of TLb0045-1 bispecific antibody was more obvious. On day 3, the expression of Free α4β7 was still not detected on the cell surface.

### Example 17 Detection of the Inhibitory Activity of Bispecific Antibodies on PanT Cell Migration

Based on the Trans-well experimental method, the inhibitory activity of multiple bispecific antibodies prepared by Examples 1-3 of the present invention on T cell migration was detected.

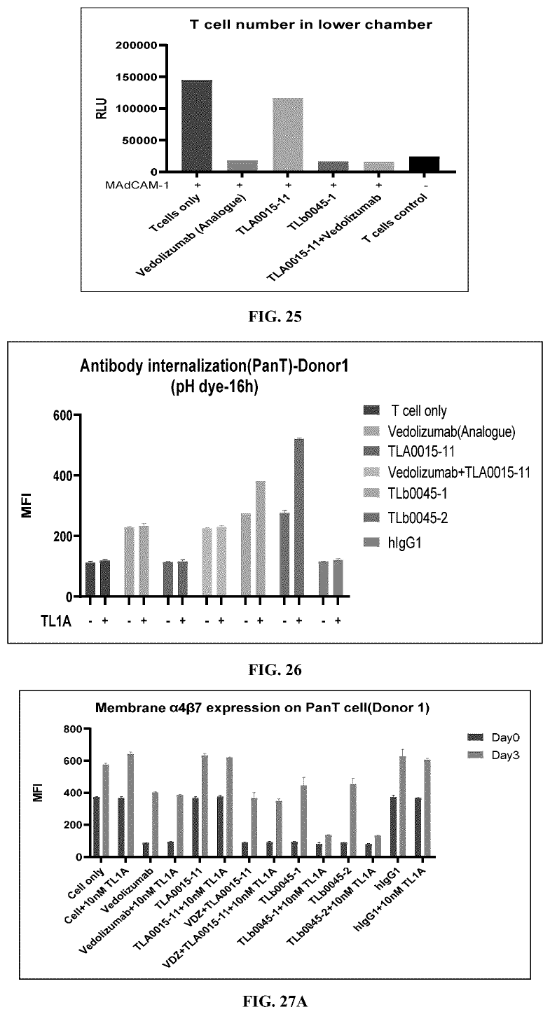

Cell adhesion and Trans-well combined experiment: MAdCAM-1 protein was diluted to 2 μg/ml with PBS, 100 μl/well, added to the chamber, and coated overnight at 4° C. The next day, the antibody was diluted with assay buffer (DPBS (w/o Ca2+, Mg2+)+1% BSA+0.5 mM MnCl2) to prepare an antibody concentration of 20 nM (final concentration of 5 nM); TL1A protein was prepared at a concentration of 40 nM (final concentration of 10 nM). According to the experimental design, 30 μl of the above-mentioned antibody and 30 μl of TL1A protein were added to each well of a 96-well round-bottom plate (30 μl assay buffer was added to the group without TL1A), and the 96-well round-bottom plate was placed on ice. Then, Pan T was resuspended in assay buffer (DPBS (w/o Ca2+, Mg2+)+1% BSA+0.5 mM MnCl2) to a density of 3.33×106 cells/mL, and 60 μl/well was inoculated in the above-mentioned 96-well round-bottom plate. After mixing, incubation was carried out on ice for 30 minutes. During the incubation process, the unbound proteins were washed away from the chamber coated with MAdCAM-1 his with PBST (PBS+0.5‰ Tween-20) for three times. After the incubation, 100 μl of the mixture in the 96-well round-bottom plate was transferred to the chamber coated with MAdCAM-1 and incubated on ice for 45 minutes. After the incubation time, the unbound Pan T-antibody mixture in the chamber was washed away with assay buffer and washed 4 times. Then add 100 μl of DPBS+2% FBS medium to the chamber, and add 600 μl of DPBS+2% FBS+500 ng/ml CCL25 medium to the 24-well plate below the chamber. Incubate the 24-well plate in a carbon dioxide incubator overnight. On the third day, carefully remove the chamber from the 24-well plate, add 300 μl of Bio-Lite Luciferase Assay reagent (Novozyme, DD1201-02) to each well, shake gently for 5 minutes, transfer 200 μl to each well in a new 96-well white plate, and then read the RLU value on a multifunctional microplate reader. Use Graphpad to make a bar graph of the sample and the measured signal value. The results of the combined cell adhesion and Trans-well experiment are shown in FIG. 25. Vedolizumab (Analogue), TLb0045-1 and TLA0015-11+Vedolizumab can all be seen to inhibit T cell migration.

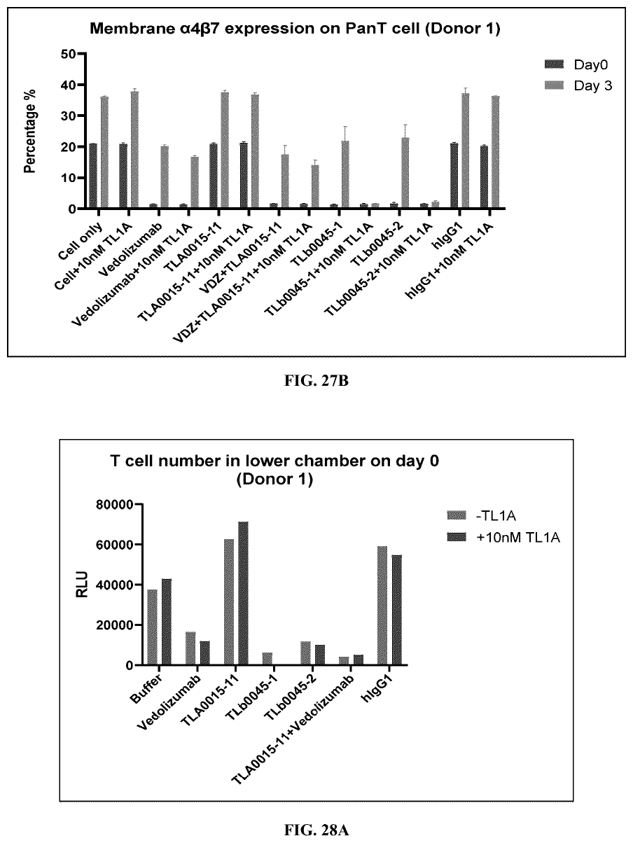

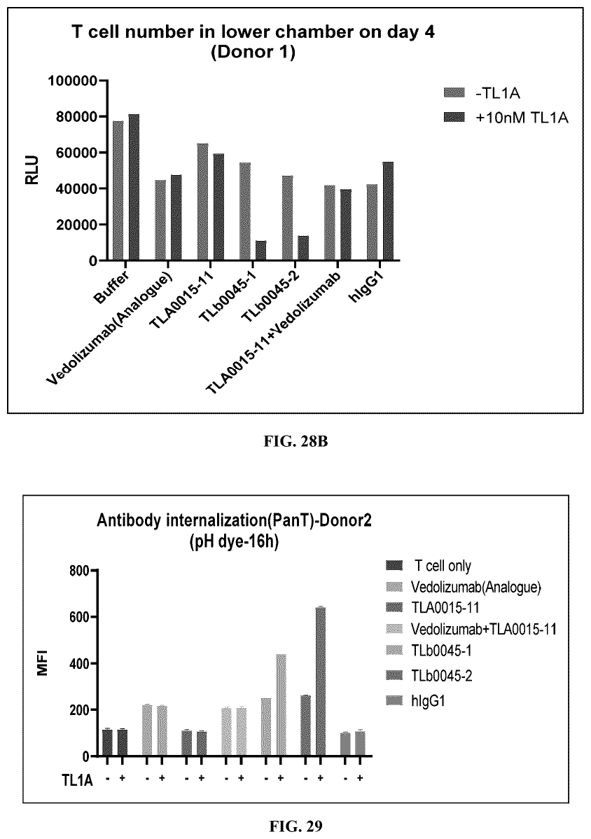

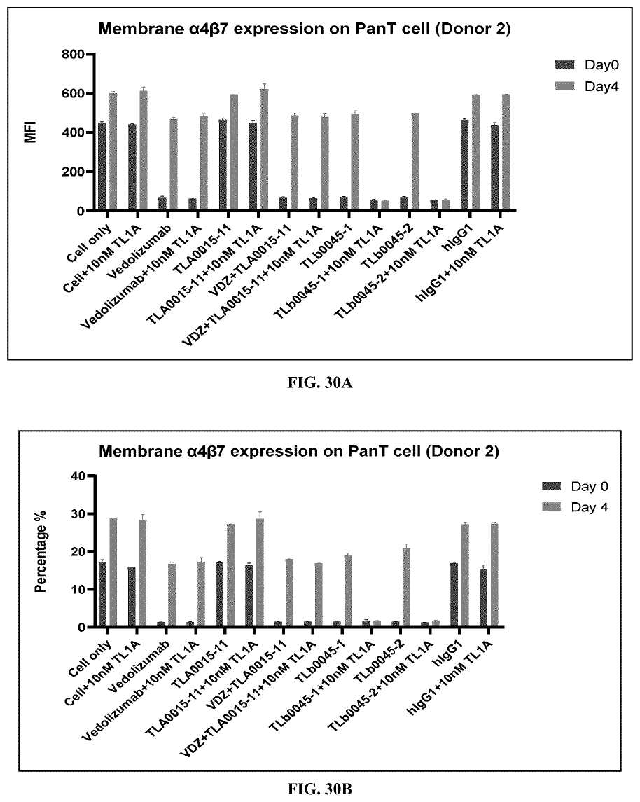

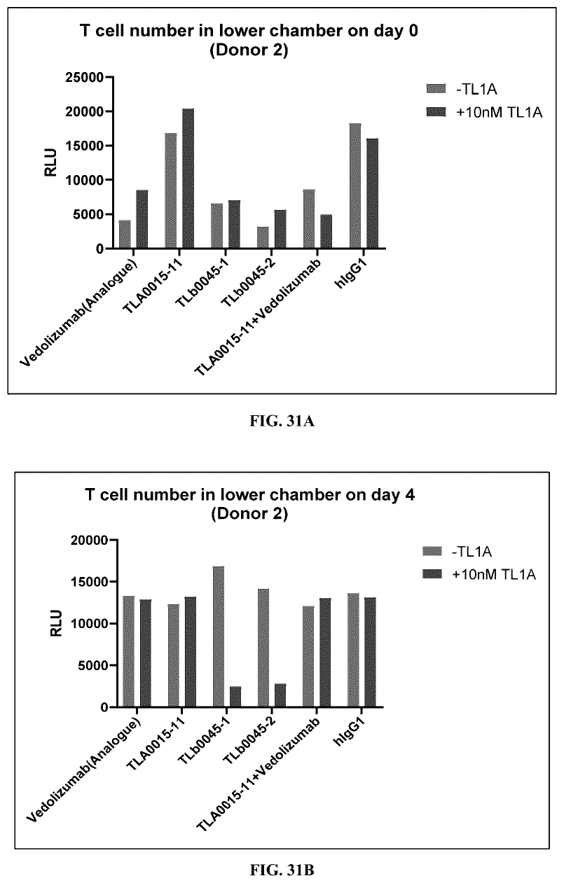

Combined experiment of α4β7 recovery expression on membrane surface and Trans-well: The α4β7 recovery experiment on membrane surface in Example 16 was combined with the Trans-well experiment. On day 0 and day 3, the α4β7 recovery expression on membrane surface was detected and the Trans-well experiment was performed. The results of the 16-hour endocytosis experiment are shown in FIG. 26, which shows that TL1A protein can specifically enhance the endocytosis activity of TLb0045-1 and TLb0045-2 bispecific antibodies; the expression levels of α4β7 on the membrane surface of PanT cells on Day 0 and Day 3 are shown in FIGS. 27A and 27B, which shows that TL1A protein can specifically enhance the inhibitory activity of TLb0045-1 and TLb0045-2 bispecific antibodies on the restoration of α4β7 expression on the membrane surface; the results of the Trans-well experiment on Day 0 are shown in FIG. 28A, which shows that on Day 0, Vedolizumab monoclonal antibody, TLA0015-11+Vedolizumab combination, TLb0045-1 and TLb0045-2 bispecific antibodies have basically the same inhibitory activity on T cell migration; The results of the Trans-well experiment on Day 4 are shown in FIG. 28B. It can be seen that the inhibitory activity on T cell migration in the monoclonal antibody group and the monoclonal antibody combination group is very weak in the presence or absence of TL1A because the expression of α4β7 on the membrane has been restored; TLb0045-1 and TLb045-2 bispecific antibodies are equivalent to monoclonal antibodies in the absence of TL1A, but in the presence of TL1A protein, it can be seen that the bispecific antibodies still have a strong inhibitory effect on T cell migration. The experiment was repeated on another PanT (Donor 2), and the results were completely consistent. The specific endocytosis results are shown in FIG. 29, the expression level of α4β7 on the membrane surface is shown in FIGS. 30A and 30B, and the results of Trans-well day 0 and day 4 are shown in FIGS. 31A and 31B, respectively.

### Example 18 Detection of the Inhibitory Activity of Bispecific Antibody on CD4T Cells

In this study, the inhibitory ability of bispecific antibodies on immune cell activation was evaluated by measuring the amount of cytokines in CD4+ T cells isolated from cryopreserved healthy human peripheral blood and their culture supernatants.

Dilute Anti-Human CD3 (Acro, CDE-M120a) with HBSS at a concentration of 5 μg/ml, add 96 cell culture flat-bottom plates, add 100 ul per well, and coat overnight at 4° C. Cryopreserved healthy human PBMCs (purchased from Saili Bio) were recovered into RPMI 1640 complete medium and incubated overnight at 37° C. The next day, after centrifugation of PBMCs (500 g for 5 min), discard the supernatant, resuspend the cells with EasySep™ Buffer (StemCell, Cat #20144), count them, and adjust the cell density to 5E7 cells/mL. CD4+ T cells were sorted according to the kit instructions using the CD4 negative selection kit (StemCell, Cat #17952). CD4+ T cells were washed twice with PBS, centrifuged at 500 g each for 5 min and discarded supernatant. Resuspend CD4+ T cells in RPMI 1640 complete medium, count, adjust cell density to 5E5 cells/mL for later use. Serial dilution of antibodies with 1640 complete medium, up to a maximum concentration of 400 nM, 10-fold dilution for a total of 5 points, set aside. Dilute huTL1A-his with 1640 complete medium at a concentration of 40 nM for later use. After the coated 96-well plate was washed once with 200 ul/well HBSS, the diluted MadCAM-1-his (Acro, MAM-H52H4-1 mg) diluted with HBSS was added at a concentration of 10 nM, 100 ul per well was added, and incubated at 37 degrees for 1 hour. After incubation, wash once with 200 ul/well HBSS, add CD4+ T cells 100 μl per well (final concentration is 5 w cells/well); Add huTL1A-his, 50 μl per well (final concentration is 10 nM); Add antibody 50 ul per well (final concentration up to 100 nM). Remove after 4 days of incubation at 37° C. Centrifuge at 500 g for 5 min, aspirate the cell supernatant and place on ice for subsequent cytokine detection.

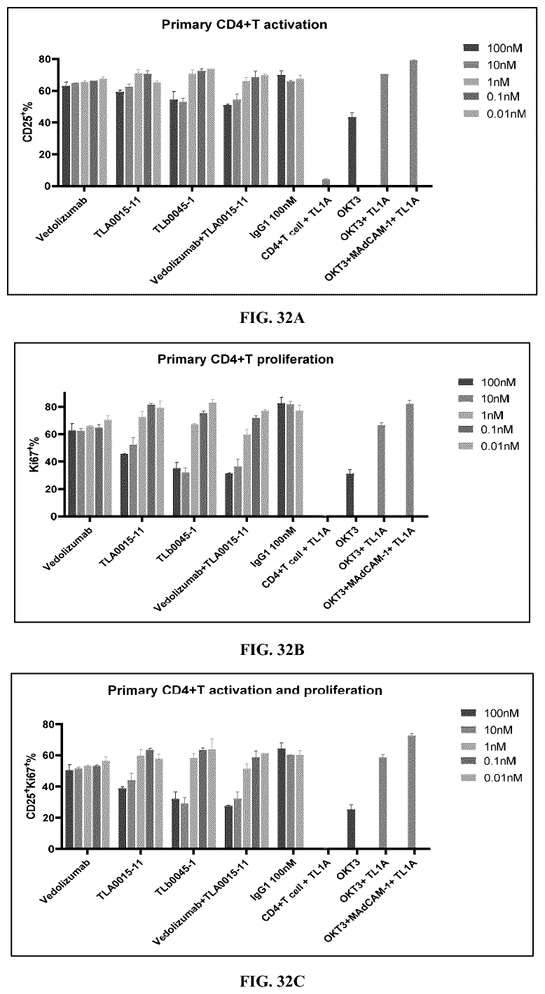

Cells were stained using PE anti-human CD25 (BioLegend, 356104) and Live/Dead dye and incubated at 4° C. for 30 min protected from light; FACS buffer was washed twice, and the membrane was fixed and ruptured using eBioscience™ Foxp3/Transcription Factor Flow Fixation Rupture Kit (Invitrogen, 00-5523-00) according to the instructions. 00-5523-00) for membrane fixation and rupture, resuspend the cells in 200 μl, incubate at 4° C. for 60 min under light protection; 1× rupture solution working solution was washed once, 1× rupture solution working solution diluted with flow-through antibody Alexa Fluor® 488 anti-human Ki-67 Antibody (BioLegend, 350508), incubate at room temperature under light protection for The cells were incubated for 30 min at room temperature and protected from light, washed twice with 1× membrane-breaking solution working solution and the supernatant was discarded, and finally the cells were resuspended with 100 μl FACS buffer, and the fluorescence signals of CD25 and Ki-67 were detected by cell flow meter. The FACS results of the assay are shown in FIG. 32A-C. TLb0045-1 bispecific antibody had a very good inhibitory effect on both proliferation and activation of CD4T cells, and was superior to Vedolizumab monoclonal antibody and TLA0015-11 monoclonal antibody.

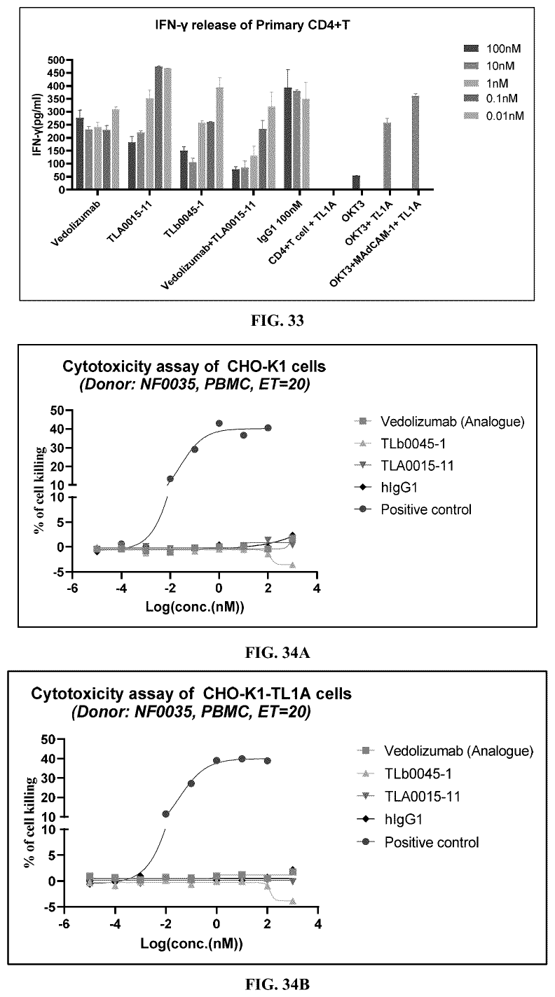

Cytokine IFN-γ levels in the supernatant were also assayed using the BD™ Cytometric Bead Array (human IFN-7) kit. Configure the standards according to the instructions, dilute the samples, 50 μl/well, and add to a 96-well plate. Configure Capture beads solution, 50 μl/well per well, add to 96-well plate and co-incubate the standard/sample with Capture beads solution for 1 hour. Dilute the Detection Antibody solution according to the instructions, 50 μl/well per well, add to the 96-well plate and co-incubate for 2 hours. Centrifuge at 300 g for 5 minutes, discard supernatant and wash again with wash buffer. After centrifugation again at 300 g for 5 minutes, the supernatant was discarded and resuspended in wash buffer, 70 μl/well, and the amount of cytokine IFN-γ was measured by Beckman CytoFLEX flow cytometer. The data obtained were fitted and analyzed by GraphPad Prism software, and the results of the IFN-γ concentration assay are shown in FIG. 33, which shows that the TLb0045-1 bispecific antibody has a good inhibitory effect on the secretion of IFN-γ.

### Example 19 LDH Method to Detect the T Cell-Dependent Cytotoxic Killing Effect Mediated by Bispecific Antibodies

This experiment uses the LDH method to detect whether bispecific antibodies will produce T cell-dependent cytotoxic killing effects. One end of the TLb0045-1 bispecific antibody targets TL1A, which is mainly expressed by endothelial cells, such as umbilical vein endothelial cells (HUVECs), monocytes, macrophages, dendritic cells (DC), T cells, chondrocytes and synovial fibroblasts, among which HUVECs have the highest expression level; the other end targets integrin α4β7, which is mainly expressed on the surface of lymphocytes and is responsible for mediating lymphocytes from the blood circulation into the intestines and central nervous system. Its abnormal function is closely related to human autoimmune diseases. In this experiment, TL1A overexpressing cell lines were co-incubated with PBMC under the condition of adding bispecific antibodies, and then LDH detection was used to evaluate whether bispecific antibodies would produce T cell-dependent cytotoxic killing effects.

The target cells used in the experiment were CHO-K1 (purchased from Nanjing Kebai Biotechnology) and CHO-K1-TL1A (overexpression of human full-length TL1A was constructed on CHO-K1 cells), and the effector cells were PBMCs from different donors. CHO-K1 and CHO-K1-TL1A cells were first digested and centrifuged (300 g, 5 min), resuspended in F-12K complete medium and counted, the cell density was adjusted to 1E5 cells/ml, and 100 μl/well was inoculated in a 96-well flat-bottom plate and cultured overnight. The next day, the antibody was diluted with 1640 complete medium to prepare an antibody of 2× analytical concentration, with a top dose of 2 μM (final of 1 μM), 10-fold gradient dilution, and a total of 9 concentration points. After the antibody was diluted, the 96-well flat-bottom plate inoculated with CHO-K1-TL1A cells was removed, the culture supernatant was aspirated, and 80 μl of the above-diluted antibody was added to each well. It is necessary to set up 2 target cell only wells (Tmax and Tmin wells) and 1 target cell+PBMC well (Buffer well). Continue to pre-incubate the 96-well flat-bottom plate in the CO2 incubator for 30 minutes. During the pre-incubation process, centrifuge the PBMCs that were revived one day in advance (300 g, 5 min), resuspend and count them with 1640 complete medium, and adjust the cell density to 2.5E6 cells/ml. After the pre-incubation time is over, take out the 96-well flat-bottom plate and inoculate the PBMCs with the adjusted density at 80 μl/well. Take out a new 96-well plate and add 80 μl of the above-diluted antibody and 80 μl of PBMCs with the adjusted density to each well. Place the three groups of 96-well plates in the CO2 incubator and continue to incubate for 48 h. 30 min-1 h before the end of incubation, add 20 μl of 10× Lysis buffer (Tmax) to one of the target cell only wells and continue to incubate in the CO2 incubator. After the incubation, the 96-well flat-bottom plate was removed from the incubator, centrifuged (400 g, 5 min), and 50 μl of the culture supernatant was aspirated from each well into a new 96-well flat-bottom plate. 50 μl of CytoTox 96® Non-Radioactive Cytotoxicity Assay (promega, G1780) substrate solution was added to each well of the 96-well flat-bottom plate. After incubation at room temperature and in the dark for 20 min, 50 μl of Stop Solution was added to each well after the color development time to terminate the reaction, and the absorbance value at 490 nm was detected by an enzyme reader. The killing ratio was calculated as follows: Lysis %=(OD490 Sample−OD490 Buffer)/(OD490 Tmax−OD490 Tmin)×100%. The logarithm of the antibody concentration was taken using Graphpad to perform a four-parameter fitting plot with the killing ratio. The killing results are shown in FIG. 33, where the killing of CHO-K1 cells after co-incubation with PBMC antibodies is shown in FIG. 34A. Except for the positive control, other antibodies have no killing effect; the killing of CHO-K1-TL1A cells after co-incubation with PBMC antibodies is shown in FIG. 34B. Except for the positive control, other antibodies have no killing effect; the results of co-incubation of simple antibodies with PBMC are shown in FIG. 34C. Except for the positive control, other antibodies have no killing effect.

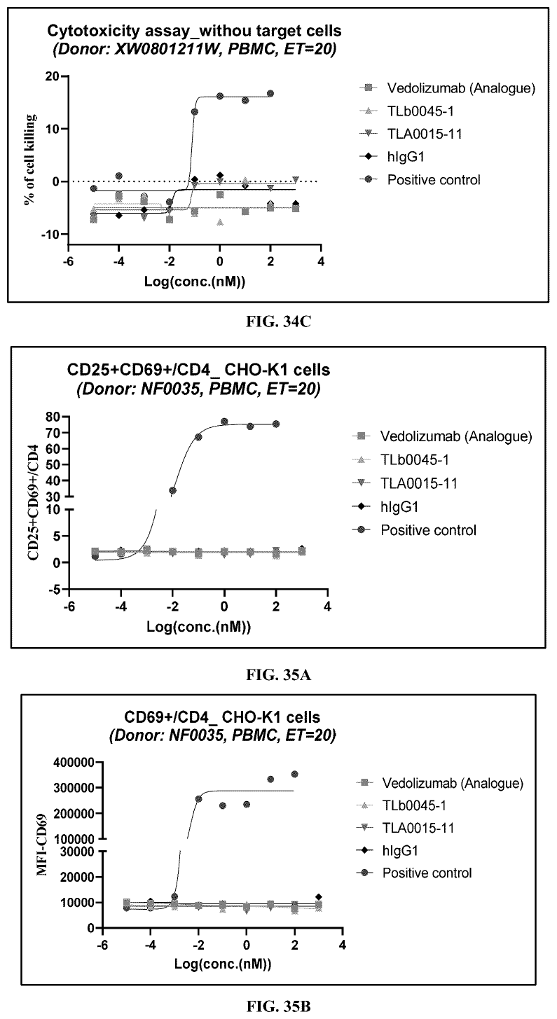

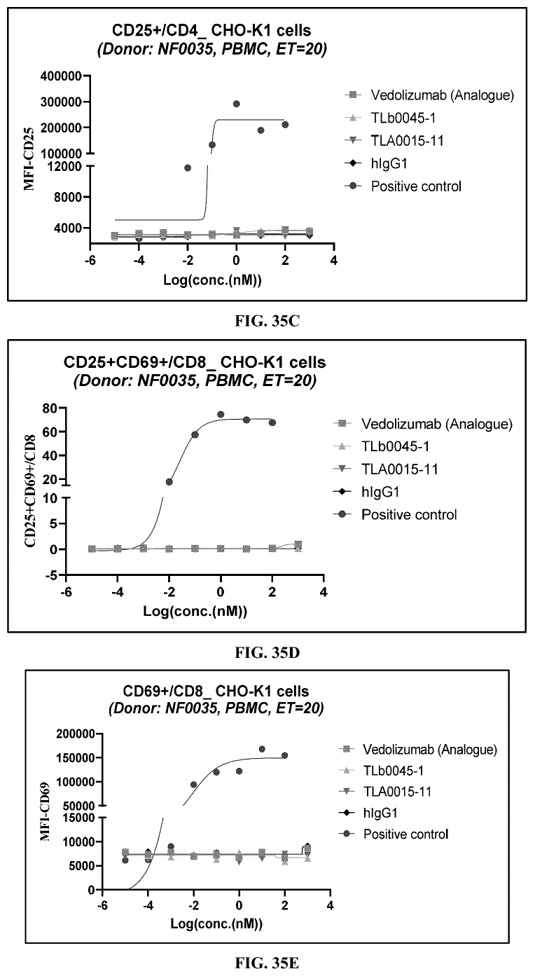

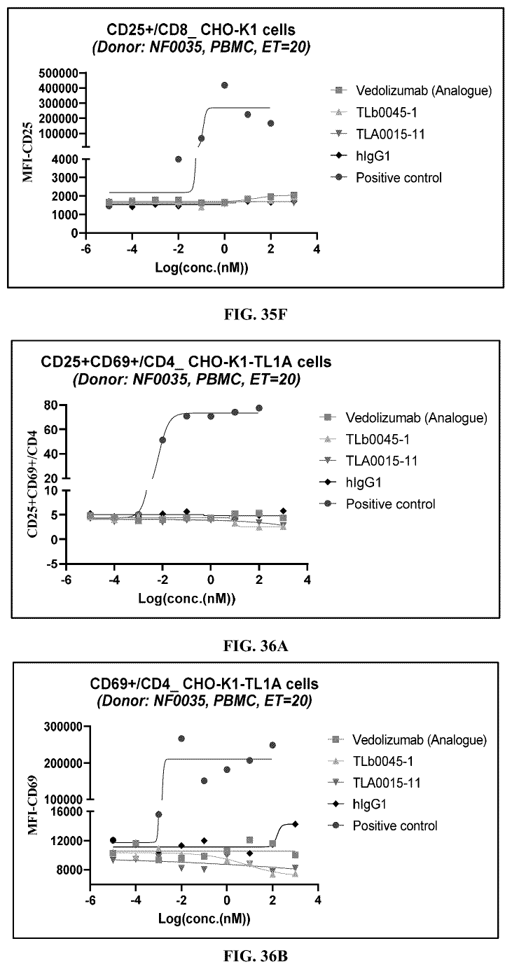

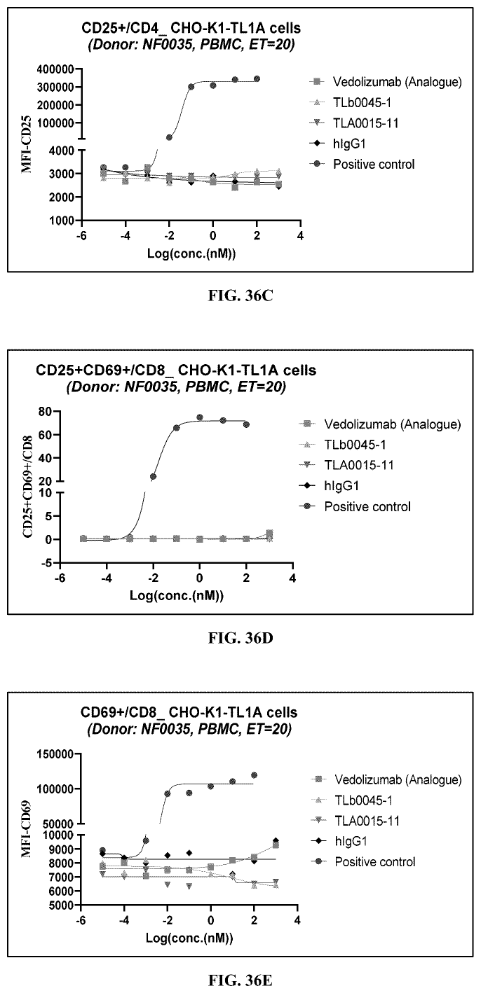

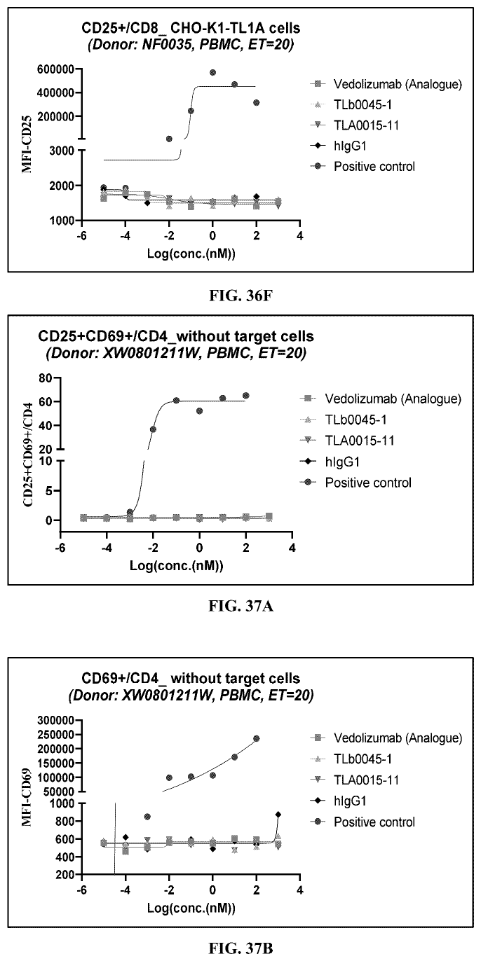

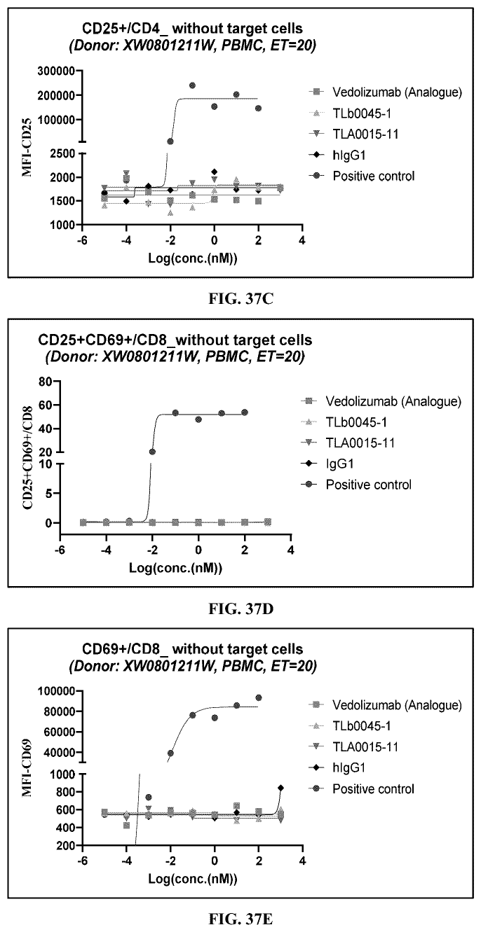

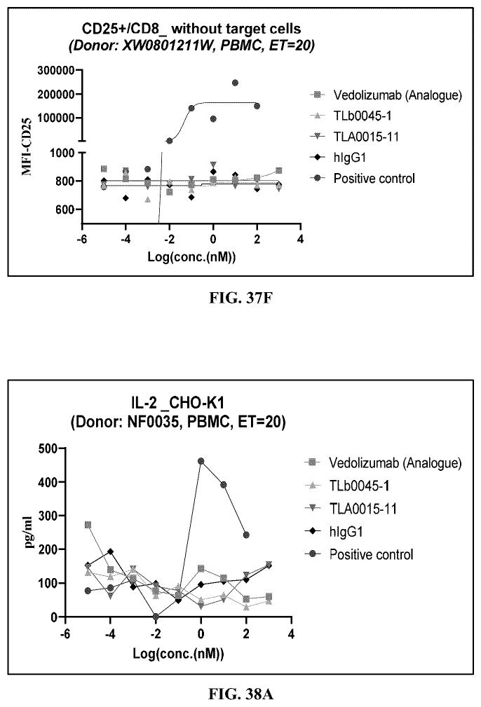

Bispecific antibody-mediated T cell activation detection: Prepare antibody mixture: Brilliant Violet 785™ anti-human CD3 Antibody (BioLegend, Cat #344842), Brilliant Violet 421™ anti-human CD8 Antibody (BioLegend, Cat #344748), BD Pharmingen™ APC Mouse Anti-Human CD69 (BD, Cat #555533), BD Pharmingen™ PE Mouse Anti-Human CD25 (BD, Cat #555432) at a ratio of 1:200, and dilute them with FACS buffer (PBS+1% FBS) for later use. In the above T cell-dependent cytotoxic killing experiment, the cell pellet after centrifugation (400 g, 5 min) was resuspended with antibody mixture, incubated at 4° C. for 30 min, washed with PBS to remove unbound antibodies 3 times, and finally resuspended with 80 μl PBS. The samples were collected on a flow cytometer (Agilent, NovoCyte Penteon) to analyze the proportion of CD25+CD69+ in the CD4+ cell population and the proportion of CD25+CD69+ in the CD8+ cell population. The four-parameter fitting plot was performed with Graphpad after taking the logarithm of the antibody concentration and the CD25+CD69+ ratio. The different T cell activation detection after CHO-K1 cells were co-incubated with PBMC antibodies is shown in FIG. 35; the different T cell activation detection after CHO-K1-TL1A cells were co-incubated with PBMC antibodies is shown in FIG. 36; the results of the simple antibody co-incubated with PBMC are shown in FIG. 37. The above experimental results are consistent. Except for the positive control, other antibodies have no function of activating T cells.

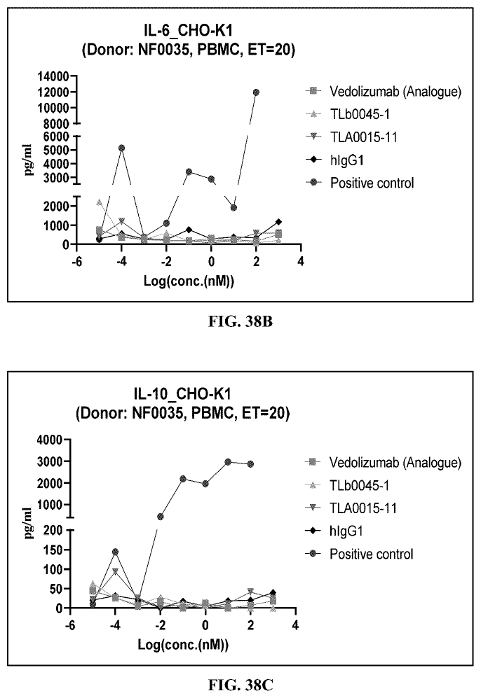

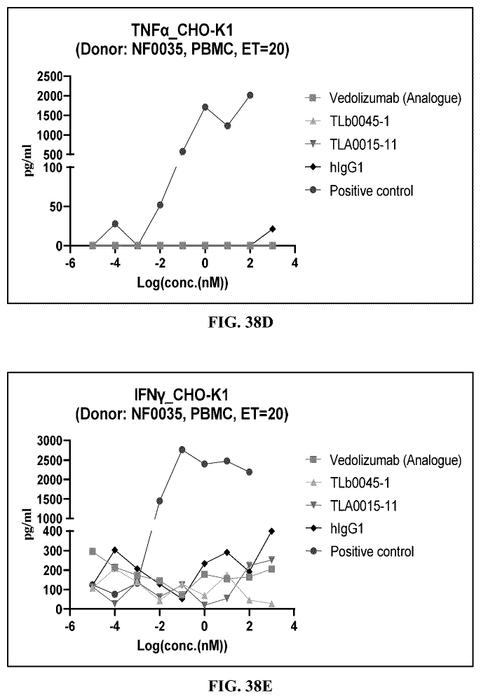

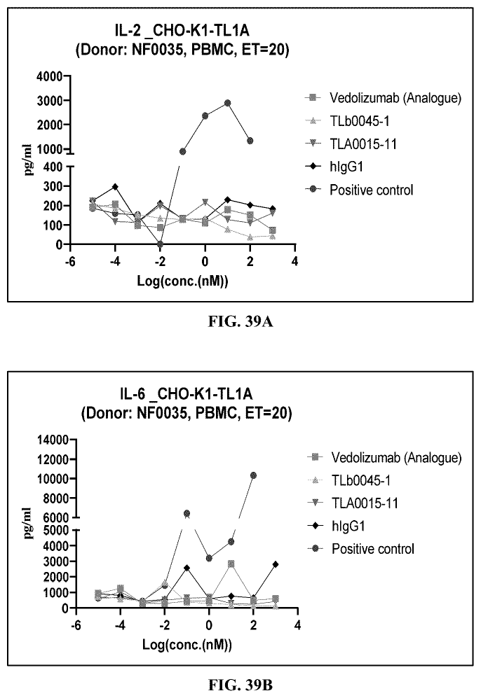

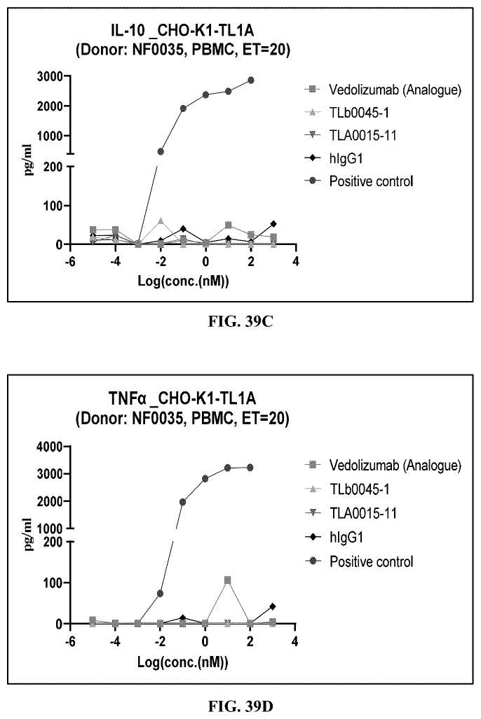

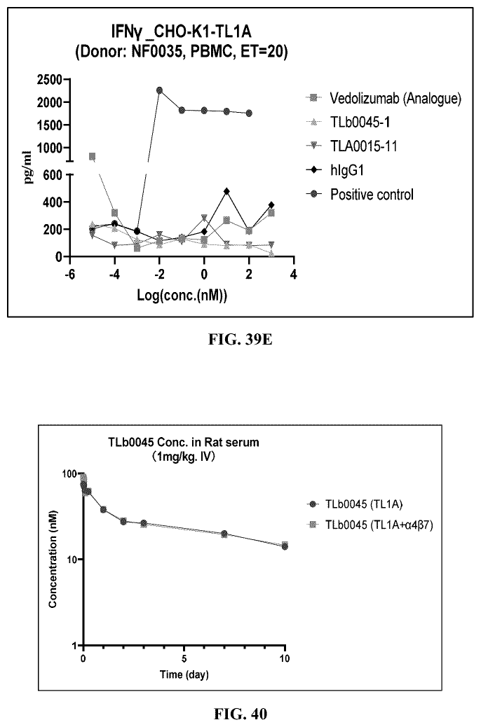

Cytokine release detection mediated by bispecific antibodies: For the above T cell-dependent cytotoxic killing experiment, after centrifugation (400 g, 5 min), 50 μl of culture supernatant was aspirated from each well into a new 96-well V-bottom plate, and then processed according to the instructions of BD™ Cytometric Bead Array (CBA) Human Th1/Th2 Cytokine Cytometric Bead Array (CBA) Kit II (BD, Cat #551809). Samples were collected on a flow cytometer (Agilent, NovoCyte Penteon), analyzed using FCAP Array v3 software, and finally Graphpad was used to perform a four-parameter fitting plot of the antibody concentration and the concentration of each cytokine. The specific results are shown in FIGS. 38 and 39. After CHO-K1 cells were co-incubated with PBMC antibodies, and after CHO-K1-TL1A cells were co-incubated with PBMC antibodies, the release of IL-2, IL-6, IL-10, TNF-α, and IFN-γ cytokines was not induced.

### Example 20 Detection of Bispecific Antibody Tm Value Detection

The experiment is based on a Panta instrument to detect the Tm values of a plurality of bispecific antibodies prepared by example 1-3 of the present invention. The Prometheus Panta instrument (NanoTemper Technologies GmbH) includes micro-differential scanning fluorescence (nanoDSF) technology, which tracks the folding state of a protein by detecting its endogenous fluorescence. The ratio of the fluorescence signal changes with increasing temperature or chemical denaturant concentration, thus determining the protein stability parameter Tm value. The specific Tm values of each antibody are shown in Table 8.

TABLE 8
The body Tm value of the anti-TL1A antibody

| | Tm(° C.) | | |
|---|---|---|---|
| sample | Tm1(° C.) | Tm2(° C.) | Ton-set(° C.) |
| TLb0045 | 66.89 | 83.59 | 59.04 |
| TLb0045-1 | 68.15 | 83.46 | 61.40 |
| TLb0065 | 61.20 | 83.58 | 55.70 |
| TLb0065-1 | 61.95 | 82.04 | 56.54 |

### Example 21 Detection of Bispecific Antibody Pharmacokinetics in Rats

In this experiment, SD rats were used to study the pharmacokinetics of bispecific antibodies in rats. At the same time, the stability of bispecific antibody molecules in rats was detected. Female rats with SPF grades were purchased from Weitong Lihua Laboratory Animal Technology Co., Ltd., and after the animals were quarantined, 5 mice were randomly assigned for each antibody. Each rat was injected with 1 mg/kg of bispecific antibodies TLb0045 and TLb0065 through the tail vein, and blood was subsequently collected at the time points in Table 9. Serum was collected by 100 μl/time point/only by jugular vein, placed at room temperature for 1 h, and centrifuged (8000 rpm, centrifugation for 5 min) at room temperature.

The first detection method: recombinant human TL1A-his protein was diluted to 1 μg/ml with PBS, added to the microplate at 100 μl/well, and incubated overnight at 4° C. Remove the coating solution, block at 200 μl/well, add 5% skim milk, and incubate for 2 h at room temperature. Remove the blocking solution and wash three times at 250 μl/well with 0.5‰ PBST and pat dry for later use. Dilute the serum sample to the appropriate ratio using the blocking solution and add it to the blocked plate. The standard curves were added sequentially to the blocked plate at 100 μl/well using TLb0045 and TLb0065 starting at 100 nM for a total of 20 concentration gradients (up to 100 nM), respectively, and incubated for 1 h at room temperature. Wash the plate 3 times with 0.5‰ PBST, add 10,000-fold dilution of HRP-labeled goat anti-human IgG antibody, 100 μl/well, and incubate for 60 min at room temperature. Wash the plate 5 times with 0.5‰ PBST, add TMB at 100 μl/well, incubate at room temperature in the dark for 5 min, at 100 μl/well, add stop solution, stop the substrate chromogenic reaction, read the OD value at 450 nm with a microplate reader, analyze the data with a GraphPad, plot and calculate the half-life (T½).

At the same time, the second method was used to quantify the antibody concentration in rat serum: first, Biotin conjugation of α4β7 protein was performed using a biotin labeling kit to obtain α4β7-Biotin protein for backup. Recombinant human TL1A-his protein is diluted to 1 μg/ml in PBS at 100 μl/well, added to the plate and incubated overnight at 4° C. Remove the coating solution, block at 200 μl/well, add 5% skim milk, and incubate at room temperature for 2 h. Remove the blocking solution and wash three times at 250 μl/well with 0.5‰ PBST and pat dry for later use. The diluted serum sample is added to the closed microplate label. The standard curves were added sequentially to the blocked plate at 100 μl/well using TLb0045 and TLb0065 starting at 100 nM for a total of 20 concentration gradients (up to 100 nM), respectively, and incubated for 1 h at room temperature. Wash the plate 3 times with 0.5‰ PBST, dilute α4β7-Biotin protein to 2 μg/ml and incubate at 100 μl/well for 60 min at room temperature; Add 5,000-fold diluted SA-HRP solution, incubate at room temperature for 60 min, wash the plate 5 times with 0.5‰ PBST at 100 μl/well, add TMB, and incubate at room temperature at 100 μl/well in the dark. The stop solution was added, the substrate chromogenic reaction was stopped, the OD value at 450 nm was read with a microplate reader, the data was analyzed with a GraphPad, plotted and the half-life (T½) was calculated.

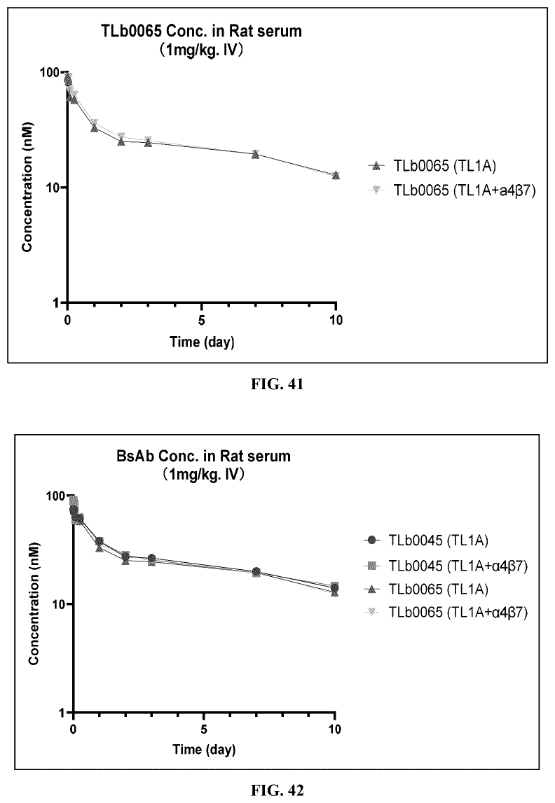

As shown in FIG. 40 and FIG. 41, the concentrations of bispecific antibody TLb0045 and TLb0065 in serum were basically the same as those detected by the two detection methods, indicating that the bispecific antibody molecule was stable in rats and was not easy to break. When TLb0045 and TLb0065 bispecific antibodies were compared together, there was little difference in serum concentrations, as shown in FIG. 42. The individual parameters of the PK calculation are shown in Table 10, and the half-life of the bispecific antibody is about 6-8 days.

TABLE 9
PK blood collection time point

| Mice | Point-in-time (5 mice/group) | | | | | | | | | | | |
|---|---|---|---|---|---|---|---|---|---|---|---|---|
| 1 | 5 min | 30 min | 1 h | 2 h | 6 h | 24 h | 48 h | 72 h | 168 h | 10 d | 14 d | 21 d |
| 2 | | | | | | | | | | | | |
| 3 | | | | | | | | | | | | |
| 4 | | | | | | | | | | | | |
| 5 | | | | | | | | | | | | |

TABLE 10
PK test of TLb0045 and TLb0065 in rats

| | N | Dose | T½ | CI | V | AUC |
|---|---|---|---|---|---|---|
| ID | Samples | (mg/kg) | (day) | (mL/d/kg) | (mL/kg) | (μg*d/mL) |
| TLb0045 | 10 | 1 | 8.40 | 11.73 | 142.13 | 85.29 |
| (TL1A) | | | | | | |
| TLb0045 | 10 | 1 | 8.81 | 11.30 | 143.61 | 88.51 |
| (TL1A + α4β7) | | | | | | |
| TLb0065 | 10 | 1 | 7.65 | 13.08 | 144.22 | 76.48 |
| (TL1A) | | | | | | |
| TLb0065 | 10 | 1 | 6.64 | 13.46 | 129.03 | 74.28 |
| (TL1A + α4β7) | | | | | | |

### Example 22 Detection of Pharmacokinetics of Bispecific Antibody in hFcRn Mice

In this experiment, B-hFcRn transgenic mice were used to study the pharmacokinetics of bispecific antibodies. FcRn is the receptor for antibody IgG, and its binding is pH-dependent. In a weakly acidic environment, the IgG-Fc segment exhibits a high affinity for FcRn, while in a neutral or alkaline environment, the IgG-Fc segment exhibits a low affinity for FcRn. When IgG is non-specific pinocytosis by cells, it enters the acidic environment of the endosome, and shows high affinity binding to FcRn on the endosomal membrane, and then is transported to the cell membrane by the circulating endosome, at this time, under alkaline conditions, the IgG-Fc segment has a very low affinity with FcRn, and IgG is released from the FcRn into the blood circulation, thereby increasing the half-life of IgG. When M428L and N434S mutations are introduced into IgG-Fc, the binding ability of Fc to FcRn receptor can be enhanced, and the half-life of IgG can be better extended. B-hFcRn transgenic mice (Catalog: 110001) were purchased from Biocytogen, and the mouse FcRn gene was knocked out on the C57BL/6 strain mice, and the human FcRn gene was knocked in, and the transgenic mouse only expressed the human FcRn receptor protein, but not the mouse FcRn receptor, so it can be used for metabolic kinetic studies of antibody drugs.

Female B-hFcRn transgenic mice with SPF grade were purchased from Biocytogen (Beijing) Pharmaceutical Technology Co., Ltd., and after the animals were quarantined, 10 mice were randomly assigned to each antibody, divided into 2 groups, 5 mice in each group, and blood was collected alternately. Each mouse is injected with 1 mg/kg of bispecific antibody via the tail vein and blood is then collected alternately at the time points in Table 11. Blood was collected by mandibular vein by collecting 20 μl/time point/piece, leaving it at room temperature for 1 h, and centrifugation (8000 rpm, centrifugation for 5 min) at room temperature.

The first detection method: recombinant human TL1A-his protein was diluted to 1 μg/ml with PBS, added to the microplate at 100 μl/well, and incubated overnight at 4° C. Remove the coating solution, block at 200 μl/well, add 5% skim milk, and incubate for 2 h at room temperature. Remove the blocking solution and wash three times at 250 μl/well with 0.5‰ PBST and pat dry for later use. Dilute the serum sample to the appropriate ratio using the blocking solution and add it to the blocked plate. The standard curves were added sequentially at 100 μl/well to the closed plate using TLb0045 and TLb0045-1, respectively, with 2-fold dilutions for a total of 20 concentration gradients (maximum concentration of 100 nM), at 100 μl/well, and incubated for 1 hour at room temperature. Wash the plate 3 times with 0.5‰ PBST, add 10,000-fold dilution of HRP-labeled goat anti-human IgG antibody, 100 μl/well, and incubate for 60 min at room temperature. Wash the plate 5 times with 0.5‰ PBST, add TMB at 100 μl/well, incubate at room temperature in the dark for 5 min, at 100 μl/well, add stop solution, stop the substrate chromogenic reaction, read the OD value at 450 nm with a microplate reader, analyze the data with a GraphPad, plot and calculate the half-life (T½).

At the same time, the second method was used to increase antibody concentrations in mouse serum: recombinant human TL1A-his protein was diluted to 1 μg/ml in PBS at 100 μl/well, added to a microplate plate, and incubated overnight at 4° C. Remove the coating solution, block at 200 μl/well, add 5% skim milk, and incubate at room temperature for 2 h. Remove the blocking solution and wash three times at 250 μl/well with 0.5‰ PBST and pat dry for later use. The diluted serum sample is added to the closed microplate label. The standard curves were added sequentially at 100 μl/well to the closed plate using TLb0045 and TLb0045-1, respectively, with 2-fold dilutions for a total of 20 concentration gradients (maximum concentration of 100 nM), at 100 μl/well, and incubated for 1 hour at room temperature. Wash the plate 3 times with 0.5‰ PBST, dilute α4β7-Biotin protein to 2 μg/ml and incubate at 100 μl/well for 60 min at room temperature; Add 5,000-fold diluted SA-HRP solution, incubate at room temperature for 60 min, wash the plate 5 times with 0.5‰ PBST at 100 μl/well, add TMB, and incubate at room temperature at 100 μl/well in the dark. The stop solution was added, the substrate chromogenic reaction was stopped, the OD value at 450 nm was read with a microplate reader, the data was analyzed with a GraphPad, plotted and the half-life (T½) was calculated.

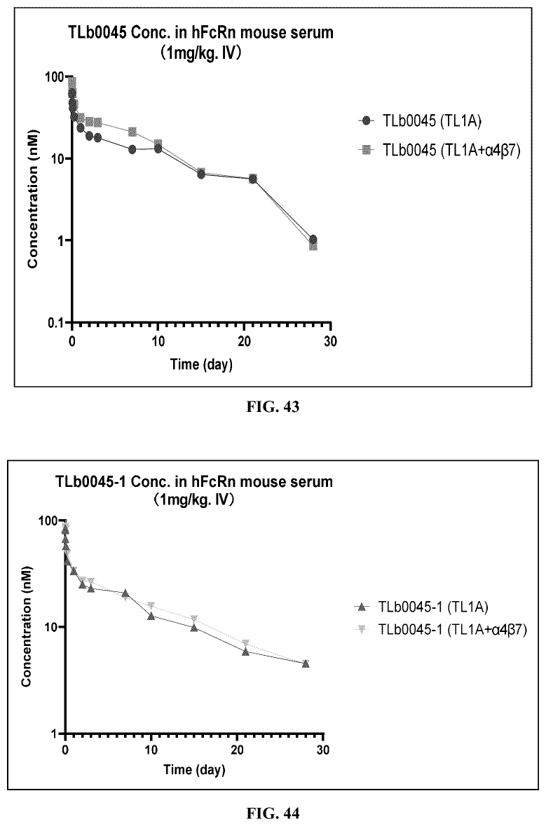

As shown in FIGS. 43 and 44, the concentrations of the bispecific antibodies TLb0045 and TLb0045-1 in serum were not significantly different. The half-life calculated by fitting the two test results of TLb0045 is 5-6 days, and the half-life calculated by fitting the two test results of TLb0045-1 is 9.7 days. The individual parameters of PK calculation are shown in Table 11.

TABLE 11
PK blood collection time point

| Mice | Point-in-time | | | | | | | | | | | | |
|---|---|---|---|---|---|---|---|---|---|---|---|---|---|
| 1 | 5 min | | 2 h | | 24 h | | 72 h | | 120 h | | 14 d | | 28 d |
| 2 | | | | | | | | | | | | | |
| 3 | | | | | | | | | | | | | |
| 4 | | | | | | | | | | | | | |
| 5 | | | | | | | | | | | | | |
| 6 | | 30 min | | 6 h | | 48 h | | 96 h | | 7 d | | 21 d | |
| 7 | | | | | | | | | | | | | |
| 8 | | | | | | | | | | | | | |
| 9 | | | | | | | | | | | | | |
| 10 | | | | | | | | | | | | | |

TABLE 12
PK test of TLb0045 and TLb0045-1 in hFcRn mice

| | N | Dose | T½ | CI | V | AUC |
|---|---|---|---|---|---|---|
| ID | Samples | (mg/kg) | (day) | (mL/d/kg) | (mL/kg) | (μg*d/mL) |
| TLb0045-1 | 13 | 1 | 9.7 | 2.23 | 31.31 | 447.44 |
| (TL1A) | | | | | | |
| TLb0045-1 | 13 | 1 | 9.7 | 2.03 | 28.54 | 491.83 |
| (TL1A + α4β7) | | | | | | |
| TLb0045 | 13 | 1 | 6.4 | 3.34 | 30.72 | 299.35 |
| (TL1A) | | | | | | |
| TLb0045 | 13 | 1 | 5.2 | 2.62 | 19.79 | 381.93 |
| (TL1A + α4β7) | | | | | | |

### Example 23 Detection of the Pharmacodynamic Activity of the Bispecific Antibody in the DSS-Induced hTL1A/hα4β7 Mouse Enteritis Model

This experiment used hTL1A/hα4β7 transgenic mice to study the in vivo pharmacodynamic activity of the bispecific antibody in the DSS-induced enteritis model. hTL1A/hα4β7 transgenic mice were purchased from Biocytogen (Beijing) Pharmaceutical Technology Co., Ltd. The humanized transgenic mice are C57BL/6 strain mice, which are hybridized from B-hTL1A transgenic mice and B-hα4β7 transgenic mice. The transgenic mice can express human TL1A and human α4β7 proteins at the same time. The mouse enteritis model induced by dextran sulfate sodium salt (DSS) is the most widely used chemically induced mouse IBD model. Acute ulcerative enteritis or chronic colitis is induced by dissolving DSS in drinking water, destroying the intestinal epithelial cells of mice, and nonspecific immune cells releasing cytokines, which ultimately leads to the destruction of the integrity of the mucosal barrier. The animals show obvious weight loss, loose stools, blood in stools, and granulocyte infiltration, which are extremely similar to human ulcerative colitis in clinical symptoms and pathological characteristics. Therefore, the DSS-induced enteritis model can be used to evaluate the in vivo pharmacodynamic activity of the bispecific antibody.

32 female hTL1A/hα4β7 transgenic mice were randomly divided into six groups according to their body weight, including two mice in the G1 negative control group, G2 modeling group, G3 TLA0015-11 drug group, G4 Vedolizumab (analog) drug group, G5 TLA0015-11+ Vedolizumab drug combination group, and G6 TLb0045-1 drug group, each with six mice. The detailed grouping of mice is shown in Table 13. The drug was injected intraperitoneally on days 0, 3, and 6, with a dose of 10 mg/kg. From day 0 to day 7, mice were given drinking water containing 3% DSS, which was replaced with normal drinking water from day 8 to day 9, and the experiment ended on day 9. During the experiment, the weight of the experimental animals was measured and recorded every two days, and the daily activities of the animals were observed; the fecal characteristics of the experimental animals were scored every day (0=normal, 1=moist/sticky, 2=soft, 3=liquid). After the experiment ended on day 9, all experimental animals were euthanized by asphyxiation with excessive carbon dioxide, and then the abdominal cavity was cut open, the colon was removed, and the colon length was recorded.

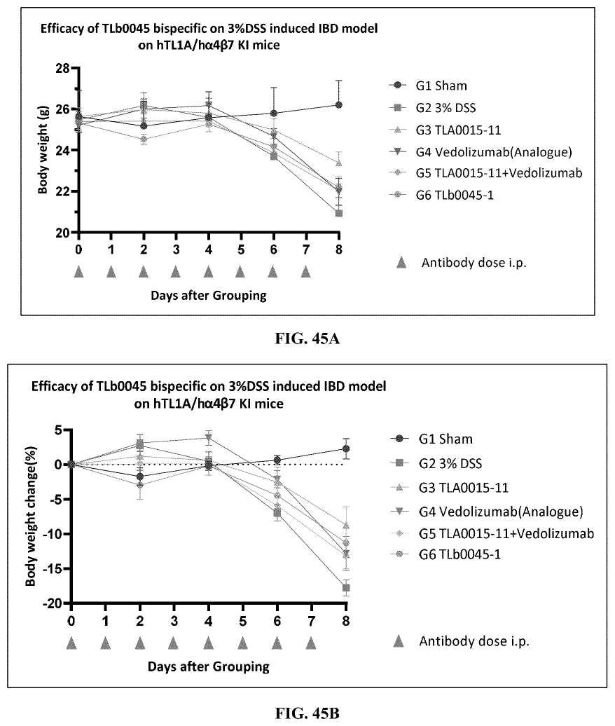

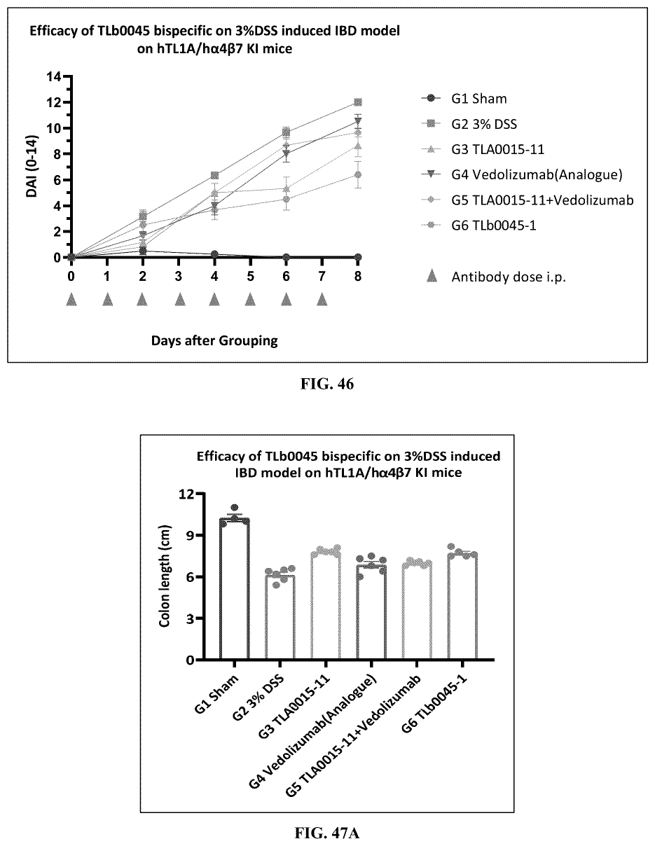

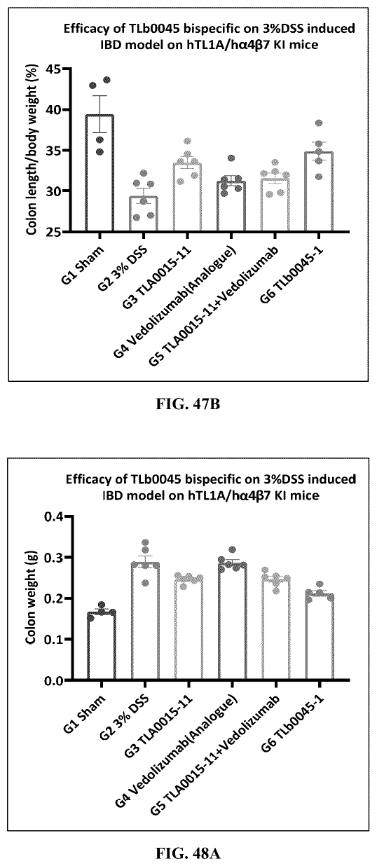

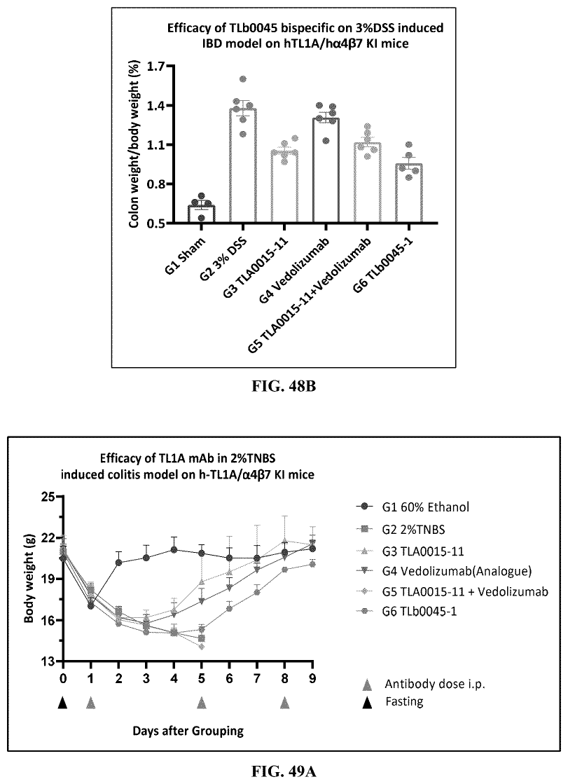

After the experiment, the results were statistically analyzed, and the results of the weight changes of mice are shown in FIGS. 45A and 45B. Compared with the G2 modeling group, the weight loss of mice in the four drug-administered groups was alleviated, and TLA0015-11 had the best efficacy. From the daily activity score results (FIG. 46), the TLb0045-1 drug-administered group had the lowest score and the best efficacy. At the end of the 9th day of the experiment, all mice were alive. The length and weight of the colorectum were measured after dissection. The results of the colorectal length of mice are shown in FIGS. 47A and 47B, and the results of the colorectal weight are shown in FIGS. 48A and 48B. The G2 modeling group can see that the colorectal length is significantly shortened and the weight is significantly increased. The TLA0015-11 and TLb0045-1 administration groups can see the efficacy of the drug in the colorectal length and weight indicators of mice.

TABLE 13
Mouse grouping and drug administration details (DSS-induced enteritis model)

| | | | Number of | | |
|---|---|---|---|---|---|
| Group | Animal | Modeling | Animals | Treatment | Dosing |
| Group 1 | hTL1A/hα4β7 | Sham | 2 | | |
| Group 2 | mice | DSS | 6 | PBS | |
| Group 3 | | | 6 | TLA0015-11 | 10 mg/kg |
| Group 4 | | | 6 | Vedolizumab | 10 mg/kg |
| | | | | (Analogue) | |
| Group 5 | | | 6 | TLA0015-11 + | 10 mg/kg + 10 mg/kg |
| | | | | Vedolizumab | |
| Group 6 | | | 6 | TLb0045-1 | 10 mg/kg |

### Example 24 Detection of the Pharmacodynamic Activity of the Bispecific Antibody in the TNBS-Induced hTL1A/hα4β7 Mouse Enteritis Model

This experiment used hTL1A/hα4β7 transgenic mice to study the in vivo pharmacodynamic activity of the bispecific antibody in the TNBS-induced enteritis model. TNBS-treated mice can establish a preclinical model that simulates clinical Crohn's disease (CD). The immune response produced is Th1-mediated, characterized by the infiltration of CD4+T cells, neutrophils, and macrophages, forming a horizontally progressive inflammation, leading to colitis. Therefore, the TNBS-induced enteritis model can be used to evaluate the in vivo pharmacodynamic activity of the bispecific antibody.

Twenty-seven female hTL1A/hα4β7 transgenic mice were randomly divided into six groups according to their body weight, including two mice in the G1 negative control group, five mice in the G2 modeling group, five mice in the G3 TLA0015-11 drug group, five mice in the G4 Vedolizumab (analog) drug group, five mice in the G5 TLA0015-11+Vedolizumab drug combination group, and five mice in the G6 TLb0045-1 drug administration group. The detailed grouping of mice is shown in Table 14. Intraperitoneal administration began on Day 1, and the drug was administered once every three days for a total of three times. All animals were fasted on Day 0; on Day 1, 100 μl 60% ethanol was given rectal administration (i.r.) to the G1 negative control group, and 100 μl 2% TNBS was given rectal administration (i.r.) to the G2 to G6 groups, and all animals were given a feed to induce acute enteritis. During the experiment, the weight of the experimental animals was measured and recorded every day, and the daily activities of the animals were observed; the fecal characteristics of the experimental animals were scored every day (0=normal, 1=moist/sticky, 2=soft, 3=liquid). After the experiment ended on the 9th day, all experimental animals were euthanized by asphyxiation with excessive carbon dioxide, and then the abdominal cavity was cut open, the colon was removed, and the colon length was recorded.

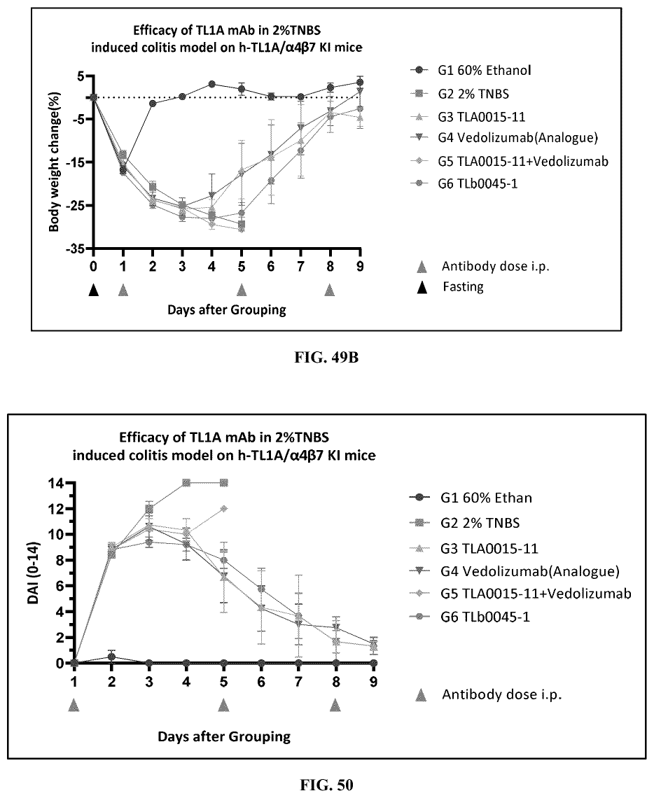

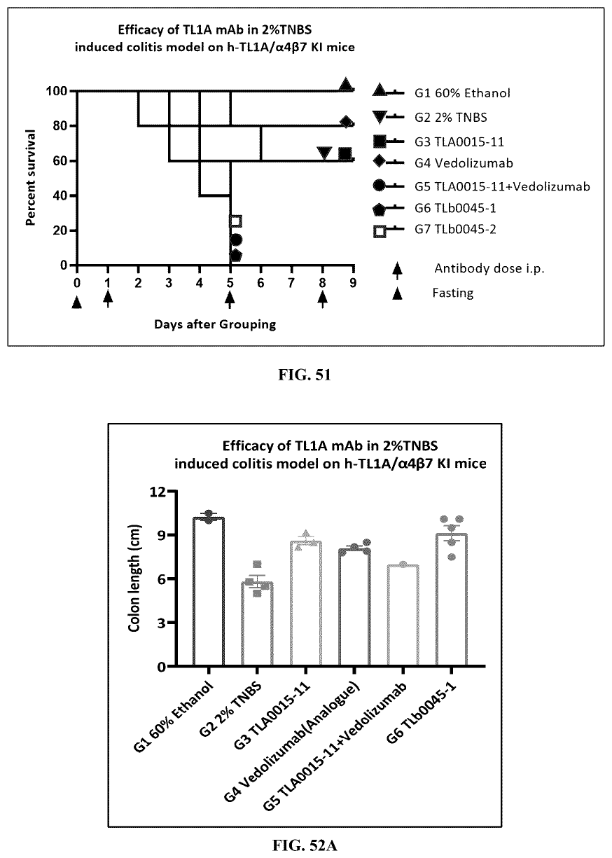

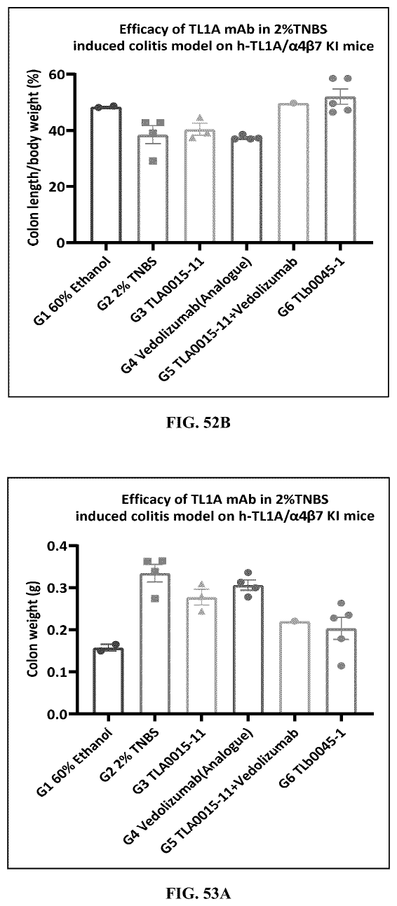

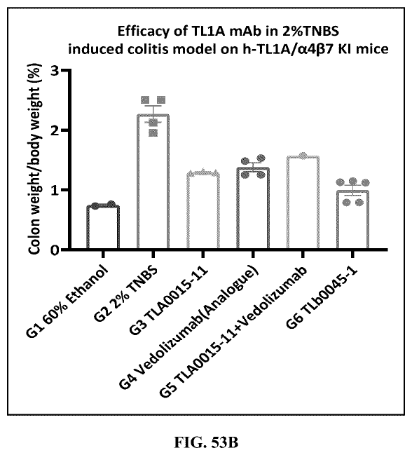

After the experiment, the results were statistically analyzed, and the results of the weight change of mice are shown in FIGS. 49A and 49B. Compared with the G2 modeling group, the weight of mice in the TLA0015-11 drug group, the Vedolizumab (analog) drug group and the TLb0045-1 drug group can be seen to have recovered. From the daily activity score results (FIG. 50), the scores of the TLA0015-11 drug group, the Vedolizumab (analog) drug group and the TLb0045-1 drug group began to decrease after the 4th day. The animal survival curve results are shown in FIG. 51, where all mice in the modeling group and the combination group died on the 5th day. At the end of the 9th day, some other mice also died. After dissection, the length and weight of the colorectum were measured. The results of the colorectal length of mice are shown in FIGS. 52A and 52B, and the results of the colorectal weight are shown in FIGS. 53A and 53B. The G2 modeling group can see that the colorectal length is significantly shortened and the weight is significantly increased. TLA0015-11, Vedolizumab (analog) and bispecific antibody TLb0045-1 can all see the efficacy.

TABLE 14
Mouse grouping and drug administration details (TNBS-induced enteritis model)

| | | | Number of | | |
|---|---|---|---|---|---|
| Group | Animal | Modeling | Animals | Treatment | Dosing |
| Group 1 | hTL1A/hα4β7 | Sham | 2 | | |
| Group 2 | mice | TNBS | 6 | PBS | |
| Group 3 | | | 6 | TLA0015-11 | 10 mg/kg |
| Group 4 | | | 6 | Vedolizumab | 10 mg/kg |
| | | | | (Analogue) | |
| Group 5 | | | 6 | TLA0015-11 + | 10 mg/kg + 10 mg/kg |
| | | | | Vedolizumab | |
| Group 6 | | | 6 | TLb0045-1 | 10 mg/kg |

In summary, the sequences used in the present application are shown in the table below, wherein [ ] represents that the position can be arbitrarily replaced by the amino acids shown in [ ].

SEQUENCE LISTING

| ID | LABEL | SEQ |
|---|---|---|
| 1 | TLA0015-HCDR1 | [STY]YGVD |
| 2 | TLA0015-HCDR1-1 | SYGVD |
| 3 | TLA0015-HCDR1-2 | YYGVD |
| 4 | TLA0015-HCDR1-3 | TYGVD |
| 5 | TLA0015-HCDR2 | [RV]IWGFG[GK]T[DHN]Y[NQ][APS]ALKS |
| 6 | TLA0015-HCDR2-1 | VIWGFGGTNYNSALKS |
| 7 | TLA0015-HCDR2-2 | VIWGFGGTNYQSALKS |
| 8 | TLA0015-HCDR2-3 | VIWGFGGTNYNAALKS |
| 9 | TLA0015-HCDR2-4 | VIWGFGGTNYNPALKS |
| 10 | TLA0015-HCDR2-5 | VIWGFGGTHYNSALKS |
| 11 | TLA0015-HCDR2-6 | RIWGFGGTNYNSALKS |
| 12 | TLA0015-HCDR2-7 | VIWGFGKTNYNSALKS |
| 13 | TLA0015-HCDR2-8 | VIWGFGGTDYNSALKS |
| 14 | TLA0015-HCDR3 | G[DLN][FY]DAMDY |
| 15 | TLA0015-HCDR3-1 | GNFDAMDY |
| 16 | TLA0015-HCDR3-2 | GNYDAMDY |
| 17 | TLA0015-HCDR3-3 | GDFDAMDY |
| 18 | TLA0015-HCDR3-4 | GLFDAMDY |
| 19 | TLA0015-HFR1 | E[IV]QL[LV]ESGGGL[IV]QPGGSLR[IL]SCAVSGF |
| | | [DS]L[IKS] |
| 20 | TLA0015-HFR1-1 | EIQLLESGGGLVQPGGSLRLSCAVSGFSLS |
| 21 | TLA0015-HFR1-2 | EIQLLESGGGLVQPGGSLRISCAVSGFSLS |
| 22 | TLA0015-HFR1-3 | EIQLVESGGGLVQPGGSLRISCAVSGFSLS |
| 23 | TLA0015-HFR1-4 | EVQLLESGGGLVQPGGSLRLSCAVSGFSLS |
| 24 | TLA0015-HFR1-5 | EIQLVESGGGLIQPGGSLRLSCAVSGFSLS |
| 25 | TLA0015-HFR1-6 | EIQLVESGGGLIQPGGSLRISCAVSGESLS |
| 26 | TLA0015-HFR1-7 | EIQLVESGGGLIQPGGSLRISCAVSGFSLI |
| 27 | TLA0015-HFR1-8 | EIQLVESGGGLIQPGGSLRISCAVSGFSLK |
| 28 | TLA0015-HFR1-9 | EIQLVESGGGLIQPGGSLRISCAVSGFDLS |
| 29 | TLA0015-HFR2 | WVRQ[AS]PGKGLEW[LV]G |
| 30 | TLA0015-HFR2-1/ | WVRQAPGKGLEWVG |
| | TLA-0238-HFR2-3 | |
| 31 | TLA0015-HFR2-2 | WVRQAPGKGLEWLG |
| 32 | TLA0015-HFR2-3 | WVRQSPGKGLEWLG |
| 33 | TLA0015-HFR3 | R[FL]TIS[AKRV]DNSKNT[LV]YLQMNSLRA |
| | | EDTAVYYCA[RS] |
| 34 | TLA0015-HFR3-1 | RLTISRDNSKNTLYLQMNSLRAEDTAVYYCAS |
| 35 | TLA0015-HFR3-2 | RLTISKDNSKNTVYLQMNSLRAEDTAVYYCAS |
| 36 | TLA0015-HFR3-3 | RFTISRDNSKNTVYLQMNSLRAEDTAVYYCAS |
| 37 | TLA0015-HFR3-4 | RLTISRDNSKNTVYLQMNSLRAEDTAVYYCAS |
| 38 | TLA0015-HFR3-5 | RLTISRDNSKNTVYLQMNSLRAEDTAVYYCAR |
| 39 | TLA0015-HFR3-6 | RLTISVDNSKNTVYLQMNSLRAEDTAVYYCAS |
| 40 | TLA0015-HFR3-7 | RLTISADNSKNTVYLQMNSLRAEDTAVYYCAS |
| 41 | TLA0015-HFR4-1/ | WGQGTLVTVSS |
| | TLA-0238-HFR4-2 | |
| 42 | TLA0015-LCDR1-1 | RASQDISNYLN |
| 43 | TL0015-LCDR2-1 | YTSRLHS |
| 44 | TL0015-LCDR3-1 | QQGNTLPFT |
| 45 | TL0015-LFR1 | DIQMTQ[ST][PT]SSLSASVGDRVTI[TS]C |
| 46 | TL0015-LFR1-1 | DIQMTQTTSSLSASLGDRVTISC |
| 47 | TL0015-LFR1- | DIQMTQSPSSLSASVGDRVTITC |
| | 2/TLA0241-LFR1-1 | |
| 48 | TL0015-LFR2 | WYQQKPGK[AV][PV]KLLIY |
| 49 | TL0015-LFR2-1 | WYQQKPDGTVKLLIY |
| 50 | TL0015-LFR2-2 | WYQQKPGKVVKLLIY |
| 51 | TL0015-LFR2-3 | WYQQKPGKAVKLLIY |
| 52 | TL0015-LFR2-4/ | WYQQKPGKAPKLLIY |
| | TLA0241-LFR2-1 | |
| 53 | TL0015-LFR3 | GVPSRFSGSGSGTD[FY]TLTISSLQPED[FV]ATY[FY] |
| | | C |
| 54 | TL0015-LFR3-1 | GVPSRFSGSGSGTDYSLTISNLEQEDIATYFC |
| 55 | TL0015-LFR3-2 | GVPSRFSGSGSGTDYTLTISSLQPEDVATYFC |
| 56 | TL0015-LFR3-3 | GVPSRFSGSGSGTDYTLTISSLQPEDFATYFC |
| 57 | TL0015-LFR3-4/ | GVPSRFSGSGSGTDFTLTISSLQPEDFATYYC |
| | TLA0238-LFR3-4/ | |
| | TLA0241-LFR3-2 | |
| 58 | TL0015-LFR4 | FG[PQ]GTKLEIK |
| 59 | TL0015-LFR4-1 | FGSGTKLEIK |
| 60 | TL0015-LFR4-2 | FGPGTKLEIK |
| 61 | TL0015-LFR4-3/ | FGQGTKLEIK |
| | TLA0238-LFR4-3 | |
| 62 | TLA0015-VH-1 | QIQLKESGPGLVAPSQSLSITCTVSGFSLSSYGVDWV |
| | | RQSPGKGLEWLGVIWGFGGTNYNSALKSRLSISKDN |
| | | SKSQVFLKMNSLQSDDSAIYYCASGNFDAMDYWGQ |
| | | GTSVTVSS |
| 63 | TLA0015-VH-2 | EIQLLESGGGLVQPGGSLRLSCAVSGFSLSSYGVDW |
| | | VRQAPGKGLEWVGVIWGFGGTNYNSALKSRLTISR |
| | | DNSKNTLYLQMNSLRAEDTAVYYCASGNFDAMDY |
| | | WGQGTLVTVSS |
| 64 | TLA0015-VH-3 | EIQLLESGGGLVQPGGSLRISCAVSGFSLSSYGVDWV |
| | | RQAPGKGLEWLGVIWGFGGTNYNSALKSRLTISKD |
| | | NSKNTVYLQMNSLRAEDTAVYYCASGNFDAMDYW |
| | | GQGTLVTVSS |
| 65 | TLA0015-VH-4 | EIQLVESGGGLVQPGGSLRISCAVSGFSLSSYGVDW |
| | | VRQSPGKGLEWLGVIWGFGGTNYNSALKSRLTISKD |
| | | NSKNTVYLQMNSLRAEDTAVYYCASGNFDAMDYW |
| | | GQGTLVTVSS |
| 66 | TLA0015-VH-5 | EVQLLESGGGLVQPGGSLRLSCAVSGFSLSSYGVDW |
| | | VRQAPGKGLEWLGVIWGFGGTNYNSALKSRFTISRD |
| | | NSKNTVYLQMNSLRAEDTAVYYCASGNFDAMDYW |
| | | GQGTLVTVSS |
| 67 | TLA0015-VH-6 | EVQLLESGGGLVQPGGSLRLSCAVSGFSLSSYGVDW |
| | | VRQAPGKGLEWLGVIWGFGGTNYNSALKSRLTISR |
| | | DNSKNTVYLQMNSLRAEDTAVYYCASGNFDAMDY |
| | | WGQGTLVTVSS |
| 68 | TLA0015-VH-7 | EVQLLESGGGLVQPGGSLRLSCAVSGFSLSSYGVDW |
| | | VRQAPGKGLEWLGVIWGFGGTNYNSALKSRLTISK |
| | | DNSKNTVYLQMNSLRAEDTAVYYCASGNFDAMDY |
| | | WGQGTLVTVSS |
| 69 | TLA0015-VH-8 | EIQLVESGGGLIQPGGSLRLSCAVSGFSLSSYGVDWV |
| | | RQAPGKGLEWLGVIWGFGGTNYNSALKSRLTISRD |
| | | NSKNTVYLQMNSLRAEDTAVYYCARGNFDAMDY |
| | | WGQGTLVTVSS |
| 70 | TLA0015-VH-9 | EIQLVESGGGLIQPGGSLRISCAVSGFSLSSYGVDWV |
| | | RQAPGKGLEWLGVIWGFGGTNYNSALKSRLTISKD |
| | | NSKNTVYLQMNSLRAEDTAVYYCASGNFDAMDYW |
| | | GQGTLVTVSS |
| 71 | TLA0015-VH-10 | EIQLVESGGGLIQPGGSLRISCAVSGFSLSSYGVDWV |
| | | RQAPGKGLEWLGVIWGFGGTNYNSALKSRLTISRD |
| | | NSKNTVYLQMNSLRAEDTAVYYCASGNFDAMDYW |
| | | GQGTLVTVSS |
| 72 | TLA0015-VH-11 | EIQLVESGGGLIQPGGSLRISCAVSGFSLSSYGVDWV |
| | | RQAPGKGLEWLGVIWGFGGTNYNSALKSRLTISVD |
| | | NSKNTVYLQMNSLRAEDTAVYYCASGNFDAMDYW |
| | | GQGTLVTVSS |
| 73 | TLA0015-VH-12 | EIQLVESGGGLIQPGGSLRISCAVSGFSLSSYGVDWV |
| | | RQAPGKGLEWLGVIWGFGGTNYQSALKSRLTISKD |
| | | NSKNTVYLQMNSLRAEDTAVYYCASGNFDAMDYW |
| | | GQGTLVTVSS |
| 74 | TLA0015-VH-13 | EIQLVESGGGLIQPGGSLRISCAVSGFSLSSYGVDWV |
| | | RQAPGKGLEWLGVIWGFGGTNYNAALKSRLTISKD |
| | | NSKNTVYLQMNSLRAEDTAVYYCASGNFDAMDYW |
| | | GQGTLVTVSS |
| 75 | TLA0015-VH-14 | EIQLVESGGGLIQPGGSLRISCAVSGFSLSSYGVDWV |
| | | RQAPGKGLEWLGVIWGFGGTNYNSALKSRLTISAD |
| | | NSKNTVYLQMNSLRAEDTAVYYCASGNFDAMDYW |
| | | GQGTLVTVSS |
| 76 | TLA0015-VH-15 | EIQLVESGGGLIQPGGSLRISCAVSGFSLSYYGVDWV |
| | | RQAPGKGLEWLGVIWGFGGTNYNSALKSRLTISKD |
| | | NSKNTVYLQMNSLRAEDTAVYYCASGNFDAMDYW |
| | | GQGTLVTVSS |
| 77 | TLA0015-VH-16 | EIQLVESGGGLIQPGGSLRISCAVSGFSLSSYGVDWV |
| | | RQAPGKGLEWLGVIWGFGGTNYNSALKSRLTISKD |
| | | NSKNTVYLQMNSLRAEDTAVYYCASGNYDAMDY |
| | | WGQGTLVTVSS |
| 78 | TLA0015-VH-17 | EIQLVESGGGLIQPGGSLRISCAVSGFSLSSYGVDWV |
| | | RQAPGKGLEWLGVIWGFGGTNYNSALKSRLTISKD |
| | | NSKNTVYLQMNSLRAEDTAVYYCASGDFDAMDYW |
| | | GQGTLVTVSS |
| 79 | TLA0015-VH-18 | EIQLVESGGGLIQPGGSLRISCAVSGFSLSSYGVDWV |
| | | RQAPGKGLEWLGVIWGFGGTNYNSALKSRLTISKD |
| | | NSKNTVYLQMNSLRAEDTAVYYCASGLEDAMDYW |
| | | GQGTLVTVSS |
| 80 | TLA0015-VH-19 | EIQLVESGGGLIQPGGSLRISCAVSGFSLSTYGVDWV |
| | | RQAPGKGLEWLGVIWGFGGTNYNSALKSRLTISKD |
| | | NSKNTVYLQMNSLRAEDTAVYYCASGNFDAMDYW |
| | | GQGTLVTVSS |
| 81 | TLA0015-VH-20 | EIQLVESGGGLIQPGGSLRISCAVSGFSLISYGVDWV |
| | | RQAPGKGLEWLGVIWGFGGTNYNSALKSRLTISKD |
| | | NSKNTVYLQMNSLRAEDTAVYYCASGNFDAMDYW |
| | | GQGTLVTVSS |
| 82 | TLA0015-VH-21 | EIQLVESGGGLIQPGGSLRISCAVSGFSLSSYGVDWV |
| | | RQAPGKGLEWLGVIWGFGGTNYNPALKSRLTISKD |
| | | NSKNTVYLQMNSLRAEDTAVYYCASGNFDAMDYW |
| | | GQGTLVTVSS |
| 83 | TLA0015-VH-22 | EIQLVESGGGLIQPGGSLRISCAVSGFSLKSYGVDWV |
| | | RQAPGKGLEWLGVIWGFGGTNYNSALKSRLTISKD |
| | | NSKNTVYLQMNSLRAEDTAVYYCASGNFDAMDYW |
| | | GQGTLVTVSS |
| 84 | TLA0015-VH-23 | EIQLVESGGGLIQPGGSLRISCAVSGFSLSSYGVDWV |
| | | RQAPGKGLEWLGVIWGFGGTHYNSALKSRLTISKD |
| | | NSKNTVYLQMNSLRAEDTAVYYCASGNFDAMDYW |
| | | GQGTLVTVSS |
| 85 | TLA0015-VH-24 | EIQLVESGGGLIQPGGSLRISCAVSGFSLSSYGVDWV |
| | | RQAPGKGLEWLGRIWGFGGTNYNSALKSRLTISKD |
| | | NSKNTVYLQMNSLRAEDTAVYYCASGNFDAMDYW |
| | | GQGTLVTVSS |
| 86 | TLA0015-VH-25 | EIQLVESGGGLIQPGGSLRISCAVSGFDLSSYGVDWV |
| | | RQAPGKGLEWLGVIWGFGGTNYNSALKSRLTISKD |
| | | NSKNTVYLQMNSLRAEDTAVYYCASGNFDAMDYW |
| | | GQGTLVTVSS |
| 87 | TLA0015-VH-26 | EIQLVESGGGLIQPGGSLRISCAVSGFSLSSYGVDWV |
| | | RQAPGKGLEWLGVIWGFGKTNYNSALKSRLTISKD |
| | | NSKNTVYLQMNSLRAEDTAVYYCASGNFDAMDYW |
| | | GQGTLVTVSS |
| 88 | TLA0015-VH-27 | EIQLVESGGGLIQPGGSLRISCAVSGFSLSSYGVDWV |
| | | RQAPGKGLEWLGVIWGFGGTDYNSALKSRLTISKD |
| | | NSKNTVYLQMNSLRAEDTAVYYCASGNFDAMDYW |
| | | GQGTLVTVSS |
| 89 | TLA0015-VH-28 | EIQLVESGGGLIQPGGSLRISCAVSGFSLSYYGVDWV |
| | | RQAPGKGLEWLGVIWGFGGTNYNSALKSRLTISKD |
| | | NSKNTVYLQMNSLRAEDTAVYYCASGNYDAMDY |
| | | WGQGTLVTVSS |
| 90 | TLA0015-VH-29 | EIQLVESGGGLIQPGGSLRISCAVSGFSLSYYGVDWV |
| | | RQAPGKGLEWLGVIWGFGGTNYNSALKSRLTISKD |
| | | NSKNTVYLQMNSLRAEDTAVYYCASGDFDAMDYW |
| | | GQGTLVTVSS |
| 91 | TLA0015-VH-30 | EIQLVESGGGLIQPGGSLRISCAVSGFSLSYYGVDWV |
| | | RQAPGKGLEWLGVIWGFGGTNYNPALKSRLTISKD |
| | | NSKNTVYLQMNSLRAEDTAVYYCASGNFDAMDYW |
| | | GQGTLVTVSS |
| 92 | TLA0015-VH-31 | EIQLVESGGGLIQPGGSLRISCAVSGFSLSYYGVDWV |
| | | RQAPGKGLEWLGVIWGFGGTHYNSALKSRLTISKD |
| | | NSKNTVYLQMNSLRAEDTAVYYCASGNFDAMDYW |
| | | GQGTLVTVSS |
| 93 | TLA0015-VH-32 | EIQLVESGGGLIQPGGSLRISCAVSGFSLSYYGVDWV |
| | | RQAPGKGLEWLGRIWGFGGTNYNSALKSRLTISKD |
| | | NSKNTVYLQMNSLRAEDTAVYYCASGNFDAMDYW |
| | | GQGTLVTVSS |
| 94 | TLA0015-VH-33 | EIQLVESGGGLIQPGGSLRISCAVSGFDLSYYGVDWV |
| | | RQAPGKGLEWLGVIWGFGGTNYNSALKSRLTISKD |
| | | NSKNTVYLQMNSLRAEDTAVYYCASGNFDAMDYW |
| | | GQGTLVTVSS |
| 95 | TLA0015-VH-34 | EIQLVESGGGLIQPGGSLRISCAVSGFSLISYGVDWV |
| | | RQAPGKGLEWLGVIWGFGGTNYNSALKSRLTISKD |
| | | NSKNTVYLQMNSLRAEDTAVYYCASGNYDAMDY |
| | | WGQGTLVTVSS |
| 96 | TLA0015-VH-35 | EIQLVESGGGLIQPGGSLRISCAVSGFSLSSYGVDWV |
| | | RQAPGKGLEWLGVIWGFGGTNYNPALKSRLTISKD |
| | | NSKNTVYLQMNSLRAEDTAVYYCASGNYDAMDY |
| | | WGQGTLVTVSS |
| 97 | TLA0015-VH-36 | EIQLVESGGGLIQPGGSLRISCAVSGFSLSSYGVDWV |
| | | RQAPGKGLEWLGVIWGFGGTHYNSALKSRLTISKD |
| | | NSKNTVYLQMNSLRAEDTAVYYCASGNYDAMDY |
| | | WGQGTLVTVSS |
| 98 | TLA0015-VH-37 | EIQLVESGGGLIQPGGSLRISCAVSGFSLSSYGVDWV |
| | | RQAPGKGLEWLGRIWGFGGTNYNSALKSRLTISKD |
| | | NSKNTVYLQMNSLRAEDTAVYYCASGNYDAMDY |
| | | WGQGTLVTVSS |
| 99 | TLA0015-VH-38 | EIQLVESGGGLIQPGGSLRISCAVSGFSLSSYGVDWV |
| | | RQAPGKGLEWLGVIWGFGKTNYNSALKSRLTISKD |
| | | NSKNTVYLQMNSLRAEDTAVYYCASGNYDAMDY |
| | | WGQGTLVTVSS |
| 100 | TLA0015-VH-39 | EIQLVESGGGLIQPGGSLRISCAVSGFSLISYGVDWV |
| | | RQAPGKGLEWLGVIWGFGGTNYNSALKSRLTISKD |
| | | NSKNTVYLQMNSLRAEDTAVYYCASGDFDAMDYW |
| | | GQGTLVTVSS |
| 101 | TLA0015-VH-40 | EIQLVESGGGLIQPGGSLRISCAVSGFSLSSYGVDWV |
| | | RQAPGKGLEWLGVIWGFGGTHYNSALKSRLTISKD |
| | | NSKNTVYLQMNSLRAEDTAVYYCASGDFDAMDYW |
| | | GQGTLVTVSS |
| 102 | TLA0015-VL-1 | DIQMTQTTSSLSASLGDRVTISCRASQDISNYLNWYQ |
| | | QKPDGTVKLLIYYTSRLHSGVPSRFSGSGSGTDYSLT |
| | | ISNLEQEDIATYFCQQGNTLPFTFGSGTKLEIK |
| 103 | TLA0015-VL-2 | DIQMTQSPSSLSASVGDRVTITCRASQDISNYLNWY |
| | | QQKPGKVVKLLIYYTSRLHSGVPSRFSGSGSGTDYT |
| | | LTISSLQPEDVATYFCQQGNTLPFTFGPGTKLEIK |
| 104 | TLA0015-VL-3 | DIQMTQSPSSLSASVGDRVTITCRASQDISNYLNWY |
| | | QQKPGKAPKLLIYYTSRLHSGVPSRFSGSGSGTDFTL |
| | | TISSLQPEDFATYYCQQGNTLPFTFGQGTKLEIK |
| 105 | TLA0015-VL-4 | DIQMTQSPSSLSASVGDRVTITCRASQDISNYLNWY |
| | | QQKPGKAPKLLIYYTSRLHSGVPSRFSGSGSGTDYTL |
| | | TISSLQPEDFATYFCQQGNTLPFTFGQGTKLEIK |
| 106 | TLA0015-VL-5 | DIQMTQSPSSLSASVGDRVTITCRASQDISNYLNWY |
| | | QQKPGKAVKLLIYYTSRLHSGVPSRFSGSGSGTDYT |
| | | LTISSLQPEDFATYFCQQGNTLPFTFGQGTKLEIK |
| 107 | TLA-0238-HCDR1 | NY[ILMYW]M[NHS] |
| 108 | TLA-0238-HCDR1-1 | NYWMN |
| 109 | TLA-0238-HCDR1-2 | NYWMH |
| 110 | TLA-0238-HCDR1-3 | NYWMS |
| 111 | TLA-0238-HCDR1-4 | NYIMN |
| 112 | TLA-0238-HCDR1-5 | NYLMN |
| 113 | TLA-0238-HCDR1-6 | NYMMN |
| 114 | TLA-0238-HCDR1-7 | NYYMN |
| 115 | TLA-0238-HCDR2 | QIRLKSDNYATHYA[ADE][PS]VKG |
| 116 | TLA-0238-HCDR2-1 | QIRLKSDNYATHYAESVKG |
| 117 | TLA-0238-HCDR2-2 | QIRLKSDNYATHYADSVKG |
| 118 | TLA-0238-HCDR2-3 | QIRLKSDNYATHYAASVKG |
| 119 | TLA-0238-HCDR2-4 | QIRLKSDNYATHYAAPVKG |
| 120 | TLA-0238-HCDR3-1 | LLLRYRDY |
| 121 | TLA-0238-HFR1 | [EQ]V[KQ][LV][EV]ESGGG[LV]V[KQ]PG[GR][AS]L[ |
| | | KR]LSC[AI]ASGFTFS |
| 122 | TLA-0238-HFR1-1 | EVKVEESGGGLVQPGGSMKLSCIASGFTFS |
| 123 | TLA-0238-HFR1-2 | EVQVVESGGGLVKPGGSLRLSCAASGFTFS |
| 124 | TLA-0238-HFR1-3 | EVQVVESGGGLVQPGGSLRLSCIASGFTFS |
| 125 | TLA-0238-HFR1-4 | EVQLVESGGGLVQPGGSLRLSCAASGFTFS |
| 126 | TLA-0238-HFR1-5 | QVQLVESGGGVVQPGRSLRLSCAASGFTFS |
| 127 | TLA-0238-HFR1-6 | EVKVEESGGGLVQPGGSLRLSCIASGFTFS |
| 128 | TLA-0238-HFR1-7 | EVKVEESGGGLVQPGGSLKLSCIASGFTFS |
| 129 | TLA-0238-HFR1-8 | EVKVEESGGGLVQPGGALRLSCIASGFTFS |
| 130 | TLA-0238-HFR1-9 | EVKLEESGGGLVQPGGSLKLSCIASGFTFS |
| 131 | TLA-0238-HFR1-10 | EVQLVESGGGLVKPGGSLRLSCAASGFTFS |
| 132 | TLA-0238-HFR1-11 | EVKLEESGGGLVQPGGALRLSCIASGFTFS |
| 133 | TLA-0238-HFR2 | WVRQ[AS]P[EG]KGLEWV[AG] |
| 134 | TLA-0238-HFR2-1 | WVRQSPEKGLEWVA |
| 135 | TLA-0238-HFR2-2 | WVRQAPGKGLEWVA |
| 136 | TLA-0238-HFR3 | [NR]F[AT]ISRD[DN][AS]K[NS][ST][ALV] |
| | | YLQMNSL[KR][ADT]ED[MT]AVYYCTP |
| 137 | TLA-0238-HFR3-1 | NFTISRDDSKSSVYLQMNNLRAEDTGIYYCTP |
| 138 | TLA-0238-HFR3-2 | NFTISRDDSKSTVYLQMNSLKTEDTAVYYCTP |
| 139 | TLA-0238-HFR3-3 | NFTISRDNAKSSVYLQMNSLRAEDTAVYYCTP |
| 140 | TLA-0238-HFR3-4 | RFTISRDDSKSTVYLQMNSLKTEDTAVYYCTP |
| 141 | TLA-0238-HFR3-5 | NFAISRDDSKSTVYLQMNSLKTEDTAVYYCTP |
| 142 | TLA-0238-HFR3-6 | RFTISRDNSKNTLYLQMNSLRAEDTAVYYCTP |
| 143 | TLA-0238-HFR3-7 | RFTISRDNSKNTLYLQMNSLKTEDTAVYYCTP |
| 144 | TLA-0238-HFR3-8 | RFTISRDDSKNSLYLQMNSLRDEDTAVYYCTP |
| 145 | TLA-0238-HFR3-9 | RFTISRDDSKNTLYLQMNSLRAEDTAVYYCTP |
| 146 | TLA-0238-HFR3-10 | RFTISRDDSKNSLYLQMNSLRAEDMAVYYCTP |
| 147 | TLA-0238-HFR3-11 | RFTISRDDSKNTAYLQMNSLRAEDTAVYYCTP |
| 148 | TLA-0238-HFR3-12 | RFTISRDDSKNTLYLQMNSLKTEDTAVYYCTP |
| 149 | TLA-0238-HFR4 | WGQGT[LMT][LV]TVSS |
| 150 | TLA-0238-HFR4-1 | WGQGTTLTVSS |
| 151 | TLA-0238-HFR4-3 | WGQGTLLTVSS |
| 152 | TLA-0238-HFR4-4 | WGQGTMVTVSS |
| 153 | TLA-0238-HFR4-5 | WGQGTTVTVSS |
| 154 | TLA0238-LCDR1 | SASSSVSY[LM][AHT] |
| 155 | TLA0238-LCDR1-1 | SASSSVSYMH |
| 156 | TLA0238-LCDR1-2 | SASSSVSYLH |
| 157 | TLA0238-LCDR1-3 | SASSSVSYLA |
| 158 | TLA0238-LCDR1-4 | SASSSVSYLT |
| 159 | TLA0238-LCDR2 | DTSN[LR]A[ST] |
| 160 | TLA0238-LCDR2-1 | DTSNLAS |
| 161 | TLA0238-LCDR2-2 | DTSNRAS |
| 162 | TLA0238-LCDR2-3 | DTSNLAT |
| 163 | TLA0238-LCDR3 | FQE[NSE][GEA]HPFT |
| 164 | TLA0238-LCDR3-1 | FQENGHPFT |
| 165 | TLA0238-LCDR3-2 | FQESGHPFT |
| 166 | TLA0238-LCDR3-3 | FQENEHPFT |
| 167 | TLA0238-LCDR3-4 | FQEEGHPFT |
| 168 | TLA0238-LCDR3-5 | FQENAHPFT |
| 169 | TLA0238-LFR1 | [DE][INTV][QV][LM]TQSP[AS][FIST][LMV]S[ALV][S |
| | | T][PV]G[DE][KR][AV]T[IM][ST]C |
| 170 | TLA0238-LFR1-1 | ENVLTQSPAIMSASPGEKVTMTC |
| 171 | TLA0238-LFR1-2 | ENVLTQSPATLSLSPGERATMSC |
| 172 | TLA0238-LFR1-3 | DNQLTQSPSSLSASVGDRVTITC |
| 173 | TLA0238-LFR1- | DIQMTQSPSSVSASVGDRVTITC |
| | 4/TLA0241-LFR1-2 | |
| 174 | TLA0238-LFR1-5 | ENVLTQSPAIMSATPGEKVTITC |
| 175 | TLA0238-LFR1-6 | EVVLTQSPAFLSVTPGEKVTITC |
| 176 | TLA0238-LFR1-7 | ENVLTQSPAFMSATPGEKVTITC |
| 177 | TLA0238-LFR1-8 | EIVLTQSPAFMSATPGEKVTITC |
| 178 | TLA0238-LFR1-9 | ETVLTQSPAFMSATPGEKVTITC |
| 179 | TLA0238-LFR1-10 | ENVLTQSPAFLSVTPGEKVTITC |
| 180 | TLA0238-LFR2 | WYQQKP[DG][KQ][AS]P[KQR]LWIY |
| 181 | TLA0238-LFR2-1 | WYQQKSSTSPKLWIY |
| 182 | TLA0238-LFR2-2 | WYQQKPGQAPRLWIY |
| 183 | TLA0238-LFR2-3 | WYQQKPGKAPKLWIY |
| 184 | TLA0238-LFR2-4 | WYQQKPGQSPQLWIY |
| 185 | TLA0238-LFR2-5 | WYQQKPGQSPRLWIY |
| 186 | TLA0238-LFR2-6 | WYQQKPGKSPKLWIY |
| 187 | TLA0238-LFR2-7 | WYQQKPDQSPKLWIY |
| 188 | TLA0238-LFR2-8 | WYQQKPGQSPKLWIY |
| 189 | TLA0238-LFR3 | GVP[ADGS]RFSGSGSG[NT][DS][FY]TLTISS[LM][EQ] |
| | | [AP]ED[AFV]A[TV]YYC |
| 190 | TLA0238-LFR3-1 | GVPGRFSGSGSGNSYSLTISSMEAEDVATYYC |
| 191 | TLA0238-LFR3-2 | GVPARFSGSGSGNDYTLTISSMEPEDFAVYYC |
| 192 | TLA0238-LFR3-3 | GVPGRFSGSGSGNDYTLTISSLQPEDFATYYC |
| 193 | TLA0238-LFR3-5 | GVPDRFSGSGSGTDYTLTISSLQPEDVATYYC |
| 194 | TLA0238-LFR3-6 | GVPDRFSGSGSGTDYTLTISSLQPEDFAVYYC |
| 195 | TLA0238-LFR3-7 | GVPSRFSGSGSGTSYTLTISSLQPEDVATYYC |
| 196 | TLA0238-LFR3-8 | GVPDRFSGSGSGTDYTLTISSLEAEDAATYYC |
| 197 | TLA0238-LFR3-9 | GVPSRFSGSGSGTDYTLTISSLQPEDFATYYC |
| 198 | TLA0238-LFR4 | FG[GQ]GTK[LV]E[IM]K |
| 199 | TLA0238-LFR4-1 | FGGGTKLEMK |
| 200 | TLA0238-LFR4-2 | FGGGTKVEMK |
| 201 | TLA0238-LFR4- | FGQGTKVEIK |
| | 4/TLA0241-LFR4-1 | |
| 202 | TLA0238-VH-1 | EVKVEESGGGLVQPGGSMKLSCIASGFTFSNYWMN |
| | | WVRQSPEKGLEWVAQIRLKSDNYATHYAESVKGNF |
| | | TISRDDSKSSVYLQMNNLRAEDTGIYYCTPLLLRYR |
| | | DYWGQGTTLTVSS |
| 203 | TLA0238-VH-2 | EVQVVESGGGLVKPGGSLRLSCAASGFTFSNYWMN |
| | | WVRQAPGKGLEWVAQIRLKSDNYATHYAESVKGN |
| | | FTISRDDSKSTVYLQMNSLKTEDTAVYYCTPLLLRY |
| | | RDYWGQGTLVTVSS |
| 204 | TLA0238-VH-3 | EVQVVESGGGLVQPGGSLRLSCIASGFTFSNYWMN |
| | | WVRQAPGKGLEWVAQIRLKSDNYATHYAESVKGN |
| | | FTISRDNAKSSVYLQMNSLRAEDTAVYYCTPLLLRY |
| | | RDYWGQGTLLTVSS |
| 205 | TLA0238-VH-4 | EVQVVESGGGLVKPGGSLRLSCAASGFTFSNYWMN |
| | | WVRQAPGKGLEWVAQIRLKSDNYATHYAESVKGR |
| | | FTISRDDSKSTVYLQMNSLKTEDTAVYYCTPLLLRY |
| | | RDYWGQGTLVTVSS |
| 206 | TLA0238-VH-5 | EVQVVESGGGLVKPGGSLRLSCAASGFTFSNYWMN |
| | | WVRQAPGKGLEWVAQIRLKSDNYATHYAESVKGN |
| | | FAISRDDSKSTVYLQMNSLKTEDTAVYYCTPLLLRY |
| | | RDYWGQGTLVTVSS |
| 207 | TLA0238-VH-6 | QVQLVESGGGVVQPGRSLRLSCAASGFTFSNYWMN |
| | | WVRQAPGKGLEWVAQIRLKSDNYATHYADSVKGR |
| | | FTISRDNSKNTLYLQMNSLRAEDTAVYYCTPLLLRY |
| | | RDYWGQGTMVTVSS |
| 208 | TLA0238-VH-7 | EVKVEESGGGLVQPGGSLRLSCIASGFTFSNYWMH |
| | | WVRQAPGKGLEWVGQIRLKSDNYATHYAASVKGR |
| | | FTISRDNSKNTLYLQMNSLKTEDTAVYYCTPLLLRY |
| | | RDYWGQGTLVTVSS |
| 209 | TLA0238-VH-8 | EVKVEESGGGLVQPGGSLKLSCIASGFTFSNYWMH |
| | | WVRQAPGKGLEWVGQIRLKSDNYATHYAASVKGR |
| | | FTISRDDSKNSLYLQMNSLRDEDTAVYYCTPLLLRY |
| | | RDYWGQGTLVTVSS |
| 210 | TLA0238-VH-9 | EVKVEESGGGLVQPGGALRLSCIASGFTFSNYWMH |
| | | WVRQAPGKGLEWVGQIRLKSDNYATHYAASVKGR |
| | | FTISRDDSKNTLYLQMNSLRAEDTAVYYCTPLLLRY |
| | | RDYWGQGTLVTVSS |
| 211 | TLA0238-VH-10 | EVKVEESGGGLVQPGGSLRLSCIASGFTFSNYWMH |
| | | WVRQAPGKGLEWVGQIRLKSDNYATHYAASVKGR |
| | | FTISRDDSKNSLYLQMNSLRAEDMAVYYCTPLLLRY |
| | | RDYWGQGTLVTVSS |
| 212 | TLA0238-VH-11 | EVQLVESGGGLVQPGGSLRLSCAASGFTFSNYWMN |
| | | WVRQAPGKGLEWVAQIRLKSDNYATHYADSVKGR |
| | | FTISRDNSKNTLYLQMNSLRAEDTAVYYCTPLLLRY |
| | | RDYWGQGTMVTVSS |
| 213 | TLA0238-VH-12 | EVKLEESGGGLVQPGGSLKLSCIASGFTFSNYWMH |
| | | WVRQAPGKGLEWVGQIRLKSDNYATHYAASVKGR |
| | | FTISRDDSKNTAYLQMNSLRAEDTAVYYCTPLLLRY |
| | | RDYWGQGTLVTVSS |
| 214 | TLA0238-VH-13 | EVQLVESGGGLVKPGGSLRLSCAASGFTFSNYWMN |
| | | WVRQAPGKGLEWVAQIRLKSDNYATHYAAPVKGR |
| | | FTISRDDSKNTLYLQMNSLKTEDTAVYYCTPLLLRY |
| | | RDYWGQGTTVTVSS |
| 215 | TLA0238-VH-14 | EVKLEESGGGLVQPGGALRLSCIASGFTFSNYWMS |
| | | WVRQAPGKGLEWVGQIRLKSDNYATHYAASVKGR |
| | | FTISRDDSKNTLYLQMNSLRAEDTAVYYCTPLLLRY |
| | | RDYWGQGTLVTVSS |
| 216 | TLA0238-VH-15 | EVQLVESGGGLVQPGGSLRLSCAASGFTFSNYIMNW |
| | | VRQAPGKGLEWVAQIRLKSDNYATHYADSVKGRFT |
| | | ISRDNSKNTLYLQMNSLRAEDTAVYYCTPLLLRYRD |
| | | YWGQGTLVTVSS |
| 217 | TLA0238-VH-16 | EVQLVESGGGLVQPGGSLRLSCAASGFTFSNYLMN |
| | | WVRQAPGKGLEWVAQIRLKSDNYATHYADSVKGR |
| | | FTISRDNSKNTLYLQMNSLRAEDTAVYYCTPLLLRY |
| | | RDYWGQGTLVTVSS |
| 218 | TLA0238-VH-17 | EVQLVESGGGLVQPGGSLRLSCAASGFTFSNYMMN |
| | | WVRQAPGKGLEWVAQIRLKSDNYATHYADSVKGR |
| | | FTISRDNSKNTLYLQMNSLRAEDTAVYYCTPLLLRY |
| | | RDYWGQGTLVTVSS |
| 219 | TLA0238-VH-18 | EVQLVESGGGLVQPGGSLRLSCAASGFTFSNYWMN |
| | | WVRQAPGKGLEWVAQIRLKSDNYATHYADSVKGR |
| | | FTISRDNSKNTLYLQMNSLRAEDTAVYYCTPLLLRY |
| | | RDYWGQGTLVTVSS |
| 220 | TLA0238-VH-19 | EVQLVESGGGLVQPGGSLRLSCAASGFTFSNYYMN |
| | | WVRQAPGKGLEWVAQIRLKSDNYATHYADSVKGR |
| | | FTISRDNSKNTLYLQMNSLRAEDTAVYYCTPLLLRY |
| | | RDYWGQGTLVTVSS |
| 221 | TLA0238-VL-1 | ENVLTQSPAIMSASPGEKVTMTCSASSSVSYMHWY |
| | | QQKSSTSPKLWIYDTSNLASGVPGRFSGSGSGNSYSL |
| | | TISSMEAEDVATYYCFQENGHPFTFGGGTKLEMK |
| 222 | TLA0238-VL-2 | ENVLTQSPATLSLSPGERATMSCSASSSVSYMHWYQ |
| | | QKPGQAPRLWIYDTSNLASGVPARFSGSGSGNDYTL |
| | | TISSMEPEDFAVYYCFQENGHPFTFGGGTKVEMK |
| 223 | TLA0238-VL-3 | DNQLTQSPSSLSASVGDRVTITCSASSSVSYMHWYQ |
| | | QKPGKAPKLWIYDTSNLASGVPGRFSGSGSGNDYTL |
| | | TISSLQPEDFATYYCFQENGHPFTFGQGTKLEIK |
| 224 | TLA0238-VL-4 | ENVLTQSPAIMSATPGEKVTITCSASSSVSYLHWYQ |
| | | QKPGQSPQLWIYDTSNRASGVPDRESGSGSGTDYTL |
| | | TISSLQPEDVATYYCFQENGHPFTFGGGTKLEMK |
| 225 | TLA0238-VL-5 | EVVLTQSPAFLSVTPGEKVTITCSASSSVSYLAWYQQ |
| | | KPGQSPRLWIYDTSNRASGVPDRFSGSGSGTDYTLTI |
| | | SSLQPEDFAVYYCFQENGHPFTFGGGTKLEMK |
| 226 | TLA0238-VL-6 | ENVLTQSPAFMSATPGEKVTITCSASSSVSYLAWYQ |
| | | QKPGKSPKLWIYDTSNLATGVPSRFSGSGSGTSYTLT |
| | | ISSLQPEDVATYYCFQENGHPFTFGGGTKLEMK |
| 227 | TLA0238-VL-7 | EIVLTQSPAFMSATPGEKVTITCSASSSVSYLHWYQQ |
| | | KPDQSPKLWIYDTSNRASGVPDRFSGSGSGTDYTLTI |
| | | SSLEAEDAATYYCFQENGHPFTFGGGTKLEMK |
| 228 | TLA0238-VL-8 | ETVLTQSPAFMSATPGEKVTITCSASSSVSYLTWYQ |
| | | QKPGQSPKLWIYDTSNLASGVPDRESGSGSGTDYTL |
| | | TISSLEAEDAATYYCFQENGHPFTFGGGTKLEMK |
| 229 | TLA0238-VL-9 | DIQMTQSPSSVSASVGDRVTITCSASSSVSYMHWYQ |
| | | QKPGKAPKLWIYDTSNLASGVPSRFSGSGSGTDFTL |
| | | TISSLQPEDFATYYCFQENGHPFTFGQGTKVEIK |
| 230 | TLA0238-VL-10 | ENVLTQSPAFLSVTPGEKVTITCSASSSVSYLHWYQQ |
| | | KPDQSPKLWIYDTSNLATGVPSRFSGSGSGTDYTLTI |
| | | SSLQPEDFATYYCFQENGHPFTFGGGTKLEMK |
| 231 | TLA0238-VL-11 | DIQMTQSPSSVSASVGDRVTITCSASSSVSYMHWYQ |
| | | QKPGKAPKLWIYDTSNLASGVPSRFSGSGSGTDFTL |
| | | TISSLQPEDFATYYCFQESGHPFTFGQGTKVEIK |
| 232 | TLA0238-VL-12 | DIQMTQSPSSVSASVGDRVTITCSASSSVSYMHWYQ |
| | | QKPGKAPKLWIYDTSNLASGVPSRFSGSGSGTDFTL |
| | | TISSLQPEDFATYYCFQENEHPFTFGQGTKVEIK |
| 233 | TLA0238-VL-13 | DIQMTQSPSSVSASVGDRVTITCSASSSVSYMHWYQ |
| | | QKPGKAPKLWIYDTSNLASGVPSRFSGSGSGTDFTL |
| | | TISSLQPEDFATYYCFQEEGHPFTFGQGTKVEIK |
| 234 | TLA0238-VL-14 | DIQMTQSPSSVSASVGDRVTITCSASSSVSYMHWYQ |
| | | QKPGKAPKLWIYDTSNLASGVPSRFSGSGSGTDFTL |
| | | TISSLQPEDFATYYCFQENAHPFTFGQGTKVEIK |
| 235 | TLA0241-HCDR1 | DYY[LM][HN] |
| 236 | TLA0241-HCDR1-1 | DYYLN |
| 237 | TLA0241-HCDR1-2 | DYYMH |
| 238 | TLA0241-HCDR2 | DINPNNGRTTY[AN][DQ][KS][FV][KQT]G |
| 239 | TLA0241-HCDR2-1 | DINPNNGRTTYNQKFTG |
| 240 | TLA0241-HCDR2-2 | DINPNNGRTTYADSVKG |
| 241 | TLA0241-HCDR2-3 | DINPNNGRTTYNQKFQG |
| 242 | TLA0241-HCDR3-1 | EGGYGWYFDV |
| 243 | TLA0241-HFR1 | [EQ][ILV]QL[LQV][EQ]SG[AGP][EG][ELV][KV][KQ]P |
| | | G[AGR]S[LV]R[IL]SC[AKS]ASGYTFT |
| 244 | TLA0241-HFR1-1 | EVQLVESGGGLVQPGGSLRLSCSASGYTFT |
| 245 | TLA0241-HFR1-2 | QVQLVESGGGVVQPGRSLRLSCAASGYTFT |
| 246 | TLA0241-HFR1-3 | EVQLQQSGPEVKKPGASVRISCKASGYTFT |
| 247 | TLA0241-HFR1-4 | ELQLQQSGPEVKKPGASVRISCKASGYTFT |
| 248 | TLA0241-HFR1-5 | EVQLLESGGGLVQPGGSLRLSCAASGYTFT |
| 249 | TLA0241-HFR1-6 | EIQLQQSGAEEKKPGASVRISCKASGYTFT |
| 250 | TLA0241-HFR1-7 | ELQLQQSGAEVKKPGASVRISCKASGYTFT |
| 251 | TLA0241-HFR2 | WV[QR]QAPG[EKQ]GLEWIG |
| 252 | TLA0241-HFR2-1 | WVRQAPGKGLEWIG |
| 253 | TLA0241-HFR2-2 | WVRQAPGQGLEWIG |
| 254 | TLA0241-HFR2-3 | WVQQAPGKGLEWIG |
| 255 | TLA0241-HFR2-4 | WVRQAPGEGLEWIG |
| 256 | TLA0241-HFR3 | R[FV]TI[ST][AR]D[KNT]S[KST][NS]T[AL]Y[LM][EQ][ |
| | | LM][NRS]SL[RT][AS][DE]D[MT]AVYYCAR |
| 257 | TLA0241-HFR3-1 | RFTISRDNSKNTLYLQMSSLRAEDTAVYYCAR |
| 258 | TLA0241-HFR3-2 | RFTISRDNSKNTLYLQMNSLRAEDTAVYYCAR |
| 259 | TLA0241-HFR3-3 | RVTITRDTSSSTAYMELRSLRSDDTAVYYCAR |
| 260 | TLA0241-HFR3-4 | RVTITADKSTSTAYMELRSLRSDDTAVYYCAR |
| 261 | TLA0241-HFR3-5 | RVTITADKSTSTAYMELRSLTSDDTAVYYCAR |
| 262 | TLA0241-HFR3-6 | RVTITADKSTSTAYMELRSLRSDDMAVYYCAR |
| 263 | TLA0241-HFR4 | WG[QRT]GT[LMT]VTVSS |
| 264 | TLA0241-HFR4-1 | WGRGTLVTVSS |
| 265 | TLA0241-HFR4-2 | WGQGTMVTVSS |
| 266 | TLA0241-HFR4-3 | WGTGTLVTVSS |
| 267 | TLA0241-HFR4-4 | WGQGTTVTVSS |
| 268 | TLA0241-HFR4-5 | WGTGTTVTVSS |
| 269 | TLA0241-LCDR1 | S[AT]SSSIISNY[LS][AH] |
| 270 | TLA0241-LCDR1-1 | STSSSIISNYSH |
| 271 | TLA0241-LCDR1-2 | SASSSIISNYLH |
| 272 | TLA0241-LCDR1-3 | SASSSIISNYSA |
| 273 | TLA0241-LCDR1-4 | SASSSIISNYLA |
| 274 | TLA0241-LCDR2 | KTSN[LR]PS |
| 275 | TLA0241-LCDR2-1 | KTSNLPS |
| 276 | TLA0241-LCDR2-2 | KTSNRPS |
| 277 | TLA0241-LCDR3-1 | QQGSDMPIT |
| 278 | TLA0241-LFR1 | DE][KR]VTITC |
| 279 | TLA0241-LFR1-3 | EIVLTQSPTTLSLSPGEKVTITC |
| 280 | TLA0241-LFR1-4 | EIVLTQSPPTLSLSPGEKVTITC |
| 281 | TLA0241-LFR1-5 | EIVLTQSPGTLSLSPGEKVTITC |
| 282 | TLA0241-LFR1-6 | AIQLTQSPSSLSASVGDRVTITC |
| 283 | TLA0241-LFR1-7 | EIVLTQSPATLSLSPGEKVTITC |
| 284 | TLA0241-LFR2 | WYQQKP[DG][KQ][AS]P[KQILLIY |
| 285 | TLA0241-LFR2-2 | WYQQKPGKSPKLLIY |
| 286 | TLA0241-LFR2-3 | WYQQKPGQSPQLLIY |
| 287 | TLA0241-LFR2-4 | WYQQKPDQSPKLLIY |
| 288 | TLA0241-LFR3 | GVP[ADS]RFSGSGSGT[DES]FTLTI[NS]SL[EQ][AP]E |
| | | D[AF]ATYYC |
| 289 | TLA0241-LFR3-1 | GVPSRFSGSGSGTEFTLTISSLQPEDFATYYC |
| 290 | TLA0241-LFR3-3 | GVPSRFSGSGSGTDFTLTINSLEAEDAATYYC |
| 291 | TLA0241-LFR3-4 | GVPDRFSGSGSGTDFTLTINSLEAEDAATYYC |
| 292 | TLA0241-LFR3-5 | GVPARFSGSGSGTDFTLTINSLEAEDAATYYC |
| 293 | TLA0241-LFR3-6 | GVPSRFSGSGSGTSFTLTINSLEAEDAATYYC |
| 294 | TLA0241-LFR4 | FG[AQ]GT[KR][LV]E[IL]K |
| 295 | TLA0241-LFR4-2 | FGAGTRLELK |
| 296 | TLA0241-LFR4-3 | FGAGTKLELK |
| 297 | TLA0241-VH-1 | EFQLQQSGPELVKPGASVRISCKASGYTFTDYYLNW |
| | | VKQSRGESLEWIGDINPNNGRTTYNQKFTGKATWT |
| | | VDKSSSTVYMELRSLTSDDSAVYYCAREGGYGWYF |
| | | DVWGTGTTVTVSS |
| 298 | TLA0241-VH-2 | EVQLVESGGGLVQPGGSLRLSCSASGYTFTDYYLN |
| | | WVRQAPGKGLEWIGDINPNNGRTTYADSVKGRFTIS |
| | | RDNSKNTLYLQMSSLRAEDTAVYYCAREGGYGWY |
| | | FDVWGRGTLVTVSS |
| 299 | TLA0241-VH-3 | QVQLVESGGGVVQPGRSLRLSCAASGYTFTDYYLN |
| | | WVRQAPGKGLEWIGDINPNNGRTTYADSVKGRFTIS |
| | | RDNSKNTLYLQMNSLRAEDTAVYYCAREGGYGWY |
| | | FDVWGQGTMVTVSS |
| 300 | TLA0241-VH-4 | EVQLQQSGPEVKKPGASVRISCKASGYTFTDYYMH |
| | | WVRQAPGQGLEWIGDINPNNGRTTYNQKFQGRVTI |
| | | TRDTSSSTAYMELRSLRSDDTAVYYCAREGGYGWY |
| | | FDVWGTGTTVTVSS |
| 301 | TLA0241-VH-5 | EVQLQQSGPEVKKPGASVRISCKASGYTFTDYYMH |
| | | WVQQAPGKGLEWIGDINPNNGRTTYNQKFQGRVTI |
| | | TADKSTSTAYMELRSLRSDDTAVYYCAREGGYGW |
| | | YFDVWGTGTLVTVSS |
| 302 | TLA0241-VH-6 | ELQLQQSGPEVKKPGASVRISCKASGYTFTDYYMH |
| | | WVQQAPGKGLEWIGDINPNNGRTTYNQKFQGRVTI |
| | | TADKSTSTAYMELRSLTSDDTAVYYCAREGGYGWY |
| | | FDVWGTGTTVTVSS |
| 303 | TLA0241-VH-7 | EVQLLESGGGLVQPGGSLRLSCAASGYTFTDYYLN |
| | | WVRQAPGKGLEWIGDINPNNGRTTYADSVKGRFTIS |
| | | RDNSKNTLYLQMNSLRAEDTAVYYCAREGGYGWY |
| | | FDVWGQGTMVTVSS |
| 304 | TLA0241-VH-8 | EVQLQQSGPEVKKPGASVRISCKASGYTFTDYYMH |
| | | WVRQAPGEGLEWIGDINPNNGRTTYNQKFQGRVTIT |
| | | ADKSTSTAYMELRSLRSDDTAVYYCAREGGYGWYF |
| | | DVWGTGTTVTVSS |
| 305 | TLA0241-VH-9 | QVQLVESGGGVVQPGRSLRLSCAASGYTFTDYYLN |
| | | WVRQAPGKGLEWIGDINPNNGRTTYADSVKGRFTIS |
| | | RDNSKNTLYLQMNSLRAEDTAVYYCAREGGYGWY |
| | | FDVWGQGTTVTVSS |
| 306 | TLA0241-VH-10 | EIQLQQSGAEEKKPGASVRISCKASGYTFTDYYMHW |
| | | VQQAPGKGLEWIGDINPNNGRTTYNQKFQGRVTITA |
| | | DKSTSTAYMELRSLRSDDTAVYYCAREGGYGWYFD |
| | | VWGTGTTVTVSS |
| 307 | TLA0241-VH-11 | ELQLQQSGAEVKKPGASVRISCKASGYTFTDYYMH |
| | | WVRQAPGQGLEWIGDINPNNGRTTYNQKFQGRVTI |
| | | TADKSTSTAYMELRSLRSDDMAVYYCAREGGYGW |
| | | YFDVWGTGTLVTVSS |
| 308 | TLA0241-VL-1 | EIVLTQSPTTMAASPGEKITITCSTSSSIISNYSHWYQ |
| | | QKPGFSPKLLIYKTSNLPSGVPARFSGSGSGTSYSLTI |
| | | GTMEAEDAATYYCQQGSDMPITFGAGTKLELK |
| 309 | TLA0241-VL-2 | DIQMTQSPSSLSASVGDRVTITCSTSSSIISNYSHWYQ |
| | | QKPGKAPKLLIYKTSNLPSGVPSRESGSGSGTEFTLTI |
| | | SSLQPEDFATYYCQQGSDMPITFGQGTKVEIK |
| 310 | TLA0241-VL-3 | EIVLTQSPTTLSLSPGEKVTITCSASSSIISNYLHWYQ |
| | | QKPGKSPKLLIYKTSNLPSGVPSRFSGSGSGTDFTLTI |
| | | NSLEAEDAATYYCQQGSDMPITFGAGTRLELK |
| 311 | TLA0241-VL-4 | EIVLTQSPPTLSLSPGEKVTITCSASSSIISNYLHWYQQ |
| | | KPGQSPQLLIYKTSNRPSGVPDRFSGSGSGTDFTLTIN |
| | | SLEAEDAATYYCQQGSDMPITFGAGTRLELK |
| 312 | TLA0241-VL-5 | EIVLTQSPGTLSLSPGEKVTITCSASSSIISNYLHWYQ |
| | | QKPDQSPKLLIYKTSNLPSGVPSRFSGSGSGTDFTLTI |
| | | NSLEAEDAATYYCQQGSDMPITFGAGTKLELK |
| 313 | TLA0241-VL-6 | AIQLTQSPSSLSASVGDRVTITCSTSSSIISNYSHWYQ |
| | | QKPGKAPKLLIYKTSNLPSGVPSRFSGSGSGTDFTLTI |
| | | SSLQPEDFATYYCQQGSDMPITFGQGTKVEIK |
| 314 | TLA0241-VL-7 | EIVLTQSPATLSLSPGEKVTITCSASSSIISNYSAWYQ |
| | | QKPGKSPKLLIYKTSNLPSGVPARFSGSGSGTDFTLTI |
| | | NSLEAEDAATYYCQQGSDMPITFGAGTRLELK |
| 315 | TLA0241-VL-8 | DIQMTQSPSSVSASVGDRVTITCSTSSSIISNYSHWYQ |
| | | QKPGKAPKLLIYKTSNLPSGVPSRFSGSGSGTDFTLTI |
| | | SSLQPEDFATYYCQQGSDMPITFGQGTKVEIK |
| 316 | TLA0241-VL-9 | EIVLTQSPTTLSLSPGEKVTITCSASSSIISNYLHWYQ |
| | | QKPDQSPKLLIYKTSNLPSGVPSRFSGSGSGTDFTLTI |
| | | NSLEAEDAATYYCQQGSDMPITFGAGTKLELK |
| 317 | TLA0241-VL-10 | EIVLTQSPPTLSLSPGEKVTITCSASSSIISNYLAWYQQ |
| | | KPGKSPKLLIYKTSNLPSGVPSRFSGSGSGTSFTLTIN |
| | | SLEAEDAATYYCQQGSDMPITFGAGTKLELK |
| 318 | CK | RTVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 319 | CH1 | ASTKGPSVFPLAPSSKSTSGGTAALGCLVKDYFPEPV |
| | | TVSWNSGALTSGVHTFPAVLQSSGLYSLSSVVTVPS |
| | | SSLGTQTYICNVNHKPSNTKVDKKVEPKSC |
| 320 | Fc(L234A, L235A) | DKTHTCPPCPAPEAAGGPSVFLFPPKPKDTLMISRTP |
| | | EVTCVVVDVSHEDPEVKFNWYVDGVEVHNAKTKP |
| | | REEQYNSTYRVVSVLTVLHQDWLNGKEYKCKVSN |
| | | KALPAPIEKTISKAKGQPREPQVYTLPPSRDELTKNQ |
| | | VSLTCLVKGFYPSDIAVEWESNGQPENNYKTTPPVL |
| | | DSDGSFFLYSKLTVDKSRWQQGNVFSCSVMHEALH |
| | | NHYTQKSLSLSPGK |
| 321 | Fc(L234A, L235A, | DKTHTCPPCPAPEAAGGPSVFLFPPKPKDTLYITREP |
| | M252Y, S254T, | EVTCVVVDVSHEDPEVKFNWYVDGVEVHNAKTKP |
| | T256E) | REEQYNSTYRVVSVLTVLHQDWLNGKEYKCKVSN |
| | | KALPAPIEKTISKAKGQPREPQVYTLPPSRDELTKNQ |
| | | VSLTCLVKGFYPSDIAVEWESNGQPENNYKTTPPVL |
| | | DSDGSFFLYSKLTVDKSRWQQGNVFSCSVMHEALH |
| | | NHYTQKSLSLSPGK |
| 322 | RVT-3101-VH | QVQLVQSGAEVKKPGASVKVSCKASGYDFTYYGIS |
| | | WVRQAPGQGLEWMGWISTYNGNTHYARMLQGRV |
| | | TMTTDTSTRTAYMELRSLRSDDTAVYYCARENYYG |
| | | SGAYRGGMDVWGQGTTVTVSS |
| 323 | RVT-3101-VL | EIVLTQSPATLSLSPGERATLSCRASQSVSSYLAWYQ |
| | | QKPGQAPRLLIYDASNRATGIPARFSGSGSGTDFTLTI |
| | | SSLEPEDFAVYYCQQRSNWPWTFGQGTKVEIK |
| 324 | MK-7240-VH | QVQLVQSGAEVKKPGASVKVSCKASGFDIQDTYMH |
| | | WVKQRPGQGLEWMGRIDPASGHTKYDPKFQVRVTI |
| | | TRDTSTSTVYLELSSLRSEDTAVYYCARSGGLPDVW |
| | | GQGTTVTVSS |
| 325 | MK-7240-VL | EIVLTQSPGTLSLSPGERATLSCRASSSVSYMYWYQ |
| | | QKPGQAPRPLIYATSNLASGIPDRFSGSGSGTDFTLTI |
| | | SRLEPEDFAVYYCQQWEGNPRTFGGGTKLEIK |
| 326 | Human TL1A | MAEDLGLSFGETASVEMLPEHGSCRPKARSSSARW |
| | (M1-L251) | ALTCCLVLLPFLAGLTTYLLVSQLRAQGEACVQFQA |
| | | LKGQEFAPSHQQVYAPLRADGDKPRAHLTVVRQTP |
| | | TQHFKNQFPALHWEHELGLAFTKNRMNYTNKFLLIP |
| | | ESGDYFIYSQVTFRGMTSECSEIRQAGRPNKPDSITV |
| | | VITKVTDSYPEPTQLLMGTKSVCEVGSNWFQPIYLG |
| | | AMFSLQEGDKLMVNVSDISLVDYTKEDKTFFGAFLL |
| 327 | Human sTL1A | LKGQEFAPSHQQVYAPLRADGDKPRAHLTVVRQTP |
| | (L72-L251) | TQHFKNQFPALHWEHELGLAFTKNRMNYTNKFLLIP |
| | | ESGDYFIYSQVTFRGMTSECSEIRQAGRPNKPDSITV |
| | | VITKVTDSYPEPTQLLMGTKSVCEVGSNWFQPIYLG |
| | | AMFSLQEGDKLMVNVSDISLVDYTKEDKTFFGAFLL |
| 328 | Cyno sTL1A | LKGQEFAPSHQQVYAPLRADGDKPRAHLTVVRQTP |
| | (L72-L251) | TQHLKNQFPALHWEHELGLAFTKNRMNYTNKFLLI |
| | | PESGDYFVYSQVTFRGMTSECSEIRQAGRPNKPDSIT |
| | | VVITKVTDSYPEPTQLLMGTKSVCEVGSNWFQPIYL |
| | | GAMFSLQEGDKLMVNVSDISLVDYTK |
| | | EDKTFFGAFLL |
| 329 | Mouse sTL1A | AGQLRVPGKDCMLRAITEERSEPSPQQVYSPPRGKP |
| | (A61-L252) | RAHLTIKKQTPAPHLKNQLSALHWEHDLGMAFTKN |
| | | GMKYINKSLVIPESGDYFIYSQITFRGTTSVCGDISRG |
| | | RRPNKPDSITVVITKVADSYPEPARLLTGSKSVCEISN |
| | | NWFQSLYLGAMFSLEEGDRLMVNVSDISLVDYTKE |
| | | DKTFFGAFLL |
| 330 | Rat sTL1A | TGQLRIPGKDCMFPTVTEERSAPSAQPVYTPSRDKPK |
| | (T61-1252) | AHLTIMRQTPVPHLKNELAALHWENNLGMAFTKNR |
| | | MNYTNKFLVIPESGDYFIYSQITFRGTTSECGDISRVR |
| | | RPKKPDSITVVITKVADSYPEPAHLLTGTKSVCEISSN |
| | | WFQPIYLGAMESLEEGDRLMVNVSDISLVDYTKEDK |
| | | TFFGAFLI |
| 331 | Human DR3-ECD | QGGTRSPRCDCAGDFHKKIGLFCCRGCPAGHYLKAP |
| | (Q25-Q199) | CTEPCGNSTCLVCPQDTFLAWENHHNSECARCQAC |
| | | DEQASQVALENCSAVADTRCGCKPGWFVECQVSQC |
| | | VSSSPFYCQPCLDCGALHRHTRLLCSRRDTDCGTCL |
| | | PGFYEHGDGCVSCPTSTLGSCPERCAAVCGWRQ |
| 332 | Vedolizumab-HCDR1 | SYWMH |
| 333 | Vedolizumab-HCDR2 | EIDPSESNTNYNQKFKG |
| 334 | Vedolizumab-HCDR3 | GGYDGWDYAIDY |
| 335 | Vedolizumab-VH | QVQLVQSGAEVKKPGASVKVSCKGSGYTFTSYWM |
| | | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSS |
| 336 | Vedolizumab-LCDR1 | RSSQSLAKSYGNTYLS |
| 337 | Vedolizumab-LCDR2 | GISNRFS |
| 338 | Vedolizumab-LCDR3 | LQGTHQPYT |
| 339 | Vedolizumab-VL | DVVMTQSPLSLPVTPGEPASISCRSSQSLAKSYGNTY |
| | | LSWYLQKPGQSPQLLIYGISNRFSGVPDRFSGSGSGT |
| | | DFTLKISRVEAEDVGVYYCLQGTHQPYTFGQGTKVE |
| | | IK |
| 340 | scTLA0015-11 VHVL | EIQLVESGGGLIQPGGSLRISCAVSGFSLSSYGVDWV |
| | | RQAPGKGLEWLGVIWGFGGTNYNSALKSRLTISKD |
| | | NSKNTVYLQMNSLRAEDTAVYYCASGNFDAMDYW |
| | | GQGTLVTVSSGGGGSGGGGSGGGGSGGGGSDIQMT |
| | | QSPSSLSASVGDRVTITCRASQDISNYLNWYQQKPG |
| | | KAVKLLIYYTSRLHSGVPSRFSGSGSGTDYTLTISSL |
| | | QPEDFATYFCQQGNTLPFTFGQGTKLEIK |
| 341 | scTLA0015-11 VLVH | DIQMTQSPSSLSASVGDRVTITCRASQDISNYLNWY |
| | | QQKPGKAVKLLIYYTSRLHSGVPSRFSGSGSGTDYT |
| | | LTISSLQPEDFATYFCQQGNTLPFTFGQGTKLEIKGG |
| | | GGSGGGGSGGGGSGGGGSEIQLVESGGGLIQPGGSL |
| | | RISCAVSGFSLSSYGVDWVRQAPGKGLEWLGVIWG |
| | | FGGTNYNSALKSRLTISKDNSKNTVYLQMNSLRAED |
| | | TAVYYCASGNFDAMDYWGQGTLVTVSS |
| 342 | scTLA0015-11cc | EIQLVESGGGLIQPGGSLRISCAVSGFSLSSYGVDWV |
| | VHVL | RQAPGKCLEWLGVIWGFGGTNYNSALKSRLTISKD |
| | | NSKNTVYLQMNSLRAEDTAVYYCASGNFDAMDYW |
| | | GQGTLVTVSSGGGGSGGGGSGGGGSGGGGSDIQMT |
| | | QSPSSLSASVGDRVTITCRASQDISNYLNWYQQKPG |
| | | KAVKLLIYYTSRLHSGVPSRFSGSGSGTDYTLTISSL |
| | | QPEDFATYFCQQGNTLPFTFGCGTKLEIK |
| 343 | scTLA0015-11cc | DIQMTQSPSSLSASVGDRVTITCRASQDISNYLNWY |
| | VLVH | QQKPGKAVKLLIYYTSRLHSGVPSRFSGSGSGTDYT |
| | | LTISSLQPEDFATYFCQQGNTLPFTFGCGTKLEIKGG |
| | | GGSGGGGSGGGGSGGGGSEIQLVESGGGLIQPGGSL |
| | | RISCAVSGFSLSSYGVDWVRQAPGKCLEWLGVIWG |
| | | FGGTNYNSALKSRLTISKDNSKNTVYLQMNSLRAED |
| | | TAVYYCASGNFDAMDYWGQGTLVTVSS |
| 344 | scTLA0238-5 VHVL | EVQLVESGGGLVQPGGSLRLSCAASGFTFSNYWMN |
| | | WVRQAPGKGLEWVAQIRLKSDNYATHYADSVKGR |
| | | FTISRDNSKNTLYLQMNSLRAEDTAVYYCTPLLLRY |
| | | RDYWGQGTLVTVSSGGGGSGGGGSGGGGSGGGGS |
| | | DIQMTQSPSSVSASVGDRVTITCSASSSVSYMHWYQ |
| | | QKPGKAPKLWIYDTSNLASGVPSRFSGSGSGTDFTL |
| | | TISSLQPEDFATYYCFQENGHPFTFGQGTKVEIK |
| 345 | scTLA0238-5cc VHVL | EVQLVESGGGLVQPGGSLRLSCAASGFTFSNYWMN |
| | | WVRQAPGKCLEWVAQIRLKSDNYATHYADSVKGR |
| | | FTISRDNSKNTLYLQMNSLRAEDTAVYYCTPLLLRY |
| | | RDYWGQGTLVTVSSGGGGSGGGGSGGGGSGGGGS |
| | | DIQMTQSPSSVSASVGDRVTITCSASSSVSYMHWYQ |
| | | QKPGKAPKLWIYDTSNLASGVPSRFSGSGSGTDFTL |
| | | TISSLQPEDFATYYCFQENGHPFTFGCGTKVEIK |
| 346 | sc Vedolizumab VHVL | QVQLVQSGAEVKKPGASVKVSCKGSGYTFTSYWM |
| | | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSGGGGSGGGGSGGGGSGG |
| | | GGSDVVMTQSPLSLPVTPGEPASISCRSSQSLAKSYG |
| | | NTYLSWYLQKPGQSPQLLIYGISNRFSGVPDRESGSG |
| | | SGTDFTLKISRVEAEDVGVYYCLQGTHQPYTFGQGT |
| | | KVEIK |
| 347 | sc Vedolizumab-cc | QVQLVQSGAEVKKPGASVKVSCKGSGYTFTSYWM |
| | VHVL | HWVRQAPGQCLEWIGEIDPSESNTNYNQKFKGRVT |
| | | LTVDISASTAYMELSSLRSEDTAVYYCARGGYDGW |
| | | DYAIDYWGQGTLVTVSSGGGGSGGGGSGGGGSGG |
| | | GGSDVVMTQSPLSLPVTPGEPASISCRSSQSLAKSYG |
| | | NTYLSWYLQKPGQSPQLLIYGISNRFSGVPDRESGSG |
| | | SGTDFTLKISRVEAEDVGVYYCLQGTHQPYTFGCGT |
| | | KVEIK |
| 348 | light chain 1 | DVVMTQSPLSLPVTPGEPASISCRSSQSLAKSYGNTY |
| | | LSWYLQKPGQSPQLLIYGISNRFSGVPDRESGSGSGT |
| | | DFTLKISRVEAEDVGVYYCLQGTHQPYTFGQGTKVE |
| | | IKRTVAAPSVFIFPPSDEQLKSGTASVVCLLNNFYPR |
| | | EAKVQWKVDNALQSGNSQESVTEQDSKDSTYSLSS |
| | | TLTLSKADYEKHKVYACEVTHQGLSSPVTKSFNRGE |
| | | C |
| 349 | light chain 2 | DIQMTQSPSSLSASVGDRVTITCRASQDISNYLNWY |
| | | QQKPGKAVKLLIYYTSRLHSGVPSRFSGSGSGTDYT |
| | | LTISSLQPEDFATYFCQQGNTLPFTFGQGTKLEIKRT |
| | | VAAPSVFIFPPSDEQLKSGTASVVCLLNNFYPREAKV |
| | | QWKVDNALQSGNSQESVTEQDSKDSTYSLSSTLTLS |
| | | KADYEKHKVYACEVTHQGLSSPVTKSFNRGEC |
| 350 | TLb0045 heavy chain | QVQLVQSGAEVKKPGASVKVSCKGSGYTFTSYWM |
| | | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPSN |
| | | TKVDKKVEPKSCDKTHTCPPCPAPEAAGGPSVFLFP |
| | | PKPKDTLYITREPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGKGGGGSGGGG |
| | | SGGGGSGGGGSEIQLVESGGGLIQPGGSLRISCAVSG |
| | | FSLSSYGVDWVRQAPGKCLEWLGVIWGFGGTNYNS |
| | | ALKSRLTISKDNSKNTVYLQMNSLRAEDTAVYYCA |
| | | SGNFDAMDYWGQGTLVTVSSGGGGSGGGGSGGGG |
| | | SGGGGSDIQMTQSPSSLSASVGDRVTITCRASQDISN |
| | | YLNWYQQKPGKAVKLLIYYTSRLHSGVPSRFSGSGS |
| | | GTDYTLTISSLQPEDFATYFCQQGNTLPFTFGCGTKL |
| | | EIK |
| 351 | TLb0046 heavy chain | DIQMTQSPSSLSASVGDRVTITCRASQDISNYLNWY |
| | | QQKPGKAVKLLIYYTSRLHSGVPSRFSGSGSGTDYT |
| | | LTISSLQPEDFATYFCQQGNTLPFTFGCGTKLEIKGG |
| | | GGSGGGGSGGGGSGGGGSEIQLVESGGGLIQPGGSL |
| | | RISCAVSGFSLSSYGVDWVRQAPGKCLEWLGVIWG |
| | | FGGTNYNSALKSRLTISKDNSKNTVYLQMNSLRAED |
| | | TAVYYCASGNFDAMDYWGQGTLVTVSSGGGGSGG |
| | | GGSGGGGSGGGGSQVQLVQSGAEVKKPGASVKVSC |
| | | KGSGYTFTSYWMHWVRQAPGQRLEWIGEIDPSESN |
| | | TNYNQKFKGRVTLTVDISASTAYMELSSLRSEDTAV |
| | | YYCARGGYDGWDYAIDYWGQGTLVTVSSASTKGP |
| | | SVFPLAPSSKSTSGGTAALGCLVKDYFPEPVTVSWN |
| | | SGALTSGVHTFPAVLQSSGLYSLSSVVTVPSSSLGTQ |
| | | TYICNVNHKPSNTKVDKKVEPKSCDKTHTCPPCPAP |
| | | EAAGGPSVFLFPPKPKDTLYITREPEVTCVVVDVSHE |
| | | DPEVKFNWYVDGVEVHNAKTKPREEQYNSTYRVV |
| | | SVLTVLHQDWLNGKEYKCKVSNKALPAPIEKTISKA |
| | | KGQPREPQVYTLPPSRDELTKNQVSLTCLVKGFYPS |
| | | DIAVEWESNGQPENNYKTTPPVLDSDGSFFLYSKLT |
| | | VDKSRWQQGNVFSCSVMHEALHNHYTQKSLSLSPG |
| | | K |
| 352 | TLb0065 heavy chain | QVQLVQSGAEVKKPGASVKVSCKGSGYTFTSYWM |
| | | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPSN |
| | | TKVDKKVEPKSCDKTHTCPPCPAPEAAGGPSVFLFP |
| | | PKPKDTLYITREPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGKGGGGSGGGG |
| | | SGGGGSGGGGSEIQLVESGGGLIQPGGSLRISCAVSG |
| | | FSLSSYGVDWVRQAPGKGLEWLGVIWGFGGTNYNS |
| | | ALKSRLTISKDNSKNTVYLQMNSLRAEDTAVYYCA |
| | | SGNFDAMDYWGQGTLVTVSSGGGGSGGGGSGGGG |
| | | SGGGGSDIQMTQSPSSLSASVGDRVTITCRASQDISN |
| | | YLNWYQQKPGKAVKLLIYYTSRLHSGVPSRESGSGS |
| | | GTDYTLTISSLQPEDFATYFCQQGNTLPFTFGQGTKL |
| | | EIK |
| 353 | TLb0066 heavy chain | DIQMTQSPSSLSASVGDRVTITCRASQDISNYLNWY |
| | | QQKPGKAVKLLIYYTSRLHSGVPSRFSGSGSGTDYT |
| | | LTISSLQPEDFATYFCQQGNTLPFTFGQGTKLEIKGG |
| | | GGSGGGGSGGGGSGGGGSEIQLVESGGGLIQPGGSL |
| | | RISCAVSGFSLSSYGVDWVRQAPGKGLEWLGVIWG |
| | | FGGTNYNSALKSRLTISKDNSKNTVYLQMNSLRAED |
| | | TAVYYCASGNFDAMDYWGQGTLVTVSSGGGGSGG |
| | | GGSGGGGSGGGGSQVQLVQSGAEVKKPGASVKVSC |
| | | KGSGYTFTSYWMHWVRQAPGQRLEWIGEIDPSESN |
| | | TNYNQKFKGRVTLTVDISASTAYMELSSLRSEDTAV |
| | | YYCARGGYDGWDYAIDYWGQGTLVTVSSASTKGP |
| | | SVFPLAPSSKSTSGGTAALGCLVKDYFPEPVTVSWN |
| | | SGALTSGVHTFPAVLQSSGLYSLSSVVTVPSSSLGTQ |
| | | TYICNVNHKPSNTKVDKKVEPKSCDKTHTCPPCPAP |
| | | EAAGGPSVFLFPPKPKDTLYITREPEVTCVVVDVSHE |
| | | DPEVKFNWYVDGVEVHNAKTKPREEQYNSTYRVV |
| | | SVLTVLHQDWLNGKEYKCKVSNKALPAPIEKTISKA |
| | | KGQPREPQVYTLPPSRDELTKNQVSLTCLVKGFYPS |
| | | DIAVEWESNGQPENNYKTTPPVLDSDGSFFLYSKLT |
| | | VDKSRWQQGNVFSCSVMHEALHNHYTQKSLSLSPG |
| | | K |
| 354 | TLb0067 heavy chain | EIQLVESGGGLIQPGGSLRISCAVSGFSLSSYGVDWV |
| | | RQAPGKGLEWLGVIWGFGGTNYNSALKSRLTISKD |
| | | NSKNTVYLQMNSLRAEDTAVYYCASGNFDAMDYW |
| | | GQGTLVTVSSASTKGPSVFPLAPSSKSTSGGTAALGC |
| | | LVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPEAAGGPSVFLFPPKPKDTLYIT |
| | | REPEVTCVVVDVSHEDPEVKFNWYVDGVEVHNAK |
| | | TKPREEQYNSTYRVVSVLTVLHQDWLNGKEYKCKV |
| | | SNKALPAPIEKTISKAKGQPREPQVYTLPPSRDELTK |
| | | NQVSLTCLVKGFYPSDIAVEWESNGQPENNYKTTPP |
| | | VLDSDGSFFLYSKLTVDKSRWQQGNVFSCSVMHEA |
| | | LHNHYTQKSLSLSPGKGGGGSGGGGSGGGGSGGGG |
| | | SQVQLVQSGAEVKKPGASVKVSCKGSGYTFTSYWM |
| | | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSGGGGSGGGGSGGGGSGG |
| | | GGSDVVMTQSPLSLPVTPGEPASISCRSSQSLAKSYG |
| | | NTYLSWYLQKPGQSPQLLIYGISNRFSGVPDRESGSG |
| | | SGTDFTLKISRVEAEDVGVYYCLQGTHQPYTFGQGT |
| | | KVEIK |
| 355 | TLb0068 heavy chain | DVVMTQSPLSLPVTPGEPASISCRSSQSLAKSYGNTY |
| | | LSWYLQKPGQSPQLLIYGISNRFSGVPDRFSGSGSGT |
| | | DFTLKISRVEAEDVGVYYCLQGTHQPYTFGQGTKVE |
| | | IKGGGGSGGGGSGGGGSGGGGSQVQLVQSGAEVKK |
| | | PGASVKVSCKGSGYTFTSYWMHWVRQAPGQRLEW |
| | | IGEIDPSESNTNYNQKFKGRVTLTVDISASTAYMELS |
| | | SLRSEDTAVYYCARGGYDGWDYAIDYWGQGTLVT |
| | | VSSGGGGSGGGGSGGGGSGGGGSEIQLVESGGGLIQ |
| | | PGGSLRISCAVSGFSLSSYGVDWVRQAPGKGLEWLG |
| | | VIWGFGGTNYNSALKSRLTISKDNSKNTVYLQMNSL |
| | | RAEDTAVYYCASGNFDAMDYWGQGTLVTVSSAST |
| | | KGPSVFPLAPSSKSTSGGTAALGCLVKDYFPEPVTVS |
| | | WNSGALTSGVHTFPAVLQSSGLYSLSSVVTVPSSSL |
| | | GTQTYICNVNHKPSNTKVDKKVEPKSCDKTHTCPPC |
| | | PAPEAAGGPSVFLFPPKPKDTLYITREPEVTCVVVDV |
| | | SHEDPEVKFNWYVDGVEVHNAKTKPREEQYNSTYR |
| | | VVSVLTVLHQDWLNGKEYKCKVSNKALPAPIEKTIS |
| | | KAKGQPREPQVYTLPPSRDELTKNQVSLTCLVKGFY |
| | | PSDIAVEWESNGQPENNYKTTPPVLDSDGSFFLYSKL |
| | | TVDKSRWQQGNVFSCSVMHEALHNHYTQKSLSLSP |
| | | GK |
| 356 | TLb0081 heavy chain | QVQLVQSGAEVKKPGASVKVSCKGSGYTFTSYWM |
| | | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPSN |
| | | TKVDKKVEPKSCDKTHTCPPCPAPEAAGGPSVFLFP |
| | | PKPKDTLYITREPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGKGGGGSGGGG |
| | | SGGGGSGGGGSEVQLVESGGGLVQPGGSLRLSCAA |
| | | SGFTFSNYWMNWVRQAPGKGLEWVAQIRLKSDNY |
| | | ATHYADSVKGRFTISRDNSKNTLYLQMNSLRAEDT |
| | | AVYYCTPLLLRYRDYWGQGTLVTVSSGGGGSGGG |
| | | GSGGGGSGGGGSDIQMTQSPSSVSASVGDRVTITCS |
| | | ASSSVSYMHWYQQKPGKAPKLWIYDTSNLASGVPS |
| | | RFSGSGSGTDFTLTISSLQPEDFATYYCFQENGHPFTF |
| | | GQGTKVEIK |
| 357 | TLb0085 heavy chain | EVQLVESGGGLVQPGGSLRLSCAASGFTFSNYWMN |
| | | WVRQAPGKGLEWVAQIRLKSDNYATHYADSVKGR |
| | | FTISRDNSKNTLYLQMNSLRAEDTAVYYCTPLLLRY |
| | | RDYWGQGTLVTVSSASTKGPSVFPLAPSSKSTSGGT |
| | | AALGCLVKDYFPEPVTVSWNSGALTSGVHTFPAVL |
| | | QSSGLYSLSSVVTVPSSSLGTQTYICNVNHKPSNTKV |
| | | DKKVEPKSCDKTHTCPPCPAPEAAGGPSVFLFPPKPK |
| | | DTLYITREPEVTCVVVDVSHEDPEVKFNWYVDGVE |
| | | VHNAKTKPREEQYNSTYRVVSVLTVLHQDWLNGK |
| | | EYKCKVSNKALPAPIEKTISKAKGQPREPQVYTLPPS |
| | | RDELTKNQVSLTCLVKGFYPSDIAVEWESNGQPENN |
| | | YKTTPPVLDSDGSFFLYSKLTVDKSRWQQGNVFSCS |
| | | VMHEALHNHYTQKSLSLSPGKGGGGSGGGGSGGG |
| | | GSGGGGSQVQLVQSGAEVKKPGASVKVSCKGSGYT |
| | | FTSYWMHWVRQAPGQRLEWIGEIDPSESNTNYNQK |
| | | FKGRVTLTVDISASTAYMELSSLRSEDTAVYYCARG |
| | | GYDGWDYAIDYWGQGTLVTVSSGGGGSGGGGSGG |
| | | GGSGGGGSDVVMTQSPLSLPVTPGEPASISCRSSQSL |
| | | AKSYGNTYLSWYLQKPGQSPQLLIYGISNRFSGVPD |
| | | RFSGSGSGTDFTLKISRVEAEDVGVYYCLQGTHQPY |
| | | TFGQGTKVEIK |
| 358 | TLb0086 heavy chain | EIQLVESGGGLIQPGGSLRISCAVSGFSLSSYGVDWV |
| | | RQAPGKGLEWLGVIWGFGGTNYNSALKSRLTISKD |
| | | NSKNTVYLQMNSLRAEDTAVYYCASGNFDAMDYW |
| | | GQGTLVTVSSASTKGPSVFPLAPSSKSTSGGTAALGC |
| | | LVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPEAAGGPSVFLFPPKPKDTLYIT |
| | | REPEVTCVVVDVSHEDPEVKFNWYVDGVEVHNAK |
| | | TKPREEQYNSTYRVVSVLTVLHQDWLNGKEYKCKV |
| | | SNKALPAPIEKTISKAKGQPREPQVYTLPPSRDELTK |
| | | NQVSLTCLVKGFYPSDIAVEWESNGQPENNYKTTPP |
| | | VLDSDGSFFLYSKLTVDKSRWQQGNVFSCSVMHEA |
| | | LHNHYTQKSLSLSPGKGGGGSGGGGSGGGGSGGGG |
| | | SQVQLVQSGAEVKKPGASVKVSCKGSGYTFTSYWM |
| | | HWVRQAPGQCLEWIGEIDPSESNTNYNQKFKGRVT |
| | | LTVDISASTAYMELSSLRSEDTAVYYCARGGYDGW |
| | | DYAIDYWGQGTLVTVSSGGGGSGGGGSGGGGSGG |
| | | GGSDVVMTQSPLSLPVTPGEPASISCRSSQSLAKSYG |
| | | NTYLSWYLQKPGQSPQLLIYGISNRFSGVPDRESGSG |
| | | SGTDFTLKISRVEAEDVGVYYCLQGTHQPYTFGCGT |
| | | KVEIK |
| 359 | TLb0087 heavy chain | QVQLVQSGAEVKKPGASVKVSCKGSGYTFTSYWM |
| | | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPSN |
| | | TKVDKKVEPKSCDKTHTCPPCPAPEAAGGPSVFLFP |
| | | PKPKDTLYITREPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGKGGGGSGGGG |
| | | SGGGGSGGGGSEVQLVESGGGLVQPGGSLRLSCAA |
| | | SGFTFSNYWMNWVRQAPGKCLEWVAQIRLKSDNY |
| | | ATHYADSVKGRFTISRDNSKNTLYLQMNSLRAEDT |
| | | AVYYCTPLLLRYRDYWGQGTLVTVSSGGGGSGGG |
| | | GSGGGGSGGGGSDIQMTQSPSSVSASVGDRVTITCS |
| | | ASSSVSYMHWYQQKPGKAPKLWIYDTSNLASGVPS |
| | | RFSGSGSGTDFTLTISSLQPEDFATYYCFQENGHPFTF |
| | | GCGTKVEIK |
| 360 | TLb0088 heavy chain | EVQLVESGGGLVQPGGSLRLSCAASGFTFSNYWMN |
| | | WVRQAPGKGLEWVAQIRLKSDNYATHYADSVKGR |
| | | FTISRDNSKNTLYLQMNSLRAEDTAVYYCTPLLLRY |
| | | RDYWGQGTLVTVSSASTKGPSVFPLAPSSKSTSGGT |
| | | AALGCLVKDYFPEPVTVSWNSGALTSGVHTFPAVL |
| | | QSSGLYSLSSVVTVPSSSLGTQTYICNVNHKPSNTKV |
| | | DKKVEPKSCDKTHTCPPCPAPEAAGGPSVFLFPPKPK |
| | | DTLYITREPEVTCVVVDVSHEDPEVKFNWYVDGVE |
| | | VHNAKTKPREEQYNSTYRVVSVLTVLHQDWLNGK |
| | | EYKCKVSNKALPAPIEKTISKAKGQPREPQVYTLPPS |
| | | RDELTKNQVSLTCLVKGFYPSDIAVEWESNGQPENN |
| | | YKTTPPVLDSDGSFFLYSKLTVDKSRWQQGNVFSCS |
| | | VMHEALHNHYTQKSLSLSPGKGGGGSGGGGSGGG |
| | | GSGGGGSQVQLVQSGAEVKKPGASVKVSCKGSGYT |
| | | FTSYWMHWVRQAPGQCLEWIGEIDPSESNTNYNQK |
| | | FKGRVTLTVDISASTAYMELSSLRSEDTAVYYCARG |
| | | GYDGWDYAIDYWGQGTLVTVSSGGGGSGGGGSGG |
| | | GGSGGGGSDVVMTQSPLSLPVTPGEPASISCRSSQSL |
| | | AKSYGNTYLSWYLQKPGQSPQLLIYGISNRFSGVPD |
| | | RFSGSGSGTDFTLKISRVEAEDVGVYYCLQGTHQPY |
| | | TFGCGTKVEIK |
| 361 | TLb0093 heavy chain | QVQLVQSGAEVKKPGASVKVSCKGSGYTFTSYWM |
| | | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPSN |
| | | TKVDKKVEPKSCDKTHTCPPCPAPEAAGGPSVFLFP |
| | | PKPKDTLYITREPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGKGGGGSGGGG |
| | | SGGGGSGGGGSDIQMTQSPSSLSASVGDRVTITCRAS |
| | | QDISNYLNWYQQKPGKAVKLLIYYTSRLHSGVPSRF |
| | | SGSGSGTDYTLTISSLQPEDFATYFCQQGNTLPFTFG |
| | | CGTKLEIKGGGGSGGGGSGGGGSGGGGSEIQLVESG |
| | | GGLIQPGGSLRISCAVSGFSLSSYGVDWVRQAPGKC |
| | | LEWLGVIWGFGGTNYNSALKSRLTISKDNSKNTVYL |
| | | QMNSLRAEDTAVYYCASGNFDAMDYWGQGTLVTV |
| | | SS |
| 362 | TLb0094 heavy chain | QVQLVQSGAEVKKPGASVKVSCKGSGYTFTSYWM |
| | | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPSN |
| | | TKVDKKVEPKSCDKTHTCPPCPAPEAAGGPSVFLFP |
| | | PKPKDTLYITREPEVTCVVVDVSHEDPEVKFNWYVD |
| | | GVEVHNAKTKPREEQYNSTYRVVSVLTVLHQDWL |
| | | NGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGKGGGGSGGGG |
| | | SGGGGSGGGGSDIQMTQSPSSLSASVGDRVTITCRAS |
| | | QDISNYLNWYQQKPGKAVKLLIYYTSRLHSGVPSRF |
| | | SGSGSGTDYTLTISSLQPEDFATYFCQQGNTLPFTFG |
| | | QGTKLEIKGGGGSGGGGSGGGGSGGGGSEIQLVESG |
| | | GGLIQPGGSLRISCAVSGFSLSSYGVDWVRQAPGKG |
| | | LEWLGVIWGFGGTNYNSALKSRLTISKDNSKNTVYL |
| | | QMNSLRAEDTAVYYCASGNFDAMDYWGQGTLVTV |
| | | SS |
| 363 | TLb0045-1 heavy | QVQLVQSGAEVKKPGASVKVSCKGSGYTFTSYWM |
| | chain | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPSN |
| | | TKVDKKVEPKSCDKTHTCPPCPAPEAAGGPSVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPAPIEKTISKAKGQPREPQVY |
| | | TLPPSRDELTKNQVSLTCLVKGFYPSDIAVEWESNG |
| | | QPENNYKTTPPVLDSDGSFFLYSKLTVDKSRWQQG |
| | | NVFSCSVLHEALHSHYTQKSLSLSPGKGGGGSGGGG |
| | | SGGGGSGGGGSEIQLVESGGGLIQPGGSLRISCAVSG |
| | | FSLSSYGVDWVRQAPGKCLEWLGVIWGFGGTNYNS |
| | | ALKSRLTISKDNSKNTVYLQMNSLRAEDTAVYYCA |
| | | SGNFDAMDYWGQGTLVTVSSGGGGSGGGGSGGGG |
| | | SGGGGSDIQMTQSPSSLSASVGDRVTITCRASQDISN |
| | | YLNWYQQKPGKAVKLLIYYTSRLHSGVPSRFSGSGS |
| | | GTDYTLTISSLQPEDFATYFCQQGNTLPFTFGCGTKL |
| | | EIK |
| 364 | TLb0045-2 heavy | QVQLVQSGAEVKKPGASVKVSCKGSGYTFTSYWM |
| | chain | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPSN |
| | | TKVDKKVEPKSCDKTHTCPPCPAPEAAGGPSVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPAPIEKTISKAKGQPREPQVY |
| | | TLPPSRDELTKNQVSLTCLVKGFYPSDIAVEWESNG |
| | | QPENNYKTTPPVLDSDGSFFLYSKLTVDKSRWQQG |
| 365 | TLb0045-3 heavy | NVFSCSVLHEALHSHYTQKSLSLSPGKGGGGSGGGG |
| | chain | SEIQLVESGGGLIQPGGSLRISCAVSGFSLSSYGVDW |
| | | VRQAPGKCLEWLGVIWGFGGTNYNSALKSRLTISK |
| | | DNSKNTVYLQMNSLRAEDTAVYYCASGNFDAMDY |
| | | WGQGTLVTVSSGGGGSGGGGSGGGGSGGGGSDIQ |
| | | MTQSPSSLSASVGDRVTITCRASQDISNYLNWYQQK |
| | | PGKAVKLLIYYTSRLHSGVPSRFSGSGSGTDYTLTISS |
| | | LQPEDFATYFCQQGNTLPFTFGCGTKLEIK |
| | | QVQLVQSGAEVKKPGASVKVSCKGSGYTFTSYWM |
| | | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPSN |
| | | TKVDKKVEPKSCDKTHTCPPCPAPEAAGGPSVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPAPIEKTISKAKGQPREPQVY |
| | | TLPPSRDELTKNQVSLTCLVKGFYPSDIAVEWESNG |
| | | QPENNYKTTPPVLDSDGSFFLYSKLTVDKSRWQQG |
| | | NVFSCSVLHEALHSHYTQKSLSLSPGKGGGGSGGGG |
| | | SGGGGSEIQLVESGGGLIQPGGSLRISCAVSGFSLSSY |
| | | GVDWVRQAPGKCLEWLGVIWGFGGTNYNSALKSR |
| | | LTISKDNSKNTVYLQMNSLRAEDTAVYYCASGNFD |
| | | AMDYWGQGTLVTVSSGGGGSGGGGSGGGGSGGGG |
| | | SDIQMTQSPSSLSASVGDRVTITCRASQDISNYLNWY |
| | | QQKPGKAVKLLIYYTSRLHSGVPSRFSGSGSGTDYT |
| | | LTISSLQPEDFATYFCQQGNTLPFTFGCGTKLEIK |
| 366 | TLb0065-1 heavy | QVQLVQSGAEVKKPGASVKVSCKGSGYTFTSYWM |
| | chain | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPSN |
| | | TKVDKKVEPKSCDKTHTCPPCPAPEAAGGPSVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPAPIEKTISKAKGQPREPQVY |
| | | TLPPSRDELTKNQVSLTCLVKGFYPSDIAVEWESNG |
| | | QPENNYKTTPPVLDSDGSFFLYSKLTVDKSRWQQG |
| | | NVFSCSVLHEALHSHYTQKSLSLSPGKGGGGSGGGG |
| | | SGGGGSGGGGSEIQLVESGGGLIQPGGSLRISCAVSG |
| | | FSLSSYGVDWVRQAPGKGLEWLGVIWGFGGTNYNS |
| | | ALKSRLTISKDNSKNTVYLQMNSLRAEDTAVYYCA |
| | | SGNFDAMDYWGQGTLVTVSSGGGGSGGGGSGGGG |
| | | SGGGGSDIQMTQSPSSLSASVGDRVTITCRASQDISN |
| | | YLNWYQQKPGKAVKLLIYYTSRLHSGVPSRFSGSGS |
| | | GTDYTLTISSLQPEDFATYFCQQGNTLPFTFGQGTKL |
| | | EIK |
| 367 | TLb0069 heavy chain- | EIQLVESGGGLIQPGGSLRISCAVSGFSLSSYGVDWV |
| | 1 | RQAPGKGLEWLGVIWGFGGTNYNSALKSRLTISKD |
| | | NSKNTVYLQMNSLRAEDTAVYYCASGNFDAMDYW |
| | | GQGTLVTVSSASTKGPSVFPLAPSSKSTSGGTAALGC |
| | | LVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPEAAGGPSVFLFPPKPKDTLMIS |
| | | RTPEVTCVVVDVSHEDPEVKFNWYVDGVEVHNAK |
| | | TKPREEQYNSTYRVVSVLTVLHQDWLNGKEYKCKV |
| | | SNKALPAPIEKTISKAKGQPREPQVYTLPPCRDELTK |
| | | NQVSLWCLVKGFYPSDIAVEWESNGQPENNYKTTP |
| | | PVLDSDGSFFLYSKLTVDKSRWQQGNVFSCSVMHE |
| | | ALHNHYTQKSLSLSPGK |
| 368 | TLb0069 heavy chain- | DVVMTQSPLSLPVTPGEPASISCRSSQSLAKSYGNTY |
| | 2 | LSWYLQKPGQSPQLLIYGISNRFSGVPDRFSGSGSGT |
| | | DFTLKISRVEAEDVGVYYCLQGTHQPYTFGCGTKVE |
| | | IKGGGGSGGGGSGGGGSGGGGSQVQLVQSGAEVKK |
| | | PGASVKVSCKGSGYTFTSYWMHWVRQAPGQCLEW |
| | | IGEIDPSESNTNYNQKFKGRVTLTVDISASTAYMELS |
| | | SLRSEDTAVYYCARGGYDGWDYAIDYWGQGTLVT |
| | | VSSEPKSADKTHTCPPCPAPEAAGGPSVFLFPPKPKD |
| | | TLMISRTPEVTCVVVDVSHEDPEVKFNWYVDGVEV |
| | | HNAKTKPREEQYNSTYRVVSVLTVLHQDWLNGKE |
| | | YKCKVSNKALPAPIEKTISKAKGQPREPQVCTLPPSR |
| | | DELTKNQVSLSCAVKGFYPSDIAVEWESNGQPENNY |
| | | KTTPPVLDSDGSFFLVSKLTVDKSRWQQGNVFSCSV |
| | | MHEALHNRFTQKSLSLSPGK |
| 369 | TLb0070 heavy chain- | QVQLVQSGAEVKKPGASVKVSCKGSGYTFTSYWM |
| | 1 | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPSN |
| | | TKVDKKVEPKSCDKTHTCPPCPAPEAAGGPSVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPAPIEKTISKAKGQPREPQVY |
| | | TLPPCRDELTKNQVSLWCLVKGFYPSDIAVEWESNG |
| | | QPENNYKTTPPVLDSDGSFFLYSKLTVDKSRWQQG |
| | | NVFSCSVMHEALHNHYTQKSLSLSPGK |
| 370 | TLb0070 heavy chain- | DIQMTQSPSSLSASVGDRVTITCRASQDISNYLNWY |
| | 2 | QQKPGKAVKLLIYYTSRLHSGVPSRFSGSGSGTDYT |
| | | LTISSLQPEDFATYFCQQGNTLPFTFGCGTKLEIKGG |
| | | GGSGGGGSGGGGSGGGGSEIQLVESGGGLIQPGGSL |
| | | RISCAVSGFSLSSYGVDWVRQAPGKCLEWLGVIWG |
| | | FGGTNYNSALKSRLTISKDNSKNTVYLQMNSLRAED |
| | | TAVYYCASGNFDAMDYWGQGTLVTVSSEPKSADK |
| | | THTCPPCPAPEAAGGPSVFLFPPKPKDTLMISRTPEV |
| | | TCVVVDVSHEDPEVKFNWYVDGVEVHNAKTKPRE |
| | | EQYNSTYRVVSVLTVLHQDWLNGKEYKCKVSNKA |
| | | LPAPIEKTISKAKGQPREPQVCTLPPSRDELTKNQVS |
| | | LSCAVKGFYPSDIAVEWESNGQPENNYKTTPPVLDS |
| | | DGSFFLVSKLTVDKSRWQQGNVFSCSVMHEALHNR |
| | | FTQKSLSLSPGK |
| 371 | Fc(LALA+LS) | DKTHTCPPCPAPEAAGGPSVFLFPPKPKDTLMISRTP |
| | | EVTCVVVDVSHEDPEVKFNWYVDGVEVHNAKTKP |
| | | REEQYNSTYRVVSVLTVLHQDWLNGKEYKCKVSN |
| | | KALPAPIEKTISKAKGQPREPQVYTLPPSRDELTKNQ |
| | | VSLTCLVKGFYPSDIAVEWESNGQPENNYKTTPPVL |
| | | DSDGSFFLYSKLTVDKSRWQQGNVFSCSVLHEALHS |
| | | HYTQKSLSLSPGK |
| 372 | light chain 3 | DIQMTQSPSSVSASVGDRVTITCSASSSVSYMHWYQ |
| | | QKPGKAPKLWIYDTSNLASGVPSRFSGSGSGTDFTL |
| | | TISSLQPEDFATYYCFQENGHPFTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| 373 | Fc-Knob | DKTHTCPPCPAPEAAGGPSVFLFPPKPKDTLMISRTP |
| | | EVTCVVVDVSHEDPEVKFNWYVDGVEVHNAKTKP |
| | | REEQYNSTYRVVSVLTVLHQDWLNGKEYKCKVSN |
| | | KALPAPIEKTISKAKGQPREPQVYTLPPCRDELTKNQ |
| | | VSLWCLVKGFYPSDIAVEWESNGQPENNYKTTPPVL |
| | | DSDGSFFLYSKLTVDKSRWQQGNVFSCSVMHEALH |
| | | NHYTQKSLSLSPGK |
| 374 | Fc-Hole | DKTHTCPPCPAPEAAGGPSVFLFPPKPKDTLMISRTP |
| | | EVTCVVVDVSHEDPEVKFNWYVDGVEVHNAKTKP |
| | | REEQYNSTYRVVSVLTVLHQDWLNGKEYKCKVSN |
| | | KALPAPIEKTISKAKGQPREPQVCTLPPSRDELTKNQ |
| | | VSLSCAVKGFYPSDIAVEWESNGQPENNYKTTPPVL |
| | | DSDGSFFLVSKLTVDKSRWQQGNVFSCSVMHEALH |
| | | NRFTQKSLSLSPGK |
| 375 | scRVT-3101 VHVL | QVQLVQSGAEVKKPGASVKVSCKASGYDFTYYGIS |
| | | WVRQAPGQGLEWMGWISTYNGNTHYARMLQGRV |
| | | TMTTDTSTRTAYMELRSLRSDDTAVYYCARENYYG |
| | | SGAYRGGMDVWGQGTTVTVSSGGGGSGGGGSGGG |
| | | GSGGGGSEIVLTQSPATLSLSPGERATLSCRASQSVS |
| | | SYLAWYQQKPGQAPRLLIYDASNRATGIPARFSGSG |
| | | SGTDFTLTISSLEPEDFAVYYCQQRSNWPWTFGQGT |
| | | KVEIK |
| 376 | scRVT-3101 VLVH | EIVLTQSPATLSLSPGERATLSCRASQSVSSYLAWYQ |
| | | QKPGQAPRLLIYDASNRATGIPARFSGSGSGTDFTLTI |
| | | SSLEPEDFAVYYCQQRSNWPWTFGQGTKVEIKGGG |
| | | GSGGGGSGGGGSGGGGSQVQLVQSGAEVKKPGAS |
| | | VKVSCKASGYDFTYYGISWVRQAPGQGLEWMGWI |
| | | STYNGNTHYARMLQGRVTMTTDTSTRTAYMELRSL |
| | | RSDDTAVYYCARENYYGSGAYRGGMDVWGQGTT |
| | | VTVSS |
| 377 | scRVT-3101cc | QVQLVQSGAEVKKPGASVKVSCKASGYDFTYYGIS |
| | | WVRQAPGQCLEWMGWISTYNGNTHYARMLQGRV |
| | | TMTTDTSTRTAYMELRSLRSDDTAVYYCARENYYG |
| | | SGAYRGGMDVWGQGTTVTVSSGGGGSGGGGSGGG |
| | | GSGGGGSEIVLTQSPATLSLSPGERATLSCRASQSVS |
| | | SYLAWYQQKPGQAPRLLIYDASNRATGIPARFSGSG |
| | | SGTDFTLTISSLEPEDFAVYYCQQRSNWPWTFGCGT |
| | | KVEIK |
| 378 | TLb0111-heavy chain | QVQLVQSGAEVKKPGASVKVSCKGSGYTFTSYWM |
| | | HWVRQAPGQRLEWIGEIDPSESNTNYNQKFKGRVT |
| | | LTVDISASTAYMELSSLRSEDTAVYYCARGGYDGW |
| | | DYAIDYWGQGTLVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKPSN |
| | | TKVDKKVEPKSCDKTHTCPPCPAPEAAGGPSVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPAPIEKTISKAKGQPREPQVY |
| | | TLPPSRDELTKNQVSLTCLVKGFYPSDIAVEWESNG |
| | | QPENNYKTTPPVLDSDGSFFLYSKLTVDKSRWQQG |
| | | NVFSCSVLHEALHSHYTQKSLSLSPGKGGGGSGGGG |
| | | SGGGGSGGGGSQVQLVQSGAEVKKPGASVKVSCKA |
| | | SGYDFTYYGISWVRQAPGQCLEWMGWISTYNGNTH |
| | | YARMLQGRVTMTTDTSTRTAYMELRSLRSDDTAVY |
| | | YCARENYYGSGAYRGGMDVWGQGTTVTVSSGGGG |
| | | SGGGGSGGGGSGGGGSEIVLTQSPATLSLSPGERATL |
| | | SCRASQSVSSYLAWYQQKPGQAPRLLIYDASNRATG |
| | | IPARFSGSGSGTDFTLTISSLEPEDFAVYYCQQRSNW |
| | | PWTFGCGTKVEIK |
| 379 | TLA0015-VH-41 | EIQLVESGGGLIQPGGSLRISCAVSGFSLSSYGVDWV |
| | | RQAPGKCLEWLGVIWGFGGTNYNSALKSRLTISKD |
| | | NSKNTVYLQMNSLRAEDTAVYYCASGNFDAMDYW |
| | | GQGTLVTVSS |
| 380 | TLA0015-VL-6 | DIQMTQSPSSLSASVGDRVTITCRASQDISNYLNWY |
| | | QQKPGKAVKLLIYYTSRLHSGVPSRESGSGSGTDYT |
| | | LTISSLQPEDFATYFCQQGNTLPFTFGCGTKLEIK |
| 381 | Fc (LALA + LS) | DKTHTCPPCPAPEAAGGPSVFLFPPKPKDTLMISRTP |
| | | EVTCVVVDVSHEDPEVKFNWYVDGVEVHNAKTKP |
| | | REEQYNSTYRVVSVLTVLHQDWLNGKEYKCKVSN |
| | | KALPAPIEKTISKAKGQPREPQVYTLPPSRDELTKNQ |
| | | VSLTCLVKGFYPSDIAVEWESNGQPENNYKTTPPVL |
| | | DSDGSFFLYSKLTVDKSRWQQGNVFSCSVLHEALHS |
| | | HYTQKSLSLSPGK |
| 382 | HFR1-6 | QVQLVQSGAEVKKPGASVKVSCKGSGYTFT |
| 383 | HFR2-2 | WVRQAPGQRLEWIG |
| 384 | HFR3-2 | RVTLTVDISASTAYMELSSLRSEDTAVYYCAR |
| 385 | HFR4-1 | WGQGTLVTVSS |
| 386 | LFR1-2 | DVVMTQSPLSLPVTPGEPASISC |
| 387 | LFR2-3 | WYLQKPGQSPQLLIY |
| 388 | LFR3-3 | GVPDRFSGSGSGTDFTLKISRVEAEDVGVYYC |
| 389 | LFR4-3 | FGQGTKVEIK |

1-35. (canceled)

36. An isolated antigen-binding protein, comprising: a first antigen-binding domain and a second antigen-binding domain, wherein the first antigen-binding domain specifically binds to TNF-like protein A (TL1A), and wherein the second antigen binding domain specifically binds to α4β7.

37. The isolated antigen-binding protein of claim 36, comprising: a first antigen-binding domain and a second antigen-binding domain, wherein the first antigen-binding domain specifically binds to TNF-like protein A (TL1A), and the second antigen binding domain specifically binds to α4β7, wherein the first antigen-binding domain comprises a heavy chain variable region (VH), and the VH comprises an HCDR3 SEQ ID NO:14.

38. The isolated antigen-binding protein according to claim 36, wherein the HCDR3 comprises an amino acid sequence of any one of SEQ ID NOs: 15-18.

39. The isolated antigen-binding protein according to claim 36, wherein the VH of the first antigen-binding domain comprises an HCDR2 comprising an amino acid sequence of SEQ ID NO:5.

40. The isolated antigen-binding protein of claim 39, wherein the HCDR2 comprises an amino acid sequence of any one of SEQ ID NOs: 6-13.

41. The isolated antigen-binding protein according to claim 36, wherein the VH of the first antigen-binding domain comprises an HCDR1 comprising an amino acid sequence of SEQ ID NO:1.

42. The isolated antigen-binding protein of claim 41, wherein the HCDR1 comprises an amino acid sequence of any one of SEQ ID NOs:2-4.

43. The isolated antigen-binding protein according to claim 37, wherein the VH of the first antigen-binding domain comprises the HCDR1 as set forth in SEQ ID NO: 2, the HCDR2 as set forth in SEQ ID NO: 6, and the HCDR3 as set forth in SEQ ID NO: 15.

44. The isolated antigen-binding protein according to claim 37, wherein the first antigen-binding domain further comprises a light chain variable region (VL), wherein the VL of the first antigen-binding domain comprises at least one light chain complementary determining region (LCDR) comprising an amino acid sequence selected from SEQ ID NO:42, SEQ ID NO:43 and SEQ ID NO:44.

45. The isolated antigen-binding protein according to claim 37, wherein the VH of the first antigen-binding domain comprises a sequence as set forth in any one of SEQ ID NOs: 62-99 or 379.

46. The isolated antigen-binding protein according to claim 37, wherein the VL of the first antigen-binding domain comprises a sequence as set forth in in any one of SEQ ID NOs: 102-106 or 380.

47. The isolated antigen-binding protein according to claim 36, wherein the second antigen binding domain comprises a heavy chain variable region (VH), and the VH of the second antigen binding domain comprises an HCDR1 as set forth in SEQ ID NO: 332, an HCDR2 as set forth in SEQ ID NO: 333, and an HCDR3 as set forth in SEQ ID NO: 334.

48. The isolated antigen-binding protein according to claim 36, wherein the second antigen binding domain comprises a light chain variable region (VL), and the VL of the second antigen binding domain comprises an LCDR1 as set forth in SEQ ID NO: 336, an LCDR2 as set forth in SEQ ID NO: 337, and an LCDR3 as set forth in SEQ ID NO: 338.

49. The isolated antigen-binding protein of claim 36, wherein the second antigen binding domain comprises a VL as set forth in SEQ ID NO: 339 and/or the second antigen binding domain comprises a VH as set forth in SEQ ID NO: 335.

50. The isolated antigen-binding protein according to claim 36, wherein the antigen-binding protein comprises the scFv of the first antigen binding domain or the scFv of the second antigen binding domain.

51. The isolated antigen-binding protein according to claim 50, wherein the scFv of the first antigen-binding domain comprises a sequence as set forth in any one of SEQ ID NOs: 340-345.

52. The isolated antigen-binding protein according to claim 51, wherein the scFv of the first antigen-binding domain comprises a sequence as set forth in SEQ ID NO: 342.

53. The isolated antigen-binding protein according to claim 36, wherein
the first antigen-binding domain comprises HCDR1 as set forth in SEQ ID NO: 2, HCDR2 as set forth in SEQ ID NO: 6, HCDR3 as set forth in SEQ ID NO: 15, LCDR1 as set forth in SEQ ID NO: 42, LCDR2 as set forth in SEQ ID NO: 43, and LCDR3 as set forth in SEQ ID NO: 44; and,
the second antigen-binding domain comprises HCDR1 as set forth in SEQ ID NO: 332, HCDR2 as set forth in SEQ ID NO: 333, HCDR3 as set forth in SEQ ID NO: 334, LCDR1 as set forth in SEQ ID NO: 336, LCDR2 as set forth in SEQ ID NO: 337, and LCDR3 as set forth in SEQ ID NO: 338.

54. The isolated antigen-binding protein according to claim 53, wherein the first antigen-binding domain comprises a VH as set forth in SEQ ID NO: 379 and a VL as set forth in SEQ ID NO: 380; and the second antigen-binding domain comprises a VH as set forth in SEQ ID NO: 335 and a VL as set forth in SEQ ID NO: 339.

55. The isolated antigen-binding protein according to claim 53, wherein the first antigen-binding domain comprises an scFv as set forth in SEQ ID NO: 342; and the second antigen-binding domain comprises the VH as set forth in SEQ ID NO: 335 and the VL as set forth in SEQ ID NO: 339.

56. The isolated antigen-binding protein according to claim 53, wherein the isolated antigen-binding protein includes a light chain as shown in SEQ ID NO: 348 and a heavy chain as shown in SEQ ID NO: 363.

57. An isolated nucleic acid molecule encoding an isolated antigen-binding protein, comprising: a first antigen-binding domain and a second antigen-binding domain, wherein the first antigen-binding domain specifically binds to TNF-like protein A (TL1A), and wherein the second antigen binding domain specifically binds to α4β7.

58. A host cell including the isolated nucleic acid molecule according to claim 57.

59. A pharmaceutical composition comprising an isolated antigen-binding protein according to claim 36 and an optionally pharmaceutical acceptable carrier.

60. A method for preparing an antigen-binding protein the method comprising culturing a host cell comprises one or more nucleic acids encoding an antigen-binding protein comprising a first antigen-binding domain and a second antigen-binding domain, wherein the first antigen-binding domain specifically binds to TNF-like protein A (TL1A), and wherein the second antigen binding domain specifically binds to α4β7, and recovering the antigen-binding protein.

61. A method of treating a TL1A-mediated disease or condition in a subject in need thereof, the method comprising: administrating an antigen-binding protein comprising a first antigen-binding domain and a second antigen-binding domain, wherein the first antigen-binding domain specifically binds to TNF-like protein A (TL1A), and wherein the second antigen binding domain specifically binds to α4β7, thereby treating the disease or condition in the subject.

62. The method according to claim 61, wherein the TL1A-mediated disease or condition is an inflammatory disease selected from one of the following: allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis (e.g., idiopathic pulmonary fibrosis), rheumatic disease, scleroderma, sinusitis, tuberculosis.

63. The method according to claim 61, wherein the TL1A-mediated disease or condition is an autoimmune disease selected from one of the following: achalasia, Addison's disease, Adult Stil's disease, agammaglobulinemia, alopecia areata, amyloidosis, ankylosing spondylitis, anti-GBM/anti-TBM nephritis, antiphospholipid syndrome, autoimmune angioedema, autoimmune familial autonomic dysfunction, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal and neuronal neuropathy (AMAN), Barlow disease (Balódisease), Behcet's disease, benign mucous membrane pemphigoid, bullous pemphigoid, Castleman's disease (Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatous disease (EGPA), cicatricial pemphigoid, and Cogan's syndrome), cold agglutinin disease, congenital heart block, coxsackienew myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, primary mixed cryoglobulinemia, Evans syndrome, fibromyalgia, fibrotic alveolitis, giant cell arteritis (temporal arteritis), giant cell myocarditis, glomerulonephritis, Goodpasture's syndrome, granulomatosis with polyangiitis, Graves' disease, Guillain-Barre syndrome syndrome), Hashimoto's thyroiditis, hemolytic anemia, Henoch-Schonlein purpura (HSP), herpes gravilens or pemphigoid gestationum (PG), hidradenitis suppurativa (HS) (paradoxical acne), hypogammaglobulinemia, IgA nephropathy, IgG4-associated sclerotic disease, immune thrombocytopenic purpura (ITP), Inclusion body myositis (IBM), interstitial cystitis (IC), juvenile arthritis, juvenile diabetes mellitus (type 1 diabetes), juvenile myositis (JM), Kawasaki disease, Lambert-Eaton syndrome, leukocytoclastic vasculitis, lichen planus, lichen sclerosus, lignoconjunctivitis, linear IgA disease (LAD), lupus, chronic Lyme disease (Lyme disease chronic), Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, recurrent rheumatism (PR), PandAS, paraneoplastic cerebellar degeneration (PCD), paroxysmal nocturnal hemoglobinuria (PNH), Parry Romberg syndrome, ciliary planitis (peripheral uveitis), Parsonage-Tumer syndrome), pemphigus, peripheral neuropathy, perivenous encephalomyelitis, pernicious anemia (PA), POEMS syndrome, polyarteritis nodosa, polyglandular syndrome type I, II, type III, polymyalgia rheumatica, polymyositis, post-myocardial infarction syndrome, postpericardiotomy syndrome, primary biliary cirrhosis, primary sclerosing cholangitis, progesterone dermatitis, psoriasis, psoriatic arthritis, pure red blood cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff-person syndrome (SPS), subacute bacterial endocarditis (SBE), Susac's syndrome syndrome), sympathetic ophthalmia (SO), Takayasu's arteritis, temporal arteritis/giant cell arteritis, thrombocytopenic purpura (TTP), Tolosa-Hunt syndrome (THS), transverse myelitis, type 1 diabetes mellitus, ulcerative colitis (UC), undifferentiated connective tissue disease (UCTD), uveitis, vasculitis, Vitiligo and Vogt-Koyanagi-Harada disease.

64. The method according to claim 61, wherein the TL1A-mediated disease or condition is cancer selected from one or more of the following: adenoid cystic carcinoma, adrenal carcinoma, amyloidosis, anal cancer, ataxia-telangiectasia, atypical nevus syndrome, basal cell carcinoma, cholangiocarcinoma, Birt Hogg Dube syndrome, bladder cancer, bone cancer, brain tumor, breast cancer, male breast cancer, carcinoid tumor, cervical cancer, colorectal cancer, ductal cancer, Endometrial Cancer, Esophageal Cancer, Gastric Cancer, Gastrointestinal Stromal Tumor (GIST), HER2-Positive Breast Cancer, Pancreatic Islet Cell Tumor, Juvenile Polyposis Syndrome, Kidney Cancer, Laryngeal Cancer, Leukemia-Acute Lymphoblastic Leukemia, Acute Lymphoblastic Leukemia (ALL), Acute Myeloid Leukemia AML, Adult Leukemia, Childhood Leukemia, Chronic Lymphocytic Leukemia (CLL), Chronic Myeloid Leukemia (CML), Liver Cancer, Lobular Carcinoma, Lung Cancer, Small Cell Lung Cancer (SCLC), Non-small cell lung cancer (NSCLC), Hodgkin's lymphoma (Lymphoma-Hodgkin's), non-Hodgkin's lymphoma (Lymphoma-Non-Hodgkin's), malignant glioma, melanoma, meningioma, multiple myeloma, myelodysplastic syndrome (MDS), nasopharyngeal carcinoma, neuroendocrine tumor, oral cancer, osteosarcoma, ovarian cancer, pancreatic cancer, pancreatic neuroendocrine tumor, parathyroid carcinoma, penile cancer, peritoneal cancer, Peutz-Jeghers syndrome, pituitary tumors, polycythemia vera, prostate cancer, renal cell carcinoma, retinoblastoma, salivary gland carcinoma, sarcoma, Sarcoma-Kaposi, skin cancer, small bowel cancer, stomach cancer, testicular cancer, thymoma, thyroid cancer, uterine (endometrial) cancer, vaginal cancer, and Wilms' Tumor.
